(19) United States
(12) Patent Application Publication
(10) Pub. No.: US 2020/0171032 A1
(43) Pub. Date: Jun. 4, 2020

(54) METHODS AND MEDICAL USES

(71) Applicants: THE INSTITUTE OF CANCER RESEARCH: ROYAL CANCER HOSPITAL, London (GB); Breast Cancer Now, London (GB)

(72) Inventors: Lesley-Ann Martin, London (GB); Joanna Nikitorwicz-Buniak, London (GB)

(21) Appl. No.: 16/623,948

(22) PCT Filed: Jun. 19, 2018
(86) PCT No.: PCT/GB18/51694

§ 371 Date: Dec. 18, 2019

(30) Foreign Application Priority Data
Jun. 20, 2017 (GB) 1709837.7
Apr. 13, 2018 (GB) 1806133.3

(51) Int. Cl. A61K 31/519; A61K 31/138; A61K 31/437; A61K 31/4375; A61K 31/4725; A61K 31/496; A61K 31/4985; A61K 31/5025; A61K 31/506; A61K 31/517; A61K 31/565; A61P 35/00

(52) U.S. Cl. CPC A61K 31/519; A61K 31/138; A61K 31/437; A61K 31/4375; A61K 31/4725; A61P 35/00; A61K 31/4985; A61K 31/5025; A61K 31/506; A61K 31/517; A61K 31/565; A61K 31/496

(57) ABSTRACT

The invention described herein provides a method for the treatment of an oestrogen receptor positive breast cancer in a subject in need thereof comprising administering to said subject a therapeutically effective amount of an MPS1 inhibitor, wherein:
(i) said subject has previously been treated with an endocrine therapy; and/or
(ii) said breast cancer is resistant to endocrine therapy.

### INTRODUCTION

The present invention provides new methods of treatment and medical uses relating to the treatment of endocrine resistant oestrogen receptor-positive breast cancer. Furthermore, the present invention provides combinations suitable for the treatment of oestrogen receptor-positive breast cancer.

### BACKGROUND OF THE INVENTION

It has long been recognised that many breast cancers are hormone dependent. Oestrogen (may be interchangeably referred to as estrogen), in particular, acts as an endocrine growth factor in a large proportion of breast cancers. Thus depriving these tumours of oestrogen is a major treatment modality in breast cancer.

The most commonly diagnosed breast cancer (BC) in the clinic is oestrogen receptor-(ER) positive and ER-positive BC accounts for over 80% of cases.

Oestrogen mediates its effects by binding to the ER. Oestrogen bound ER associates classically with oestrogen response elements (EREs) on target genes controlling proliferation and cell survival. ER has two distinct activation domains, AF-1 and AF-2. AF-2 is integral to the ligand-binding domain and is regulated by the binding of oestrogen. AF-1 activity is regulated by phosphorylation whilst AF-2 associates with coactivators of the p160 family, controlling the ER transcriptional complex.

Classically, patients with ER-positive BC are treated with endocrine agents such as tamoxifen or aromatase inhibitors (AI), which impede oestrogen signalling.

Although over 50% of patients show response to endocrine therapy, a large proportion relapse with de novo or acquired resistant disease, making it one of the greatest challenges for breast cancer research (reviewed by Ma et al. 2015). One striking feature of endocrine-resistant BC is the fact that the majority of patients continue to express ER. To date, multiple molecular mechanisms have been implicated in the resistant phenotype, all of which converge at the level of cyclin D, forcing cell cycle progression. This high degree of heterogeneity in adaptive mechanisms during the course of ER-positive BC progression highlights the importance of finding common nodes attributed to therapeutic failure.

As uncontrolled proliferation is a hallmark of cancer (reviewed by Hanahan & Weinberg 2011) direct targeting of cell cycle with CDK inhibitors has provided an attractive proposition but until recently few have shown specificity and associated clinical toxicities have been unacceptable (Asghar et al. 2015). The CDK4/6-RB axis is critical for cell cycle entry and, not surprisingly, most cancers subvert this axis to promote proliferation, for instance 19% of breast cancers show amplification of CDK4 whilst, CCND1 amplification is associated with endocrine resistance (reviewed Musgrove et al. 2011).

Recently, the combination of CDK4/6 inhibitors with endocrine therapy has been shown to improve clinical outcome in ER+ breast cancer patients. However, not all patients will benefit from such combination therapy and many will eventually relapse with acquired resistance.

There is a need in the art for new treatment options in respect of ER+ breast cancer. In particular, there is a need in the art for new, effective therapies for treating endocrine-resistant ER+ breast cancer.

### SUMMARY OF THE INVENTION

Herein, it is shown for the first time that MPS1 is surprisingly associated with resistance to endocrine therapy, and furthermore that MPS1 provides a rational target for the treatment of breast cancers which are resistant to endocrine therapy.

In one aspect, the present invention relates to a method for the treatment of an oestrogen receptor-positive breast cancer in a subject in need thereof comprising administering to said subject a therapeutically effective amount of an MPS1 inhibitor, wherein:
(i) said subject has previously been treated with an endocrine therapy; and/or
(ii) said breast cancer is resistant to endocrine therapy.

In one aspect, the present invention relates to an MPS1 inhibitor for use in the treatment of an oestrogen receptor-positive breast cancer in a subject in need thereof, wherein:
(i) the subject has previously been treated with an endocrine therapy; and/or
(ii) said breast cancer is resistant to endocrine therapy.

In one aspect, the present invention relates to a use of an MPS1 inhibitor in the manufacture of a medicament for the treatment of an oestrogen receptor positive breast cancer in a subject in need thereof, wherein:
(i) the subject has been previously treated with an endocrine therapy; and/or
(ii) said breast cancer is resistant to endocrine therapy.

In one aspect, the present invention relates to a combination comprising an MPS1 inhibitor and an endocrine agent.

In one aspect, the present invention relates to a pharmaceutical product comprising an MPS1 inhibitor and an endocrine agent.

In one aspect, the present invention relates to a method for the treatment of oestrogen receptor positive breast cancer in a subject in need thereof comprising administering to said subject, either separately, sequentially or in combination, a therapeutically effective amount of an MPS1 inhibitor and a therapeutically effective amount of an endocrine agent.

In one aspect, the present invention relates to a pharmaceutical product comprising an MPS1 inhibitor and an endocrine agent for use in the treatment of an oestrogen receptor positive breast cancer, wherein the compound capable of inhibiting MPS1 and the endocrine agent are for separate, sequential or combined administration.

In one aspect, the present invention relates to the use of a pharmaceutical product comprising an MPS1 inhibitor and an endocrine agent in the manufacture of a medicament for the treatment of oestrogen receptor positive breast cancer, wherein the compound capable of inhibiting MPS1 and the endocrine agent are for separate, sequential or combined administration.

In one aspect, the present invention relates to an MPS1 inhibitor and an endocrine agent for use in the treatment of estrogen receptor-positive breast cancer.

In one aspect, the present invention relates to an MPS1 inhibitor for use in the treatment of estrogen receptor-positive breast cancer, wherein said MPS1 inhibitor is for separate, sequential or combined administration with an endocrine agent.

In one aspect, the present invention relates to an endocrine agent for use in the treatment of estrogen receptor-positive breast cancer, wherein said endocrine agent is for separate, sequential or combined administration with an MPS1 inhibitor.

In one aspect, the present invention relates to a use of an MPS1 inhibitor and an endocrine agent in the manufacture of a medicament for the treatment of estrogen receptor-positive breast cancer.

In one aspect, the present invention relates to a use of an MPS1 inhibitor in the manufacture of a medicament for the treatment of estrogen receptor-positive breast cancer, wherein said MPS1 inhibitor is for separate, sequential or combined administration with an endocrine agent.

In one aspect, the present invention relates to a use of an endocrine agent in the manufacture of a medicament for the treatment of estrogen receptor-positive breast cancer, wherein said endocrine agent is for separate, sequential or combined administration with an MPS1 inhibitor.

Preferred, suitable, and optional features of any one particular aspect of the present invention are also preferred, suitable, and optional features of any other aspect.

### BRIEF DESCRIPTION OF THE DRAWINGS

FIG. 1A shows A a schematic representation of the methodology.

FIG. 1B shows B the intersection of kinome screens from several LTED cell lines either ER+ or ER− identifies G2/M checkpoint proteins as common determinants of resistance; and C. validation of the role of MPS1 in the LTED resistant phenotype was assessed by targeted knockdown with an siRNA against MPS1. PLK1 was used as a positive control.

FIG. 10 shows D an analysis of RNA-seq. data from MCF7 versus MCF7-LTED shows increased expression in the endocrine resistant model; and E. global analysis of protein abundance shows MPS1 is increased in MCF7-LTED cells.

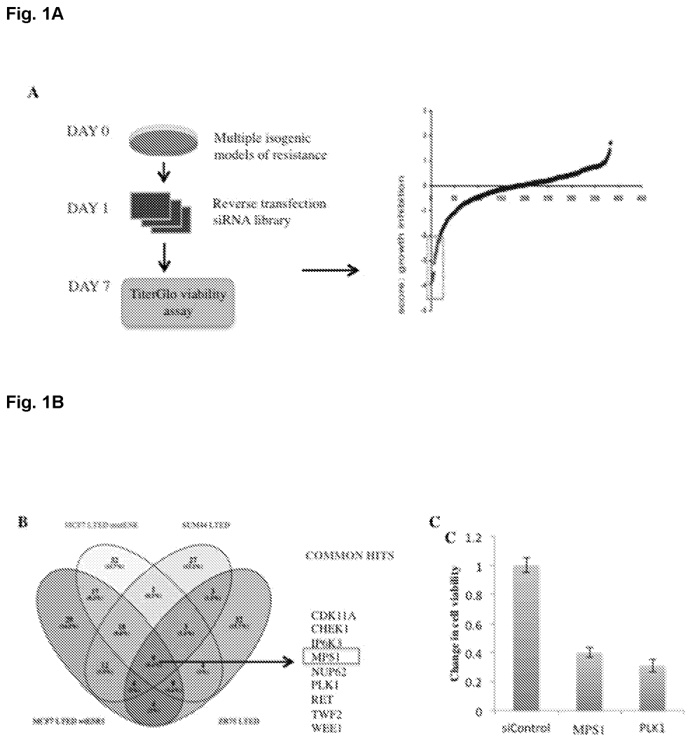

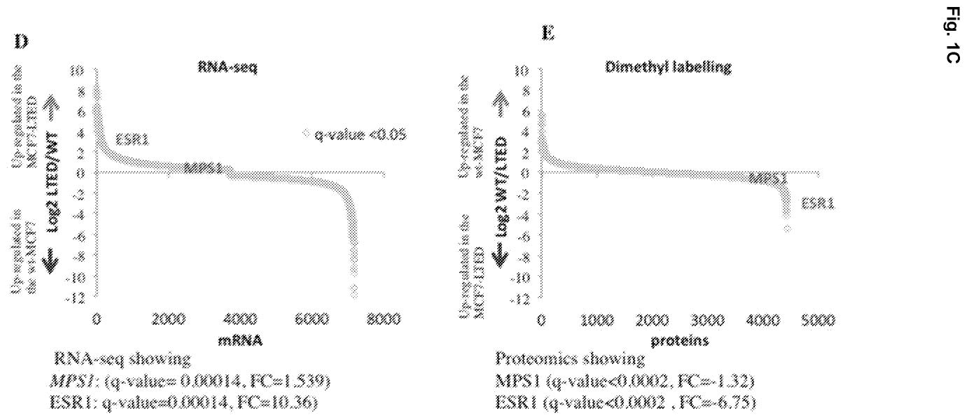

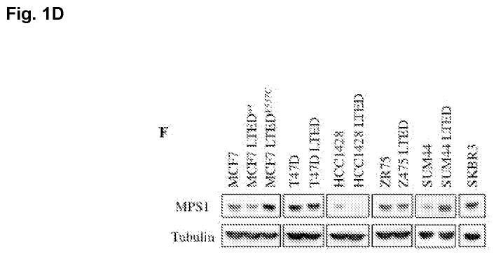

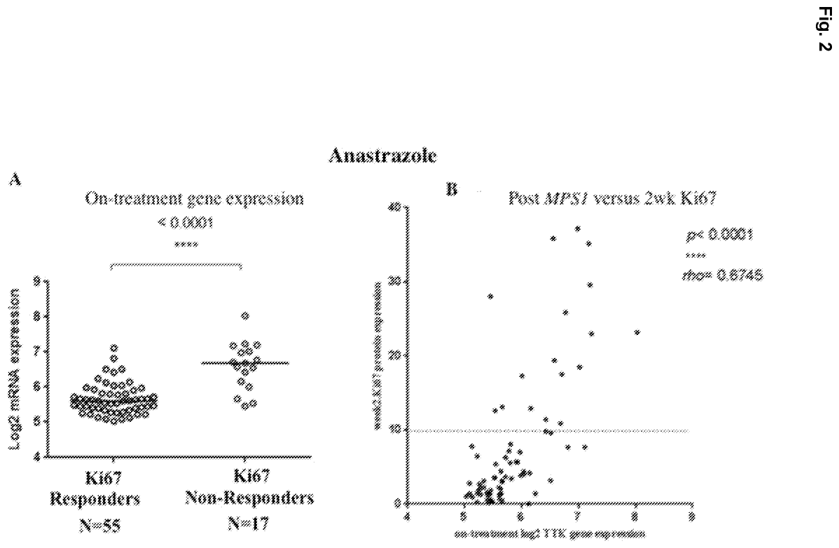

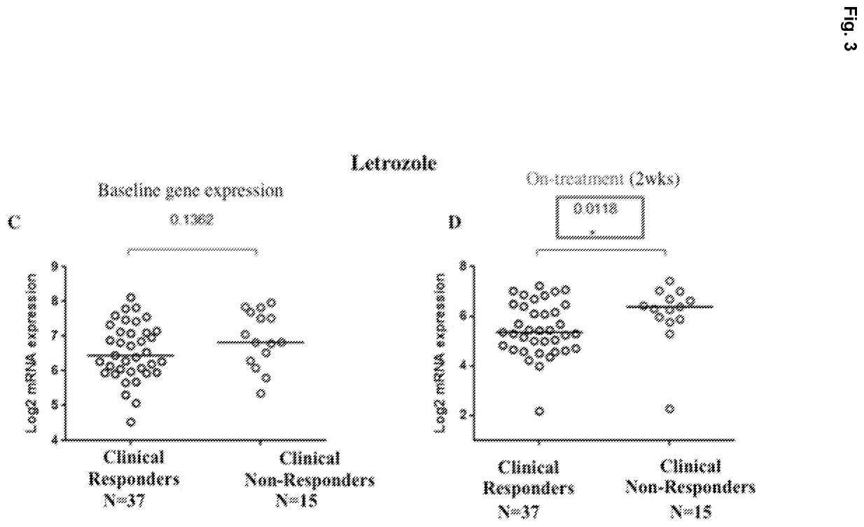

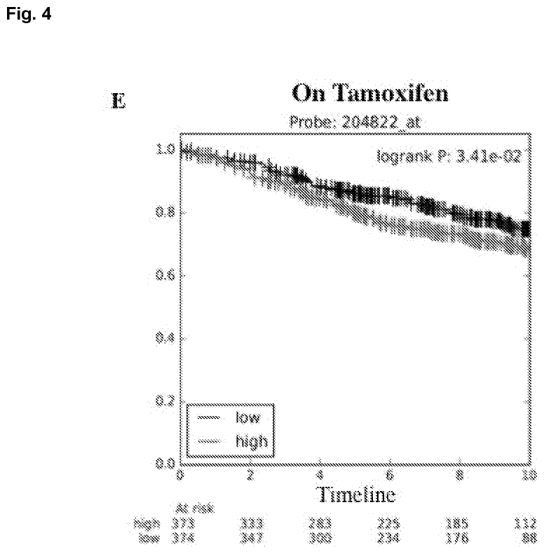

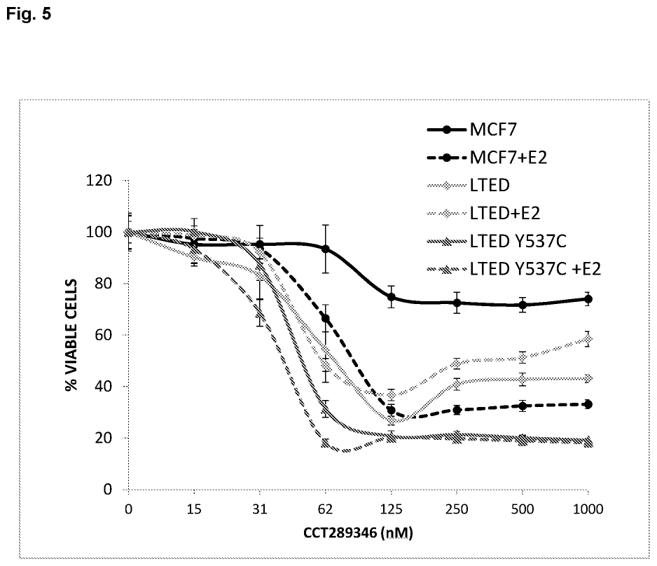

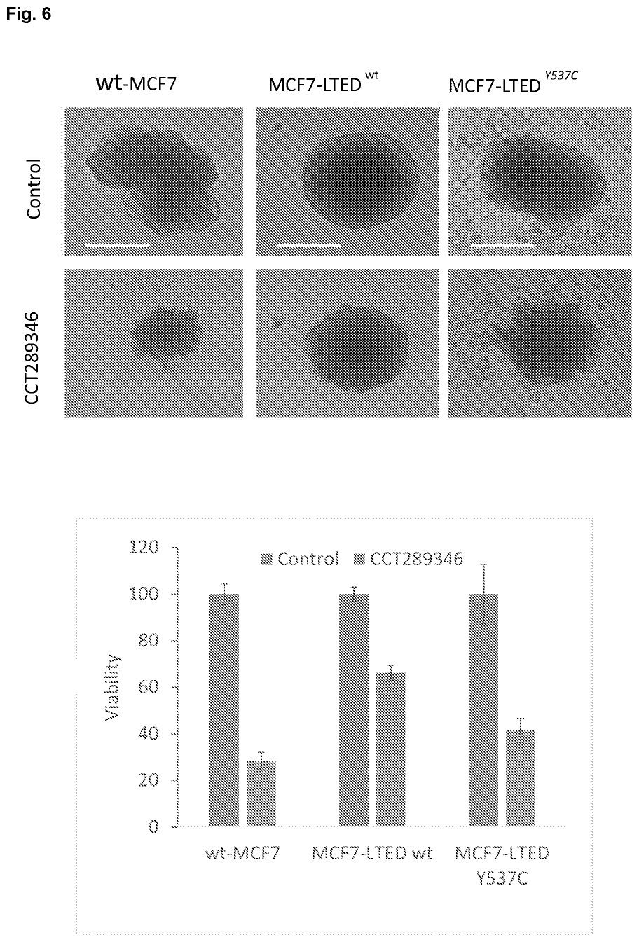

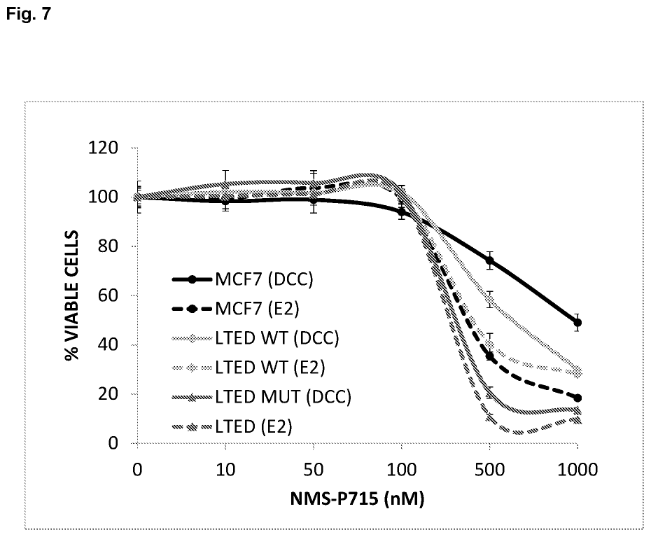

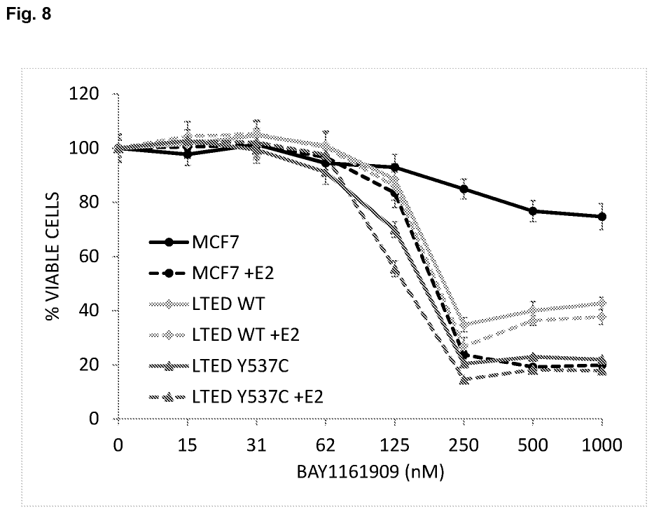

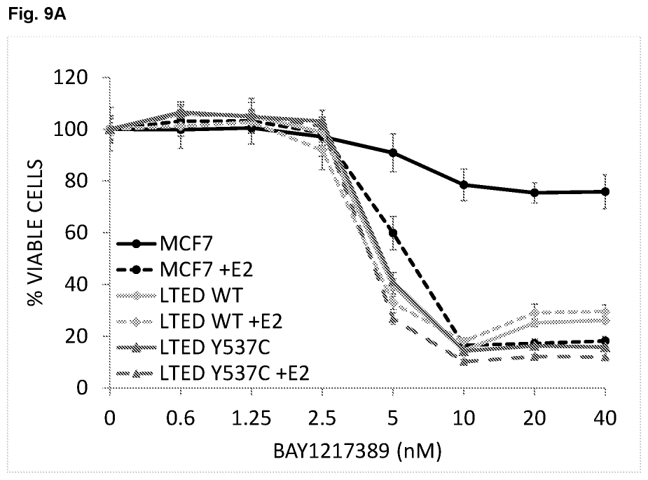

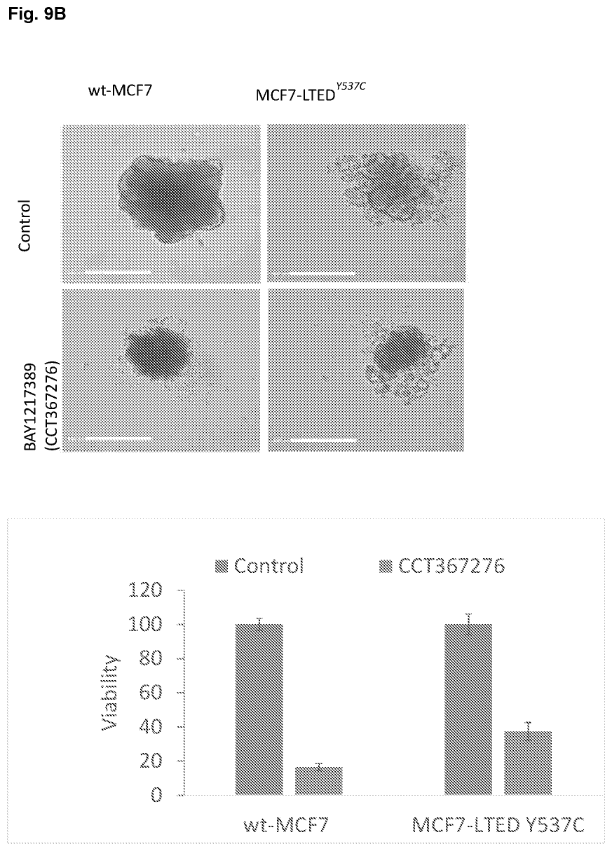

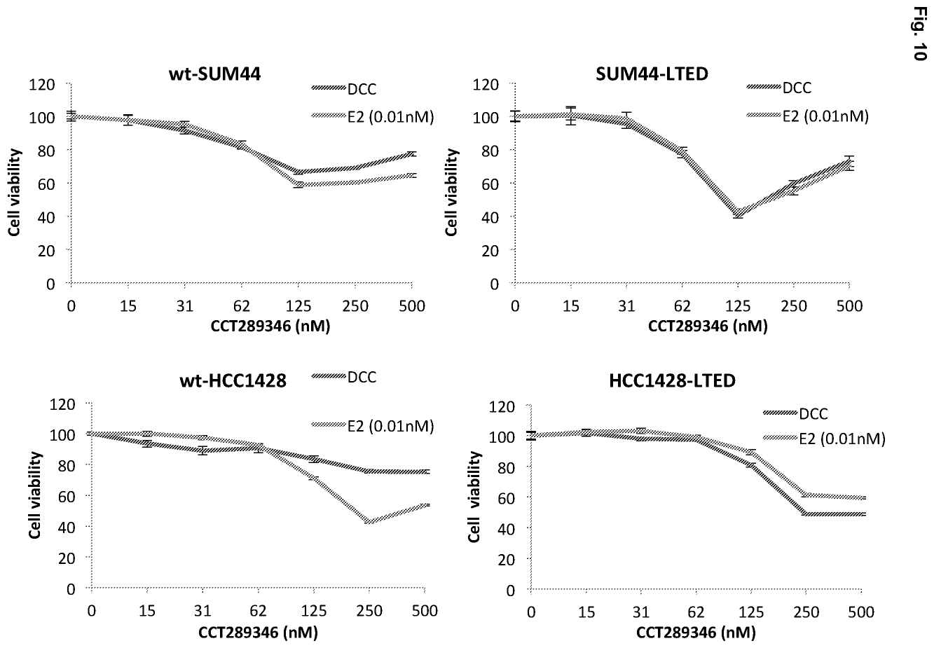

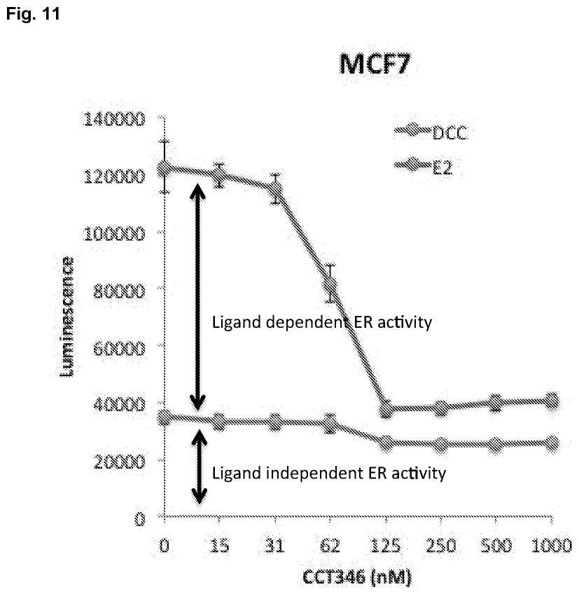

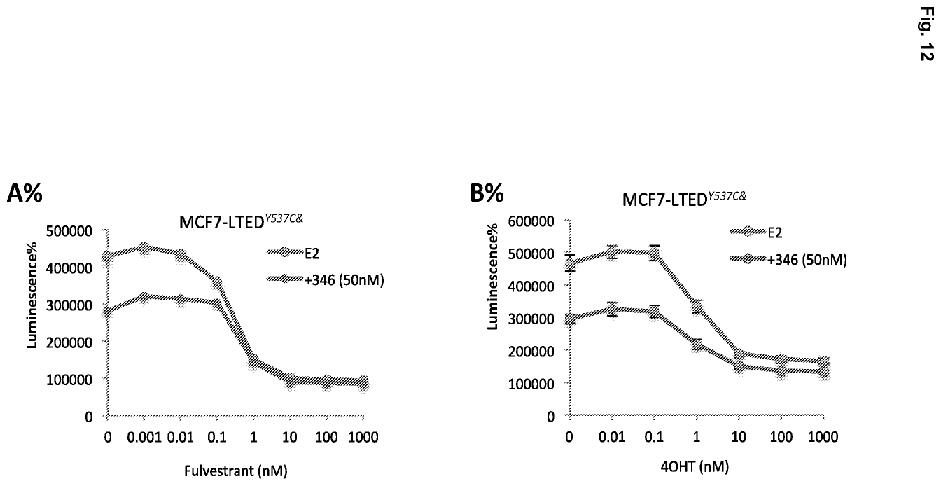

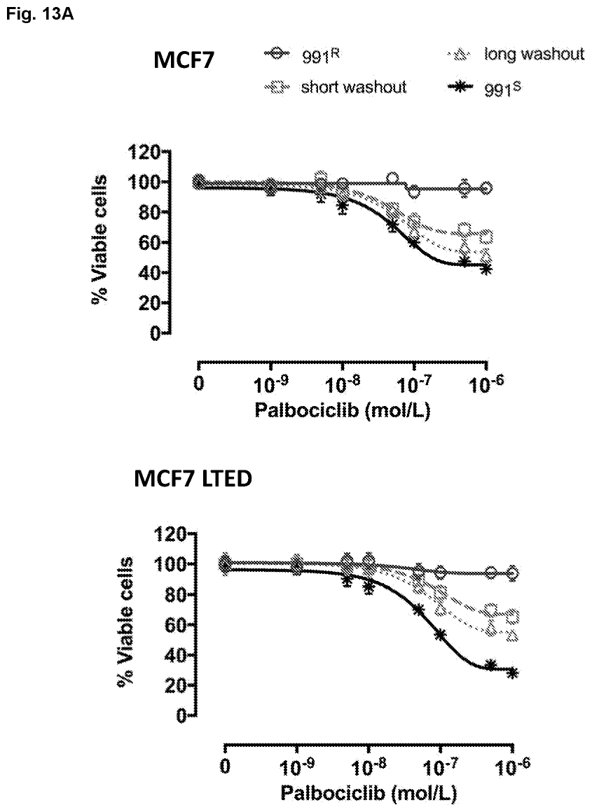

FIG. 1D shows F an immunoblot analysis showing increased expression of MPS1 in MCF7-LTED and SUM44-LTED cell lines.

FIG. 2A shows on treatment gene expression of MPS1 in patients treated with anastrazole and B shows the association of on-treatment gene expression of MPS1 and 2-wk Ki67 value. Spearman's rank correlation coefficients (rho) and P-values are depicted.

FIG. 3C shows the baseline expression of MPS1 and D shows on-treatment gene expression of MPS1 in patients treated with letrozole.

FIG. 4 shows a Kaplan-Meier plot revealing the association of high or low pre-treatment gene expression of MPS1 in ER+ BC patients treated with tamoxifen, from publicly available data collected over 10 years.

FIG. 5 shows the results of treating various breast cancer cell, in the presence or absence of E2, with escalating concentrations of CCT289346.

FIG. 6 shows spheroid cultures of wt-MCF7 and MCF7-LTED cells harbouring wt or mutant ESR1 were treated with MPS1 inhibitor CCT289346. Bar graph presents cell viability determined using TitreGlo.

FIG. 7 shows the response of breast cancer cell lines to escalating concentration of MPS1 inhibitor NMS-P175.

FIG. 8 shows the response of breast cancer cell lines to escalating concentration of MPS1 inhibitor BAY 1161909.

FIGS. 9A and B shows the response of breast cancer cell lines to escalating concentration of MPS1 inhibitor BAY 1217389 in 2D (FIG. 9A) and spheroid culture (FIG. 9B). The bar graph presents a measure of spheroid viability using TiterGlo.

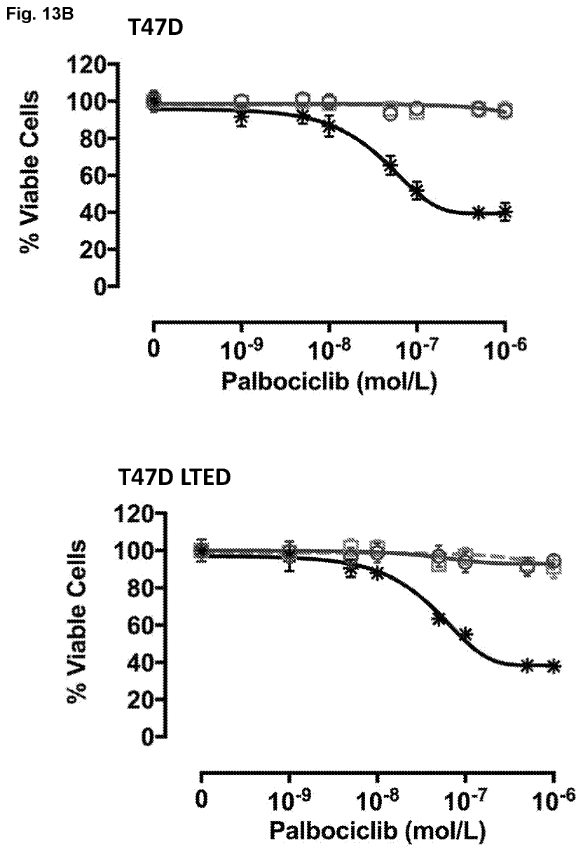

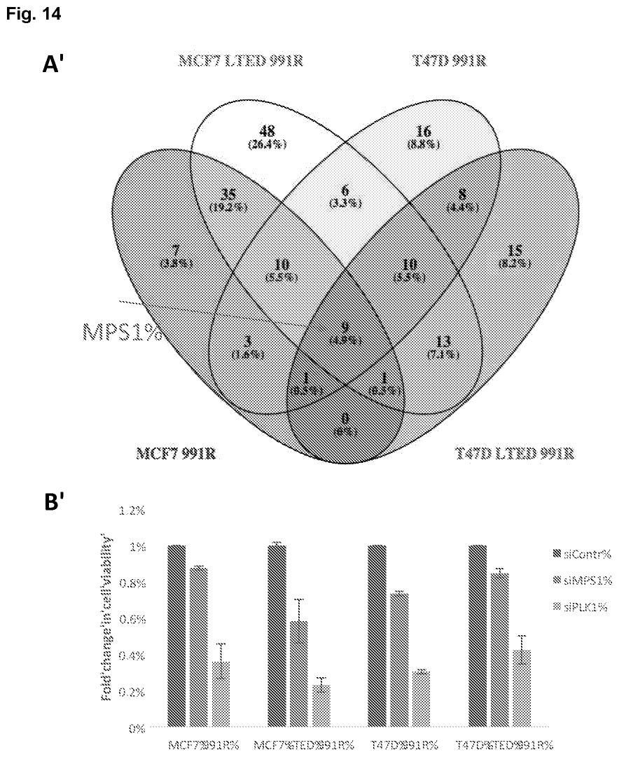

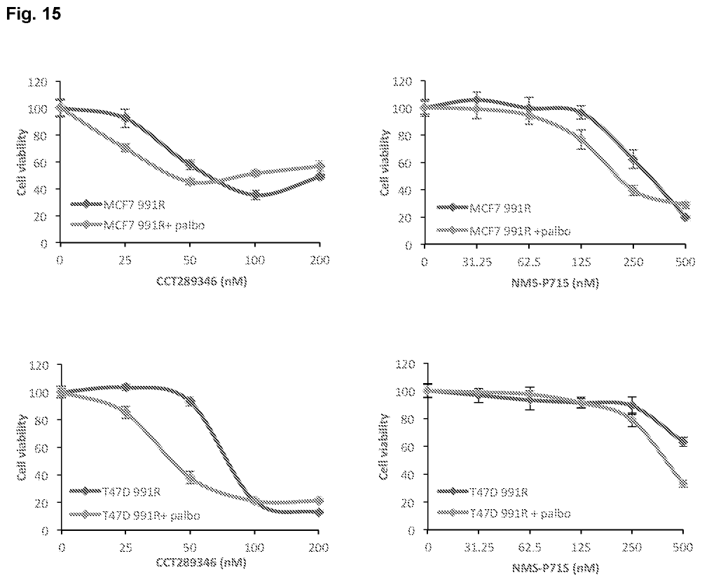

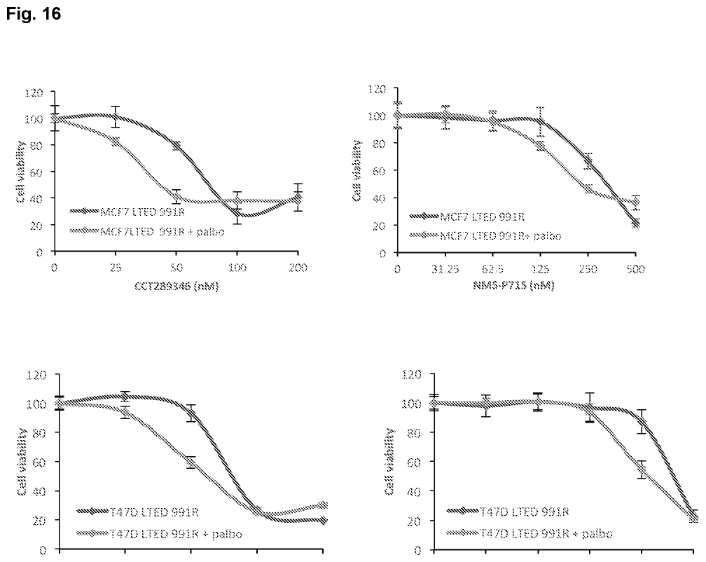

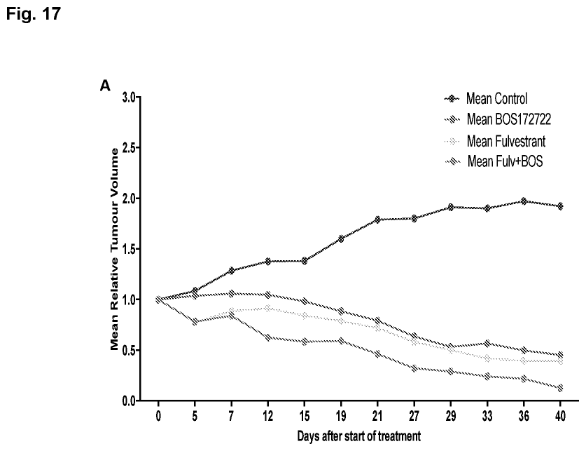

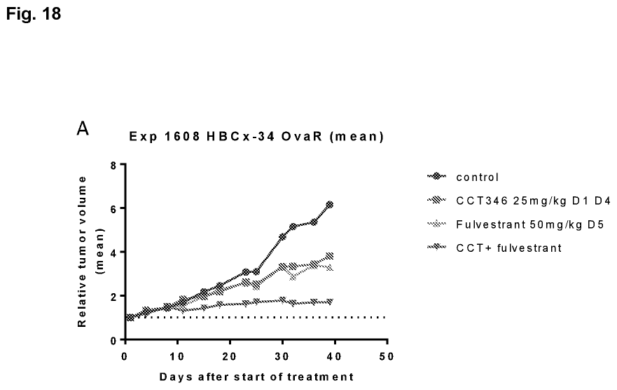

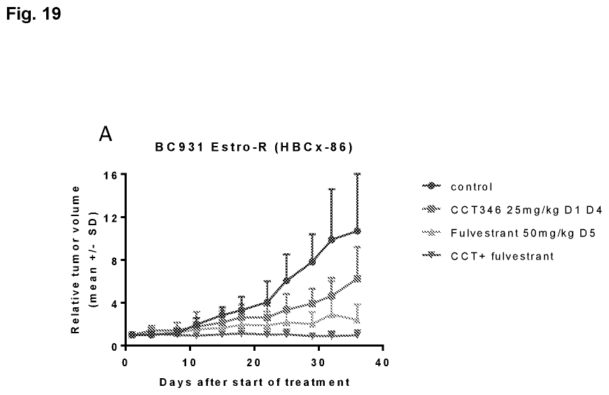

FIG. 10 shows the results of four cell lines: Wt-SUM44, SUM44 LTED, HCC1428 and HCC1428 LTED which were cultured in the presence or absence estradiol (E2) and escalating concentration of MPS1 inhibitor CCT289346 for 6 days with a medium change on day 3. Cell viability was assessed using TitreGlo. Data was normalised to vehicle control for each condition.

FIG. 11 shows the results of treating wt-MCF7 cells were treated with escalating concentrations of CCT289346 in the presence of E2 or the absence of E2.

FIG. 12 shows the results of treating cells with escalating concentrations of A. fulvestrant alone or in combination with 50nM CCT289346. B. 4-OHT alone or in combination with 50 nM CCT289346 for 6 days with a medium change on day 3. Cell viability was measured using TitreGlo.

FIGS. 13A and 13B show the results of proliferation assays in palbociclib resistant and sensitive cell lines.

FIG. 14 shows the results of a kinome siRNA library screen in palbociclib resistant models. A. Venn diagram identified MPS1 as one of the common targets in all resistant models. B. Bar chart showing change in cell viability (mean±standard error of the mean) normalised to non-targeting siControl generated from library screens.

FIG. 15 shows wt-MCF7 and wt-T47D palbociclib-resistant cell lines were treated with escalating concentration of CCT289346 or NMS-P715 in the presence or absence of palbociclib for 6 days with a medium change on day 3. Cell viability was determined using TiterGlo and data expressed as fold cell viability.

FIG. 16 shows MCF7-LTED and T47D-LTED palbociclib-resistant cell lines were treated with escalating concentration of CCT289346 or NMS-P715 in the presence or absence of palbociclib for 6 days with a medium change on day 3. Cell viability was determined using TiterGlo and data expressed as fold cell viability.

FIG. 17 shows the effect of CCT289346 (also referred to as BOS172722) in MCF7-LTED tumour growth.

FIG. 18 shows the effect of CCT289346 alone or in combination with fulvestrant in PDX model HBCx-34.

FIG. 19 shows the effect of CCT289346 alone or in combination with fulvestrant in PDX model HBCx-34.

### DETAILED DESCRIPTION OF THE INVENTION

### Definitions

As used herein, “oestrogen receptor-positive (ER+) breast cancer” refers to a breast cancer which naturally expresses oestrogen receptors (suitably nuclear oestrogen receptors; suitably ER-alpha). Any suitable technique known in the art may be used to identify if a breast cancer expresses oestrogen receptors, including ligand-binding assays and immunohistochemical techniques.

As used herein the term “endocrine therapy” refers to any treatment capable of removing oestrogen, blocking generation of oestrogen, reducing levels of oestrogen, blocking the effect of oestrogen, reducing the effect of oestrogen and/or can lead to instability, degradation and/or down regulation of the oestrogen receptor. Suitably, the endocrine therapy comprises/essentially consists of/consists of administration of an endocrine agent.

As used herein, the term “endocrine agent” refers to any chemical compound or biological agent capable of removing oestrogen, blocking generation of oestrogen and/or reducing levels of oestrogen. Suitably, the endocrine agent is a chemical compound, e.g. a drug or a drug-like molecule.

As used herein, “failed previous treatment” may mean said subject has been determined by a relevant skilled person to have failed treatment with an endocrine therapy. A relevant skilled person would readily be able to determine when a subject has failed treatment with an endocrine therapy. For instance, failure of treatment with an endocrine therapy may manifest as one or more of the following during or following therapy: disease progression (e.g. growth of the cancer, relapse, recurrence, spread of the cancer, increased tumour grade, increased proliferation), lack of response (i.e. no pathological change in the cancer, no change in tumour size) and insufficient response (insufficient decrease in tumour size, insufficient pathological response).

As used herein “combined administration” refers to therapy in which the both agents (e.g. an MPS1 inhibitor and an endocrine agent) are administered simultaneously.

As used herein “sequential administration” means that one agent is administered after the other, however, the time period between the administration of each agent is such that both agents are capable of acting therapeutically concurrently. Thus, administration “sequentially” may permit one agent to be administered within 5 minutes, 10 minutes or a matter of hours after the other provided the circulatory half-life of the first administered agent is such that they are both concurrently present in therapeutically effective amounts. The time delay between the administration of the agents may vary depending on the exact nature of the agents, the interaction there between, and their respective half-lives.

As used herein, “separate administration” means that one agent is administered after the other, however, the time period between administration is such that the first administered agent is no longer present a therapeutically effective amount when the second agent is administered. Accordingly, the two agents exert their therapeutic effects separately. Nevertheless, the overall therapeutic effect observed when the two agents separately act therapeutically may be greater than either agent used alone.

As used herein the, “subject(s)” and/or “patient(s)”, suitably refer to mammals (e.g. humans and non-human mammals such as livestock (cows, sheep, goats) or companion animals (cats, dogs, horses, rabbits). Suitably, the subject(s) and/or patient(s) are human(s).

As used herein “resistant to endocrine therapy” or “endocrine-resistant” cancer may mean said cancer has been determined by a relevant skilled person to be resistant to endocrine therapy. A relevant skilled person would readily be able to determine when a cancer is resistant to endocrine therapy. For instance, clinically, resistance can manifest as relapse or cancer recurrence during or following endocrine therapy. Alternatively, resistance can be observed as clinical progression of primary disease, usually constituting an increase in primary tumour size or disease spread to regional nodes or beyond to more distant metastatic sites. Pathological changes such as increased tumour grade or increased proliferation are indicators of potential resistance to therapy. In the neoadjuvant setting, resistance occurs as either a primary lack of response (no change or an increase in tumour size and no evidence of pathological response) early in treatment, implying innate or de novo resistance, or later following a period of response, suggesting acquired resistance. Alternatively, resistance to endocrine therapy may be determined in endocrine therapy naïve patients by reference to genotypic and/or phenotypic markers of resistance.

As used herein, the term CDK4/6 inhibitor refers to chemical or biological agents capable of inhibiting CDK4 and CDK6. Suitably, the CDK4/6 inhibitors are selective for CDK4/6 over other kinases, particularly over other CDKs. Suitably, the CDK4/6 inhibitors herein have nanomolar IC₅₀s at CDK4 and CDK6. Suitably, the CDK4/6 inhibitors are chemical compounds, e.g. a drug or a drug-like molecules.

As used herein, the term MPS1 inhibitor refers to a chemical or biological agent capable of inhibiting MPS1 (monopolar spindle) kinase. Suitably, the MPS1 inhibitors are selective for MPS1 over other kinases. Suitably, the MPS1 inhibitors herein have nanomolar IC₅₀s at MPS1. Suitably, the MPS1 inhibitors are chemical compounds, e.g. a drug or a drug-like molecule.

As used herein the term “BAY 1161909” refers to the following compound:

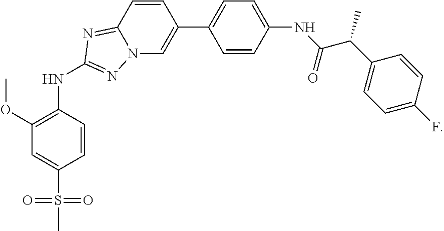 [SMILES: COc1cc(S(C)(=O)=O)ccc1Nc1nc2ccc(-c3ccc(NC(=O)[C@H](C)c4ccc(F)cc4)cc3)cn2n1]

As used herein the term “BAY 1217389” refers to the following compound:

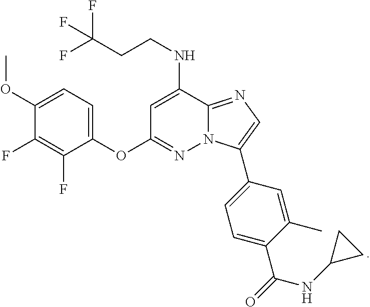 [SMILES: COc1ccc(Oc2cc(NCCC(F)(F)F)c3ncc(-c4ccc(C(=O)NC5CC5)c(C)c4)n3n2)c(F)c1F]

As used herein the term “NMS-P715” refers to the following compound:

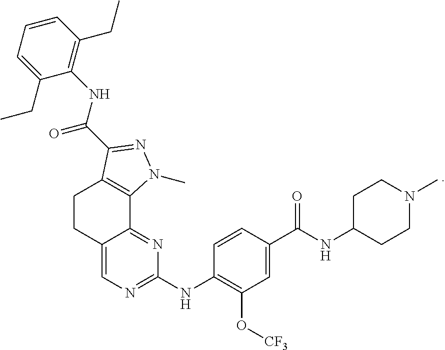 [SMILES: CCc1cccc(CC)c1NC(=O)c1nn(C)c2c1CCc1cnc(Nc3ccc(C(=O)NC4CCN(C)CC4)cc3OC(F)(F)F)nc1-2]

As used herein the term “AZ3146” refers to the following compound:

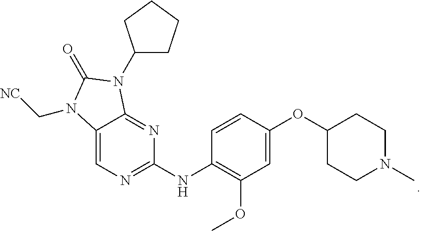 [SMILES: COc1cc(OC2CCN(C)CC2)ccc1Nc1ncc2c(n1)n(C1CCCC1)c(=O)n2CC#N]

As used herein the term “MPS1-IN-3” refers to the following compound:

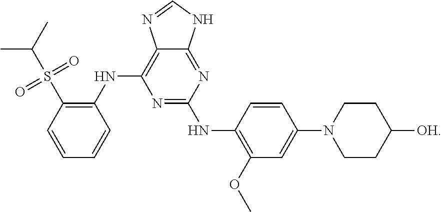 [SMILES: COc1cc(N2CCC(O)CC2)ccc1Nc1nc(Nc2ccccc2S(=O)(=O)C(C)C)c2nc[nH]c2n1]

As used herein the term “MPS1-IN-2” refers to the following compound:

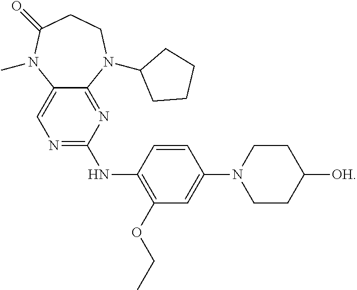 [SMILES: CCOc1cc(N2CCC(O)CC2)ccc1Nc1ncc2c(n1)N(C1CCCC1)CCC(=O)N2C]

As used herein the term “CFI-402257” refers to the following compound:

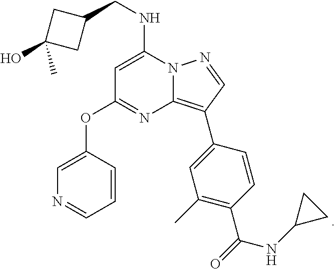

As used herein the term “CCT289346” refers to N2-(2-ethoxy-4-(4-methyl-4H-1,2,4-triazol-3-yl)phenyl)-6-methyl-N8-neopentylpyrido[3,4-d]pyrimidine-2,8-diamine.

As used herein by themselves or in conjunction with another term or terms, “therapeutic” and “therapeutically effective amount” refer to an amount a compound, composition or medicament that (a) inhibits or causes an improvement in a particular disease, condition or disorder; (b) attenuates, ameliorates or eliminates one or more symptoms of a particular disease, condition or disorder; (c) or delays the onset of one or more symptoms of a particular disease, condition or disorder described herein. It should be understood that the terms “therapeutic” and “therapeutically effective” encompass any one of the aforementioned effects (a)-(c), either alone or in combination with any of the others (a)-(c). It should be understood that in, for example, a human or other mammal, a therapeutically effective amount can be determined experimentally in a laboratory or clinical setting, or a therapeutically effective amount may be the amount required by the guidelines of the United States Food and Drug Administration (FDA) or equivalent foreign regulatory body, for the particular disease and subject being treated. It should be appreciated that determination of proper dosage forms, dosage amounts, and routes of administration is within the level of ordinary skill in the pharmaceutical and medical arts.

As used herein whether by themselves or in conjunction with another term or terms, “treating”, “treated” and “treatment”, may refer to medical actions and results and includes prophylactic, ameliorative, palliative, and curative actions and results. In some embodiments, the terms “treating”, “treated”, and “treatment” refer to curative actions and results as well as actions and results that diminish or reduce the severity of a particular condition, characteristic, symptom, disorder, or disease described herein. For example, treatment can include diminishment of several symptoms of a condition or disorder or complete eradication of said condition or disorder. It should be understood that the term “prophylactic” as used herein is not absolute but rather refers to actions and results where the administration of a compound or composition diminishes the likelihood or seriousness of a condition, symptom, or disease state, and/or delays the onset of a condition, symptom, or disease state for a period of time.

The compounds and intermediates described herein may be named according to either the IUPAC (International Union for Pure and Applied Chemistry) or CAS (Chemical Abstracts Service) nomenclature systems. It should be understood that unless expressly stated to the contrary, the terms “compounds of Formula I” and the more general term “compounds” refer to and include any and all compounds described by and/or with reference to Formula I. As applies mutatis mutandis to the terms “compounds of Formula II”, “compounds of Formula III”, “compounds of Formula IV” and “compounds of Formula V”. It should also be understood that these terms encompasses all stereoisomers, i.e. cis and trans isomers, as well as optical isomers, i.e. R and S enantiomers, of such compounds and all salts thereof, in substantially pure form and/or any mixtures of the foregoing in any ratio. This understanding extends to pharmaceutical compositions and methods of treatment that employ or comprise one or more compounds of the Formulae I, II, Ill, IV and V either by themselves or in combination with additional agents.

The various hydrocarbon-containing moieties provided herein may be described using a prefix designating the minimum and maximum number of carbon atoms in the moiety, e.g. “(C_{a-b})” or “Cₐ-C_{b}” or “(a-b)C”. For example, (C_{a-b})alkyl indicates an alkyl moiety having the integer “a” to the integer “b” number of carbon atoms, inclusive. Certain moieties may also be described according to the minimum and maximum number of members with or without specific reference to a particular atom or overall structure. For example, the terms “a to b membered ring” or “having between a to b members” refer to a moiety having the integer “a” to the integer “b” number of atoms, inclusive.

“About” when used herein in conjunction with a measurable value such as, for example, an amount or a period of time and the like, is meant to encompass reasonable variations of the value, for instance, to allow for experimental error in the measurement of said value.

As used herein by themselves or in conjunction with another term or terms, “alkyl” and “alkyl group” refer to a branched or unbranched saturated hydrocarbon chain. Unless specified otherwise, alkyl groups typically contain 1-10 carbon atoms, such as 1-6 carbon atoms or 1-4 carbon atoms or 1-3 carbon atoms, and can be substituted or unsubstituted. Representative examples include, but are not limited to, methyl, ethyl, n-propyl, i-propyl, n-butyl, i-butyl, s-butyl, t-butyl, n-pentyl, n-hexyl, n-heptyl, n-octyl, n-nonyl, n-decyl, isopropyl, tert-butyl, isobutyl, etc.

As used herein by themselves or in conjunction with another term or terms, “alkylene” and “alkylene group” refer to a branched or unbranched saturated hydrocarbon chain. Unless specified otherwise, alkylene groups typically contain 1-10 carbon atoms, such as 1-6 carbon atoms or 1-3 carbon atoms, and can be substituted or unsubstituted. Representative examples include, but are not limited to, methylene (—CH₂—), the ethylene isomers (—CH(CH₃)— and —CH₂CH₂—), the propylene isomers (—CH(CH₃)CH₂—, —CH(CH₂CH₃—), —C(CH₃)₃—, and —CH₂CH₂CH₂—), etc.

As used herein by themselves or in conjunction with another term or terms, “alkenyl” and “alkenyl group” refer to a branched or unbranched hydrocarbon chain containing at least one double bond. Unless specified otherwise, alkenyl groups typically contain 2-10 carbon atoms, such as 2-6 carbon atoms or 2-4 carbon atoms, and can be substituted or unsubstituted. Representative examples include, but are not limited to, ethenyl, 3-buten-1-yl, 2-ethenylbutyl, and 3-hexen-1-yl.

As used herein by themselves or in conjunction with another term or terms, “alkynyl” and “alkynyl group” refer to a branched or unbranched hydrocarbon chain containing at least one triple bond. Unless specified otherwise, alkynyl groups typically contain 2-10 carbon atoms, such as 2-6 carbon atoms or 2-4 carbon atoms, and can be substituted or unsubstituted. Representative examples include, but are not limited to, ethynyl, 3-butyn-1-yl, propynyl, 2-butyn-1-yl, and 3-pentyn-1-yl.

As used herein by itself or in conjunction with another term or terms, “aromatic” refers to monocyclic and polycyclic ring systems containing 4n+2 pi electrons, where n is an integer. Aromatic should be understood as referring to and including ring systems that contain only carbon atoms (i.e. “aryl”) as well as ring systems that contain at least one heteroatom selected from N, O or S (i.e. “heteroaromatic” or “heteroaryl”). An aromatic ring system can be substituted or unsubstituted.

As used herein by itself or in conjunction with another term or terms, “non-aromatic” refers to a monocyclic or polycyclic ring system having at least one double bond that is not part of an extended conjugated pi system. As used herein, non-aromatic refers to and includes ring systems that contain only carbon atoms as well as ring systems that contain at least one heteroatom selected from N, O or S. A non-aromatic ring system can be substituted or unsubstituted.

As used herein by themselves or in conjunction with another term or terms, “aryl” and “aryl group” refer to phenyl and 7-15 membered bicyclic or tricyclic hydrocarbon ring systems, including bridged, spiro, and/or fused ring systems, in which at least one of the rings is aromatic. Aryl groups can be substituted or unsubstituted. Unless specified otherwise, an aryl group may contain 6 ring atoms (i.e., phenyl) or a ring system containing 9 to 15 atoms, such as 9 to 11 ring atoms, or 9 or 10 ring atoms. Representative examples include, but are not limited to, naphthyl, indanyl, 1,2,3,4-tetrahydronaphthalenyl, 6,7,8,9-tetrahydro-5H-benzocycloheptenyl, and 6,7,8,9-tetrahydro-5H-benzocycloheptenyl. Suitably an aryl group is phenyl and naphthyl, suitably phenyl.

As used herein by themselves or in conjunction with another term or terms, “arylene” and “arylene group” refer to a phenylene (—C₆H₄—) or to 7 to 15 membered bicyclic or tricyclic hydrocarbon ring systems, including bridged, spiro, and/or fused ring systems, in which at least one of the rings is aromatic. Arylene groups can be substituted or unsubstituted. In some embodiments, an arylene group may contain 6 (i.e., phenylene) ring atoms or be a ring system containing 9 to 15 atoms; such as 9 to 11 ring atoms; or 9 or 10 ring atoms. Arylene groups can be substituted or unsubstituted.

As used herein by themselves or in conjunction with another term or terms, “alkylaryl” and “alkylaryl group” refer to an alkyl group in which a hydrogen atom is replaced by an aryl group, wherein alkyl group and aryl group are as previously defined, such as, for example, benzyl (C₆H₅CH₂—). Alkylaryl groups can be substituted or unsubstituted.

As used herein by themselves or in conjunction with another term or terms, “carbocyclic group” and “carbocycle” refer to monocyclic and polycyclic ring systems that contain only carbon atoms in the ring(s), i.e., hydrocarbon ring systems, without regard or reference to aromaticity or degree of unsaturation. Thus, carbocyclic group should be understood as referring to and including ring systems that are fully saturated (such as, for example, a cyclohexyl group), ring systems that are aromatic (such as, for example, a phenyl group), as well as ring systems having fully saturated, aromatic and/or unsaturated portions (such as, for example, cyclohexenyl, 2,3-dihydro-indenyl, and 1,2,3,4-tetrahydro-naphthalenyl). The terms carbocyclic and carbocycle further include bridged, fused, and spirocyclic ring systems.

As used herein by themselves or in conjunction with another term or terms, “cycloalkyl” and “cycloalkyl group” refer to a non-aromatic carbocyclic ring system, that may be monocyclic, bicyclic, or tricyclic, saturated or unsaturated, and may be bridged, spiro, and/or fused. A cycloalkyl group may be substituted or unsubstituted. Unless specified otherwise, a cycloalkyl group typically contains from 3 to 12 ring atoms. In some instances a cycloalkyl group may contain 4 to 10 ring atoms (e.g., 4 ring atoms, 5 ring atoms, 6 ring atoms, 7 ring atoms, etc.). Representative examples include, but are not limited to, cyclopropyl, cyclopropenyl, cyclobutyl, cyclobutenyl, cyclopentyl, cyclopentenyl, cyclohexyl, cyclohexenyl, norbornyl, norbornenyl, bicyclo[2.2.1]hexane, bicyclo[2.2.1]heptane, bicyclo[2.2.1]heptene, bicyclo[3.1.1]heptane, bicyclo[3.2.1]octane, bicyclo[2.2.2]octane, bicyclo[3.2.2]nonane, bicyclo[3.3.1]nonane, and bicyclo[3.3.2]decane. Suitably, cycloalkyl groups are selected from cyclopropyl, cyclobutyl, cyclopentyl and cyclohexyl groups.

As used herein by themselves or in conjunction with another term or terms, “alkylcycloalkyl” and “alkylcycloalkyl group” refer to an alkyl group in which a hydrogen atom is replaced by a cycloalkyl group, wherein alkyl group and cycloalkyl group are as previously defined, such as, for example, cyclohexylmethyl (C₆H₁₁CH₂—). Alkylcycloalkyl groups can be substituted or unsubstituted.

As used herein by themselves or in conjunction with another term or terms, “haloalkyl” and “haloalkyl group” refer to alkyl groups in which one or more hydrogen atoms are replaced by halogen atoms. Haloalkyl includes both saturated alkyl groups as well as unsaturated alkenyl and alkynyl groups. Representative examples include, but are not limited to, —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CF═CF₂, —CCl═CH₂, —CBr═CH₂, —Cl═CH₂, —C═C—CF₃, —CHFCH₂CH₃ and —CHFCH₂CF₃. Haloalkyl groups can be substituted or unsubstituted. Suitably, a haloalkyl group is selected from CHF₂ and CF₃, suitably CF₃.

As used herein by themselves or in conjunction with another term or terms, “haloalkoxy” and “haloalkoxy group” refer to alkoxy groups (i.e. O-alkyl groups) in which one or more hydrogen atoms are replaced by halogen atoms. Haloalkoxy includes both saturated alkoxy groups as well as unsaturated alkenyl and alkynyl groups. Representative examples include, but are not limited to, —OCF₃, —OCHF₂, —OCH₂F, —OCF₂CF₃, —OCHFCF₃, —OCH₂CF₃, —OCF₂CH₃, —OCHFCH₃, —OCF₂CF₂CF₃, —OCF₂CH₂CH₃, —OCF═CF₂, —OCCl═CH₂, —OCBr═CH₂, —OCHFCH₂CH₃ and —OCHFCH₂CF₃. Haloalkoxy groups can be substituted or unsubstituted. Suitably, a haloalkyoxy group is selected from —OCHF₂ and —OCF₃, suitably —OCF₃.

As used herein by themselves or in conjunction with another term or terms, “halo” and “halogen” include fluorine, chlorine, bromine and iodine atoms and substituents.

As used herein by themselves or in conjunction with another term or terms, “heteroaryl” and “heteroaryl group” refer to (a) 5 and 6 membered monocyclic aromatic rings, which contain, in addition to carbon atom(s), at least one heteroatom, such as nitrogen, oxygen or sulfur, and (b) 7 to 15 membered bicyclic and tricyclic rings, which contain, in addition to carbon atom(s), at least one heteroatom, such as nitrogen, oxygen or sulfur, and in which at least one of the rings is aromatic. In some instances, a heteroaryl group can contain two or more heteroatoms, which may be the same or different. Heteroaryl groups can be substituted or unsubstituted, and may be bridged, spiro, and/or fused. In some instances, a heteroaryl group may contain 5, 6, or 8 to 15 ring atoms. In other instances, a heteroaryl group may contain 5 to 10 ring atoms, such as 5, 6, 9, or 10 ring atoms. Representative examples include, but are not limited to, 2,3-dihydrobenzofuranyl, 1,2-dihydroquinolinyl, 3,4-dihydroisoquinolinyl, 1,2,3,4-tetrahydroisoquinolinyl, 1,2,3,4-tetrahydroquinolinyl, benzoxazinyl, benzthiazinyl, chromanyl, furanyl, 2-furanyl, 3-furanyl, imidazolyl, isoxazolyl, isothiazolyl, oxadiazolyl, oxazolyl, pyridinyl, 2-, 3-, or 4-pyridinyl, pyrimidinyl, 2-, 4-, or 5-pyrimidinyl, pyrazolyl, pyrrolyl, 2- or 3-pyrrolyl, pyrazinyl, pyridazinyl, 3- or 4-pyridazinyl, 2-pyrazinyl, thienyl, 2-thienyl, 3-thienyl, tetrazolyl, thiazolyl, thiadiazolyl, triazinyl, triazolyl, pyridin-2-yl, pyridin-4-yl, pyrimidin-2-yl, pyridazin-4-yl, pyrazin-2-yl, naphthyridinyl, pteridinyl, phthalazinyl, purinyl, alloxazinyl, benzimidazolyl, benzofuranyl, benzofurazanyl, 2H-1-benzopyranyl, benzothiadiazine, benzothiazinyl, benzothiazolyl, benzothiophenyl, benzoxazolyl, cinnolinyl, furopyridinyl, indolinyl, indolizinyl, indolyl, or 2-, 3-, 4-, 5-, 6-, or 7-indolyl, 3H-indolyl, quinazolinyl, quinoxalinyl, isoindolyl, isoquinolinyl, 10-aza-tricyclo[6.3.1.0^{2,7}]dodeca-2(7),3,5-trienyl, 12-oxa-10-aza-tricyclo[6. 3. 1. 0^{2,7}]dodeca-2(7), 3,5-trienyl, 12-aza-tricyclo[7.2.1.0^{2,7}]dodeca-2(7), 3,5-trienyl, 10-aza-tricyclo[6.3.2. 0^{2,7}]trideca-2(7), 3,5-trienyl, 2,3,4,5-tetrahydro-1H-benzo[d]azepinyl, 1,3,4,5-tetrahydro-benzo[d]azepin-2-onyl, 1,3,4,5-tetrahydro-benzo[b]azepin-2-onyl, 2,3,4,5-tetrahydro-benzo[c]azepin-1-onyl, 1,2,3,4-tetrahydro-benzo[e][1,4]diazepin-5-onyl, 2,3,4,5-tetrahydro-1H-benzo[e][1,4]diazepinyl, 5,6,8,9-tetrahydro-7-oxa-benzocycloheptenyl, 2,3,4,5-tetrahydro-1H-benzo[b]azepinyl, 1,2,4,5-tetrahydro-benzo[e][1,3]diazepin-3-onyl, 3,4-dihydro-2H-benzo[b][1,4]dioxepinyl, 3,4-dihydro-2H-benzo[f][1,4]oxazepin-5-onyl, 6,7,8,9-tetrahydro-5-thia-8-aza-benzocycloheptenyl, 5,5-dioxo-6,7,8,9-tetrahydro-5-thia-8-aza-benzocycloheptenyl, and 2,3,4,5-tetrahydro-benzo[f][1,4]oxazepinyl. Suitably, a heteroaryl is a 5- or 6-membered heteroaryl ring comprising one, two or three heteroatoms selected from N, O or S.

As used herein by themselves or in conjunction with another term or terms, “alkylheteroaryl” and “alkylheteroaryl group” refer to an alkyl group in which a hydrogen atom is replaced by a heteroaryl group, wherein alkyl group and heteroaryl group are as previously defined. Alkylheteroaryl groups can be substituted or unsubstituted. Where carbon numbers are provided, e.g. (Cₙ₋ₘ)alkylheteroaryl, the range refers to the whole group. Suitably, the consitutent alkyl group has 1-6 carbons, suitable 1-3 carbons.

As used herein by themselves or in conjunction with another term or terms, “heterocyclic group” and “heterocycle” refer to monocyclic and polycyclic ring systems that contain carbon atoms and at least one heteroatom selected from nitrogen, oxygen, sulfur or phosphorus in the ring(s), without regard or reference to aromaticity or degree of unsaturation. Thus, a heterocyclic group should be understood as referring to and including ring systems that are fully saturated (such as, for example, a piperidinyl group), ring systems that are aromatic (such as, for example, a pyrindinyl group), as well as ring systems having fully saturated, aromatic and/or unsaturated portions (such as, for example, 1,2,3,6-tetrahydropyridinyl and 6,8-dihydro-5H-[1,2,4]triazolo[4,3-a]pyrizinyl). The terms heterocyclic and heterocycle further include bridged, fused, and spirocyclic ring systems.

As used herein by themselves or in conjunction with another term or terms, “heterocycloalkyl” and “heterocycloalkyl group” refer to 3 to15 membered monocyclic, bicyclic, and tricyclic non-aromatic ring systems, which contain, in addition to carbon atom(s), at least one heteroatom, such as nitrogen, oxygen, sulfur or phosphorus. Heterocycloalkyl groups may be fully saturated or contain unsaturated portions and may be bridged, spiro, and/or fused ring systems. In some instances a heterocycloalkyl group may contain at least two or heteroatoms, which may be the same or different. Heterocycloalkyl groups can be substituted or unsubstituted. In some instances a heterocycloalkyl group may contain from 3 to 10 ring atoms or from 3 to 7 ring atoms or from 5 to 7 ring atoms, such as 5 ring atoms, 6 ring atoms, or 7 ring atoms. Representative examples include, but are not limited to, tetrahydrofuranyl, pyrrolidinyl, pyrrolinyl, imidazolidinyl, imidazolinyl, pyrazolidinyl, pyrazolinyl, piperidyl, piperazinyl, indolinyl, isoindolinyl, morpholinyl, thiomorpholinyl, homomorpholinyl, homopiperidyl, homopiperazinyl, thiomorpholinyl-5-oxide, thiomorpholinyl-S,S-dioxide, pyrrolidinyl, tetrahydropyranyl, piperidinyl, tetrahydrothienyl, homopiperidinyl, homothiomorpholinyl-S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl-5-oxide, tetrahydrothienyl-S,S-dioxide, homothiomorpholinyl-5-oxide, quinuclidinyl, 2-oxa-5-azabicyclo[2.2.1]heptanyl, 8-oxa-3-aza-bicyclo[3.2.1]octanyl, 3,8-diaza-bicyclo[3.2.1]octanyl, 2,5-diaza-bicyclo[2.2.1]heptanyl, 3,8-diaza-bicyclo[3.2.1]octanyl, 3,9-diaza-bicyclo[4.2.1]nonanyl, 2,6-diaza-bicyclo[3.2.2]nonanyl, [1,4]oxaphosphinanyl-4-oxide, [1,4]azaphosphinanyl-4-oxide, [1,2]oxaphospholanyl-2-oxide, phosphinanyl-1-oxide, [1,3]azaphospholidinynl-3-oxide, [1,3]oxaphospholanyl-3-oxide, 7-oxabicyclo[2.2.1]heptanyl, 6,8-dihydro-5H-[1,2,4]triazolo[4,3-a]pyrazin-7-yl, 6,8-dihydro-5H-imidazo[1,5-a]pyrazin-7-yl, 6,8-dihydro-5H-imidazo[1,2-a]pyrazin-7-yl, 5,6,8,9-tetrahydro-[1,2,4]triazolo[4,3-d][1,4]diazepin-7-yl and 6,8-dihydro-5H-[1,2,4]triazolo[4,3-a]pyrazin-7-yl. Suitably, a heterocyclylalkyl group as defined herein is a monocyclic, bicyclic or spiro heterocyclyl group comprising one, two or three heteroatoms selected from N, O or S.

As used herein by themselves or in conjunction with another term or terms, “heterocycloalkylene” and “heterocycloalkylene group” refer to 3 to15 membered monocyclic, bicyclic, or tricyclic non-aromatic ring systems, which contain, in addition to carbon atom(s), at least one heteroatom, such as nitrogen, oxygen, sulfur or phosphorus. Heterocycloalkylene groups may be fully saturated or contain unsaturated portions and may be bridged, spiro, and/or fused. Heterocycloalkylene groups can be substituted or unsubstituted. In some instances, a heterocycloalkylene group may contain from 3 to 10 ring atoms; such as from 3 to 7 ring atoms. In other instances a heterocycloalkylene group may contain from 5 to 7 ring atoms, such as 5 ring atoms, 6 ring atoms, or 7 ring atoms.

As used herein by themselves or in conjunction with another term or terms, “alkylheterocycloalkyl” and “alkylheterocycloalkyl group” refer to an alkyl group in which a hydrogen atom is replaced by a heterocycloalkyl group, wherein alkyl group and heterocycloalkyl group are as previously defined, such as, for example, pyrrolidinylmethyl (C₄H₈NCH₂—). Alkylheteroycloalkyl groups can be substituted or unsubstituted. Where carbon numbers are provided, e.g. (Cₙ₋ₘ)alkylheterocycloalkyl, the range refers to the whole group. Suitably, the consitutent alkyl group has 1-6 carbons, suitable 1-3 carbons.

As used herein by itself or in conjunction with another term or terms, “pharmaceutically acceptable” refers to materials that are generally chemically and/or physically compatible with other ingredients (such as, for example, with reference to a formulation), and/or is generally physiologically compatible with the recipient (such as, for example, a subject) thereof.

As used herein by itself or in conjunction with another term or terms, “pharmaceutical composition” refers to a composition that can be used to treat a disease, condition, or disorder in a subject, including a human.

As used herein by themselves or in conjunction with another term or terms, “stable” and “chemically stable” refer to a compound that is sufficiently robust to be isolated from a reaction mixture with a useful degree of purity. The present application is directed solely to the preparation of stable compounds. When lists of alternative substituents include members which, owing to valency requirements, chemical stability, or other reasons, cannot be used to substitute a particular group, the list is intended to be read in context to include those members of the list that are suitable for substituting the particular group. For example, when considering the degree of optional substitution of a particular moiety, it should be understood that the number of substituents does not exceed the valency appropriate for that moiety. For example, if R¹ is a methyl group (—CH₃), it can be optionally substituted by 1 to 3 R⁵.

As used herein by itself or in conjunction with another term or terms, “substituted” indicates that a hydrogen atom on a molecule has been replaced with a different atom or group of atoms and the atom or group of atoms replacing the hydrogen atom is a “substituent.” It should be understood that the terms “substituent”, “substituents”, “moiety”, “moieties”, “group”, or “groups” refer to substituent(s).

As used herein, a “pharmaceutical product” refers to a product comprising a pharmaceutical. For instance, examples of a pharmaceutical product include a medical device, a pharmaceutical composition and a kit comprising one or more medical device and/or pharmaceutical composition. Suitably, the pharmaceutical product is a pharmaceutical composition.

### Methods of Treatment and Medical Uses

In one aspect, the present invention provides a method for the treatment of an oestrogen receptor positive breast cancer in a subject in need thereof comprising administering to said subject a therapeutically effective amount of an MPS1 inhibitor, wherein:
(ii) said breast cancer is resistant to endocrine therapy.

In another aspect, the present invention provides an MPS1 inhibitor for use in the treatment of an oestrogen receptor-positive breast cancer in a subject in need thereof, wherein:
(i) the subject has previously been treated with an endocrine therapy; and/or
(ii) said breast cancer is resistant to endocrine therapy.

In another aspect, the present invention provides a use of an MPS1 inhibitor in the manufacture of a medicament for the treatment of an oestrogen receptor positive breast cancer in a subject in need thereof, wherein:
(i) the subject has been previously treated with an endocrine therapy; and/or
(ii) said breast cancer is resistant to endocrine therapy.

In one embodiment of each of the above aspects, the subject has been previously treated with an endocrine therapy. Typically, the need for further treatment implies that the previous endocrine therapy has failed.

The relevant skilled person would be readily able to determine treatment failure. In one embodiment, failure of endocrine therapy in a subject manifests as relapse and/or cancer recurrence during or following endocrine therapy.

In one embodiment of each of the aspects or embodiments herein, failure of endocrine therapy treatment in a subject is observed as disease progression during or following endocrine therapy, for example, an increase in primary tumour size and/or spread of disease; for example, to regional nodes or beyond to more distant metastatic sites.

In one embodiment of each of the aspects or embodiments herein, failure of endocrine therapy treatment in a subject is determined by pathological changes, such as increased tumour grade.

In one embodiment of each of the aspects or embodiments herein, failure of endocrine therapy treatment in a subject is determined by increased proliferation of the cancer.

In one embodiment of each of the aspects or embodiments herein, failure of endocrine therapy treatment in the subject is observed as a lack of response of the cancer;

for example, no significant/insufficient change in tumour size and/or no significant/insufficient level of pathological response.

In one embodiment of each of the aspects or embodiments herein, the subject has developed an endocrine therapy-resistant breast cancer during or following endocrine therapy. This phenomenon may be referred to as acquired resistance.

In one embodiment of each of the aspects and embodiments herein, the breast cancer is resistant to endocrine therapy. In one embodiment, the endocrine therapy resistance is observed during or following endocrine therapy, which may have initially resulted in a positive response (i.e. acquired resistance). In another embodiment, the endocrine therapy resistance is observed early in endocrine therapy without a period of positive response, implying innate or de novo resistance. In another embodiment, the subject is endocrine therapy-naïve and breast cancer endocrine-resistance is indicated by phenotypic or genotypic markers.

In one embodiment of each of the aspects herein, the breast cancer is resistant to endocrine therapy and the subject is endocrine therapy-naïve.

In one embodiment of each of the aspects herein, the breast cancer is de novo resistant to endocrine therapy.

In one embodiment of each of the aspects herein, the breast cancer is resistant to endocrine therapy and continues to express oestrogen receptors, in particular, ER-alpha.

In one embodiment of each of the aspects herein, the breast cancer harbours an ESR1-activating mutation. Suitably, the ESR1-activating mutation is selected from Y537S, Y537N, Y537C, D538G, E380Q, S463P, L536R. Suitably, the ESR1-activating mutation is selected from Y537S, Y537N and Y537C. Suitably, the ESR/-activating mutation is Y537C.

In one embodiment of each of the aspects herein, the breast cancer is resistant to endocrine therapy and harbours an ESR /-activating mutation. Suitably, the ESR1-activating mutation is selected from Y537S, Y537N, Y537C, D538G, E380Q, S463P, L536R. Suitably, the ESR1-activating mutation is selected from Y537S, Y537N and Y537C. Suitably, the ESR1-activating mutation is Y537C.

In one embodiment of each of the aspects herein, the breast cancer overexpresses phospho-KNL1 protein.

In one embodiment of each of the aspects herein, the breast cancer is resistant to endocrine therapy and the breast cancer overexpresses phospho-KNL1 protein.

In one embodiment of each of the aspects and embodiments herein, the subject has additionally been previously treated with a CDK4/6 inhibitor. Typically, the need for further treatment implies that the previous treatment with a CDK4/6 inhibitor has failed.

The relevant skilled person would be readily able to determine treatment failure. In one embodiment, failure of treatment with a CDK4/6 inhibitor in a subject manifests as relapse and/or cancer recurrence during or following treatment with a CDK4/6 inhibitor.

In one embodiment of each of the aspects or embodiments herein, failure of treatment with a CDK4/6 inhibitor in a subject is observed as disease progression during or following treatment with a CDK4/6 inhibitor, for example, an increase in primary tumour size and/or spread of disease; for example, to regional nodes or beyond to more distant metastatic sites.

In one embodiment of each of the aspects or embodiments herein, failure of treatment with a CDK4/6 inhibitor in a subject is determined by pathological changes, such as increased tumour grade.

In one embodiment of each of the aspects or embodiments herein, failure of treatment with a CDK4/6 inhibitor in a subject is determined by increased proliferation of the cancer.

In one embodiment of each of the aspects or embodiments herein, failure of treatment with a CDK4/6 inhibitor in the subject is observed as a lack of response of the cancer; for example, no change in tumour size and/or no evidence of pathological response.

In one embodiment of each of the aspects or embodiments herein, the subject has developed a CDK4/6 inhibitor-resistant breast cancer during or following treatment with a CDK4/6 inhibitor. This phenomenon may be referred to as acquired resistance.

In one embodiment of each of the aspects and embodiments herein, the breast cancer is resistant to treatment with a CDK4/6 inhibitor. In one embodiment, the CDK4/6 inhibitor-resistance is observed during or following treatment with a CDK4/6 inhibitor, which may have initially resulted in a positive response (i.e. acquired resistance). In another embodiment, the CDK4/6-inhibitor-resistance is observed early in treatment with a CDK4/6 inhibitor without a period positive response, implying innate or de novo resistance. In another embodiment, the subject is CDK4/6 inhibitor-naïve and breast cancer CDK4/6-inhibitor resistance is indicated by phenotypic or genotypic markers.

In one embodiment of each of the aspects herein, the breast cancer is resistant to CDK4/6 inhibitors and the subject is CDK4/6 inhibitor-naïve.

In one embodiment of each of the aspects herein, the breast cancer is de novo resistant to CDK4/6 inhibitors.

In one embodiment of each of the aspects and embodiments herein, the MPS1 inhibitor is administered/for administration either separately, sequentially and/or combination with an endocrine therapy. Suitably, the endocrine therapy comprises/essentially consists of/consists of treatment with an endocrine agent.

In one aspect, the present invention provides the use of an MPS1 inhibitor as a second or third-line therapy for the treatment of estrogen receptor position breast cancer, in particular, endocrine therapy-resistant ER+breast cancer.

In one embodiment, the MPS1 inhibitor is used as a third line therapy and the first line therapy comprised treatment with an endocrine agent and the second line therapy comprised treatment with a CDK4/6 inhibitor.

In one embodiment, the MPS1 inhibitor is used as a second line therapy and the first line therapy comprised treatment with an endocrine agent. In another embodiment, the MPS1 inhibitor is used as a third line therapy and the first and second line therapies each comprised treatment with an endocrine agent.

In one embodiment of each of the aspects and embodiments herein, the subject is a human. Suitably, the subject is a female human.

In one embodiment of each of the aspects and embodiments herein, the subject is post-menopausal. In another embodiment of each of the aspects and embodiments herein, the subject is pre-menopausal.

In the methods and medical uses of the invention the MPS1 inhibitors and/or the endocrine agents may be administered/for administration to the subject by any convenient route of administration.

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly. Suitably, the route of administration is selected from oral and parenteral injection.

The therapeutic agents (i.e. MPS1 inhibitors, endocrine agents, CDK4/6 inhibitors) for use in the methods herein may be in a form suitable for administration to a subject. For instance for oral use tablets, lozenges, hard or soft capsules, aqueous or oily suspensions, emulsions, dispersible powders or granules, syrups or elixirs; for topical use creams, ointments, gels, or aqueous or oily solutions or suspensions); for administration by inhalation a finely divided powder or a liquid aerosol; for administration by insufflation a finely divided powder); or for parenteral administration a sterile aqueous or oily solution for intravenous, subcutaneous, intramuscular, intraperitoneal or intramuscular dosing; or as a suppository for rectal dosing.

Suitable pharmaceutical compositions may be obtained by conventional procedures optionally using conventional pharmaceutical excipients, well known in the art. Thus, compositions intended for oral use may contain, for example, one or more colouring, sweetening, flavouring and/or preservative agents.

An effective amount of the agents (i.e. MPS1 inhibitors, endocrine agents, CDK4/6 inhibitors) of the methods herein is an amount sufficient to treat or prevent said breast cancer referred to herein, slow disease progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of an agent will naturally vary according to the nature and severity of the conditions, the age and sex of the animal or patient and the route of administration, according to well known principles of medicine.

It is to be noted that dosages and dosing regimens may vary with the type and severity of the condition to be alleviated, and may include the administration of single or multiple doses, i.e. QD (once daily), BID (twice daily), etc., over a particular period of time (days or hours). It is to be further understood that for any particular subject or patient, specific dosage regimens may need to be adjusted over time according to the individual need and the professional judgment of the person administering or supervising the administration of the pharmaceutical compositions. For example, doses may be adjusted based on pharmacokinetic or pharmacodynamic parameters, which may include clinical effects such as toxic effects and/or laboratory values. Thus, the present application encompasses intra-patient dose-escalation as determined by the person skilled in the art. Procedures and processes for determining the appropriate dosage(s) and dosing regimen(s) are well-known in the relevant art and would readily be ascertained by the skilled artisan. As such, one of ordinary skill would readily appreciate and recognize that the dosage ranges set forth herein are exemplary only and are not intended to limit the scope or practice of the pharmaceutical compositions described herein.

### Endocrine Therapy

In one embodiment of each of the aspects and embodiments herein, the endocrine therapy comprises/essentially consists of/consists of treatment with an endocrine agent.

In one embodiment of each of the aspects and embodiments herein, the endocrine agent is selected from one or more of an aromatase inhibitor, a selective oestrogen receptor modulator (SERM) and a selective oestrogen receptor degrader/downregulator (SERD).

In one embodiment of each of the aspects and embodiments herein, endocrine therapy comprises/essentially consists of/consists of separate, sequential or combined treatment with an aromatase inhibitor and a SERD.

In one embodiment of each of the aspects and embodiments herein, endocrine therapy comprises/essentially consists of/consists of separate, sequential or combined treatment with an aromatase inhibitor and a SERM.

In one embodiment of each of the aspects and embodiments herein, endocrine therapy comprises/essentially consists of/consists of separate, sequential or combined treatment with a SERM and a SERD.

In one embodiment of each of the aspects and embodiments herein, the SERM is selected from the group consisting of tamoxifen, afimoxifene, raloxifene, toremifene, bazedoxifene and lasofoxifene, or pharmaceutically acceptable salts or solvates thereof. Suitably, the SERM is selected from the group consisting of tamoxifen, raloxifene, toremifene, bazedoxifene and lasofoxifene, or pharmaceutically acceptable salts or solvates thereof. Suitably, the SERM is selected from the group consisting of tamoxifen, raloxifene and toremifene, or pharmaceutically acceptable salts or solvates thereof. Suitably, the SERM is selected from the group consisting of tamoxifen and toremifene, or pharmaceutically acceptable salts or solvates thereof. Suitably, the SERM is selected from the group consisting of tamoxifen and raloxifene, or pharmaceutically acceptable salts or solvates thereof. Suitably, the SERM is tamoxifen, or pharmaceutically acceptable salts or solvates thereof.

In one embodiment of each of the aspects and embodiments herein, the aromatase inhibitor is selected from the group consisting of anastrozole, exemestane, letrozole, fadrozole and formestane, or pharmaceutically acceptable salts or solvates thereof. Suitably, the aromatase inhibitor is selected from the group consisting of anastrozole, exemestane, letrozole and fadrozole, or pharmaceutically acceptable salts or solvates thereof. Suitably, the aromatase inhibitor is selected from the group consisting of anastrozole, exemestane, and letrozole, or pharmaceutically acceptable salts or solvates thereof. Suitably, the aromatase inhibitor is selected from the group consisting of anastrozole and letrozole, or pharmaceutically acceptable salts or solvates thereof. Suitably, the aromatase inhibitor is selected from the group consisting of anastrozole and exemestane, or pharmaceutically acceptable salts or solvates thereof. Suitably, the aromatase inhibitor is selected from the group consisting of exemestane and letrozole, or pharmaceutically acceptable salts or solvates thereof.

In one embodiment of each of the aspects and embodiments herein, the SERD is selected from the group consisting of fulvestrant, brilanestrant and elacestrant, or pharmaceutically acceptable salts or solvates thereof. Suitably, the SERD is selected from the group consisting of fulvestrant and elacestrant, or pharmaceutically acceptable salts or solvates thereof. Suitably, the SERD is selected from the group consisting of fulvestrant and briianestrant, or pharmaceutically acceptable salts or solvates thereof. Suitably, the SERD is fulvestrant, or pharmaceutically acceptable salts or solvates thereof.

In one embodiment of each of the aspects and embodiments herein, the endocrine therapy comprises/essentially consists of/consists of separate, sequential or combined treatment with anastrozole and fulvestrant.

In one embodiment of each of the aspects and embodiments herein, the endocrine therapy comprises/essentially consists of/consists of separate, sequential or combined treatment with tamoxifen and fulvestrant.

### MPS1 Inhibitors

In one embodiment of the aspects and embodiments herein, the MPS1 inhibitor is a compound capable of inhibiting MPS1 kinase. Suitably, the compound has an IC₅₀ at MPS1 kinase of 100 nM or less. Suitably, the compound has an IC₅₀ at MPS1 kinase of 75 nM or less. Suitably, the compound has an IC₅₀ at MPS1 kinase of 50 nM or less. Suitably, the compound has an IC₅₀ at MPS1 kinase of 25 nM or less. Suitably, the compound has an IC₅₀ at MPS1 kinase of 10 nM or less. Suitably, the compound has an IC₅₀ at MPS1 kinase of 8 nM or less. Suitably, the compound has an IC₅₀ at MPS1 kinase of 5 nM or less. Suitably, the compound has an IC₅₀ at MPS1 kinase of 3 nM or less.

The IC₅₀ at MPS1 kinase may be determined by any suitable method. For example, the IC₅₀ may be determined by in vitro enzyme inhibition assay comprising full length MPS1, a suitable fluorophore, test compound and an assay buffer.

Suitably, IC₅₀s are determined by testing the compounds at a range of concentrations.

Suitably, the fluorophore can be a fluorescent labelled peptide, for example, H236, which has the sequence: 5FAM-DHTGFLTEYVATR-CONH₂.

Suitably, the enzyme inhibition assay is carried out at room temperature (21° C.±3° C.) for about one hour.

In one embodiment, the enzyme inhibition assay (total volume 10 μl) was carried out in black 384-well low volume plates containing full length MPS1 (12.5 nM or 3 nM), fluorescent labelled peptide [known as H236, which has the sequence: 5FAM-DHTGFLTEYVATR-CONH₂] (5 μM), ATP(10 μM), either DMSO (1% v/v) or the test compound (in the range 0.25 nM-100 μM in 1% DMSO) and assay buffer (50 mM HEPES (pH 7.0), 0.02% NaN₃, 0.01% BSA, 0.1 mM Orthovandate, 10 μM MgCl₂, 1 μM DTT, Roche protease inhibitor). The reaction was carried out for 60 min at room temperature and stopped by the addition of buffer (10 μl) containing 20 mM EDTA, 0.05% (v/v) Brij-35, in 0.1M HEPES-buffered saline (Free acid, Sigma, UK). The plate was read on a Caliper EZ reader II (Caliper Life Sciences).

The reader provides a Software package (‘Reviewer’) which converts the peak heights into % conversion by measuring both product and substrate peak and also allows selection of control well which represent 0% and 100% inhibition, respectively. The % inhibition of the compounds is calculated relative to the means of selected control wells. IC₅₀s are determined by testing the compounds at a range of concentrations from 0.25 nM-100 μM. The % inhibitions at each concentration are then fitted to a 4 parameter logistic fit :

y=(a+((b−a)/(1+((c/x\hat{ }d))))

where a=asym min, b=asym max, c=IC₅₀ and d=hill coefficient

In one embodiment of each of the aspects and embodiment herein, the MPS1 inhibitor is selected from the group consisting of NMS-P715, S 81694 (NMS-P153), AZ3146, BAY 1217389, BAY 1161909, MPS1-IN-3, MPS1-IN-2, CFI-402257, CCT289346, a compound of formula I, a compound of formula II, a compound of formula III and a compound of formula IV, or a pharmaceutically acceptable salt or solvate thereof;

wherein formula I is:

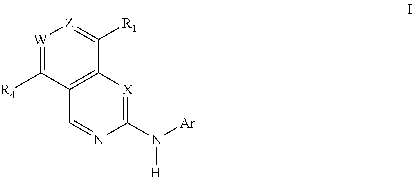

wherein:
W is N or C—R₃;
X is CH or N;
Z is N or C—H;
R₁ is selected from chloro, (1-6C)alkyl, (1-8C)heteroalkyl, aryl, aryl(1-2C)alkyl, heteroaryl, heteroaryl(1-2C)alkyl, heterocyclyl, heterocyclyl(1-2C)alkyl, (3-8C)cycloalkyl, (3-8C)cycloalkyl(1-2C)alkyl, NR₇R₈, OR₉, C(O)R₉, C(O)OR₉, OC(O)R₉, N(R₁₀)OR₉, N(R₁₀)C(O)OR₉, C(O)N(R₁₀)R₉, N(R₁₀)C(O)R₉, S(O)ₚR₉ (where p is 0, 1 or 2), SO₂N(R₁₀)R₉, N(R₁₀)SO₂R₉, N(R₁₀)SOR₉ or SON(R₁₀)R₉;
and wherein R₁ is optionally substituted by one or more substituent groups selected from fluoro, chloro, trifluoromethyl, trifluoromethoxy, cyano, nitro, hydroxy, amino, carboxy, carbamoyl, sulphamoyl, (1-4C)alkyl, (1-4C)alkoxy, S(O)_{q}CH₃ (where q is 0, 1 or 2), methylamino or dimethylamino, aryl, aryl(1-2C)alkyl, heteroaryl, heteroaryl(1-2C)alkyl, heterocyclyl, heterocyclyl(1-2C)alkyl, (3-8C)cycloalkyl, or (3-8C)cycloalkyl(1-2C)alkyl,
and wherein any (1-4C)alkyl, (1-4C)alkoxy, aryl, heteroaryl, heterocyclyl, or (3-8C)cycloalkyl moiety present within a substituent group on R₁ is optionally further substituted by fluoro, chloro, trifluoromethyl, trifluoromethoxy, cyano, nitro, hydroxy, amino, carboxy, carbamoyl, sulphamoyl, (1-4C)alkyl, NRₐR_{b}, ORₐ, C(O)Rₐ, C(O)ORₐ, OC(O)Rₐ, N(R_{b})ORₐ, C(O)N(R_{b})Rₐ, N(R_{b})C(O)Rₐ, S(O)ₚRₐ (where p is 0, 1 or 2), SO₂N(R_{b})Rₐ, or N(R_{b})SO₂Rₐ, wherein Rₐ and R_{b} are each independently selected from H or (1-4C)alkyl;
R₃ is hydrogen, (1-4C)alkyl, (3-6C)cycloalkyl, halo, CF₃, CN and (1-4C)alkoxy;
R₄ is hydrogen, (1-3C)alkyl, (1-3C)alkoxy, fluoro, chloro or CF₃;
Ar has the formula:

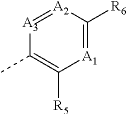

wherein:
(i) all of A₁, A₂ and A₃ are CH;
(ii) one of A₁, A₂ and A₃ is N and the others are CH; or
(iii) two of A₁, A₂ and A₃ are N and the other is CH;
R₅ is selected from hydrogen, cyano, (1-3C)alkyl, (1-3C)fluoroalkyl, (1-3C)alkoxy, (1-3C)fluoroalkoxy, halo, (1-3C)alkanoyl, C(O)NR₁₅R₁₆ or S(O)₂NR₁₅R₁₆, and wherein R₁₅ and R₁₆ are each independently selected from H or (1-3C)alkyl,
and wherein any alkyl or alkoxy moities present within a R₅ substituent group are optionally further substituted by hydroxy or methoxy;
R₆ is selected from halo, trifluoromethyl, trifluoromethoxy, cyano, nitro, hydroxy, amino, carboxy, carbamoyl, sulphamoyl, ureido, (1-6C)alkyl, (2-6C)alkenyl, (2-6C)alkynyl, or R₆ is a group of the formula:

-L¹-L²-R₁₇

wherein
L¹ is absent or a linker group of the formula —[CR₁₈R₁₉]ₙ— in which n is an integer selected from 1, 2, 3 or 4, and R₁₉ and R₁₉ are each independently selected from hydrogen or (1-2C)alkyl;
L² is absent or is selected from O, S, SO, SO₂, N(R₂₀), C(O), C(O)O, OC(O), CH(OR₂₀), C(O)N(R₂₀), N(R₂₀)C(O), N(R₂₀)C(O)N(R₂₁), S(O)₂N(R₂₀), or N(R₂₁)SO₂, wherein R₂₀ and R₂₁ are each independently selected from hydrogen or (1-2C)alkyl; and
R₁₇ is (1-6C)alkyl, aryl, aryl-(1-6C)alkyl, (3-6C)cycloalkyl, (3-6C)cycloalkyl-(1-4C)alkyl, heteroaryl, heteroaryl-(1-4C)alkyl, heterocyclyl, heterocyclyl-(1-4C)alkyl,
and wherein R₁₇ is optionally further substituted by one or more substituent groups independently selected from oxo, halo, cyano, nitro, hydroxy, NR₂₂R₂₃, (1-4C)alkoxy, (1-4C)alkyl, (3-8C)cycloalkyl, (3-8C)cycloalkyl-(1-3C)alkyl, (1-5C)alkanoyl, (1-5C)alkylsulphonyl, heterocyclyl, heterocyclyl-(1-2C)alkyl, heteroaryl, heteroaryl-(1-2C)alkyl, CONR₂₂R₂₃, and SO₂NR₂₂R₂₃; wherein R₂₂ and R₂₃ are each independently selected from hydrogen, (1-4C)alkyl or (3-6C)cycloalkyl or (3-6C)cycloalkyl(1-2C)alkyl;
and wherein when said substituent group comprises an alkyl, cycloalkyl, heterocyclyl or heteroaryl moiety then said moiety is optionally further substituted by hydroxy, fluoro, chloro, cyano, CF₃, OCF₃, (1-2C)alkyl, (1-2C)alkoxy, SO₂(1-2C)alkyl or NRₑR_{f} (where Rₑ and R_{f} are each independently selected from hydrogen, (1-3C)alkyl, (3-6C)cycloalkyl, or (3-6C)cycloalkyl(1-2C)alkyl);
or R₁₇ is a group having the formula:

-L³-L⁴-R₂₄

L³ is absent or a linker group of the formula —[CR₂₅R₂₆]ₙ— in which n is an integer selected from 1, 2, 3 or 4, and R₂₅ and R₂₆ are each independently selected from hydrogen or (1-2C)alkyl;

L⁴ is absent or is selected from O, S, SO, SO₂, N(R₂₇), C(O), C(O)O, OC(O), CH(OR₂₇), C(O)N(R₂₇), N(R₂₇)C(O), N(R₂₇)C(O)N(R₂₈), S(O)₂N(R₂₇), or N(R₂₈)SO₂, wherein R₂₇ and R₂₈ are each independently selected from hydrogen or (1-2C)alkyl; and

R₂₄ is (1-6C)alkyl, aryl, aryl-(1-6C)alkyl, (3-6C)cycloalkyl, (3-6C)cycloalkyl-(1-4C)alkyl, heteroaryl, heteroaryl-(1-4C)alkyl, heterocyclyl, heterocyclyl-(1-4C)alkyl;
R₈ and R₉ are each independently selected from hydrogen, (1-6C)alkyl, (1-6C)alkoxy, (3-9C)cycloalkyl, (3-9C)cycloalkyl-(1-2C)alkyl, aryl, aryl-(1-2C)alkyl, heterocyclyl, heterocyclyl-(1-2C)alkyl, heteroaryl, heteroaryl-(1-2C)alkyl, and wherein R₈ and R₉ are optionally further substituted by one or more substituents selected from hydroxy, fluoro, chloro, cyano, CF₃, OCF₃ (1-2C)alkyl or (1-2C)alkoxy;
R₇ and R₁₀ are independently selected from hydrogen, (1-6C)alkyl, (3-6C)cycloalkyl, (3-6C)cycloalkyl-(1-2C)alkyl, and wherein R₇ and R₁₀ are optionally further substituted by one or more substituents selected from hydroxy, fluoro, chloro, cyano, CF₃, OCF₃, (1-2C)alkyl or (1-2C)alkoxy;
optionally subject to the proviso that:

X is only N when Z is N;

W is only N when X and Z are both N; and

R₆ is not methoxy when R₁ is S(O)₂R₉ and R₉ is heterocyclyl;

wherein formula II is:

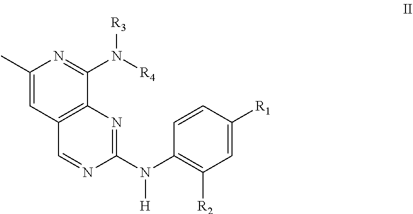

wherein:
R₁ is selected from:

(i) a 5- or 6-membered heteroaryl optionally substituted with one or more substituents independently selected from halo, trifluoromethyl, difluoromethyl, trifluoromethoxy, difluoromethoxy, cyano, nitro, (1-4C)alkyl, NRₐR_{b}, ORₐ, C(O)Rₐ, C(O)ORₐ, OC(O)Rₐ, N(R_{b})ORₐ, C(O)N(R_{b})Rₐ, N(R_{b})C(O)Rₐ, S(O)ₚRₐ (where p is 0, 1 or 2), SO₂N(R_{b})Rₐ, or N(R_{b})SO₂Rₐ,
wherein Rₐ and R_{b} are each independently selected from H or (1-4C)alkyl, and wherein any alkyl moiety present in the substituent group is optionally further substituted with one or more substituents selected from halo, trifluoromethyl, difluoromethyl, trifluoromethoxy, difluoromethoxy, cyano, nitro, (1-4C)alkyl, 4-7-membered heterocyclyl, NR_{c}R_{d}, OR_{c}, C(O)R_{c}, C(O)OR_{c}, OC(O)R_{c}, N(R_{d})OR_{c}, C(O)N(R_{d})R_{c}, N(R_{d})C(O)R_{c}, S(O)_{q}R_{c} (where q is 0, 1 or 2), SO₂N(R_{d})R_{c}, or N(R_{d})SO₂R_{c}, wherein R_{c} and R_{d} are each independently selected from H or (1-4C)alkyl; or
wherein the 5- or 6-membered heteroaryl is optionally fused to a 4-, 5-, 6- or 7-membered heterocyclic ring, wherein the fused ring system is optionally substituted by one or more substituents independently selected from halo, trifluoromethyl, difluoromethyl, trifluoromethoxy, difluoromethoxy, cyano, nitro, (1-4C)alkyl, NRₖRₗ, ORₖ, C(O)Rₖ, C(O)ORₖ, OC(O)Rₖ, N(Rₗ)ORₖ, C(O)N(Rₗ)Rₖ, N(Rₗ)C(O)Rₖ, S(O)ₚRₖ (where p is 0, 1 or 2), SO₂N(Rₖ)Rₗ, or N(Rₖ)SO₂Rₗ, wherein Rₖ and Rₗ are each independently selected from H or (1-4C)alkyl, and wherein any alkyl moiety present in the substituent group is optionally further substituted with one or more substituents selected from halo, trifluoromethyl, difluoromethyl, trifluoromethoxy, difluoromethoxy, cyano, nitro, (1-4C)alkyl, 4-7-membered heterocyclyl, NRₘRₙ, ORₘ, C(O)Rₘ, C(O)ORₘ, OC(O)Rₘ, N(Rₐ)ORₘ, C(O)N(Rₙ)Rₘ, N(Rₙ)C(O)Rₘ, S(O)_{q}Rₘ (where q is 0, 1 or 2), SO₂N(Rₙ)Rₘ, or N(Rₙ)SO₂Rₘ, wherein Rₘ and Rₙ are each independently selected from H or (1-4C)alkyl; or

(ii) a group —C(O)N(R_{f})Rₑ— or —S(O)₂N(R_{f})Rₑ—;
wherein Rₑ and R_{f} are each independently selected from H or (1-4C)alkyl which is optionally substituted by halo or (1-2C)alkoxy;
or Rₑ and R_{f} are linked such that, together with the nitrogen atom to which they are attached, they form a 4-, 5- or 6-membered heterocyclic ring, wherein said ring is optionally substituted with one or more substituents independently selected from halo, trifluoromethyl, difluoromethyl, trifluoromethoxy, difluoromethoxy, cyano, nitro, (1-4C)alkyl, NR_{g}Rₕ, OR_{g}, C(O)R_{g}, C(O)OR_{g}, OC(O)R_{g}, N(Rₕ)OR_{g}, C(O)N(Rₕ)R_{g}, N(Rₕ)C(O)R_{g}, S(O)ₚRₕ (where p is 0, 1 or 2), SO₂N(Rₕ)R_{g}, or N(Rₕ)SO₂R_{g}, wherein R_{g} and Rₕ are each independently selected from H or (1-4C)alkyl;
R₂ is selected from hydrogen, fluoro, chloro, (1-3C)alkoxy or (1-3C)fluoroalkoxy;
and either:

(i) R₃ is selected from hydrogen or (1-3C)alkyl and R₄ is selected from (1-6C)alkyl, (3-9C)cycloalkyl, (3-9C)cycloalkyl-(1-4C)alkyl, aryl, aryl-(1-4C)alkyl, heterocyclyl, heterocyclyl-(1-4C)alkyl, heteroaryl, heteroaryl-(1-4C)alkyl, and wherein R₄ is optionally further substituted by one or more substituents selected from hydroxy, fluoro, chloro, cyano, CF₃, CHF₂, OCF₃, OCHF₂, (1-4C)alkyl, NRₒRₚ, ORₒ, C(O)Rₒ, C(O)ORₚ, OC(O)Rₒ, N(Rₚ)ORₒ, C(O)N(Rₚ)Rₒ, N(Rₚ)C(O)Rₒ, S(O)ₚRₒ (where p is 0, 1 or 2), SO₂N(Rₚ)Rₒ, or N(Rₚ)SO₂Rₒ or (3-6C)cycloalkyl, (3-6C)cycloalkyl-(1-2C)alkyl, a 4, 5 or 6-membered heterocyclyl, a 4, 5 or 6-membered heterocyclyl-(1-2C)alkyl, wherein Rₒ and Rₚ are each independently selected from H or (1-4C)alkyl, (3-6C)cycloalkyl or (3-6C)cycloalkyl-(1-4C)alkyl; or

(ii) R₃ and R₄ are linked such that, together with the nitrogen atom to which they are attached, they form a nitrogen-linked 4-, 5- 6- or 7-membered heterocyclic ring,
wherein said ring is optionally fused to a further 3-, 4-, 5- or 6-membered ring carbocyclic or heterocyclic ring, a 5- or 6-membered heteroaryl ring or a phenyl ring to form a bi-cyclic heterocyclic system, or
linked through a spiro carbon atom to a further 4-, 5- or 6-membered ring carbocyclic or heterocyclic ring to form a spiro bicyclic ring system;
and wherein the heterocyclic ring, bicyclic ring system or spiro bicyclic ring system is optionally substituted by one or more substituents independently selected from halo, trifluoromethyl, difluoromethyl, trifluoromethoxy, difluoromethoxy, cyano, nitro, (1-4C)alkyl, NRᵢRⱼ, ORᵢ, C(O)Rⱼ, C(O)ORⱼ, OC(O)Rⱼ, N(Rⱼ)ORᵢ, C(O)N(Rⱼ)Rᵢ, N(Rⱼ)C(O)Rᵢ, S(O)_{q}Rᵢ (where q is 0, 1 or 2), SO₂N(Rⱼ)Rᵢ, or N(Rⱼ)SO₂Rᵢ, wherein Rᵢ and Rⱼ are each independently selected from H or (1-4C)alkyl;
optionally with the proviso that said compound is not one of the following:
N2-(2-methoxy-4-(1-methyl-1H-pyrazol-4-yl)phenyl)-6-methyl-N8-neopentylpyrido[3,4-d]pyrimidine-2,8-diamine;
N2-(4-(1,2-dimethyl-1H-imidazol-5-yl)-2-methoxyphenyl)-N8-(2-methoxy-2-methylpropyl)-6-methylpyrido[3,4-d]pyrimidine-2,8-diamine;
N2-(4-(1,2-dimethyl-1H-imidazol-5-yl)-2-methoxyphenyl)-6-methyl-N8-neopentylpyrido[3,4-d]pyrimidine-2,8-diamine;
N2-(2-methoxy-4-(4-methyl-4H-1,2,4-triazol-3-yl)phenyl)-6-methyl-N8-neopentylpyrido[3,4-d]pyrimidine-2,8-diamine;
N8-(2-methoxy-2-methylpropyl)-N2-(2-methoxy-4-(4-methyl-4H-1,2,4-triazol-3-yl)phenyl)-6-methylpyrido[3,4-d]pyrimidine-2,8-diamine;
N2-(2-methoxy-4-(1-(2-methoxyethyl)-2-methyl-1H-imidazol-5-yl)phenyl)-6-methyl-N8-neopentylpyrido[3,4-d]pyrimidine-2,8-diamine;
N2-(2-ethoxy-4-(4-methyl-4H-1,2,4-triazol-3-yl)phenyl)-6-methyl-N8-neopentylpyrido[3,4-d]pyrimidine-2,8-diamine;
N2-(2-methoxy-4-(4-(methylsulfonyl)piperazin-1-yl)phenyl-6-methyl-N8-neopentylpyrido[3,4-d]pyrimidine-2,8-diamine;
N2-(4-(1,3-dimethyl-1H-pyrazol-4-yl)-2-methoxyphenyl)-N8-(2-methoxy-2-methylpropyl)-6-methylpyrido[3,4-d]pyrimidine-2,8-diamine;
N-(2-ethoxy-4-(4-methyl-4H-1,2,4-triazol-3-yl)phenyl)-6-methyl-8-(6-oxa-2-azaspiro[3.4]octan-2-yl)pyrido[3,4-d]pyrimidin-2-amine;
N8-(2-methoxy-2-methylpropyl)-N2-(2-methoxy-4-(1-methyl-1H-1,2,4-triazol-5-yl)phenyl)-6-methylpyrido[3,4-d]pyrimidine-2,8-diamine;
N2-(2-(difluoromethoxy)-4-(1-methyl-1H-pyrazol-4-yl)phenyl)-N8-(2-methoxy-2-methylpropyl)-6-methylpyrido[3,4-d]pyrimidine-2,8-diamine;
(4-(3-methoxy-4-((8-((2-methoxy-2-methylpropyl)amino)-6-methylpyrido[3,4-d]pyrimidin-2-yl)amino)phenyl)-1-methyl-1H-pyrazol-5-yl)methanol;

wherein formula III is

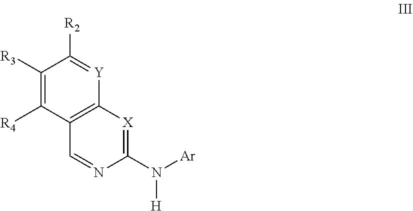

wherein:
X is CH or N;
Y is N or C—H;
R₂ is selected from (1-6C)alkyl, (1-8C)heteroalkyl, aryl, aryl(1-2C)alkyl, a 5 or 6 membered heteroaryl, a 5 or 6 membered heteroaryl(1-2C)alkyl, a 3 to 6 membered heterocyclyl, a 3 to 6 membered heterocyclyl(1-2C)alkyl, (3-8C)cycloalkyl, (3-8C)cycloalkyl(1-2C)alkyl, NR₁₁R₁₂, OR₁₃, C(O)R₁₃, C(O)OR₁₃, OC(O)R₁₃, N(R₁₄)OR₁₃, N(R₁₄)C(O)OR₁₃, C(O)N(R₁₄)R₁₃, N(R₁₄)C(O)R₁₃, S(O)ₓR₁₃ (where x is 0, 1 or 2), SO₂N(R₁₄)R₁₃, or N(R₁₄)SO₂R₁₃;

and wherein R₂ is optionally substituted by one or more substituent groups selected from fluoro, chloro, trifluoromethyl, trifluoromethoxy, cyano, nitro, hydroxy, amino, carboxy, carbamoyl, sulphamoyl, (1-4C)alkyl, (1-4C)alkoxy, S(O),CH₃ (where x is 0, 1 or 2), methylamino or dimethylamino, aryl, aryl(1-2C)alkyl, heteroaryl, heteroaryl(1-2C)alkyl, heterocyclyl, heterocyclyl(1-2C)alkyl, (3-8C)cycloalkyl, or (3-8C)cycloalkyl(1-2C)alkyl,

and wherein any (1-4C)alkyl, (1-4C)alkoxy, aryl, heteroaryl, heterocyclyl, or (3-8C)cycloalkyl moiety present within a substituent group on R₂ is optionally further substituted by fluoro, chloro, trifluoromethyl, trifluoromethoxy, cyano, nitro, hydroxy, amino, carboxy, carbamoyl, sulphamoyl, (1-4C)alkyl, NR_{c}R_{d}, OR_{c}, C(O)R_{c}, C(O)OR_{c}, OC(O)R_{c}, N(R_{d})OR_{c}, C(O)N(R_{d})R_{c}, N(R_{d})C(O)R_{c}, S(O)_{y}R_{c} (where y is 0, 1 or 2), SO₂N(R_{d})R_{c}, or N(R_{d})SO₂R_{c}, wherein R_{c} and R_{d} are each independently selected from H or (1-4C)alkyl;
R₃ is hydrogen, (1-4C)alkyl, (3-6C)cycloalkyl, halo, CF₃, CN and (1-4C)alkoxy;
R₄ is hydrogen, (1-3C)alkyl, fluoro, chloro or CF₃;
Ar has the formula:

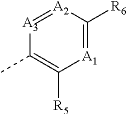

wherein:

(i) all of A₁, A₂ and A₃ are CH; or

(ii) A₃ is CH and A₁ or A₂ are selected from N or CH;

R₅ is hydrogen, cyano, (1-3C)alkyl, (1-3C)fluoroalkyl, (1-3C)alkoxy, (1-3C)fluoroalkoxy, halo, (1-3C)alkanoyl, C(O)NR₁₅R₁₆ or S(O)₂NR₁₅R₁₆, and wherein R₁₅ and R₁₆ are each independently selected from H or (1-3C)alkyl, and wherein any alkyl or alkoxy moities present within a R₅ substituent group are optionally further substituted by hydroxy or methoxy;

R₆ is halogeno, trifluoromethyl, trifluoromethoxy, cyano, nitro, hydroxy, amino, carboxy, carbamoyl, sulphamoyl, ureido, (1-6C)alkyl, (2-6C)alkenyl, (2-6C)alkynyl, or R₆ is a group of the formula:

-L¹-L²-R¹⁷
wherein
   L¹ is absent or a linker group of the formula —[CR₁₈R₁₉]ₙ— in which n is an integer selected from 1, 2, 3 or 4, and R₁₈ and R₁₉ are each independently selected from hydrogen or (1-2C)alkyl;
   L² is absent or is selected from O, S, SO, SO₂, N(R₂₀), C(O), C(O)O, OC(O), CH(OR₂₀), C(O)N(R₂₀), N(R₂₀)C(O), N(R₂₀)C(O)N(R₂₁), S(O)₂N(R₂₀), or N(R₂₁)SO₂, wherein R₂₀ and R₂₁ are each independently selected from hydrogen or (1-2C)alkyl; and
   R₁₇ is (1-6C)alkyl, aryl, aryl-(1-6C)alkyl, (3-6C)cycloalkyl, (3-6C)cycloalkyl-(1-4C)alkyl, heteroaryl, heteroaryl-(1-4C)alkyl, heterocyclyl, heterocyclyl-(1-4C)alkyl,
      and wherein R₁₇ is optionally further substituted by one or more substituent groups independently selected from oxo, halo, cyano, nitro, hydroxy, NR₂₂R₂₃, (1-4C)alkoxy, (1-4C)alkyl, (3-8C)cycloalkyl, (3-8C)cycloalkyl-(1-3C)alkyl, (1-5C)alkanoyl, (1-5C)alkylsulphonyl, heterocyclyl, heterocyclyl-(1-2C)alkyl, heteroaryl, heteroaryl-(1-2C)alkyl, CONR₂₂R₂₃, and SO₂NR₂₂R₂; wherein R₂₂ and R₂₃ are each independently selected from hydrogen, (1-4C)alkyl or (3-6C)cycloalkyl or (3-6C)cycloalkyl(1-2C)alkyl; or R₂₂ and R₂₃ can be linked such that, together with the nitrogen atom to which they are attached, they form a 4-6 membered heterocyclic ring ring;
      and wherein when said substituent group comprises an alkyl, cycloalkyl, heterocyclyl or heteroaryl moiety then said moiety is optionally further substituted by hydroxy, fluoro, chloro, cyano, CF₃, OCF₃, (1-2C)alkyl, (1-2C)alkoxy, SO₂(1-2C)alkyl or NRₑR_{f} (where Rₑ and R_{f} are each independently selected from hydrogen, (1-3C)alkyl, (3-6C)cycloalkyl, or (3-6C)cycloalkyl(1-2C)alkyl);
   or R₇ is a group having the formula:

-L³-L⁴-R₂₄
wherein
L³ is absent or a linker group of the formula —[CR₂₅R₂₆]ₙ— in which n is an integer selected from 1, 2, 3 or 4, and R₂₅ and R₂₆ are each independently selected from hydrogen or (1-2C)alkyl;
L⁴ is absent or is selected from O, S, SO, SO₂, N(R₂₇), C(O), C(O)O, OC(O), CH(OR₂₇), C(O)N(R₂₇), N(R₂₇)C(O), N(R₂₇)C(O)N(R₂₈), S(O)₂N(R₂₇), or N(R₂₈)SO₂, wherein R₂₇ and R₂₈ are each independently selected from hydrogen or (1-2C)alkyl; and
R₂₄ is (1-6C)alkyl, aryl, aryl-(1-6C)alkyl, (3-6C)cycloalkyl, (3-6C)cycloalkyl-(1-4C)alkyl, heteroaryl, heteroaryl-(1-4C)alkyl, heterocyclyl, heterocyclyl-(1-4C)alkyl;
R₁₂ is selected from hydrogen, (1-6C)alkyl, (1-6C)alkoxy, (3-6C)cycloalkyl, (3-6C)cycloalkyl-(1-2C)alkyl, aryl, aryl-(1-2C)alkyl, heterocyclyl, heterocyclyl-(1-2C)alkyl, heteroaryl, heteroaryl-(1-2C)alkyl, and wherein R₁₂ is optionally further substituted by one or more substituents selected from hydroxy, fluoro, chloro, cyano, CF₃, OCF₃ (1-2C)alkyl or (1-2C)alkoxy;
R₁₃ is selected from hydrogen, (1-6C)alkyl, (1-6C)alkoxy, (3-6C)cycloalkyl, (3-6C)cycloalkyl-(1-2C)alkyl, aryl, aryl-(1-2C)alkyl, heteroaryl, heteroaryl-(1-2C)alkyl, and wherein R₁₃ is optionally further substituted by one or more substituents selected from hydroxy, fluoro, chloro, cyano, CF₃, OCF₃ (1-2C)alkyl or (1-2C)alkoxy;
R₁₁ and R₁₄ are independently selected from hydrogen, (1-6C)alkyl, (3-6C)cycloalkyl, (3-6C)cycloalkyl-(1-2C)alkyl, and wherein R₁₁ and R₁₄ are optionally further substituted by one or more substituents selected from hydroxy, fluoro, chloro, cyano, CF₃, OCF₃, (1-2C)alkyl or (1-2C)alkoxy;
optionally subject to the proviso that:

X can only be N when Y is N; and

when X and Y are both N, R₃ is selected from H or fluoro and R₂ is not a NR₁₁R₁₂ group;

wherein formula IV is:

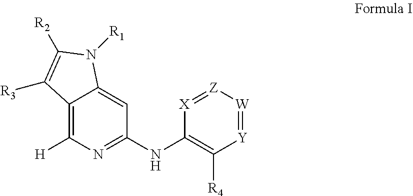

wherein:

R₁ is hydrogen, (1-5C)alkyl, (1-5C)fluoroalkyl, (3-8C)cycloalkyl, (3-8C)cycloalkyl-(1-4C)alkyl, aryl, aryl-(1-4C)alkyl, heteroaryl, heteroaryl-(1-4C)alkyl, —S(O)₂—R^{a},

—C(O)—R^{a}, or —C(O)—O—R^{a}, wherein R^{a} is (1-5C)alkyl, (3-8C)cycloalkyl, (3-8C)cycloalkyl-(1-4C)alkyl, aryl, aryl-(1-4C)alkyl, heteroaryl or heteroaryl-(1-4C)alkyl, and wherein any (1-5C)alkyl, (3-8C)cycloalkyl, (3-8C)cycloalkyl-(1-4C)alkyl, aryl, aryl-(1-4C)alkyl, heteroaryl, heteroaryl-(1-4C)alkyl group present in a Ri substituent group is optionally substituted by methyl, trifluoromethyl, methoxy, trifluoromethoxy, halo, cyano, nitro, hydroxy, mercapto, amino, carboxy, carbamoyl, or sulphamoyl;

R₂ is an aryl, aryl(1-2C)alkyl, 5- or 6-membered heteroaryl or a 5- or 6-membered heteroaryl(1-2C)alkyl,
wherein R₂ is optionally substituted by one or more substituents selected from halogeno, trifluoromethyl, trifluoromethoxy, cyano, nitro, hydroxy, mercapto, amino, carboxy, carbamoyl, sulphamoyl,
or a group of the formula:

L-L⁰-R^{b}
wherein
   L is absent or a linker group of the formula —[CR_{g}Rₕ]ₙ— in which n is an integer selected from 1, 2, 3 or 4, and R_{g} and Rₕ are each independently selected from hydrogen or (1-2C)alkyl;
   L⁰ is absent or is selected from O, S, SO, SO₂, N(R^{c}), C(O), C(O)O, OC(O), CH(OR^{c}), C(O)N(R^{c}), N(R^{c})C(O), N(R^{c})C(O)N(R^{d}), SO₂N(R^{c}), or N(R^{c})SO₂, wherein R^{c} and R^{d} are each independently selected from hydrogen or (1-2C)alkyl; and
   R^{b} is (1-4C)alkyl, aryl, aryl-(1-4C)alkyl, (3-6C)cycloalkyl, (3-6C)cycloalkyl-(1-4C)alkyl, heteroaryl, heteroaryl-(1-4C)alkyl, heterocyclyl, or heterocyclyl-(1-4C)alkyl;
   and wherein R^{b} is optionally further substituted by one or more substituents independently selected from oxo, halogeno, cyano, nitro, hydroxy, NR^{e}R^{f}, (1-5C)alkyl, (1-5C)alkoxy, (1-5C)alkanoyl, (1-5C)sulphonyl or aryl; and wherein R^{e} and R^{f} are each independently selected from hydrogen or (1-4C)alkyl or (3-6C)cycloalkyl-(1-4C)alkyl; or R^{e} and R^{f} can be linked such that, together with the nitrogen atom to which they are attached, they form a 4-7 membered heterocyclic, heteroaryl or carbocyclic ring;

R₃ is H, (1-3C)alkyl, halogeno or CF₃;

R₄ is cyano, (1-3C)alkyl, (1-3C)fluoroalkyl, (1-3C)alkoxy, (1-3C)perfluoroalkoxy, halo, (1-3C)alkanoyl, C(O)NRⁱR^{j}, or S(O)₂NRⁱR^{j}; wherein Rⁱ and R^{j} are each independently selected from H or (1-3C)alkyl;

X is CH or CR₅;

W, Y and Z are each independently selected from N, CH, or CR₅;

R₅ is halogeno, trifluoromethyl, trifluoromethoxy, cyano, nitro, hydroxy, mercapto, amino, carboxy, carbamoyl, sulphamoyl, ureido, (1-6C)alkyl, (2-6C)alkenyl, (2-6C)alkynyl,

or R₅ is a group of the formula:

-L¹-L²-R₇
wherein
   L¹ is absent or a linker group of the formula —[CR₈R₉]ₙ— in which n is an integer selected from 1, 2, 3 or 4, and R₈ and R₉ are each independently selected from hydrogen or (1-2C)alkyl;
   L² is absent or is selected from O, S, SO, SO₂, N(R₁₀), C(O), C(O)O, OC(O), CH(OR₁₀), C(O)N(R₁₀), N(R₁₀)C(O), N(R₁₀)C(O)N(R₁₁), S(O)₂N(R₁₀), or N(R₁₃)SO₂, wherein R₁₀ and R₁₁ are each independently selected from hydrogen or (1-2C)alkyl; and
   R₇ is (1-6C)alkyl, aryl, aryl-(1-6C)alkyl, (3-6C)cycloalkyl, (3-6C)cycloalkyl-(1-6C)alkyl, heteroaryl, heteroaryl-(1-6C)alkyl, heterocyclyl, heterocyclyl-(1-6C)alkyl,
   and wherein R₇ is optionally further substituted by one or more substituents independently selected from hydrogen, oxo, halogeno, cyano, nitro, hydroxy, NR₁₂R₁₃, (1-4C)alkoxy, (1-5C)alkyl, (3-8C)cycloalkyl, (3-8C)cycloalkyl-(1-5C)alkyl, aryl, aryl-(1-5C)alkyl, (1-5C)alkanoyl, (1-5C)alkylsulphonyl, heterocyclyl, heterocyclyl-(1-5C)alkyl, heteroaryl, heteroaryl-(1-5C)alkyl, CONR₁₂R₁₃ and SO₂NR₁₂R₁₃;
   R₁₂ and R₁₃ are each independently selected from hydrogen or (1-2C)alkyl; or R₁₂ and R₁₃ can be linked such that, together with the nitrogen atom to which they are attached, they form a 4-7 membered heterocyclic or heteroaryl ring;
or either W and Z, W and Y or Z and X are both CR₅ and the R₅ groups on the adjacent carbon atoms are linked such that, together with the carbon atoms to which they are attached, they form a fused 4-7 membered heterocyclic, heteroaryl or carbocyclic ring.

In one embodiment of each of the aspects and embodiment herein, the MPS1 inhibitor is selected from the group consisting of NMS-P715, S 81694 (NMS-P153), AZ3146, BAY 1217389, BAY 1161909, MPS1-IN-3, MPS1-IN-2 and CFI-402257.

In one embodiment of each of the aspects and embodiment herein, the MPS1 inhibitor is selected from the group consisting of NMS-P715, BAY 1217389 and BAY 1161909.

In one embodiment of each of the aspects and embodiment herein, the MPS1 inhibitor is selected from the group consisting of NMS-P715, S 81694 (NMS-P153), AZ3146, BAY 1217389, BAY 1161909, CFI-402257, CCT289346, a compound of formula I, a compound of formula II, a compound of formula III and a compound of formula IV, or a pharmaceutically acceptable salt or solvate thereof.

In one embodiment of each of the aspects and embodiment herein, the MPS1 inhibitor is selected from the group consisting of NMS-P715, AZ3146, BAY 1217389, BAY 1161909, CFI-402257, CCT289346, a compound of formula I, a compound of formula II, a compound of formula III and a compound of formula IV, or a pharmaceutically acceptable salt or solvate thereof.

In one embodiment of each of the aspects and embodiments herein, the MPS1 inhibitor is selected from the group consisting of NMS-P715, BAY 1217389, BAY 1161909, CCT289346, a compound of formula I, a compound of formula II, a compound of formula III and a compound of formula IV, or pharmaceutically acceptable salts or solvates thereof.

In one embodiment of each of the aspects and embodiments herein, the MPS1 inhibitor is not selected from the group consisting of a compound of formula I, a compound of formula II, a compound of formula III and a compound of formula IV, or pharmaceutically acceptable salts or solvates thereof.

In one embodiment of each of the aspects and embodiments herein, the MPS1 inhibitor is selected from the group consisting of a compound of formula I, a compound of formula II, a compound of formula III and a compound of formula IV, or pharmaceutically acceptable salts or solvates thereof.

In one embodiment of each of the aspects and embodiments herein, the MPS1 inhibitor is selected from the group consisting of NMS-P715, BAY 1217389, BAY 1161909 and CCT289346.

In one embodiment of each of the aspects and embodiments herein, the MPS1 inhibitor is selected from the group consisting of a compound of formula I or a compound of formula II, or pharmaceutically acceptable salts or solvates thereof.

In one embodiment of each of the aspects and embodiments herein, the MPS1 inhibitor is selected from a compound of formula V:

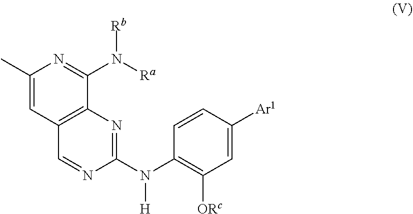

wherein

R^{a} is hydrogen;

R^{b} is C₁₋₆ alkyl, optionally substituted with halogen; or

R^{a} and R^{b} together with the nitrogen to which they are attached from a 4 to 10 membered heterocyclic ring optionally substituted by one or more groups selected from hydrogen, C₁₋₆ alkyl, O—C₁₋₆ alkyl, CN, C₁₋₆ haloalkyl and O—C₁₋₆ haloalkyl;

R^{c} is C₁₋₃ alkyl; and

Ar¹ is a 5- or 6-membered heteroaryl ring optionally substituted with one or more substituents independently selected from C₁₋₆ alkyl, O—C₁₋₆ alkyl, CN, C₁₋₆ haloalkyl and O—C₁₋₆ haloalkyl.

In one embodiment of formula V, R^{b} is C₁₋₆ alkyl, suitably C₅ and C₆ alkyl.

In another embodiment of formula V, R^{a} and R^{b} together with the nitrogen to which they are attached form an azetidinyl group which may optionally be substituted with one or more groups selected from C₁₋₆ alkyl, O—C₁₋₆ alkyl, CN, C₁₋₆ haloalkyl and O—C₁₋₆ haloalkyl, or linked through a spiro carbon atom to a further 4-, 5- or 6-membered carbocyclic or heterocyclic ring to form a spiro bicyclic ring system, which may optionally be substituted with one or more groups selected from hydrogen, C₁₋₆ alkyl, O—C₁₋₆ alkyl, CN, O—C₁₋₆ haloalkyl and O—C₁₋₆ haloalkyl.

In one embodiment of formula V, R^{c} is selected from methyl and ethyl, suitably ethyl.

In one embodiment of formula V, Ar¹ is a 5-membered heteraryl group, suitably 1,2,4-triazole, optionally substituted with one or more substituents independently selected from C₁₋₆ alkyl, O—C₁₋₆ alkyl, CN, C₁₋₆ haloalkyl and O—C₁₋₆ haloalkyl.

In one embodiment of formula V, Ar¹ is a 1,2,4-triazole, substituted with one or more C₁₋₃ alkyl substituents, suitably methyl.

In one embodiment of each of the aspects and embodiments herein, the MPS1 inhibitor is selected from NMS-P715, BAY 1217389, BAY 1161909 and a compound of formula V, or pharmaceutically acceptable salts thereof.

In one embodiment of each of the aspects and embodiments herein, the MPS1 inhibitor is selected from NMS-P715, BAY 1217389, BAY 1161909 and
5-(furan-2-yl)-N-(4-methoxyphenyl)isoquinolin-3-amine;
N-(4-methoxyphenyl)-5-(1-methyl-1H-pyrazol-4-yl)isoquinolin-3-amine;
N-(2-methoxy-4-((1-methylpiperidin-4-yl)oxy)phenyl)-5-(1-methyl-1H-pyrazol-4-yl)isoquinolin-3-amine;
N-(2,4-dimethoxyphenyl)-5-(1-methyl-1H-pyrazol-4-yl)isoquinolin-3-amine;
3-chloro-N,N-dimethyl-4-((5-(1-methyl-1H-pyrazol-4-yl)isoquinolin-3-yl)amino)benzamide;
3-methoxy-N,N-dimethyl-4-((5-(1-methyl-1H-pyrazol-4-yl)isoquinolin-3-yl)amino)benzamide;
(3-methoxy-4-((5-(1-methyl-1H-pyrazol-4-yl)isoquinolin-3-yl)amino)phenyl)(3-methoxyazetidin-1-yl)methanone;
N-(2-chloro-4-(1-methyl-1H-pyrazol-4-yl)phenyl)-5-(1-methyl-1H-pyrazol-4-yl)isoquinolin-3-amine;
(3-chloro-4-((5-(1-methyl-1H-pyrazol-4-yl)isoquinolin-3-yl)amino)phenyl)(3-methoxyazetidin-1-yl)methanone;
(3-methoxy-4-((5-(pyridin-3-yl)isoquinolin-3-yl)amino)phenyl)(3-methoxyazetidin-1-yl)methanone;
N-(4-(3,5-dimethylisoxazol-4-yl)-2-methoxyphenyl)-5-(1-methyl-1H-pyrazol-4-yl)isoquinolin-3-amine;
(3-methoxy-4-((8-(1-methyl-1H-pyrazol-4-yl)pyrido[3,4-d]pyrimidin-2-yl)amino)phenyl)(3-methoxyazetidin-1-yl)methanone;
N-(2-methoxy-4-(1-methyl-1H-pyrazol-4-yl)phenyl)-8-(1-methyl-1H-pyrazol-4-yl)pyrido[3,4-d]pyrimidin-2-amine;
N-(2-chloro-4-(1-methyl-1H-pyrazol-4-yl)phenyl)-8-(1-methyl-1H-pyrazol-4-yl)pyrido[3,4-d]pyrimidin-2-amine;
N-(2-chloro-4-(1-methyl-1H-imidazol-5-yl)phenyl)-5-(1-methyl-1H-pyrazol-4-yl)isoquinolin-3-amine;
N-(2-methoxy-4-(1-methyl-1H-pyrazol-4-yl)phenyl)-5-(1-methyl-1H-pyrazol-4-yl)isoquinolin-3-amine;
(3-methoxy-4-((5-(pyrimidin-5-yl)isoquinolin-3-yl)amino)phenyl)(3-methoxyazetidin-1-yl)methanone;
N-(2-methoxy-4-(1-methyl-1H-imidazol-5-yl)phenyl)-5-(1-methyl-1H-pyrazol-4-yl)isoquinolin-3-amine;
(4-((5-(1,5-dimethyl-1H-pyrazol-4-yl)isoquinolin-3-yl)amino)-3-methoxyphenyl)(3-methoxyazetidin-1-yl)methanone;
(3-methoxy-4-((5-(1-methyl-1H-pyrazol-3-yl)isoquinolin-3-yl)amino)phenyl)(3-methoxyazetidin-1-yl)methanone;
N-(2-chloro-4-(1,2-dimethyl-1H-imidazol-5-yl)phenyl)-5-(1-methyl-1H-pyrazol-4-yl)isoquinolin-3-amine;
N-(4-(1,2-dimethyl-1H-imidazol-5-yl)-2-methoxyphenyl)-5-(1-methyl-1H-pyrazol-4-yl)isoquinolin-3-amine;
N-(2-methoxy-4-(1-methyl-1H-pyrazol-4-yl)phenyl)-8-phenylpyrido[3,4-d]pyrimidin-2-amine;
8-cyclopropyl-N-(2-methoxy-4-(1-methyl-1H-pyrazol-4-yl)phenyl)pyrido[3,4-d]pyrimidin-2-amine;
N-(2-methoxy-5-methyl-4-(1-methyl-1H-pyrazol-4-yl)phenyl)-8-(1-methyl-1H-pyrazol-4-yl)pyrido[3,4-d]pyrimidin-2-amine;
(3-methoxy-4-((5-(1-methyl-1H-pyrazol-5-yl)isoquinolin-3-yl)amino)phenyl)(3-methoxyazetidin-1-yl)methanone;
(4-((5-(1,3-dimethyl-1H-pyrazol-4-yl)isoquinolin-3-yl)amino)-3-methoxyphenyl)(3-methoxyazetidin-1-yl)methanone;
(4-((5-(1-isopropyl-1H-pyrazol-4-yl)isoquinolin-3-yl)amino)-3-methoxyphenyl)(3-methoxyazetidin-1-yl)methanone;
4-((5-(1-methyl-1H-pyrazol-4-yl)isoquinolin-3-yl)amino)-N-(1-methylpiperidin-4-yl)-3-(trifluoromethoxy)benzamide;
(4-((5-(3,5-dimethylisoxazol-4-yl)isoquinolin-3-yl)amino)-3-methoxyphenyl)(3-methoxyazetidin-1-yl)methanone;
(3-methoxy-4-((5-(1-methyl-1H-imidazol-5-yl)isoquinolin-3-yl)amino)phenyl)(3-methoxyazetidin-1-yl)methanone;
N-(2-methoxy-4-(1-methyl-1H-pyrazol-4-yl)phenyl)-8-(pyrrolidin-1-yl)pyrido[3,4-d]pyrimidin-2-amine;
N-(4-(1,2-dimethyl-1H-imidazol-5-yl)-2-methoxyphenyl)-8-(1-methyl-1H-pyrazol-4-yl)pyrido[3,4-d]pyrimidin-2-amine;
tert-butyl 4-(4-(3-((2-methoxy-4-(3-methoxyazetidine-1-carbonyl)phenyl)amino)isoquinolin-5-yl)-1H-pyrazol-1-yl)piperidine-1-carboxylate;
(3-methoxy-4-((5-(1-(piperidin-4-yl)-1H-pyrazol-4-yl)isoquinolin-3-yl)amino)phenyl)(3-methoxyazetidin-1-yl)methanone;
(3-methoxy-4-((5-(1-(1-methylpiperidin-4-yl)-1H-pyrazol-4-yl)isoquinolin-3-yl)amino)phenyl)(3-methoxyazetidin-1-yl)methanone;
(3-methoxy-4-((5-(1-(2-methoxyethyl)-1H-pyrazol-4-yl)isoquinolin-3-yl)amino)phenyl)(3-methoxyazetidin-1-yl)methanone;
N8,N8-diethyl-N2-(2-methoxy-4-(1-methyl-1H-pyrazol-4-yl)phenyl)pyrido[3,4-d]pyrimidine-2,8-diamine;
N8-cyclopentyl-N2-(2-methoxy-4-(1-methyl-1H-pyrazol-4-yl)phenyl)pyrido[3,4-d]pyrimidine-2,8-diamine;
(4-((5-(1-(2-(dimethylamino)ethyl)-1H-pyrazol-4-yl)isoquinolin-3-yl)amino)-3-methoxyphenyl)(3-methoxyazetidin-1-yl)methanone;
N-(2-methoxy-4-(1-methyl-1H-pyrazol-4-yl)phenyl)-5-(1-methyl-1H-pyrazol-4-yl)-2,6-naphthyridin-3-amine;
N-(2-methoxy-4-(1-methyl-1H-pyrazol-4-yl)phenyl)-8-(pi peridin-1-yl)pyrido[3,4-d]pyrimidin-2-amine;
N8-cyclohexyl-N2-(2-methoxy-4-(1-methyl-1H-pyrazol-4-yl)phenyl)pyrido[3,4-d]pyrimidine-2,8-diamine;
N-(2-methoxy-4-(1-methyl-1H-pyrazol-4-yl)phenyl)-8-(3-methylpyrrolidin-1-yl)pyrido[3,4-d]pyrimidin-2-amine;
8-(3,3-difluoropyrrolidin-1-yl)-N-(2-methoxy-4-(1-methyl-1H-pyrazol-4-yl)phenyl)pyrido[3,4-d]pyrimidin-2-amine;
N-(4-(1 ,2-dimethyl-1H-imidazol-5-yl)-2-methoxyphenyl)-5-(1-methyl-1H-pyrazol-4-yl)-2,6-naphthyridin-3-amine;
N8-(cyclopropylmethyl)-N2-(2-methoxy-4-(1-methyl-1H-pyrazol-4-yl)phenyl)pyrido[3,4-d]pyrimidine-2,8-diamine;
8-(1-methyl-1H-pyrazol-4-yl)-N-(2-methyl-4-(1-methyl-1H-pyrazol-4-yl)phenyl)pyrido[3,4-d]pyrimidin-2-amine;
N8-cyclopentyl-N2-(2-methoxy-4-(1-methyl-1H-pyrazol-4-yl)phenyl)-N8-methylpyrido[3,4-d]pyrimidine-2,8-diamine;
N-(2-ethoxy-4-(1-methyl-1H-pyrazol-4-yl)phenyl)-8-(1-methyl-1H-pyrazol-4-yl)pyrido[3,4-d]pyrimidin-2-amine;
N-(2-isopropoxy-4-(1-methyl-1H-pyrazol-4-yl)phenyl)-8-(1-methyl-1H-pyrazol-4-yl)pyrido[3,4-d]pyrimidin-2-amine;
N-(2-(2-methoxyethoxy)-4-(1-methyl-1H-pyrazol-4-yl)phenyl)-8-(1-methyl-1H-pyrazol-4-yl)pyrido[3,4-d]pyrimidin-2-amine;
N8-isopentyl-N2-(2-methoxy-4-(1-methyl-1H-pyrazol-4-yl)phenyl)pyrido[3,4-d]pyrimidine-2,8-diamine;
N-(2-methoxy-4-(1-methyl-1H-pyrazol-4-yl)phenyl)-8-morpholinopyrido[3,4-d]pyrimidin-2-amine;
N-(2-methoxy-4-(1-methyl-1H-pyrazol-4-yl)phenyl)-8-(4-methylpiperazin-1-yl)pyrido[3,4-d]pyrimidin-2-amine;
8-(3,3-difluoroazetidin-1-yl)-N-(2-methoxy-4-(1-methyl-1H-pyrazol-4-yl)phenyl)pyrido[3,4-d]pyrimidin-2-amine;
N-(2-methoxy-4-(1-methyl-1H-pyrazol-4-yl)phenyl)-8-(2-methylpyrrolidin-1-yl)pyrido[3,4-d]pyrimidin-2-amine;
N8-isobutyl-N2-(2-methoxy-4-(1-methyl-1H-pyrazol-4-yl)phenyl)pyrido[3,4-d]pyrimidine-2,8-diamine;
8-(cyclohexylthio)-N-(2-methoxy-4-(1-methyl-1H-pyrazol-4-yl)phenyl)pyrido[3,4-d]pyrimidin-2-amine;
N-(2-methoxy-4-(1-methyl-1H-pyrazol-4-yl)phenyl)-8-(6-azaspiro[3.4]octan-6-yl)pyrido[3,4-d]pyrimidin-2-amine;
N8-cyclohexyl-N2-(2-methoxy-4-(1-(2-(4-methylpiperazin-1-yl)ethyl)-1H-pyrazol-4-yl)phenyl)pyrido[3,4-d]pyrimidine-2,8-diamine;
8-(1-ethyl-1H-pyrazol-4-yl)-N-(2-methoxy-4-(1-methyl-1H-pyrazol-4-yl)phenyl)pyrido[3,4-d]pyrimidin-2-amine;
8-(1-isopropyl-1H-pyrazol-4-yl)-N-(2-methoxy-4-(1-methyl-1H-pyrazol-4-yl)phenyl)pyrido[3,4-d]pyrimidin-2-amine;
N-(2-methoxy-4-(1-methyl-1H-pyrazol-4-yl)phenyl)-8-(3-methoxyazetidin-1-yl)pyrido[3,4-d]pyrimidin-2-amine;
N1-(cyclopropylmethyl)-N7-(2-methoxy-4-(1-methyl-1H-pyrazol-4-yl)phenyl)-2,6-naphthyridine-1,7-diamine;
N1-cyclohexyl-N7-(2-methoxy-4-(1-methyl-1H-pyrazol-4-yl)phenyl)-2,6-naphthyridine-1,7-diamine;
N8-cyclohexyl-N2-(4-(1-(2-(dimethylamino)ethyl)-1H-pyrazol-4-yl)-2-methoxyphenyl)pyrido[3,4-d]pyrimidine-2,8-diamine;
N2-(2-methoxy-4-(1-methyl-1H-pyrazol-4-yl)phenyl)-N8-neopentylpyrido[3,4-d]pyrimidine-2,8-diamine;
N2-(2-methoxy-4-(1-methyl-1H-pyrazol-4-yl)phenyl)-N8-(tetrahydro-2H-pyran-4-yl)pyrido[3,4-d]pyrimidine-2,8-diamine;
N8-(cyclopropylmethyl)-N2-(2-methyl-4-(1-methyl-1H-pyrazol-4-yl)phenyl)pyrido[3,4-d]pyrimidine-2,8-diamine;
N8-cyclohexyl-N2-(2-methyl-4-(1-methyl-1H-pyrazol-4-yl)phenyl)pyrido[3,4-d]pyrimidine-2,8-diamine;
N8-(cyclopropylmethyl)-N2-(2-ethoxy-4-(1-methyl-1H-pyrazol-4-yl)phenyl)pyrido[3,4-d]pyrimidine-2,8-diamine;
N8-(cyclohexylmethyl)-N2-(2-methoxy-4-(1-methyl-1H-pyrazol-4-yl)phenyl)pyrido[3,4-d]pyrimidine-2,8-diamine;
2-(4-(4-((8-(cyclohexylamino)pyrido[3,4-d]pyrimidin-2-yl)amino)-3-methoxyphenyl)-1H-pyrazol-1-yl)ethanol;
8-(cyclopropylmethoxy)-N-(2-methoxy-4-(1-methyl-1H-pyrazol-4-yl)phenyl)pyrido[3,4-d]pyrimidin-2-amine;
1-((2-((2-methoxy-4-(1-methyl-1H-pyrazol-4-yl)phenyl)amino)pyrido[3,4-d]pyrimidin-8-yl)amino)-2-methylpropan-2-ol;
N2-(2-methoxy-4-(1-methyl-1H-pyrazol-4-yl)phenyl)-N8-(oxetan-3-ylmethyl)pyrido[3,4-d]pyrimidine-2,8-diamine;
N8-(3,3-dimethylbutan-2-yl)-N2-(2-methoxy-4-(1-methyl-1H-pyrazol-4-yl)phenyl)pyrido[3,4-d]pyrimidine-2,8-diamine;
3-((2-((2-methoxy-4-(1-methyl-1H-pyrazol-4-yl)phenyl)amino)pyrido[3,4-d]pyrimidin-8-yl)amino)-2,2-dimethylpropan-1-ol;
N2-(2-ethoxy-4-(1-methyl-1H-pyrazol-4-yl)phenyl)-N8-(tetrahydro-2H-pyran-4-yl)pyrido[3,4-d]pyrimidine-2,8-diamine;
N2-(2-methoxy-6-morpholinopyridin-3-yl)-N8-neopentylpyrido[3,4-d]pyrimidine-2,8-diamine;
N2-(2-methoxy-6-(methylsulfonyl)pyridin-3-yl)-N8-neopentylpyrido[3,4-d]pyrimidine-2,8-diamine;
N2-(2-methoxy-4-(1-methyl-1H-imidazol-5-yl)phenyl)-N8-neopentylpyrido[3,4-d]pyrimidine-2,8-diamine;
N2-(4-(1,3-dimethyl-1H-pyrazol-4-yl)-2-methoxyphenyl)-N8-neopentylpyrido[3,4-d]pyrimidine-2,8-diamine;
N8-(1-cyclopropylethyl)-N2-(2-methoxy-4-(1-methyl-1H-pyrazol-4-yl)phenyl)pyrido[3,4-d]pyrimidine-2,8-diamine;
2-(4-(3-methoxy-4-((8-((tetrahydro-2H-pyran-4-yl)amino)pyrido[3,4-d]pyrimidin-2-yl)amino)phenyl)-1H-pyrazol-1-yl)ethanol;
N2-(4-(1,5-dimethyl-1H-pyrazol-4-yl)-2-methoxyphenyl)-N8-neopentylpyrido[3,4-d]pyrimidine-2,8-diamine;
(R)-N8-(3,3-dimethylbutan-2-yl)-N2-(2-methoxy-4-(1-methyl-1H-pyrazol-4-yl)phenyl)pyrido[3,4-d]pyrimidine-2,8-diamine;
(S)-N8-(3,3-dimethylbutan-2-yl)-N2-(2-methoxy-4-(1-methyl-1H-pyrazol-4-yl)phenyl)pyrido[3,4-d]pyrimidine-2,8-diamine;
N2-(2-methoxy-4-(1-methyl-1H-pyrazol-4-yl)phenyl)-N8-(tetrahydrofuran-3-yl)pyrido[3,4-d]pyrimidine-2,8-diamine;
N2-(2-methoxy-4-(1-methyl-1H-pyrazol-4-yl)phenyl)-N8-((tetrahydrofuran-3-yl)methyl)pyrido[3,4-d]pyrimidine-2,8-diamine;
1-(2-((2-methoxy-4-(1-methyl-1H-pyrazol-4-yl)phenyl)amino)pyrido[3,4-d]pyrimidin-8-yl)pyrrolidin-3-ol;
N2-(2-methoxy-4-(1-methyl-1H-pyrazol-4-yl)phenyl)-N8-methyl-N8-(tetrahydro-2H-pyran-4-yl)pyrido[3,4-d]pyrimidine-2,8-diamine;
N8-(tert-butyl)-N2-(2-methoxy-4-(1-methyl-1H-pyrazol-4-yl)phenyl)pyrido[3,4-d]pyrimidine-2,8-diamine;
N2-(2-methoxy-4-(1-methyl-1H-pyrazol-4-yl)phenyl)-N8-(1-methylcyclohexyl)pyrido[3,4-d]pyrimidine-2,8-diamine;
8-(1-(2,2-difluoroethyl)-1H-pyrazol-4-yl)-N-(2-methoxy-4-(1-methyl-1H-pyrazol-4-yl)phenyl)pyrido[3,4-d]pyrimidin-2-amine;
N2-(4-(1,2-dimethyl-1H-imidazol-5-yl)-2-methoxyphenyl)-N8-neopentylpyrido[3,4-d]pyrimidine-2,8-diamine;
N2-(2-methoxy-4-(1-methyl-1H-pyrazol-4-yl)phenyl)-6-methyl-N8-neopentylpyrido[3,4-d]pyrimidine-2,8-diamine;
N2-(2-methoxy-4-morpholinophenyl)-N8-neopentylpyrido[3,4-d]pyrimidine-2,8-diamine;
N8-(2,2-difluoropropyl)-N2-(2-methoxy-4-(1-methyl-1H-pyrazol-4-yl)phenyl)pyrido[3,4-d]pyrimidine-2,8-diamine;
N8-(3-methoxy-2,2-dimethylpropyl)-N2-(2-methoxy-4-(1-methyl-1H-pyrazol-4-yl)phenyl)pyrido[3,4-d]pyrimidine-2,8-diamine;
N8-(2-methoxy-2-methylpropyl)-N2-(2-methoxy-4-(1-methyl-1H-pyrazol-4-yl)phenyl)pyrido[3,4-d]pyrimidine-2,8-diamine;
N2-(2-methoxy-4-(1-methyl-1H-pyrazol-4-yl)phenyl)-N8-(2,2,2-trifluoroethyl)pyrido[3,4-d]pyrimidine-2,8-diamine;
N-(2-methoxy-4-(1-methyl-1H-pyrazol-4-yl)phenyl)-8-(2-oxa-6-azaspiro[3.4]octan-6-yl)pyrido[3,4-d]pyrimidin-2-amine;
1-(((2-((2-methoxy-4-(1-methyl-1H-pyrazol-4-yl)phenyl)amino)pyrido[3,4-d]pyrimidin-8-yl)amino)methyl)cyclobutanol;
8-chloro-N-(2-methoxy-4-(1-methyl-1H-pyrazol-4-yl)phenyl)pyrido[3,4-d]pyrimidin-2-amine;
N2-(2-ethyl-4-(1-methyl-1H-pyrazol-4-yl)phenyl)-N8-neopentylpyrido[3,4-d]pyrimidine-2,8-diamine;
N2-(4-(1-methyl-1H-pyrazol-4-yl)-2-(trifluoromethoxy)phenyl)-N8-neopentylpyrido[3,4-d]pyrimidine-2,8-diamine;
N2-(2-methoxy-4-(1-methyl-1H-pyrazol-4-yl)phenyl)-N8-methylpyrido[3,4-d]pyrimidine-2,8-diamine;
N2-(2-methoxy-4-(1-methyl-1H-pyrazol-4-yl)phenyl)-N8,N8-dimethylpyrido[3,4-d]pyrimidine-2,8-diamine;
N-(2-((2-methoxy-4-(1-methyl-1H-pyrazol-4-yl)phenyl)amino)pyrido[3,4-d]pyrimidin-8-yl)-2-methylpropane-2-sulfinamide;
N2-(2-methoxy-4-(4-morpholinopiperidin-1-yl)phenyl)-N8-neopentylpyrido[3,4-d]pyrimidine-2,8-diamine;
N2-(2-methoxy-4-(1-methyl-1H-pyrazol-4-yl)phenyl)-N8-((3-methyloxetan-3-yl)methyl)pyrido[3,4-d]pyrimidine-2,8-diamine;
N2-(2-methoxy-4-(piperidin-1-yl)phenyl)-N8-neopentylpyrido[3,4-d]pyrimidine-2,8-diamine;
N-(2-((2-methoxy-4-(1-methyl-1H-pyrazol-4-yl)phenyl)amino)pyrido[3,4-d]pyrimidin-8-yl)-2-methylpropane-2-sulfonamide;
N2-(2-methoxy-4-(1-methyl-1H-pyrazol-4-yl)phenyl)-N8-(oxetan-3-yl)pyrido[3,4-d]pyrimidine-2,8-diamine;
(1-(3-methoxy-4-((8-(neopentylamino)pyrido[3,4-d]pyrimidin-2-yl)amino)phenyl)piperidin-4-yl)(morpholino)methanone;
N2-(2-methoxy-4-(4-methylpiperazin-1-yl)phenyl)-N8-neopentylpyrido[3,4-d]pyrimidine-2,8-diamine;
1-(((2-((2-methoxy-4-(1-methyl-1H-pyrazol-4-yl)phenyl)amino)pyrido[3,4-d]pyrimidin-8-yl)amino)methyl)cyclopropanol;
N2-(2-methoxy-4-(1-methyl-1H-pyrazol-4-yl)phenyl)-N8-(1-methylpiperidin-4-yl)pyrido[3,4-d]pyrimidine-2,8-diamine;
2-((2-((2-methoxy-4-(1-methyl-1H-pyrazol-4-yl)phenyl)amino)pyrido[3,4-d]pyrimidin-8-yl)amino)-2-methylpropan-1-ol;
N-(2-methoxy-4-(1-methyl-1H-pyrazol-4-yl)phenyl)-8-(2-oxa-6-azaspiro[3.3]heptan-6-yl)pyrido[3,4-d]pyrimidin-2-amine;
N2-(2-methoxy-4-(1-methyl-1H-pyrazol-4-yl)phenyl)-N8-(oxetan-2-ylmethyl)pyrido[3,4-d]pyrimidine-2,8-diamine;
N2-(2-chloro-4-morpholinophenyl)-N8-neopentylpyrido[3,4-d]pyrimidine-2,8-diamine
N2-(2-methoxy-4-(4-methyl-4H-1,2,4-triazol-3-yl)phenyl)-N8-neopentylpyrido[3,4-d]pyrimidine-2,8-diamine;
N2-(2-methoxy-4-(4-(methylsulfonyl)piperazin-1-yl)phenyl)-N8-neopentylpyrido[3,4-d]pyrimidine-2,8-diamine;
N2-(2-methoxy-4-(1-methyl-1H-pyrazol-4-yl)phenyl)-N8-((3-methyltetrahydrofuran-3-yl)methyl)pyrido[3,4-d]pyrimidine-2,8-diamine;
N-(4-(1,5-dimethyl-1H-pyrazol-4-yl)-2-methoxyphenyl)-8-(2-oxa-6-azaspiro[3.4]octan-6-yl)pyrido[3,4-d]pyrimidin-2-amine;
N2-(4-(1,5-dimethyl-1H-pyrazol-4-yl)-2-methoxyphenyl)-N8-((3-methyloxetan-3-yl)methyl)pyrido[3,4-d]pyrimidine-2,8-diamine;
N2-(4-(1,5-dimethyl-1H-pyrazol-4-yl)-2-methoxyphenyl)-N8-(2-methoxy-2-methylpropyl)pyrido[3,4-d]pyrimidine-2,8-diamine;
N2-(4-(1,2-dimethyl-1H-imidazol-5-yl)-2-methoxyphenyl)-N8-(2-methoxy-2-methylpropyl)-6-methylpyrido[3,4-d]pyrimidine-2,8-diamine;
N2-(4-(1,2-dimethyl-1H-imidazol-5-yl)-2-methoxyphenyl)-6-methyl-N8-neopentylpyrido[3,4-d]pyrimidine-2,8-diamine;
N2-(2-methoxy-4-(4-methyl-4H-1,2,4-triazol-3-yl)phenyl)-6-methyl-N8-neopentylpyrido[3,4-d]pyrimidine-2,8-diamine;
N-(2-ethoxy-4-(1-methyl-1H-pyrazol-4-yl)phenyl)-8-(2-oxa-6-azaspiro[3.4]octan-6-yl)pyrido[3,4-d]pyrimidin-2-amine;
2-((2-((2-methoxy-4-(1-methyl-1H-pyrazol-4-yl)phenyl)amino)pyrido[3,4-d]pyrimidin-8-yl)amino)ethanol;
N2-(2-methoxy-4-(1-methyl-1H-pyrazol-4-yl)phenyl)-N8-(2-methoxyethyl)pyrido[3,4-d]pyrimidine-2,8-diamine;

1-((2-((2-methoxy-4-(1-methyl-1H-pyrazol-4-yl)phenyl)amino)pyrido[3,4-d]pyrimidin-8-yl)amino)propan-2-ol;
2-((2-((2-methoxy-4-(1-methyl-1H-pyrazol-4-yl)phenyl)amino)pyrido[3,4-d]pyrimidin-8-yl)amino)propan-1-ol;
N2-(4-(1,5-dimethyl-1H-pyrazol-4-yl)-2-methoxyphenyl)-N8-((3-methyltetrahydrofuran-3-yl)methyl)pyrido[3,4-d]pyrimidine-2,8-diamine;
4-(2-((2-methoxy-4-(1-methyl-1H-pyrazol-4-yl)phenyl)amino)pyrido[3,4-d]pyrimidin-8-yl)thiomorpholine 1,1-dioxide;
N-(2-methoxy-4-(1-methyl-1H-pyrazol-4-yl)phenyl)-8-(7-oxa-2-azaspiro[3.5]nonan-2-yl)pyrido[3,4-d]pyrimidin-2-amine;
N2-(2-methoxy-4-(1-methyl-1H-pyrazol-4-yl)phenyl)-5-methyl-N8-neopentylpyrido[3,4-d]pyrimidine-2,8-diamine;
N-(2-methoxy-4-(1-methyl-1H-pyrazol-4-yl)phenyl)-8-(6-oxa-2-azaspiro[3.4]octan-2-yl)pyrido[3,4-d]pyrimidin-2-amine;
1-(2-((2-methoxy-4-(1-methyl-1H-pyrazol-4-yl)phenyl)amino)pyrido[3,4-d]pyrimidin-8-yl)azetidine-3-carbonitrile;
N-(2-methoxy-4-(1-methyl-1H-pyrazol-4-yl)phenyl)-8-(2-oxa-7-azaspiro[4.4]nonan-7-yl)pyrido[3,4-d]pyrimidin-2-amine;
N-(2-methoxy-4-(1-methyl-1H-pyrazol-4-yl)phenyl)-8-(2-oxa-6-azaspiro[3.5]nonan-6-yl)pyrido[3,4-d]pyrimidin-2-amine;
N8-((3-fluorooxetan-3-yl)methyl)-N2-(2-methoxy-4-(1-methyl-1H-pyrazol-4-yl)phenyl)pyrido[3,4-d]pyrimidine-2,8-diamine;
N-(4-chloro-2-methoxyphenyl)-8-(2-oxa-6-azaspiro[3.4]octan-6-yl)pyrido[3,4-d]pyrimidin-2-amine;
N-(2,4-dichlorophenyl)-8-(2-oxa-6-azaspiro[3.4]octan-6-yl)pyrido[3,4-d]pyrimidin-2-amine;
4-((8-(2-oxa-6-azaspiro[3.4]octan-6-yl)pyrido[3,4-d]pyrimidin-2-yl)amino)-3-methoxybenzonitrile;
N-(2-chloro-4-(methylsulfonyl)phenyl)-8-(2-oxa-6-azaspiro[3.4]octan-6-yl)pyrido[3,4-d]pyrimidin-2-amine;
N-(2-chloro-4-(pyrimidin-5-yl)phenyl)-8-(2-oxa-6-azaspiro[3.4]octan-6-yl) pyrido[3,4-d]pyrimidin-2-amine;
N-(2-chloro-4-(5-methyl-1,3,4-oxadiazol-2-yl)phenyl)-8-(2-oxa-6-azaspiro[3.4]octan-6-yl)pyrido[3,4-d]pyrimidin-2-amine;
N8-(2-methoxy-2-methylpropyl)-N2-(2-methoxy-4-(4-methyl-4H-1,2,4-triazol-3-yl)phenyl)-6-methylpyrido[3,4-d]pyrimidine-2,8-diamine;
6-cyclopropyl-N-(2-methoxy-4-(1-methyl-1H-pyrazol-4-yl)phenyl)-8-(2-oxa-6-azaspiro[3.4]octan-6-yl)pyrido[3,4-d]pyrimidin-2-amine;
2-((2-((2-methoxy-4-(1-methyl-1H-pyrazol-4-yl)phenyl)amino)pyrido[3,4-d]pyrimidin-8-yl)amino)propane-1,3-diol;
3-methoxy-2-((2-((2-methoxy-4-(1-methyl-1H-pyrazol-4-yl)phenyl)amino)pyrido[3,4-d]pyrimidin-8-yl)amino)propan-1-ol;
(3-(((2-((2-methoxy-4-(1-methyl-1H-pyrazol-4-yl)phenyl)amino)pyrido[3,4-d]pyrimidin-8-yl)amino)methyl)oxetan-3-yl)methanol;
(S)-N2-(2-methoxy-4-(1-methyl-1H-pyrazol-4-yl)phenyl)-N8-((3-methyltetrahydrofuran-3-yl)methyl)pyrido[3,4-d]pyrimidine-2,8-diamine;
(R)-N2-(2-methoxy-4-(1-methyl-1H-pyrazol-4-yl)phenyl)-N8-((3-methyltetrahydrofuran-3-yl)methyl)pyrido[3,4-d]pyrimidine-2,8-diamine;
N-(4-chloro-2-fluorophenyl)-8-(2-oxa-6-azaspiro[3.4]octan-6-yl) pyrido[3,4-d]pyrimidin-2-amine;
4-((8-(2-oxa-6-azaspiro[3.4]octan-6-yl)pyrido[3,4-d]pyrimidin-2-yl)amino)-3-chlorobenzonitrile;
N2-(2-methoxy-4-(1-(2-methoxyethyl)-2-methyl-1H-imidazol-5-yl)phenyl)-6-methyl-N8-neopentylpyrido[3,4-d]pyrimidine-2,8-diamine;
N2-(2-ethoxy-4-(4-methyl-4H-1,2,4-triazol-3-yl)phenyl)-6-methyl-N8-neopentylpyrido[3,4-d]pyrimidine-2,8-diamine;
N2-(2-methoxy-4-(4-(methylsulfonyl)piperazin-1-yl)phenyl)-6-methyl-N8-neopentylpyrido[3,4-d]pyrimidine-2,8-diamine;
N-(2-methoxy-4-(1-methyl-1H-pyrazol-4-yl)phenyl)-8-(pyridin-4-yl)pyrido[3,4-d]pyrimidin-2-amine;
N8-(2-methoxy-2-methylpropyl)-N2-(2-methoxy-4-(4-methyl-4H-1,2,4-triazol-3-yl)phenyl)-5-methylpyrido[3,4-d]pyrimidine-2,8-diamine;
N2-(2-methoxy-4-(4-methyl-4H-1,2,4-triazol-3-yl)phenyl)-5-methyl-N8-neopentylpyrido[3,4-d]pyrimidine-2,8-diamine;
N-(2-methoxy-4-(1-methyl-1H-pyrazol-4-yl)phenyl)-8-(2-methylmorpholino)pyrido[3,4-d]pyrimidin-2-amine;
(4-(3-methoxy-4-((8-((2-methoxy-2-methylpropyl)amino)pyrido[3,4-d]pyrimidin-2-yl)amino)phenyl)-1-methyl-1H-pyrazol-5-yl)methanol;
(4-(3-methoxy-4-((8-(((3-methyltetrahydrofuran-3-yl)methyl)amino)pyrido[3,4-d]pyrimidin-2-yl)amino)phenyl)-1-methyl-1H-pyrazol-5-yl)methanol;
N8-(2-methoxy-2-methylpropyl)-N2-(2-methoxy-4-(1-(2-methoxyethyl)-2-methyl-1H-imidazol-5-yl)phenyl)pyrido[3,4-d]pyrimidine-2,8-diamine;
N2-(2-methoxy-4-(1-(2-methoxyethyl)-2-methyl-1H-imidazol-5-yl)phenyl)-N8-((3-methyltetrahydrofuran-3-yl)methyl)pyrido[3,4-d]pyrimidine-2,8-diamine;
N2-(2-ethoxy-4-(4-methyl-4H-1,2,4-triazol-3-yl)phenyl)-N8-(2-methoxy-2-methylpropyl)pyrido[3,4-d]pyrimidine-2,8-diamine;
N2-(2-ethoxy-4-(4-methyl-4H-1,2,4-triazol-3-yl)phenyl)-N8-((3-methyltetrahydrofuran-3-yl)methyl)pyrido[3,4-d]pyrimidine-2,8-diamine;
8-(3,6-dihydro-2H-pyran-4-yl)-N-(2-methoxy-4-(1-methyl-1H-pyrazol-4-yl)phenyl)pyrido[3,4-d]pyrimidin-2-amine;
N8-(2-methoxy-2-methylpropyl)-N2-(2-methoxy-6-(1-methyl-1H-tetrazol-5-yl)pyridin-3-yl)pyrido[3,4-d]pyrimidine-2,8-diamine;
N2-(6-(1,2-dimethyl-1H-imidazol-5-yl)-2-methoxypyridin-3-yl)-N8-(2-methoxy-2-methylpropyl)pyrido[3,4-d]pyrimidine-2,8-diamine;
N8-(2-methoxy-2-methylpropyl)-N2-(2-methoxy-4-(1-methyl-1H-tetrazol-5-yl)phenyl)pyrido[3,4-d]pyrimidine-2,8-diamine;
N-(2-methoxy-4-(1-methyl-1H-pyrazol-4-yl)phenyl)-8-(pyrimidin-5-yl)pyrido[3,4-d]pyrimidin-2-amine;
N2-(2-methoxy-4-(1-methyl-1H-pyrazol-4-yl)phenyl)-N8-(1-(tetrahydrofuran-3-yl)ethyl)pyrido[3,4-d]pyrimidine-2,8-diamine;
N2-(4-(1,3-dimethyl-1H-pyrazol-4-yl)-2-methoxyphenyl)-N8-(2-methoxy-2-methylpropyl)pyrido[3,4-d]pyrimidine-2,8-diamine;
N2-(4-(1,3-dimethyl-1H-pyrazol-4-yl)-2-methoxyphenyl)-N8-((3-methyltetrahydrofuran-3-yl)methyl)pyrido[3,4-d]pyrimidine-2,8-diamine;
N-(2-methoxy-4-(1-methyl-1H-pyrazol-4-yl)phenyl)-8-(4-methoxypiperidin-1-yl)pyrido[3,4-d]pyrimidin-2-amine;
1-(2-((2-methoxy-4-(1-methyl-1H-pyrazol-4-yl)phenyl)amino)pyrido[3,4-d]pyrimidin-8-yl)piperidine-4-carbonitrile;
N-(2-methoxy-4-(1-methyl-1H-pyrazol-4-yl)phenyl)-8-(4-(methylsulfonyl)piperazin-1-yl)pyrido[3,4-d]pyrimidin-2-amine;
N2-(4-(1,3-dimethyl-1H-pyrazol-4-yl)-2-methoxyphenyl)-N8-(2-methoxy-2-methylpropyl)-6-methylpyrido[3,4-d]pyrimidine-2,8-diamine;
N-(2-ethoxy-4-(4-methyl-4H-1,2,4-triazol-3-yl)phenyl)-6-methyl-8-(6-oxa-2-azaspiro[3.4]octan-2-yl)pyrido[3,4-d]pyrimidin-2-amine;
N2-(6-(1,3-dimethyl-1H-pyrazol-4-yl)-2-methoxypyridin-3-yl)-N8-(2-methoxy-2-methylpropyl)pyrido[3,4-d]pyrimidine-2,8-diamine;
N2-(6-(1,5-dimethyl-1H-pyrazol-4-yl)-2-methoxypyridin-3-yl)-N8-(2-methoxy-2-methylpropyl)pyrido[3,4-d]pyrimidine-2,8-diamine;
N8-(2-methoxy-2-methylpropyl)-N2-(2-methoxy-6-(1-methyl-1H-1,2,3-triazol-5-yl)pyridin-3-yl)pyrido[3,4-d]pyrimidine-2,8-diamine;
N8-(2-methoxy-2-methylpropyl)-N2-(2-methoxy-6-(2-methyl-2H-1,2,3-triazol-4-yl)pyridin-3-yl)pyrido[3,4-d]pyrimidine-2,8-diamine;
(3-methoxy-4-((8-((2-methoxy-2-methylpropyl)amino)pyrido[3,4-d]pyrimidin-2-yl)amino)phenyl)(3-methoxyazetidin-1-yl)methanone;
3-methoxy-4-((8-((2-methoxy-2-methylpropyl)amino)pyrido[3,4-d]pyrimidin-2-yl)amino)-N,N-dimethylbenzamide;
(3-methoxy-4-((8-((2-methoxy-2-methylpropyl)amino)pyrido[3,4-d]pyrimidin-2-yl)amino)phenyl)(4-methylpiperazin-1-yl)methanone;
(1-(2-((2-methoxy-4-(1-methyl-1H-pyrazol-4-yl)phenyl)amino)pyrido[3,4-d]pyrimidin-8-yl)pyrrolidin-3-yl)methanol;
(1-(2-((2-methoxy-4-(1-methyl-1H-pyrazol-4-yl)phenyl)amino)pyrido[3,4-d]pyrimidin-8-yl)piperidin-3-yl)methanol;
(4-(2-((2-methoxy-4-(1-methyl-1H-pyrazol-4-yl)phenyl)amino)pyrido[3,4-d]pyrimidin-8-yl)morpholin-2-yl)methanol;
N2-(2-(difluoromethoxy)-4-(1-methyl-1H-pyrazol-4-yl)phenyl)-N8-(2-methoxy-2-methylpropyl)pyrido[3,4-d]pyrimidine-2,8-diamine;
N2-(2-(difluoromethoxy)-4-fluorophenyl)-N8-(2-methoxy-2-methylpropyl)pyrido[3,4-d]pyrimidine-2,8-diamine;
N2-(4-(1-ethyl-1H-pyrazol-4-yl)-2-methoxyphenyl)-N8-(2-methoxy-2-methylpropyl)pyrido[3,4-d]pyrimidine-2,8-diamine;
(3-methoxy-4-((8-(neopentylamino)pyrido[3,4-d]pyrimidin-2-yl)amino)phenyl)(3-methoxyazetidin-1-yl)methanone;
N2-(2-methoxy-4-(tetrahydro-2H-pyran-4-yl)phenyl)-N8-((3-methyltetrahydrofuran-3-yl)methyl)pyrido[3,4-d]pyrimidine-2,8-diamine;
N2-(4-chloro-2-(difluoromethoxy)phenyl)-N8-(2-methoxy-2-methylpropyl)pyrido[3,4-d]pyrimidine-2,8-diamine;
N2-(2-methoxy-4-(4-methyl-4H-1,2,4-triazol-3-yl)phenyl)-5-methyl-N8-((3-methyloxetan-3-yl)methyl)pyrido[3,4-d]pyrimidine-2,8-diamine;
N2-(2-methoxy-4-(4-methyl-4H-1,2,4-triazol-3-yl)phenyl)-5-methyl-N8-((3-methyltetrahydrofuran-3-yl)methyl)pyrido[3,4-d]pyrimidine-2,8-diamine;
N-(2-methoxy-4-(4-methyl-4H-1,2,4-triazol-3-yl)phenyl)-5-methyl-8-(6-oxa-2-azaspiro[3.4]octan-2-yl)pyrido[3,4-d]pyrimidin-2-amine;
N8-(2-methoxy-2-methylpropyl)-N2-(2-methoxy-4-(1-methyl-1H-1,2,4-triazol-3-yl)phenyl)-6-methylpyrido[3,4-d]pyrimidine-2,8-diamine;
N2-(2-(difluoromethoxy)-4-(1-methyl-1H-pyrazol-4-yl)phenyl)-N8-(2-methoxy-2-methylpropyl)-6-methylpyrido[3,4-d]pyrimidine-2,8-diamine;
(4-(3-methoxy-4-((84(2-methoxy-2-methylpropyl)amino)-6-methylpyrido[3,4-d]pyrimidin-2-yl)amino)phenyl)-1-methyl-1H-pyrazol-5-yl)methanol;
N2-(2-methoxy-4-(1-methyl-1H-1,2,3-triazol-5-yl)phenyl)-6-methyl-N8-neopentylpyrido[3,4-d]pyrimidine-2,8-diamine;
N2-(2-ethoxy-4-(4-methyl-4H-1,2,4-triazol-3-yl)phenyl)-6-methyl-N8-((3-methyltetrahydrofuran-3-yl)methyl)pyrido[3,4-d]pyrimidine-2,8-diamine;
1-(((2-((2-ethoxy-4-(4-methyl-4H-1,2,4-triazol-3-yl)phenyl)amino)-6-methylpyrido[3,4-d]pyrimidin-8-yl)amino)methyl)cyclobutanol;
1-(2-((2-ethoxy-4-(4-methyl-4H-1,2,4-triazol-3-yl)phenyl)amino)-6-methylpyrido[3,4-d]pyrimidin-8-yl)-3-methylazetidine-3-carbonitrile;
N-(2-ethoxy-4-(4-methyl-4H-1,2,4-triazol-3-yl)phenyl)-6-methyl-8-(7-oxa-2-azaspiro[3.5]nonan-2-yl)pyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)piperidine-4-carbonitrile;
1-(2-((2-ethoxy-4-(4-methyl-4H-1,2,4-triazol-3-yl)phenyl)amino)-6-methylpyrido[3,4-d]pyrimidin-8-yl)-3-methylazetidin-3-ol;
N2-(2-ethoxy-4-(4-methyl-4H-1,2,4-triazol-3-yl)phenyl)-6-methyl-N8-((3-methyloxetan-3-yl)methyl)pyrido[3,4-d]pyrimidine-2,8-diamine;
N2-(2-ethoxy-4-(4-methyl-4H-1,2,4-triazol-3-yl)phenyl)-6-methyl-N8-(tetrahydro-2H-pyran-4-yl)pyrido[3,4-d]pyrimidine-2,8-diamine;
N-(2-ethoxy-4-(4-methyl-4H-1,2,4-triazol-3-yl)phenyl)-6-methyl-8-(2-oxa-7-azaspiro[4.4]nonan-7-yl)pyrido[3,4-d]pyrimidin-2-amine;
1-(((2-((2-ethoxy-4-(4-methyl-4H-1,2,4-triazol-3-yl)phenyl)amino)-6-methylpyrido[3,4-d]pyrimidin-8-yl)amino)methyl)cyclopropanol;
N2-(4-(1,2-dimethyl-1H-imidazol-5-yl)-2-methoxyphenyl)-6-methyl-N8-((3-methyltetrahydrofuran-3-yl)methyl)pyrido[3,4-d]pyrimidine-2,8-diamine;
N-(4-(1,2-dimethyl-1H-imidazol-5-yl)-2-methoxyphenyl)-6-methyl-8-(2-oxa-7-azaspiro[4.4]nonan-7-yl)pyrido[3,4-d]pyrimidin-2-amine;
1-(2-((4-(1,2-dimethyl-1H-imidazol-5-yl)-2-methoxyphenyl)amino)-6-methylpyrido[3,4-d]pyrimidin-8-yl)piperidine-4-carbonitrile;
1-(2-((4-(1,2-dimethyl-1H-imidazol-5-yl)-2-methoxyphenyl)amino)-6-methylpyrido[3,4-d]pyrimidin-8-yl)-3-methylazetidine-3-carbonitrile;
N-(2-ethoxy-4-(4-methyl-4H-1,2,4-triazol-3-yl)phenyl)-6-methyl-8-(1-oxa-6-azaspiro[3.3]heptan-6-yl)pyrido[3,4-d]pyrimidin-2-amine;
N2-(2-methoxy-4-(1-methyl-1H-pyrazol-3-yl)phenyl)-6-methyl-N8-neopentylpyrido[3,4-d]pyrimidine-2,8-diamine;
8-(3,3-difluoroazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(4-methoxypiperidin-1-yl)-6-methylpyrido[3,4-d]pyrimidin-2-amine;
N-(2-ethoxy-4-(4-methyl-4H-1,2,4-triazol-3-yl)phenyl)-8-(3-methoxypiperidin-1-yl)-6-methylpyrido[3,4-d]pyrimidin-2-amine;
N-(2-ethoxy-4-(4-methyl-4H-1,2,4-triazol-3-yl)phenyl)-6-methyl-8-(2-methylmorpholino)pyrido[3,4-d]pyrimidin-2-amine;
N-(2-ethoxy-4-(4-methyl-4H-1,2,4-triazol-3-yl)phenyl)-8-(4-methoxy-4-methylpiperidin-1-yl)-6-methylpyrido[3,4-d]pyrimidin-2-amine;
8-(3-azabicyclo[3.1.0]hexan-3-yl)-N-(2-ethoxy-4-(4-methyl-4H-1,2,4-triazol-3-yl)phenyl)-6-methylpyrido[3,4-d]pyrimidin-2-amine;
8-(3-(dimethylamino)azetidin-1-yl)-N-(2-ethoxy-4-(4-methyl-4H-1,2,4-triazol-3-yl)phenyl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)piperidin-4-ol;
N-(2-ethoxy-4-(4-methyl-4H-1,2,4-triazol-3-yl)phenyl)-8-(3-methoxy-3-methylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylpyrrolidin-3-ol;
1-(2-((2-ethoxy-4-(4-methyl-4H-1,2,4-triazol-3-yl)phenyl)amino)-6-methylpyrido[3,4-d]pyrimidin-8-yl)-4-methylpiperidine-4-carbonitrile;
1-(2-((2-ethoxy-4-(4-methyl-4H-1,2,4-triazol-3-yl)phenyl)amino)-6-methylpyrido[3,4-d]pyrimidin-8-yl)pyrrolidine-3-carbonitrile;
N2-(2-methoxy-4-(1-methyl-1H-imidazol-2-yl)phenyl)-6-methyl-N8-neopentylpyrido[3,4-d]pyrimidine-2,8-diamine;
N2-(2-methoxy-4-(1-methyl-1H-pyrazol-5-yl)phenyl)-6-methyl-N8-neopentylpyrido[3,4-d]pyrimidine-2,8-diamine;
N2-(2-methoxy-4-(oxazol-2-yl) phenyl)-6-methyl-N8-neopentylpyrido[3,4-d]pyrimidine-2,8-diamine;
N-(2-ethoxy-4-(4-methyl-4H-1,2,4-triazol-3-yl)phenyl)-8-(3-methoxypyrrolidin-1-yl)-6-methylpyrido[3,4-d]pyrimidin-2-amine;
8-(3,3-dimethylazetidin-1-yl)-N-(2-ethoxy-4-(4-methyl-4H-1,2,4-triazol-3-yl)phenyl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylpyrrolidine-3-carbonitrile;
8-(2,2-dimethylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-6-methyl-8-(3-(trifluoromethyl)azetidin-1-yl)pyrido[3,4-d]pyrimidin-2-amine;
N-(2-ethoxy-4-(4-methyl-4H-1,2,4-triazol-3-yl)phenyl)-6-methyl-8-(2-azaspiro[3.3]heptan-2-yl)pyrido[3,4-d]pyrimidin-2-amine;
(R)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
(S)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
N2-(2-ethoxy-4-(4-methyl-4H-1,2,4-triazol-3-yl)phenyl)-N8-((1-methoxycyclobutyl)methyl)-6-methylpyrido[3,4-d]pyrimidine-2,8-diamine;
N2-(2-ethoxy-4-(4-methyl-4H-1,2,4-triazol-3-yl)phenyl)-6-methyl-N8-(1-methylazetidin-3-yl)pyrido[3,4-d]pyrimidine-2,8-diamine;
N2-(2-ethoxy-4-(4-methyl-4H-1,2,4-triazol-3-yl)phenyl)-6-methyl-N8-(oxetan-3-ylmethyl)pyrido[3,4-d]pyrimidine-2,8-diamine;
N-(2-ethoxy-4-(4-methyl-4H-1,2,4-triazol-3-yl)phenyl)-6-methyl-8-(pyrrolidin-1-yl)pyrido[3,4-d]pyrimidin-2-amine;
N-(2-ethoxy-4-(4-methyl-4H-1,2,4-triazol-3-yl)phenyl)-6-methyl-8-(2-azaspiro[3.4]octan-2-yl)pyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-ethylazetidin-3-ol;
N2-(2-ethoxy-4-(4-methyl-4H-1,2,4-triazol-3-yl)phenyl)-6-methyl-N8-(1-methylpiperidin-4-yl)pyrido[3,4-d]pyrimidine-2,8-diamine;
8-(4-(dimethylamino)piperidin-1-yl)-N-(2-ethoxy-4-(4-methyl-4H-1,2,4-triazol-3-yl)phenyl)-6-methylpyrido[3,4-d]pyrimidin-2-amine;
N2-(2-ethoxy-4-(4-methyl-4H-1,2,4-triazol-3-yl)phenyl)-6-methyl-N8-((tetrahydro-2H-pyran-4-yl)methyl)pyrido[3,4-d]pyrimidine-2,8-diamine;
N2-(2-ethoxy-4-(4-methyl-4H-1,2,4-triazol-3-yl)phenyl)-6-methyl-N8-((4-methyltetrahydro-2H-pyran-4-yl)methyl)pyrido[3,4-d]pyrimidine-2,8-diamine;
1-(2-((2-ethoxy-4-(4-methyl-4H-1,2,4-triazol-3-yl)phenyl)amino)-6-methylpyrido[3,4-d]pyrimidin-8-yl)-4-ethylpiperidine-4-carbonitrile;
N2-(2-ethoxy-4-(4-methyl-4H-1,2,4-triazol-3-yl)phenyl)-6-methyl-N8-(2-(3-methyltetrahydrofuran-3-yl)ethyl)pyrido[3,4-d]pyrimidine-2,8-diamine;
N2-(2-ethoxy-4-(4-methyl-4H-1,2,4-triazol-3-yl)phenyl)-6-methyl-N8-(1-(tetrahydro-2H-pyran-4-yl)ethyl)pyrido[3,4-d]pyrimidine-2,8-diamine;
N2-(2-ethoxy-4-(4-methyl-4H-1,2,4-triazol-3-yl)phenyl)-6-methyl-N8-(pentan-3-yl)pyrido[3,4-d]pyrimidine-2,8-diamine;
N2-(2-ethoxy-4-(4-methyl-4H-1,2,4-triazol-3-yl)phenyl)-6-methyl-N8-(tetrahydrofuran-3-yl)pyrido[3,4-d]pyrimidine-2,8-diamine;
8-(3-ethoxy-3-methylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(3-ethyl-3-methoxyazetidin-1-yl)-6-methylpyrido[3,4-c]pyrimidin-2-amine;
8-(3-ethoxy-3-ethylazetidin-1-yl)-N-(2-ethoxy-4-(4-methyl-4H-1,2,4-triazol-3-yl)phenyl)-6-methylpyrido[3,4-c]pyrimidin-2-amine;
N-(2-ethoxy-4-(4-methyl-4H-1,2,4-triazol-3-yl)phenyl)-8-(3-isopropyl-3-methoxyazetidin-1-yl)-6-methylpyrido[3,4-c]pyrimidin-2-amine;
8-(3-ethoxy-3-isopropylazetidin-1-yl)-N-(2-ethoxy-4-(4-methyl-4H-1,2,4-triazol-3-yl)phenyl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-c]pyrimidin-8-yl)-3-ethylazetidine-3-carbonitrile;
1-(2-((2-ethoxy-4-(4-methyl-4H-1,2,4-triazol-3-yl)phenyl)amino)-6-methylpyrido[3,4-c]pyrimidin-8-yl)-3-isopropylazetidine-3-carbonitrile;
1-(2-((2-ethoxy-4-(4-methyl-4H-1,2,4-triazol-3-yl)phenyl)amino)-6-methylpyrido[3,4-c]pyrimidin-8-yl)-2,2,3-trimethylazetidine-3-carbonitrile;
N-(2-ethoxy-4-(4-methyl-4H-1,2,4-triazol-3-yl)phenyl)-8-(3-methoxy-2,2-dimethylazetidin-1-yl)-6-methylpyrido[3,4-c]pyrimidin-2-amine;
N-(2-ethoxy-4-(4-methyl-4H-1,2,4-triazol-3-yl)phenyl)-8-(3-methoxy-2,2,3-trimethylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-c]pyrimidin-8-yl)-2,2-dimethylazetidine-3-carbonitrile;
N2-(2-methoxy-4-(4-methyl-4H-1,2,4-triazol-3-yl)phenyl)-6-methyl-N8-(1-methylpiperidin-4-yl)pyrido[3,4-c]pyrimidine-2,8-diamine;
8-(4-(dimethylamino)piperidin-1-yl)-N-(2-methoxy-4-(4-methyl-4H-1,2,4-triazol-3-yl)phenyl)-6-methylpyrido[3,4-d]pyrimidin-2-amine;
N2-(2-methoxy-4-(4-methyl-4H-1,2,4-triazol-3-yl)phenyl)-6-methyl-N8-((tetrahydro-2H-pyran-4-yl)methyl)pyrido[3,4-c]pyrimidine-2,8-diamine;
N2-(2-methoxy-4-(4-methyl-4H-1,2,4-triazol-3-yl)phenyl)-6-methyl-N8-((4-methyltetrahydro-2H-pyran-4-yl)methyl)pyrido[3,4-d]pyrimidine-2,8-diamine;
4-ethyl-1-(2-((2-methoxy-4-(4-methyl-4H-1,2,4-triazol-3-yl)phenyl)amino)-6-methylpyrido[3,4-d]pyrimidin-8-yl)piperidine-4-carbonitrile;
N2-(2-methoxy-4-(4-methyl-4H-1,2,4-triazol-3-yl)phenyl)-6-methyl-N8-(2-(3-methyltetrahydrofuran-3-yl)ethyl)pyrido[3,4-d]pyrimidine-2,8-diamine;
N2-(2-methoxy-4-(4-methyl-4H-1,2,4-triazol-3-yl)phenyl)-6-methyl-N8-(1-(tetrahydro-2H-pyran-4-yl)ethyl)pyrido[3,4-d]pyrimidine-2,8-diamine;
N2-(2-methoxy-4-(4-methyl-4H-1,2,4-triazol-3-yl)phenyl)-6-methyl-N8-(pentan-3-yl)pyrido[3,4-d]pyrimidine-2,8-diamine;
N2-(2-methoxy-4-(4-methyl-4H-1,2,4-triazol-3-yl)phenyl)-6-methyl-N8-(tetrahydrofuran-3-yl)pyrido[3,4-d]pyrimidine-2,8-diamine;
8-(3-ethoxy-3-methylazetidin-1-yl)-N-(2-methoxy-4-(4-methyl-4H-1,2,4-triazol-3-yl)phenyl)-6-methylpyrido[3,4-d]pyrimidin-2-amine;
8-(3-ethyl-3-methoxyazetidin-1-yl)-N-(2-methoxy-4-(4-methyl-4H-1,2,4-triazol-3-yl)phenyl)-6-methylpyrido[3,4-d]pyrimidin-2-amine;
8-(3-ethoxy-3-ethylazetidin-1-yl)-N-(2-methoxy-4-(4-methyl-4H-1,2,4-triazol-3-yl)phenyl)-6-methylpyrido[3,4-d]pyrimidin-2-amine;
8-(3-isopropyl-3-methoxyazetidin-1-yl)-N-(2-methoxy-4-(4-methyl-4H-1,2,4-triazol-3-yl)phenyl)-6-methylpyrido[3,4-d]pyrimidin-2-amine;
8-(3-ethoxy-3-isopropylazetidin-1-yl)-N-(2-methoxy-4-(4-methyl-4H-1,2,4-triazol-3-yl)phenyl)-6-methylpyrido[3,4-d]pyrimidin-2-amine;
3-ethyl-1-(2-((2-methoxy-4-(4-methyl-4H-1,2,4-triazol-3-yl)phenyl)amino)-6-methylpyrido[3,4-d]pyrimidin-8-yl)azetidine-3-carbonitrile;
3-isopropyl-1-(2-((2-methoxy-4-(4-methyl-4H-1,2,4-triazol-3-yl)phenyl)amino)-6-methylpyrido[3,4-d]pyrimidin-8-yl)azetidine-3-carbonitrile;
1-(2-((2-methoxy-4-(4-methyl-4H-1,2,4-triazol-3-yl)phenyl)amino)-6-methylpyrido[3,4-d]pyrimidin-8-yl)-2,2,3-trimethylazetidine-3-carbonitrile;
8-(3-methoxy-2,2-dimethylazetidin-1-yl)-N-(2-methoxy-4-(4-methyl-4H-1,2,4-triazol-3-yl)phenyl)-6-methylpyrido[3,4-d]pyrimidin-2-amine;
8-(3-methoxy-2,2,3-trimethylazetidin-1-yl)-N-(2-methoxy-4-(4-methyl-4H-1,2,4-triazol-3-yl)phenyl)-6-methylpyrido[3,4-d]pyrimidin-2-amine;
1-(2-((2-methoxy-4-(4-methyl-4H-1,2,4-triazol-3-yl)phenyl)amino)-6-methylpyrido[3,4-d]pyrimidin-8-yl)-2,2-dimethylazetidine-3-carbonitrile;
8-(3,3-dimethylazetidin-1-yl)-N-(2-methoxy-4-(1-methyl-1H-tetrazol-5-yl)phenyl)-6-methylpyrido[3,4-d]pyrimidin-2-amine;
1-(2-((2-methoxy-4-(1-methyl-1H-tetrazol-5-yl)phenyl)amino)-6-methylpyrido[3,4-d]pyrimidin-8-yl)-4-methylpiperidine-4-carbonitrile;
8-(3-methoxy-3-methylazetidin-1-yl)-N-(2-methoxy-4-(1-methyl-1H-tetrazol-5-yl)phenyl)-6-methylpyrido[3,4-d]pyrimidin-2-amine;
N-(2-methoxy-4-(1-methyl-1H-tetrazol-5-yl)phenyl)-8-(4-methoxypiperidin-1-yl)-6-methylpyrido[3,4-d]pyrimidin-2-amine;
N-(2-methoxy-4-(1-methyl-1H-tetrazol-5-yl)phenyl)-8-(4-methoxy-4-methylpiperidin-1-yl)-6-methylpyrido[3,4-d]pyrimidin-2-amine;
N-(2-methoxy-4-(1-methyl-1H-tetrazol-5-yl)phenyl)-6-methyl-8-(1-oxa-6-azaspiro[3.3]heptan-6-yl)pyrido[3,4-d]pyrimidin-2-amine;
1-(2-((2-methoxy-4-(1-methyl-1H-tetrazol-5-yl)phenyl)amino)-6-methylpyrido[3,4-d]pyrimidin-8-yl)-3-methylazetidine-3-carbonitrile;
N2-(2-methoxy-4-(1-methyl-1H-tetrazol-5-yl)phenyl)-6-methyl-N8-((3-methyltetrahydrofuran-3-yl)methyl)pyrido[3,4-d]pyrimidine-2,8-diamine;
N-(2-methoxy-4-(1-methyl-1H-tetrazol-5-yl)phenyl)-6-methyl-8-(2-oxa-7-azaspiro[4.4]nonan-7-yl)pyrido[3,4-d]pyrimidin-2-amine;
N2-(2-methoxy-4-(1-methyl-1H-tetrazol-5-yl)phenyl)-6-methyl-N8-(tetrahydro-2H-pyran-4-yl)pyrido[3,4-d]pyrimidine-2,8-diamine;
N-(2-methoxy-4-(1-methyl-1H-tetrazol-5-yl)phenyl)-6-methyl-8-(7-oxa-2-azaspiro[3.5]nonan-2-yl)pyrido[3,4-d]pyrimidin-2-amine;
N2-(2-methoxy-4-(1-methyl-1H-tetrazol-5-yl)phenyl)-6-methyl-N8-neopentylpyrido[3,4-d]pyrimidine-2,8-diamine;
N-(2-(difluoromethoxy)-4-(4-methyl-4H-1,2,4-triazol-3-yl)phenyl)-8-(3,3-dimethylazetidin-1-yl)-6-methylpyrido[3,4-d]pyrimidin-2-amine;
1-(2-((2-(difluoromethoxy)-4-(4-methyl-4H-1,2,4-triazol-3-yl)phenyl)amino)-6-methylpyrido[3,4-d]pyrimidin-8-yl)-4-methylpiperidine-4-carbonitrile;
N-(2-(difluoromethoxy)-4-(4-methyl-4H-1,2,4-triazol-3-yl)phenyl)-8-(3-methoxy-3-methylazetidin-1-yl)-6-methylpyrido[3,4-d]pyrimidin-2-amine;
N-(2-(difluoromethoxy)-4-(4-methyl-4H-1,2,4-triazol-3-yl)phenyl)-8-(4-methoxypiperidin-1-yl)-6-methylpyrido[3,4-d]pyrimidin-2-amine;
N-(2-(difluoromethoxy)-4-(4-methyl-4H-1,2,4-triazol-3-yl)phenyl)-8-(4-methoxy-4-methylpiperidin-1-yl)-6-methylpyrido[3,4-d]pyrimidin-2-amine;
N-(2-(difluoromethoxy)-4-(4-methyl-4H-1,2,4-triazol-3-yl)phenyl)-6-methyl-8-(1-oxa-6-azaspiro[3.3]heptan-6-yl)pyrido[3,4-d]pyrimidin-2-amine;
1-(2-((2-(difluoromethoxy)-4-(4-methyl-4H-1,2,4-triazol-3-yl)phenyl)amino)-6-methylpyrido[3,4-d]pyrimidin-8-yl)-3-methylazetidine-3-carbonitrile;
N2-(2-(difluoromethoxy)-4-(4-methyl-4H-1,2,4-triazol-3-yl)phenyl)-6-methyl-N8-((3-methyltetrahydrofuran-3-yl)methyl)pyrido[3,4-d]pyrimidine-2,8-diamine;
N-(2-(difluoromethoxy)-4-(4-methyl-4H-1,2,4-triazol-3-yl)phenyl)-6-methyl-8-(2-oxa-7-azaspiro[4.4]nonan-7-yl)pyrido[3,4-d]pyrimidin-2-amine;
N2-(2-(difluoromethoxy)-4-(4-methyl-4H-1,2,4-triazol-3-yl)phenyl)-6-methyl-N8-(tetrahydro-2H-pyran-4-yl)pyrido[3,4-d]pyrimidine-2,8-diamine;
N-(2-(difluoromethoxy)-4-(4-methyl-4H-1,2,4-triazol-3-yl)phenyl)-6-methyl-8-(7-oxa-2-azaspiro[3.5]nonan-2-yl)pyrido[3,4-d]pyrimidin-2-amine;
N2-(2-(difluoromethoxy)-4-(4-methyl-4H-1,2,4-triazol-3-yl)phenyl)-6-methyl-N8-neopentylpyrido[3,4-d]pyrimidine-2,8-diamine;
8-(3,3-dimethylazetidin-1-yl)-N-(4-(4-ethyl-4H-1,2,4-triazol-3-yl)-2-methoxyphenyl)-6-methylpyrido[3,4-d]pyrimidin-2-amine;
1-(2-((4-(4-ethyl-4H-1,2,4-triazol-3-yl)-2-methoxyphenyl)amino)-6-methylpyrido[3,4-d]pyrimidin-8-yl)-4-methylpiperidine-4-carbonitrile;
N-(4-(4-ethyl-4H-1,2,4-triazol-3-yl)-2-methoxyphenyl)-8-(3-methoxy-3-methylazetidin-1-yl)-6-methylpyrido[3,4-d]pyrimidin-2-amine;
N-(4-(4-ethyl-4H-1,2,4-triazol-3-yl)-2-methoxyphenyl)-8-(4-methoxypiperidin-1-yl)-6-methylpyrido[3,4-d]pyrimidin-2-amine;
N-(4-(4-ethyl-4H-1,2,4-triazol-3-yl)-2-methoxyphenyl)-8-(4-methoxy-4-methylpiperidin-1-yl)-6-methylpyrido[3,4-d]pyrimidin-2-amine;
N-(4-(4-ethyl-4H-1,2,4-triazol-3-yl)-2-methoxyphenyl)-6-methyl-8-(1-oxa-6-azaspiro[3.3]heptan-6-yl)pyrido[3,4-d]pyrimidin-2-amine;
1-(2-((4-(4-ethyl-4H-1,2,4-triazol-3-yl)-2-methoxyphenyl)amino)-6-methylpyrido[3,4-d]pyrimidin-8-yl)-3-methylazetidine-3-carbonitrile;
N2-(4-(4-ethyl-4H-1,2,4-triazol-3-yl)-2-methoxyphenyl)-6-methyl-N8-((3-methyltetrahydrofuran-3-yl)methyl)pyrido[3,4-d]pyrimidine-2,8-diamine;
N-(4-(4-ethyl-4H-1,2,4-triazol-3-yl)-2-methoxyphenyl)-6-methyl-8-(2-oxa-7-azaspiro[4.4]nonan-7-yl)pyrido[3,4-d]pyrimidin-2-amine;
N2-(4-(4-ethyl-4H-1,2,4-triazol-3-yl)-2-methoxyphenyl)-6-methyl-N8-(tetrahydro-2H-pyran-4-yl)pyrido[3,4-d]pyrimidine-2,8-diamine;
N-(4-(4-ethyl-4H-1,2,4-triazol-3-yl)-2-methoxyphenyl)-6-methyl-8-(7-oxa-2-azaspiro[3.5]nonan-2-yl)pyrido[3,4-d]pyrimidin-2-amine;
N2-(4-(4-ethyl-4H-1,2,4-triazol-3-yl)-2-methoxyphenyl)-6-methyl-N8-neopentylpyrido[3,4-d]pyrimidine-2,8-diamine;
8-(3,3-dimethylazetidin-1-yl)-N-(2-ethoxy-4-(4-ethyl-4H-1,2,4-triazol-3-yl)phenyl)-6-methylpyrido[3,4-d]pyrimidin-2-amine;
1-(2-((2-ethoxy-4-(4-ethyl-4H-1,2,4-triazol-3-yl)phenyl)amino)-6-methylpyrido[3,4-d]pyrimidin-8-yl)-4-methylpiperidine-4-carbonitrile;
N-(2-ethoxy-4-(4-ethyl-4H-1,2,4-triazol-3-yl)phenyl)-8-(3-methoxy-3-methylazetidin-1-yl)-6-methylpyrido[3,4-d]pyrimidin-2-amine;
N-(2-ethoxy-4-(4-ethyl-4H-1,2,4-triazol-3-yl)phenyl)-8-(4-methoxypiperidin-1-yl)-6-methylpyrido[3,4-d]pyrimidin-2-amine;
N-(2-ethoxy-4-(4-ethyl-4H-1,2,4-triazol-3-yl)phenyl)-8-(4-methoxy-4-methylpiperidin-1-yl)-6-methylpyrido[3,4-d]pyrimidin-2-amine;
N-(2-ethoxy-4-(4-ethyl-4H-1,2,4-triazol-3-yl)phenyl)-6-methyl-8-(1-oxa-6-azaspiro[3.3]heptan-6-yl)pyrido[3,4-d]pyrimidin-2-amine;
1-(2-((2-ethoxy-4-(4-ethyl-4H-1,2,4-triazol-3-yl)phenyl)amino)-6-methylpyrido[3,4-d]pyrimidin-8-yl)-3-methylazetidine-3-carbonitrile;
N2-(2-ethoxy-4-(4-ethyl-4H-1,2,4-triazol-3-yl)phenyl)-6-methyl-N8-((3-methyltetrahydrofuran-3-yl)methyl)pyrido[3,4-d]pyrimidine-2,8-diamine;
N-(2-ethoxy-4-(4-ethyl-4H-1,2,4-triazol-3-yl)phenyl)-6-methyl-8-(2-oxa-7-azaspiro[4.4]nonan-7-yl)pyrido[3,4-d]pyrimidin-2-amine;
N2-(2-ethoxy-4-(4-ethyl-4H-1,2,4-triazol-3-yl)phenyl)-6-methyl-N8-(tetrahydro-2H-pyran-4-yl)pyrido[3,4-d]pyrimidine-2,8-diamine;
N-(2-ethoxy-4-(4-ethyl-4H-1,2,4-triazol-3-yl)phenyl)-6-methyl-8-(7-oxa-2-azaspiro[3.5]nonan-2-yl)pyrido[3,4-d]pyrimidin-2-amine;
N2-(2-ethoxy-4-(4-ethyl-4H-1,2,4-triazol-3-yl)phenyl)-6-methyl-N8-neopentylpyrido[3,4-d]pyrimidine-2,8-diamine;
N-(4-(4,5-dimethyl-4H-1,2,4-triazol-3-yl)-2-methoxyphenyl)-8-(3,3-dimethylazetidin-1-yl)-6-methylpyrido[3,4-d]pyrimidin-2-amine;
1-(2-((4-(4,5-dimethyl-4H-1,2,4-triazol-3-yl)-2-methoxyphenyl)amino)-6-methylpyrido[3,4-d]pyrimidin-8-yl)-4-methylpiperidine-4-carbonitrile;
N-(4-(4,5-dimethyl-4H-1,2,4-triazol-3-yl)-2-methoxyphenyl)-8-(3-methoxy-3-methylazetidin-1-yl)-6-methylpyrido[3,4-d]pyrimidin-2-amine;
N-(4-(4,5-dimethyl-4H-1,2,4-triazol-3-yl)-2-methoxyphenyl)-8-(4-methoxypiperidin-1-yl)-6-methylpyrido[3,4-d]pyrimidin-2-amine;
N-(4-(4,5-dimethyl-4H-1,2,4-triazol-3-yl)-2-methoxyphenyl)-8-(4-methoxy-4-methylpiperidin-1-yl)-6-methylpyrido[3,4-d]pyrimidin-2-amine;
N-(4-(4,5-dimethyl-4H-1,2,4-triazol-3-yl)-2-methoxyphenyl)-6-methyl-8-(1-oxa-6-azaspiro[3.3]heptan-6-yl)pyrido[3,4-d]pyrimidin-2-amine;
1-(2-((4-(4,5-dimethyl-4H-1,2,4-triazol-3-yl)-2-methoxyphenyl)amino)-6-methylpyrido[3,4-d]pyrimidin-8-yl)-3-methylazetidine-3-carbonitrile;
N2-(4-(4,5-dimethyl-4H-1,2,4-triazol-3-yl)-2-methoxyphenyl)-6-methyl-N8-((3-methyltetrahydrofuran-3-yl)methyl)pyrido[3,4-d]pyrimidine-2,8-diamine;
N-(4-(4,5-dimethyl-4H-1 ,2,4-triazol-3-yl)-2-methoxyphenyl)-6-methyl-8-(2-oxa-7-azaspiro[4.4]nonan-7-yl)pyrido[3,4-d]pyrimidin-2-amine;
N2-(4-(4,5-dimethyl-4H-1,2,4-triazol-3-yl)-2-methoxyphenyl)-6-methyl-N8-(tetrahydro-2H-pyran-4-yl)pyrido[3,4-d]pyrimidine-2,8-diamine;
N-(4-(4,5-dimethyl-4H-1,2,4-triazol-3-yl)-2-methoxyphenyl)-6-methyl-8-(7-oxa-2-azaspiro[3.5]nonan-2-yl)pyrido[3,4-d]pyrimidin-2-amine;
N2-(4-(4,5-dimethyl-4H-1,2,4-triazol-3-yl)-2-methoxyphenyl)-6-methyl-N8-neopentylpyrido[3,4-d]pyrimidine-2,8-diamine;
N-(4-(4,5-dimethyl-4H-1 ,2,4-triazol-3-yl)-2-ethoxyphenyl)-8-(3,3-dimethylazetidin-1-yl)-6-methylpyrido[3,4-d]pyrimidin-2-amine;
1-(2-((4-(4,5-dimethyl-4H-1 ,2,4-triazol-3-yl)-2-ethoxyphenyl)amino)-6-methylpyrido[3,4-d]pyrimidin-8-yl)-4-methylpiperidine-4-carbonitri le;
N-(4-(4,5-dimethyl-4H-1 ,2,4-triazol-3-yl)-2-ethoxyphenyl)-8-(3-methoxy-3-methylazetidin-1-yl)-6-methylpyrido[3,4-d]pyrimidin-2-amine;
N-(4-(4,5-dimethyl-4H-1 ,2,4-triazol-3-yl)-2-ethoxyphenyl)-8-(4-methoxypiperidin-1-yl)-6-methylpyrido[3,4-d]pyrimidin-2-amine;
N-(4-(4,5-dimethyl-4H-1 ,2,4-triazol-3-yl)-2-ethoxyphenyl)-8-(4-methoxy-4-methylpiperidin-1-yl)-6-methylpyrido[3,4-d]pyrimidin-2-amine;
N-(4-(4,5-dimethyl-4H-1 ,2,4-triazol-3-yl)-2-ethoxyphenyl)-6-methyl-8-(1-oxa-6-azaspiro[3.3]heptan-6-yl)pyrido[3,4-d]pyrimidin-2-amine;
1-(2-((4-(4,5-dimethyl-4H-1,2,4-triazol-3-yl)-2-ethoxyphenyl)amino)-6-methylpyrido[3,4-d]pyrimidin-8-yl)-3-methylazetidine-3-carbonitrile;
N2-(4-(4,5-dimethyl-4H-1,2,4-triazol-3-yl)-2-ethoxyphenyl)-6-methyl-N8-((3-methyltetrahydrofuran-3-yl)methyl)pyrido[3,4-d]pyrimidine-2,8-diamine;
N-(4-(4,5-dimethyl-4H-1 ,2,4-triazol-3-yl)-2-ethoxyphenyl)-6-methyl-8-(2-oxa-7-azaspiro[4.4]nonan-7-yl)pyrido[3,4-d]pyrimidin-2-amine;
N2-(4-(4,5-dimethyl-4H-1,2,4-triazol-3-yl)-2-ethoxyphenyl)-6-methyl-N8-(tetrahydro-2H-pyran-4-yl)pyrido[3,4-d]pyrimidine-2,8-diamine;
N-(4-(4,5-dimethyl-4H-1,2,4-triazol-3-yl)-2-ethoxyphenyl)-6-methyl-8-(7-oxa-2-azaspiro[3.5]nonan-2-yl)pyrido[3,4-d]pyrimidin-2-amine;
N2-(4-(4,5-dimethyl-4H-1,2,4-triazol-3-yl)-2-ethoxyphenyl)-6-methyl-N8-neopentylpyrido[3,4-d]pyrimidine-2,8-diamine;
8-(3,3-dimethylazetidin-1-yl)-N-(2-methoxy-4-(1-methyl-1H-1,2,3-triazol-5-yl)phenyl)-6-methylpyrido[3,4-d]pyrimidin-2-amine;
1-(2-((2-methoxy-4-(1-methyl-1H-1 ,2,3-triazol-5-yl)phenyl)amino)-6-methyl pyrido[3,4-d]pyrimidin-8-yl)-4-methylpiperidine-4-carbonitrile;
8-(3-methoxy-3-methylazetidin-1-yl)-N-(2-methoxy-4-(1-methyl-1H-1 ,2 , 3-triazol-5-yl)phenyl)-6-methylpyrido[3,4-d]pyrimidin-2-amine;
N-(2-methoxy-4-(1-methyl-1H-1,2,3-triazol-5-yl)phenyl)-8-(4-methoxypiperidin-1-yl)-6-methylpyrido[3,4-d]pyrimidin-2-amine;
N-(2-methoxy-4-(1-methyl-1H-1,2,3-triazol-5-yl)phenyl)-8-(4-methoxy-4-methylpiperidin-1-yl)-6-methylpyrido[3,4-d]pyrimidin-2-amine;
N-(2-methoxy-4-(1-methyl-1H-1,2,3-triazol-5-yl)phenyl)-6-methyl-8-(1-oxa-6-azaspiro[3.3]heptan-6-yl)pyrido[3,4-d]pyrimidin-2-amine;
1-(2-((2-methoxy-4-(1-methyl-1H-1,2,3-triazol-5-yl)phenyl)amino)-6-methylpyrido[3,4-d]pyrimidin-8-yl)-3-methylazetidine-3-carbonitrile;
N2-(2-methoxy-4-(1-methyl-1H-1,2,3-triazol-5-yl)phenyl)-6-methyl-N8-((3-methyltetrahydrofuran-3-yl)methyl)pyrido[3,4-d]pyrimidine-2,8-diamine;
N-(2-methoxy-4-(1-methyl-1H-1,2,3-triazol-5-yl)phenyl)-6-methyl-8-(2-oxa-7-azaspiro[4.4]nonan-7-yl)pyrido[3,4-d]pyrimidin-2-amine;
N2-(2-methoxy-4-(1-methyl-1H-1,2,3-triazol-5-yl)phenyl)-6-methyl-N8-(tetrahydro-2H-pyran-4-yl)pyrido[3,4-d]pyrimidine-2,8-diamine;
N-(2-methoxy-4-(1-methyl-1H-1,2,3-triazol-5-yl)phenyl)-6-methyl-8-(7-oxa-2-azaspiro[3.5]nonan-2-yl)pyrido[3,4-d]pyrimidin-2-amine;
N-(4-(1,2-dimethyl-1H-imidazol-5-yl)-2-methoxyphenyl)-8-(3,3-dimethylazetidin-1-yl)-6-methylpyrido[3,4-d]pyrimidin-2-amine;
1-(2-((4-(1,2-dimethyl-1H-imidazol-5-yl)-2-methoxyphenyl)amino)-6-methylpyrido[3,4-d]pyrimidin-8-yl)-4-methylpiperidine-4-carbonitrile;
N-(4-(1,2-dimethyl-1H-imidazol-5-yl)-2-methoxyphenyl)-8-(3-methoxy-3-methylazetidin-1-yl)-6-methylpyrido[3,4-d]pyrimidin-2-amine;
N-(4-(1,2-dimethyl-1H-imidazol-5-yl)-2-methoxyphenyl)-8-(4-methoxypiperidin-1-yl)-6-methylpyrido[3,4-d]pyrimidin-2-amine;
N-(4-(1,2-dimethyl-1H-imidazol-5-yl)-2-methoxyphenyl)-8-(4-methoxy-4-methylpiperidin-1-yl)-6-methylpyrido[3,4-d]pyrimidin-2-amine;
N-(4-(1,2-dimethyl-1H-imidazol-5-yl)-2-methoxyphenyl)-6-methyl-8-(1-oxa-6-azaspiro[3.3]heptan-6-yl)pyrido[3,4-d]pyrimidin-2-amine;
1-(2-((4-(1,2-dimethyl-1H-imidazol-5-yl)-2-methoxyphenyl)amino)-6-methylpyrido[3,4-d]pyrimidin-8-yl)-3-methylazetidine-3-carbonitrile;
N-(4-(1,2-dimethyl-1H-imidazol-5-yl)-2-methoxyphenyl)-6-methyl-8-(2-oxa-7-azaspiro[4.4]nonan-7-yl)pyrido[3,4-d]pyrimidin-2-amine;
N2-(4-(1,2-dimethyl-1H-imidazol-5-yl)-2-methoxyphenyl)-6-methyl-N8-(tetrahydro-2H-pyran-4-yl)pyrido[3,4-d]pyrimidine-2,8-diamine;
N-(4-(1,2-dimethyl-1H-imidazol-5-yl)-2-methoxyphenyl)-6-methyl-8-(7-oxa-2-azaspiro[3.5]nonan-2-yl)pyrido[3,4-d]pyrimidin-2-amine;
N-(4-(1,5-dimethyl-1H-imidazol-2-yl)-2-methoxyphenyl)-8-(3,3-dimethylazetidin-1-yl)-6-methylpyrido[3,4-d]pyrimidin-2-amine;
1-(2-((4-(1,5-dimethyl-1H-imidazol-2-yl)-2-methoxyphenyl)amino)-6-methylpyrido[3,4-d]pyrimidin-8-yl)-4-methylpiperidine-4-carbonitrile;
N-(4-(1,5-dimethyl-1H-imidazol-2-yl)-2-methoxyphenyl)-8-(3-methoxy-3-methylazetidin-1-yl)-6-methylpyrido[3,4-d]pyrimidin-2-amine;
N-(4-(1,5-dimethyl-1H-imidazol-2-yl)-2-methoxyphenyl)-8-(4-methoxypiperidin-1-yl)-6-methylpyrido[3,4-d]pyrimidin-2-amine;
N-(4-(1,5-dimethyl-1H-imidazol-2-yl)-2-methoxyphenyl)-8-(4-methoxy-4-methylpiperidin-1-yl)-6-methylpyrido[3,4-d]pyrimidin-2-amine;
N-(4-(1,5-dimethyl-1H-imidazol-2-yl)-2-methoxyphenyl)-6-methyl-8-(1-oxa-6-azaspiro[3.3]heptan-6-yl)pyrido[3,4-d]pyrimidin-2-amine;
1-(2-((4-(1,5-dimethyl-1H-imidazol-2-yl)-2-methoxyphenyl)amino)-6-methylpyrido[3,4-d]pyrimidin-8-yl)-3-methylazetidine-3-carbonitrile;
N2-(4-(1,5-dimethyl-1H-imidazol-2-yl)-2-methoxyphenyl)-6-methyl-N8-((3-methyltetrahydrofuran-3-yl)methyl)pyrido[3,4-d]pyrimidine-2,8-diamine;
N-(4-(1,5-dimethyl-1H-imidazol-2-yl)-2-methoxyphenyl)-6-methyl-8-(2-oxa-7-azaspiro[4.4]nonan-7-yl)pyrido[3,4-d]pyrimidin-2-amine;
N2-(4-(1,5-dimethyl-1H-imidazol-2-yl)-2-methoxyphenyl)-6-methyl-N8-(tetrahydro-2H-pyran-4-yl)pyrido[3,4-d]pyrimidine-2,8-diamine;
N-(4-(1,5-dimethyl-1H-imidazol-2-yl)-2-methoxyphenyl)-6-methyl-8-(7-oxa-2-azaspiro[3.5]nonan-2-yl)pyrido[3,4-d]pyrimidin-2-amine;
N2-(4-(1,5-dimethyl-1H-imidazol-2-yl)-2-methoxyphenyl)-6-methyl-N8-neopentylpyrido[3,4-d]pyrimidine-2,8-diamine;
8-(3,3-dimethylazetidin-1-yl)-N-(2-methoxy-4-(1-methyl-1H-imidazol-2-yl)phenyl)-6-methylpyrido[3,4-d]pyrimidin-2-amine;
1-(2-((2-methoxy-4-(1-methyl-1H-imidazol-2-yl)phenyl)amino)-6-methylpyrido[3,4-d]pyrimidin-8-yl)-4-methylpiperidine-4-carbonitri le;
8-(3-methoxy-3-methylazetidin-1-yl)-N-(2-methoxy-4-(1-methyl-1H-imidazol-2-yl)phenyl)-6-methylpyrido[3,4-d]pyrimidin-2-amine;
N-(2-methoxy-4-(1-methyl-1H-imidazol-2-yl)phenyl)-8-(4-methoxypiperidin-1-yl)-6-methylpyrido[3,4-d]pyrimidin-2-amine;
N-(2-methoxy-4-(1-methyl-1H-imidazol-2-yl)phenyl)-8-(4-methoxy-4-methylpiperidin-1-yl)-6-methylpyrido[3,4-d]pyrimidin-2-amine;
N-(2-methoxy-4-(1-methyl-1H-imidazol-2-yl)phenyl)-6-methyl-8-(1-oxa-6-azaspiro[3.3]heptan-6-yl)pyrido[3,4-d]pyrimidin-2-amine;
1-(2-((2-methoxy-4-(1-methyl-1H-imidazol-2-yl)phenyl)amino)-6-methylpyrido[3,4-d]pyrimidin-8-yl)-3-methylazetidine-3-carbonitrile;
N2-(2-methoxy-4-(1-methyl-1H-imidazol-2-yl)phenyl)-6-methyl-N8-((3-methyltetrahydrofuran-3-yl)methyl)pyrido[3,4-d]pyrimidine-2,8-diamine;
N-(2-methoxy-4-(1-methyl-1H-imidazol-2-yl)phenyl)-6-methyl-8-(2-oxa-7-azaspiro[4.4]nonan-7-yl)pyrido[3,4-d]pyrimidin-2-amine;
N2-(2-methoxy-4-(1-methyl-1H-imidazol-2-yl)phenyl)-6-methyl-N8-(tetrahydro-2H-pyran-4-yl)pyrido[3,4-d]pyrimidine-2,8-diamine;
N-(2-methoxy-4-(1-methyl-1H-imidazol-2-yl)phenyl)-6-methyl-8-(7-oxa-2-azaspiro[3.5]nonan-2-yl)pyrido[3,4-d]pyrimidin-2-amine;
8-(3,3-dimethylazetidin-1-yl)-N-(4-(2,4-dimethyloxazol-5-yl)-2-methoxyphenyl)-6-methylpyrido[3,4-d]pyrimidin-2-amine;
1-(2-((4-(2,4-dimethyloxazol-5-yl)-2-methoxyphenyl)amino)-6-methylpyrido[3,4-d]pyrimidin-8-yl)-4-methylpiperidine-4-carbonitrile;
N-(4-(2,4-dimethyloxazol-5-yl)-2-methoxyphenyl)-8-(3-methoxy-3-methylazetidin-1-yl)-6-methylpyrido[3,4-d]pyrimidin-2-amine;
N-(4-(2,4-dimethyloxazol-5-yl)-2-methoxyphenyl)-8-(4-methoxypiperidin-1-yl)-6-methylpyrido[3,4-d]pyrimidin-2-amine;
N-(4-(2,4-dimethyloxazol-5-yl)-2-methoxyphenyl)-8-(4-methoxy-4-methylpiperidin-1-yl)-6-methylpyrido[3,4-d]pyrimidin-2-amine;
N-(4-(2,4-dimethyloxazol-5-yl)-2-methoxyphenyl)-6-methyl-8-(1-oxa-6-azaspiro[3.3]heptan-6-yl)pyrido[3,4-d]pyrimidin-2-amine;
1-(2-((4-(2,4-dimethyloxazol-5-yl)-2-methoxyphenyl)amino)-6-methylpyrido[3,4-d]pyrimidin-8-yl)-3-methylazetidine-3-carbonitrile;
N2-(4-(2,4-dimethyloxazol-5-yl)-2-methoxyphenyl)-6-methyl-N8-((3-methyltetrahydrofuran-3-yl)methyl)pyrido[3,4-d]pyrimidine-2,8-diamine;
N-(4-(2,4-dimethyloxazol-5-yl)-2-methoxyphenyl)-6-methyl-8-(2-oxa-7-azaspiro[4.4]nonan-7-yl)pyrido[3,4-d]pyrimidin-2-amine;
N2-(4-(2,4-dimethyloxazol-5-yl)-2-methoxyphenyl)-6-methyl-N8-(tetrahydro-2H-pyran-4-yl)pyrido[3,4-d]pyrimidine-2,8-diamine;
N-(4-(2,4-dimethyloxazol-5-yl)-2-methoxyphenyl)-6-methyl-8-(7-oxa-2-azaspiro[3.5]nonan-2-yl)pyrido[3,4-d]pyrimidin-2-amine;
N2-(4-(2,4-dimethyloxazol-5-yl)-2-methoxyphenyl)-6-methyl-N8-neopentylpyrido[3,4-d]pyrimidine-2,8-diamine;
8-(3,3-dimethylazetidin-1-yl)-N-(4-(2,4-dimethyloxazol-5-yl)-2-ethoxyphenyl)-6-methylpyrido[3,4-d]pyrimidin-2-amine;
1-(2-((4-(2,4-dimethyloxazol-5-yl)-2-ethoxyphenyl)amino)-6-methylpyrido[3,4-d]pyrimidin-8-yl)-4-methylpiperidine-4-carbonitrile;
N-(4-(2,4-dimethyloxazol-5-yl)-2-ethoxyphenyl)-8-(3-methoxy-3-methylazetidin-1-yl)-6-methylpyrido[3,4-d]pyrimidin-2-amine;
N-(4-(2,4-dimethyloxazol-5-yl)-2-ethoxyphenyl)-8-(4-methoxypiperidin-1-yl)-6-methylpyrido[3,4-d]pyrimidin-2-amine;
N-(4-(2,4-dimethyloxazol-5-yl)-2-ethoxyphenyl)-8-(4-methoxy-4-methylpiperidin-1-yl)-6-methylpyrido[3,4-d]pyrimidin-2-amine;
N-(4-(2,4-dimethyloxazol-5-yl)-2-ethoxyphenyl)-6-methyl-8-(1-oxa-6-azaspiro[3.3]heptan-6-yl)pyrido[3,4-d]pyrimidin-2-amine;
1-(2-((4-(2,4-dimethyloxazol-5-yl)-2-ethoxyphenyl)amino)-6-methylpyrido[3,4-d]pyrimidin-8-yl)-3-methylazetidine-3-carbonitrile;
N2-(4-(2,4-dimethyloxazol-5-yl)-2-ethoxyphenyl)-6-methyl-N8-((3-methyltetrahydrofuran-3-yl)methyl)pyrido[3,4-d]pyrimidine-2,8-diamine;
N-(4-(2,4-dimethyloxazol-5-yl)-2-ethoxyphenyl)-6-methyl-8-(2-oxa-7-azaspiro[4.4]nonan-7-yl)pyrido[3,4-d]pyrimidin-2-amine;
N2-(4-(2,4-dimethyloxazol-5-yl)-2-ethoxyphenyl)-6-methyl-N8-(tetrahydro-2H-pyran-4-yl)pyrido[3,4-d]pyrimidine-2,8-diamine;
N-(4-(2,4-dimethyloxazol-5-yl)-2-ethoxyphenyl)-6-methyl-8-(7-oxa-2-azaspiro[3.5]nonan-2-yl)pyrido[3,4-d]pyrimidin-2-amine;
N2-(4-(2,4-dimethyloxazol-5-yl)-2-ethoxyphenyl)-6-methyl-N8-neopentylpyrido[3,4-d]pyrimidine-2,8-diamine;
8-(3,3-dimethylazetidin-1-yl)-N-(4-(2,5-dimethyloxazol-4-yl)-2-methoxyphenyl)-6-methylpyrido[3,4-d]pyrimidin-2-amine;
1-(2-((4-(2,5-dimethyloxazol-4-yl)-2-methoxyphenyl)amino)-6-methylpyrido[3,4-d]pyrimidin-8-yl)-4-methylpiperidine-4-carbonitrile;
N-(4-(2,5-dimethyloxazol-4-yl)-2-methoxyphenyl)-8-(3-methoxy-3-methylazetidin-1-yl)-6-methylpyrido[3,4-d]pyrimidin-2-amine;
N-(4-(2,5-dimethyloxazol-4-yl)-2-methoxyphenyl)-8-(4-methoxypiperidin-1-yl)-6-methylpyrido[3,4-d]pyrimidin-2-amine;
N-(4-(2,5-dimethyloxazol-4-yl)-2-methoxyphenyl)-8-(4-methoxy-4-methylpiperidin-1-yl)-6-methylpyrido[3,4-d]pyrimidin-2-amine;
N-(4-(2,5-dimethyloxazol-4-yl)-2-methoxyphenyl)-6-methyl-8-(1-oxa-6-azaspiro[3.3]heptan-6-yl)pyrido[3,4-d]pyrimidin-2-amine;
1-(2-((4-(2,5-dimethyloxazol-4-yl)-2-methoxyphenyl)amino)-6-methylpyrido[3,4-d]pyrimidin-8-yl)-3-methylazetidine-3-carbonitrile;
N2-(4-(2,5-dimethyloxazol-4-yl)-2-methoxyphenyl)-6-methyl-N8-((3-methyltetrahydrofuran-3-yl)methyl)pyrido[3,4-d]pyrimidine-2,8-diamine;
N-(4-(2,5-dimethyloxazol-4-yl)-2-methoxyphenyl)-6-methyl-8-(2-oxa-7-azaspiro[4.4]nonan-7-yl)pyrido[3,4-d]pyrimidin-2-amine;
N2-(4-(2,5-dimethyloxazol-4-yl)-2-methoxyphenyl)-6-methyl-N8-(tetrahydro-2H-pyran-4-yl)pyrido[3,4-d]pyrimidine-2,8-diamine;
N-(4-(2,5-dimethyloxazol-4-yl)-2-methoxyphenyl)-6-methyl-8-(7-oxa-2-azaspiro[3.5]nonan-2-yl)pyrido[3,4-d]pyrimidin-2-amine;
N2-(4-(2,5-dimethyloxazol-4-yl)-2-methoxyphenyl)-6-methyl-N8-neopentylpyrido[3,4-d]pyrimidine-2,8-diamine;
8-(3,3-dimethylazetidin-1-yl)-N-(4-(2,5-dimethyloxazol-4-yl)-2-ethoxyphenyl)-6-methylpyrido[3,4-d]pyrimidin-2-amine;
1-(2-((4-(2,5-dimethyloxazol-4-yl)-2-ethoxyphenyl)amino)-6-methylpyrido[3,4-d]pyrimidin-8-yl)-4-methylpiperidine-4-carbonitrile;
N-(4-(2,5-dimethyloxazol-4-yl)-2-ethoxyphenyl)-8-(3-methoxy-3-methylazetidin-1-yl)-6-methylpyrido[3,4-d]pyrimidin-2-amine;
N-(4-(2,5-dimethyloxazol-4-yl)-2-ethoxyphenyl)-8-(4-methoxypiperidin-1-yl)-6-methylpyrido[3,4-d]pyrimidin-2-amine;
N-(4-(2,5-dimethyloxazol-4-yl)-2-ethoxyphenyl)-8-(4-methoxy-4-methylpiperidin-1-yl)-6-methylpyrido[3,4-d]pyrimidin-2-amine;
N-(4-(2,5-dimethyloxazol-4-yl)-2-ethoxyphenyl)-6-methyl-8-(1-oxa-6-azaspiro[3.3]heptan-6-yl)pyrido[3,4-d]pyrimidin-2-amine;
1-(2-((4-(2,5-dimethyloxazol-4-yl)-2-ethoxyphenyl)amino)-6-methylpyrido[3,4-d]pyrimidin-8-yl)-3-methylazetidine-3-carbonitrile;
N2-(4-(2,5-dimethyloxazol-4-yl)-2-ethoxyphenyl)-6-methyl-N8-((3-methyltetrahydrofuran-3-yl)methyl)pyrido[3,4-d]pyrimidine-2,8-diamine;
N-(4-(2,5-dimethyloxazol-4-yl)-2-ethoxyphenyl)-6-methyl-8-(2-oxa-7-azaspiro[4.4]nonan-7-yl)pyrido[3,4-d]pyrimidin-2-amine;
N2-(4-(2,5-dimethyloxazol-4-yl)-2-ethoxyphenyl)-6-methyl-N8-(tetrahydro-2H-pyran-4-yl)pyrido[3,4-d]pyrimidine-2,8-diamine;
N-(4-(2,5-dimethyloxazol-4-yl)-2-ethoxyphenyl)-6-methyl-8-(7-oxa-2-azaspiro[3.5]nonan-2-yl)pyrido[3,4-d]pyrimidin-2-amine;
N2-(4-(2,5-dimethyloxazol-4-yl)-2-ethoxyphenyl)-6-methyl-N8-neopentylpyrido[3,4-d]pyrimidine-2,8-diamine,
or pharmaceutically acceptable salts thereof.

In one embodiment of each aspect and embodiment herein, the MPS1 inhibitor is selected from the group consisting of NMS-P715 and
N-cyclopropyl-4-(6-(2,3-difluoro-4-methoxyphenoxy)-8-((3,3,3-trifluoropropyl)amino)imidazo[1,2-b]pyridazin-3-yl)-2-methylbenzamide;
(R)-2-(4-fluorophenyl)-N-(4-(2-((2-methoxy-4-(methylsulfonyl)phenyl)amino)-[1,2,4]triazolo[1,5-a]pyridin-6-yl)phenyl)propanamide;
N2-(4-(4,5-dimethyl-4H-1,2,4-triazol-3-yl)-2-ethoxyphenyl)-6-methyl-N8-neopentylpyrido[3,4-d]pyrimidine-2,8-diamine;
(S)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
8-(3,3-dimethylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(3-methoxy-3-methylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylazetidine-3-carbonitrile;
N2-(2-ethoxy-4-(4-methyl-4H-1,2,4-triazol-3-yl)phenyl)-6-methyl-N8-neopentylpyrido[3,4-d]pyrimidine-2,8-diamine;
or a pharmaceutically acceptable salt or solvate thereof.

In one embodiment of each aspect and embodiment herein, the MPS1 inhibitor is selected from the group consisting of NMS-P715, BAY 1217389, BAY 1161909 and N2-(2-ethoxy-4-(4-methyl-4H-1,2,4-triazol-3-yl)phenyl)-6-methyl-N8-neopentylpyrido[3,4-d]pyrimidine-2,8-diamine, or pharmaceutically acceptable salts thereof.

In one embodiment of each aspect and embodiment herein, the MPS1 inhibitor is selected from the group consisting of:
N-cyclopropyl-4-(6-(2,3-difluoro-4-methoxyphenoxy)-8-((3,3,3-trifluoropropyl)amino)imidazo[1,2-b]pyridazin-3-yl)-2-methylbenzamide;
(R)-2-(4-fluorophenyl)-N-(4-(2-((2-methoxy-4-(methylsulfonyl)phenyl)amino)-[1,2,4]triazolo[1,5-a]pyridin-6-yl)phenyl)propanamide;
N2-(4-(4,5-dimethyl-4H-1,2,4-triazol-3-yl)-2-ethoxyphenyl)-6-methyl-N8-neopentylpyrido[3,4-d]pyrimidine-2,8-diamine;
(S)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
8-(3,3-dimethylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(3-methoxy-3-methylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylazetidine-3-carbonitrile;
N2-(2-ethoxy-4-(4-methyl-4H-1,2,4-triazol-3-yl)phenyl)-6-methyl-N8-neopentylpyrido[3,4-d]pyrimidine-2,8-diamine;
or a pharmaceutically acceptable salt or solvate thereof.

In one embodiment of each aspect and embodiment herein, the MPS1 inhibitor is selected from the group consisting of:
N-cyclopropyl-4-(6-(2,3-difluoro-4-methoxyphenoxy)-8-((3,3,3-trifluoropropyl)amino)imidazo[1,2-b]pyridazin-3-yl)-2-methylbenzamide; and
(R)-2-(4-fluorophenyl)-N-(4-(2-((2-methoxy-4-(methylsulfonyl)phenyl)amino)-[1,2,4]triazolo[1,5-a]pyridin-6-yl)phenyl)propanamide;
or a pharmaceutically acceptable salt or solvate thereof.

In one embodiment of each aspect and embodiment herein, the MPS1 inhibitor is selected from the group consisting of :
N2-(4-(4,5-dimethyl-4H-1,2,4-triazol-3-yl)-2-ethoxyphenyl)-6-methyl-N8-neopentylpyrido[3,4-d]pyrimidine-2,8-diamine;
(S)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
8-(3,3-dimethylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(3-methoxy-3-methylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylazetidine-3-carbonitrile;
N2-(2-ethoxy-4-(4-methyl-4H-1,2,4-triazol-3-yl)phenyl)-6-methyl-N8-neopentylpyrido[3,4-d]pyrimidine-2,8-diamine;
or pharmaceutically acceptable salts thereof.

In one embodiment of each aspect and embodiment herein, the MPS1 inhibitor is N2-(2-ethoxy-4-(4-methyl-4H-1,2,4-triazol-3-yl)phenyl)-6-methyl-N8-neopentylpyrido[3,4-d]pyrimidine-2,8-diamine, or pharmaceutically acceptable salts thereof.

### CDK4/6 Inhibitor

In one embodiment of each of the aspects and embodiments herein, the CDK4/6 inhibitor is selected from one or more of palbociclib, abemaciclib and ribociclib, or pharmaceutically acceptable salts or solvates thereof.

Suitably, the CDK4/6 inhibitor is selected from one or more of palbociclib and ribociclib, or pharmaceutically acceptable salts or solvates thereof.

Suitably, the CDK4/6 inhibitor is selected from one or more of palbociclib and abemaciclib, or pharmaceutically acceptable salts or solvates thereof.

Suitably, the CDK4/6 inhibitor is selected from one or more of ribociclib and abemaciclib, or pharmaceutically acceptable salts or solvates thereof.

Suitably, the CDK4/6 inhibitor is palbociclib, or pharmaceutically acceptable salts or solvates thereof.

### Combinations

In one aspect, the present invention relates to a combination comprising an MPS1 inhibitor and an endocrine agent.

In one aspect, the present invention relates to a pharmaceutical product comprising an MPS1 inhibitor and an endocrine agent.

In one aspect, the present invention relates to a method for the treatment of oestrogen receptor positive breast cancer in a subject in need thereof comprising administering to said subject, either separately, sequentially or in combination, a therapeutically effective amount of an MPS1 inhibitor and a therapeutically effective amount of an endocrine agent.

In one aspect, the present invention relates to a pharmaceutical product comprising an MPS1 inhibitor and an endocrine agent for use in the treatment of an oestrogen receptor positive breast cancer, wherein the MPS1 inhibitor and the endocrine agent are for separate, sequential or combined administration.

In one aspect, the present invention relates to the use of a pharmaceutical product comprising an MPS1 inhibitor and an endocrine agent in the manufacture of a medicament for the treatment of oestrogen receptor positive breast cancer, wherein the MPS1 inhibitor and the endocrine agent are for separate, sequential or combined administration.

In one aspect, the present invention relates to an MPS1 inhibitor and an endocrine agent for use in the treatment of estrogen receptor-positive breast cancer.

In one aspect, the present invention relates to an MPS1 inhibitor for use in the treatment of estrogen receptor-positive breast cancer, wherein said MPS1 inhibitor is for separate, sequential or combined administration with an endocrine agent.

In one aspect, the present invention relates to an endocrine agent for use in the treatment of estrogen receptor-positive breast cancer, wherein said endocrine agent is for separate, sequential or combined administration with an MPS1 inhibitor.

In one aspect, the present invention relates to a use of an MPS1 inhibitor and an endocrine agent in the manufacture of a medicament for the treatment of estrogen receptor-positive breast cancer.

In one aspect, the present invention relates to a use of an MPS1 inhibitor in the manufacture of a medicament for the treatment of estrogen receptor-positive breast cancer, wherein said MPS1 inhibitor is for separate, sequential or combined administration with an endocrine agent.

In one aspect, the present invention relates to a use of an endocrine agent in the manufacture of a medicament for the treatment of estrogen receptor-positive breast cancer, wherein said endocrine agent is for separate, sequential or combined administration with an MPS1 inhibitor.

Suitably, the MPS1 inhibitor is as defined any of the above embodiments. Suitably, the endocrine agent is as defined in any the above embodiments.

In one embodiment, the MPS1 inhibitor is selected from:
N-cyclopropyl-4-(6-(2,3-difluoro-4-methoxyphenoxy)-8-((3,3,3-trifluoropropyl)amino)imidazo[1,2-b]pyridazin-3-yl)-2-methylbenzamide;
(R)-2-(4-fluorophenyl)-N-(4-(2-((2-methoxy-4-(methylsulfonyl)phenyl)amino)-[1,2,4]triazolo[1,5-a]pyridin-6-yl)phenyl)propanamide;
N2-(4-(4,5-dimethyl-4H-1,2,4-triazol-3-yl)-2-ethoxyphenyl)-6-methyl-N8-neopentylpyrido[3,4-d]pyrimidine-2,8-diamine;
(S)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
8-(3,3-dimethylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(3-methoxy-3-methylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylazetidine-3-carbonitrile;
N2-(2-ethoxy-4-(4-methyl-4H-1,2,4-triazol-3-yl)phenyl)-6-methyl-N8-neopentylpyrido[3,4-d]pyrimidine-2,8-diamine;
or a pharmaceutically acceptable salt or solvate thereof, and the endocrine agent is selected from anastrazole, letraozole, exemestane, tamoxifen and fulvestrant or a pharmaceutically acceptable salt or solvate thereof.

In one embodiment, the MPS1 inhibitor is selected from:
N2-(4-(4,5-dimethyl-4H-1,2,4-triazol-3-yl)-2-ethoxyphenyl)-6-methyl-N8-neopentylpyrido[3,4-d]pyrimidine-2,8-diamine;
(S)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
8-(3,3-dimethylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(3-methoxy-3-methylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylazetidine-3-carbonitrile;
N2-(2-ethoxy-4-(4-methyl-4H-1,2,4-triazol-3-yl)phenyl)-6-methyl-N8-neopentylpyrido[3,4-d]pyrimidine-2,8-diamine;
or a pharmaceutically acceptable salt or solvate thereof, and the endocrine agent is selected from anastrazole, letraozole, exemestane, tamoxifen and fulvestrant, or a pharmaceutically acceptable salt or solvate thereof.

In one embodiment, the MPS1 inhibitor is selected from:
N2-(2-ethoxy-4-(4-methyl-4H-1,2,4-triazol-3-yl)phenyl)-6-methyl-N8-neopentylpyrido[3,4-d]pyrimidine-2,8-diamine;
or a pharmaceutically acceptable salt or solvate thereof, and the endocrine agent is selected from anastrazole, letraozole, exemestane, tamoxifen and fulvestrant, or a pharmaceutically acceptable salt or solvate thereof.

In one embodiment, the MPS1 inhibitor is selected from:
N2-(2-ethoxy-4-(4-methyl-4H-1,2,4-triazol-3-yl)phenyl)-6-methyl-N8-neopentylpyrido[3,4-d]pyrimidine-2,8-diamine;
or a pharmaceutically acceptable salt or solvate thereof, and the endocrine agent is selected from tamoxifen and fulvestrant, or a pharmaceutically acceptable salt or solvate thereof.

### EXAMPLES

### Materials and Methods

17-β-estradiol (E2) and 4-hydroxytamoxifen (4-OHT) were purchased from Sigma-Aldrich, (Dorset, UK); fulvestrant (IC182780) from Tocris Bioscience (Bristol, UK); paclitaxol from Selleckchem (Suffolk, UK); palbociclib (PD-0332991) was synthesized and supplied by Pfizer (Tadworth, UK). MPS1 inhibitors: CCT289346, BAY 1161909 and BAY 1217389 were synthesized and supplied by ICR Cancer Therapeutics (Sutton, UK); MPS1 inhibitor NMS-P715 was purchased from Calbiochem (Hertfordshire, UK).

### Tissue Culture

ER+ BC lines, wild type (wt) MCF7, wt-HCC1428, wt-SUM44, wt-T47D and wt-ZR75.1 cells were obtained from ATCC and cultured in phenol red-free RPMI 1640 medium (Gibco, Thermo Fisher Scientific, Loughborough, UK) supplemented with 10% fetal bovine serum (Gibco, Thermo Fisher Scientific) and 1 nM E2 at 37° C. in 5% CO₂. Cell lines identity was confirmed by short tandem repeat profiling (Promega, Madison, Wis., USA). Long-term-estrogen-deprived cells (MCF7-LTED^{wt} ESR1 and MCF7-LTED^{Y537C} mutants, HCC1428-LTED, SUM44-LTED and ZR75.1-LTED) modelling resistance to an endocrine therapy were cultured in phenol red free RPMI 1640 supplemented with 10% dextran charcoal-stripped fetal bovine serum (DCC-FBS).

### Cell Viability Assays (2D and 3D)

In order to assess effect of MPS1 inhibition on BC cells grown 2D, cells were seeded into white 96-well plates (Greiner Bio-One, Gloucestershire, UK) in RPMI 1640 supplemented with 10% DCC-FBS. Wt cells were stripped of E2 for 72 hrs prior to experiment. After 24 hrs cells were treated with escalating doses of MPS1 inhibitors±4-OHT or fulvestrant in presence or absence of E2. Media was replaced after 3 days and cell viability was assessed after 6 days using the CellTitre-Glo® Luminescent Cell Viability Assay (Promega) according to manufacturer's instructions.

For assessment of MPS1 inhibitors on viability of ER+ BC in 3D spheroid culture wt-MCF7 and MCF7-LTED^{wt} ESR1 and MCF7-LTED^{Y537C} cells were seeded in basal media into the ultralow attachment 96-well plates (Corning) at density of 2500 cells/well and centrifuged for 10 min at 900 rpm. After 3 days of incubation newly formed spheres were treated with escalating doses of CCT289346 or BAY 1217389, and re-treated every 3-4 days. The effect of treatment on cell viability was assessed after 10 days. Images of spheres were taken using CeligoS (Nexcelome Bioscience, Lawrence, Mass., US) and as a quantitative read out CellTitre-Glo® Luminescent 3D Cell Viability Assay (Promega) was performed according to manufacturer's protocol. Each experiment was performed at least twice with eight technical replicates per treatment. Luminescent signal was read on Victor spectrophotometer (Perkin Elmer, Wokingham, UK) and values were expressed as fold-change relative to the vehicle-treated control.

siRNA Kinome Library

A panel of LTED cell lines, including MCF7-LTED, SUM44-LTED, HCC1428-LTED, ZR75-LTED and T47D-LTED, as well as, MCF7-991R, MCF7-LTED 991R, T47D-991R, T47D-LTED 991R and MCF7-LTED ICI R 991R was transfected with ON-TARGETplus siRNA Library-Human Protein Kinase (G-103505, GE Dharmacon, Buckinghamshire, UK). The siRNA library consisting of nine 96-well plates containing SMART pool siRNA targeting 709 protein kinases was transferred onto three 384 well plates white-walled with clear bottoms (Greiner Bio-One) using Hamilton Microlab Star liquid handling robot (Hamilton, Bonaduz, Switzerland). The library was supplemented with non-targeting siRNA and PLK1 siRNA (both SMART pools from GE Dharmacon), as positive and negative controls respectively and plates frozen.

For reverse transfection, the plates containing 200 nM of siRNA were defrosted, 10 μl of RNAiMax (Invitrogen, Paisley, UK) per well was added using Multidrop Combi (Thermo Fisher Scientific) and incubated for 30 min at room temperature. Next, cells were seeded in 35 μl of basal growth media per well using Multidrop Combi. After 6 days cells viability was assessed using CellTitre-Glo® Luminescent Cell Viability Assay (Promega) according to manufacturer's protocol and luminescence was measured using Victor spectrophotometer (Perkin Elmer, Wokingam. UK). The luminescence reading for each well of the plates was log transformed, centered to the plates median and then median of replicates was calculated. This was finally used to calculate Z score using median absolute deviation for a cell line. The dynamics of each library screen was assessed by calculating Z prime values. The threshold of acceptance was set as Z′>0.3 (Brough et al., 2011). Each screen consisted of at least two biological experiments, which included a technical replicate library. Venn diagram combining targets from different BC models was then generated using Venny 2.1.0 (Computational Genomics, CNB-CSIC, Spain).

### Immunoblotting

In order to compare expression levels of MPS1 across our BC lines cell extracts were prepared from wt-MCF7, wt-HCC1428, wt-SUM44, wt-T47D, wt-ZR75.1, MCF7-LTED^{wt} ESR1 and MCF7-LTED^{Y537C}, HCC1428-LTED, SUM44-LTED and ZR75.1-LTED and SKBR3 grown in basal media at 70% confluence.

Protein extracts were generated as described previously (Martin et al., 2003). Equal amounts of protein (25 μg) were resolved by SDS-PAGE and immunoblot analysis was carried out. Proteins were detected using the antibodies against MPS1 (NT clone 3-472-1 IgG1, 05-682, 1:1000, Millipore, Walford, UK), and α-tubulin (T9026, mouse IgG1, 1:4000, Sigma-Aldrich). Secondary antibodies (Dako/Agilent, Santa Clara, Calif., UK) were used at 1:2000 dilution and antigenantibody interactions were detected with ECL reagent (Amersham).

For detection of PARP cleavage cells were seeded into 10 cm dishes and allowed to acclimatize overnight before 72 hrs treatment with CCT289346 or vehicle control. Proteins were resolved by SDS-PAGE, immunoblot was performed and membranes were incubated with antibodies against cleaved poly ADP-ribose polymerase (PARP) (Santa Cruz Biotechnology, Santa Cruz, Calif., UK) and α-tubulin (T9026, mouse IgG1, 1:4000, Sigma-Aldrich).

### Knockdown of 709 Kinases Identified MPS1 as a Common Determinant of Resistance to E-Deprivation

To identify targets that drive hormone-independent growth in oestrogen deprived (LTED) cells, a screen was performed using a library with siRNA pools targeting 709 kinases (FIG. 1A). Cell viability was measured using titerGlo. In this screen, we identified and validated MPS1/TTK as the top common hit in our panel of LTED cell lines irrespective of ER expression status (FIG. 1B). Assessment of global gene expression data and protein abundance using dimethyl-labelling of MCF7 versus MCF7-LTED cells showed increases of MPS1 at both the mRNA and protein level (FIG. 10) which was validated by immunoblot analysis (FIG. 1D). Of note SUM44-LTED showed a similar increase in MPS1 abundance compared to parental control, whilst T47D and T47D-LTED appeared similar and HCC1428-LTED appeared to have lower levels of MPS1 compared to their parental cell line.

Expression of MPS1 Associates with Poor Response to Endocrine Therapy

To assess the clinical relevance of MPS1 global gene expression datasets for ER+ BC patients treated with neoadjuvant anastrozole or letrozole were assessed. Clinical response data was available for 72 patients treated with anastrozole, of which 55 were classed as responders based on a 2-week residual Ki67 score<10% and 17 were classified as non-responders.

In this setting, on-treatment gene expression of MPS1 (p<0.0001) was significantly associated with poor response to anastrozole (FIG. 2A). It was also found that increased on-treatment gene expression of MPS1 (p<0.0001) correlated with higher Ki67 expression after 2 weeks of therapy, when expressed as a continuous variable (FIG. 2B).

In the second cohort, data were available for 52 tumours, of which 37 were classified as responders on the basis of tumour shrinkage ≥50%. Whilst baseline MPS1 expression showed a trend towards poor response (p=0.13) (FIG. 3C), increased on-treatment expression MPS1 (p=0.018) was significantly associated with poor response to letrozole (FIG. 3D).

Next it was assessed whether expression of MPS1 was also related to long-term outcome on adjuvant tamoxifen. After 10 years of follow-up of 747 ER+ patients treated with tamoxifen, MPS1 (p=3.14e-02) was shown to strongly associate with poor relapse free survival (RFS) (FIG. 4).

### MPS1 Inhibition Suppresses Proliferation of Endocrine Resistant Cell Lines in 2D and Spheroid Culture

The sensitivity of wt-MCF7 and MCF7-LTED cells to escalating concentrations of the MPS1 inhibitor CCT289346 in the presence or absence of estradiol (E2). In the presence of E2, CCT289346 caused a concentration dependent decrease in proliferation of wt-MCF7 cells (IC₅₀ 100 nM) (FIG. 5). Moreover, CCT289346 caused a profound decrease in proliferation of two MCF7-LTED models, one harbouring wt-ESR1 and the other a hotspot mutation (Y537C) (IC₅₀ values of 60 nM and 40 nM respectively).

Next the efficacy of CCT289346 in spheroid culture (FIG. 6) was assessed. Concordant with the previous observation, CCT289346 decreased sphere size, which was confirmed using TiterGlo to assess viability.

Subsequently the wt-MCF7 and MCF7-LTED models were treated with three alternative MPS1 inhibitors: NMS-P715, BAY 1161909 and BAY 1217389. All three agents caused a similar concentration dependent decrease in proliferation of wt-MCF7 cells in the presence of E2 and both MCF7-LTED models in the presence and absence of E2 (FIGS. 7 to 9).

Of note the IC₅₀ values in response to BAY 1217389 were far lower than the other agents tested by c. 20-fold. Furthermore, BAY 1217389 was particularly potent in spheroid cultures (FIG. 9). In order to address the possibility that these observations were cell line specific, we treated two further LTED models, SUM44 LTED and HCC1428 LTED, and these showed a similar pattern of sensitivity (FIG. 10). In the presence of E2, both wt-SUM44 and wt-HCC1428 showed a concentration dependent decrease in proliferation. Concentrations in excess of 250 nM appeared to induce a feedback loop and associated rise in proliferation. In the absence of E2, modelling addition of an endocrine therapy; note the data is normalised to vehicle and therefore expressed as 100%), CCT289346 caused a 30% drop in proliferation again supporting the ability of CCT289346 to target de novo resistance. Both HCC1428 LTED and SUM44 LTED showed a concentration dependent decrease in proliferation in the absence or presence of E2.

Taken together these data show a class specific effect of MPS1 inhibition confirming it as a clinically relevant target in endocrine therapy-resistant breast cancer.

We next investigated the potential role of MPS1 as a target in de-novo resistance. Previously, we showed using an E2F “on-treatment” signature score in patients treated with anastrozole, that we could identify patients whose breast cancer had ligand-independent ER activity (Miller et al. 2011). To address this we cultured wt-MCF7 cells in the presence or absence of E2 modelling the addition of an endocrine therapy. CCT289346 in the presence of E2 caused a concentration dependent decrease in proliferation. Addition of CCT289346 to DCC (modelling combination with an endocrine therapy) showed a further 20% drop in proliferation, suggesting MPS1 inhibition targeted de-novo resistance (FIG. 11).

Effect of CCT289346 in Combination with Endocrine Therapy

The potential of combining CCT289346 with fulvestrant or 4-hydroxytamoxifen (4-OHT) to improve efficacy was investigated. MCF7-LTED cells were treated with escalating concentrations of the endocrine agents alone or in combination with CCT289346 (established IC₅₀ concentration). As expected both endocrine agents in combination with CCT289346 caused a concentration dependent decrease in cell proliferation confirming dependence on ER for proliferation (FIG. 12). Taken together this data suggest CCT289346 combined with either an endocrine agent may provide clinical utility in endocrine therapy-resistant breast cancer patients.

Genomic Profiling Reveals Loss of RB is Associated with Irreversible Resistance to CDK4/6 Inhibition

A panel of breast cancer cell lines (wt-MCF7, MCF7 LTED, wt-T47D and T47D LTED) with varying phenotypic backgrounds, were treated long-term in the presence of a CDK4/6 inhibitor (palbociclib, 1 μM). Resistance was authenticated by culturing the resistant cell lines with escalating concentrations of palbociclib in comparison with their wild-type progenitor (FIG. 13).

Subsequently, the palbociclib resistant cell lines were cultured short and long-term in the absence of drug to assess the stability of the resistant phenotype. Of note, “washout” of wt-MCF7^{991R} and MCF7 LTED^{991R} derivatives re-sensitised them to the antiproliferative effect of palbociclib suggesting the phenotype was plastic. In contrast, the wt-T47D^{991R} and T47D LTED^{991R} cell lines remained resistant (FIG. 13).

### Targeting Resistance to Palbociclib

To investigate which kinases were commonly associated with the CDK4/6 resistant phenotype, a kinome knockdown screen (siRNA) targeting 709 kinases in the palbociclib resistant cell lines (FIG. 14) was used. All cell lines showed dependency on G2/M checkpoint regulators to varying degrees, however, MPS1 was a common determinant in all cell line models, irrespective of RB or ESR1 status. Next validated this observation was validated by comparing the effect of siRNA targeting MPS1 versus PLK in the cell lines. Inhibition varied between the cell lines with reduction in proliferation ranging between 20-40%.

### Palbociclib Resistant Cell Lines are Sensitive to the Anti-Proliferative Effects of CCT289346

To address the validity of MPS1 as a common determinant of resistance to CDK4/6 inhibition, proliferation assays were performed using MPS1 inhibitors CCT289346 and NMS-P715 in the presence or absence of palbociclib in variety of breast cancer cell lines, including wild type (FIG. 15) and LTED cell lines (FIG. 16).

All cell lines showed a concentration dependent decrease in proliferation in response to both MPS1 inhibitors (FIGS. 15 and 16) to varying degrees. The addition of CCT289346 to palbociclib showed an enhanced effect, reflected by the lower IC₅₀ values (c.50 nM for wt-MCF7⁹⁹¹R, MCF7 LTED^{991R} and wt-T47D^{991R}). The T47D-LTED^{991R} appeared the most sensitive to MPS1 inhibition with an IC₅₀ value of 25 nM.

To date MPS1 inhibitors have only been studied in triple negative breast cancer models in combination with chemotherapy. As demonstrated herein, MPS1 is also a suitable target in ER+ breast cancer models of resistance to endocrine therapy and/or palbociclib. The data herein shows that MPS1 inhibitors reduce tumour cell growth, especially in endocrine resistant cancer models and endocrine resistant models of palbociclib resistance disease. These findings are of significant clinical importance, at least in part because currently very little is known of suitable therapy for patients who relapse on CDK4/6 inhibitors.

### In Vivo Studies

### Methods

### In Vivo Efficacy Studies—MCF7-LTED

In vivo studies were carried out in ovariectomized 8- to 12-week-old female NCr out-bred nude mice in accordance with Home Office guidelines and approved by the Institute of Cancer Research Ethics Committee. Xenografts modelling patients resistant to aromatase treatment were initiated by innoculating MCF7-LTED (5×10⁶) cells in basement membrane matrix (1:1 Matrigel; BD Biosciences) into the right flank of each animal. Once tumours reached 7 mm in size, mice were randomized and treated with either 200 mg/kg fulvestrant (formulated in peanut oil) injected subcutaneously once a week (day 5), 50 mg/kg CCT289346 administered twice weekly by oral gavage (day 1 and day 4) or a combination of the two agents for 40 days. The control arm was treated with both vehicles. Tumours were measured twice weekly for 40 days.

Tumor growth was assessed twice weekly in all arms by caliper measurements of the two largest diameters. Volumes were then calculated according to the formula: a×b²×π/6, where a and b are orthogonal tumour diameters. For each tumor, volumes were reported to the initial volume as relative tumor volume (RTV). Means (and SE) of RTV in the same treatment group were calculated, and growth curves were established as a function of time.

### PDX Establishment

Female Swiss nude mice were purchased from Charles River (Les Arbresles, France) and maintained under specific pathogen-free conditions. Their care and housing were in accordance with institutional guidelines and the rules of the French Ethics Committee (project authorization no. 02163.02). PDX were established from primary surgical specimens with patient informed consent, as described elsewhere (Marangoni et al. 2007). HBCx-34 OvaR PDX was established from a ER+ breast cancer xenografts with acquired resistance to estrogen deprivation (absence of estrogen supplementation associated to ovariectomy) as previously published (Cottu et al. 2014). HBCx-86 PDX was established from an early stage ER+ PI3KCA mutated breast cancer as described in Hatem et al. (Hatem et al 2016) which was rendered estrogen-independent through successive tumor passages without estrogen supplementation.

### In Vivo Efficacy Studies in PDX

Fulvestrant (Faslodex, AstraZeneca, Macclesfield, UK) was administered by intramuscular injection with a 273/4 gauge needle at a dose of 50 mg/kg once a week (day 5). CCT289346 was administered by oral gavage at 25 mg/kg twice a week (days 1 and 4). When tumors reached a volume of 60 to 200 mm3, mice were individually identified and randomly assigned to the control or treated groups (10 mice per group), and the treatments were started. Treatments were administered during 5-6 weeks. Tumor growth was evaluated by measurement of two perpendicular diameters of tumors with a caliper twice per week. Individual tumor volumes were calculated as V=a b2/2, a being the largest diameter, b the smallest. For each tumor, Vs were reported to the initial volume as relative tumor volume (RTV). Means (and SE) of RTV in the same treatment group were calculated, and growth curves were established as a function of time.

### Results

Analysis of the MCF7-LTED model showed both fulvestrant and CCT289346 as mono therapies significantly reduced tumour volume compared to control but that there was no statistical difference between the drugs. The combination showed a trend towards statistical significance compared to fulvestrant alone (p=0.066) (FIG. 17).

Assessment of the PDX model HBCx-34 showed a significant reduction in tumour volume in response to both fulvestrant and CCT289346. Strikingly, the sub-optimal concentration of CCT289346 (25 mg/kg), as a monotherapy, reduced tumour volume to a similar degree as fulvestrant. The combination of the CCT289346 and fulvestrant showed a significant reduction (p=0.024) compared to fulvestrant alone (FIG. 18).

Assessment of the PDX model HBCx-86 showed a significant reduction in tumour volume with both CCT289346 and fulvestrant although fulvestrant as a monotherapy appeared superior. Strikingly the combination of fulvestrant and CCT289346 showed a significant reduction in tumour volume (p<0.0001) compared to fulvestrant alone (FIG. 19).

In summary, the above shows that MPS1 inhibitors are effective in vivo in models of resistance to estrogen-deprivation

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise paragraphed. No language in the specification should be construed as indicating any non-paragraphed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the paragraphs appended hereto as permitted by applicable law.

### REFERENCES

Asghar U, Witkiewicz A K, Turner N C, Knudsen E S. The history and future of targeting cyclin-dependent kinases in cancer therapy.Nat Rev Drug Discov. 2015 Feb; 14(2):130-46.
Brough R, Frankum J R, Sims D, Mackay A, Mendes-Pereira A M, Bajrami I, et al. Functional viability profiles of breast cancer. Cancer discovery. 2011; 1:260-73.
Hanahan D, Weinberg R A. Hallmarks of cancer: the next generation. Cell. 2011; 144:646-74.
Ma C X, Reinert T, Chmielewska I, Ellis M J. Mechanisms of aromatase inhibitor resistance. Nat Rev Cancer. 2015 May; 15(5):261-75.
Musgrove E A, Caldon C E, Barraclough J, Stone A, Sutherland R L. Cyclin D as a therapeutic target in cancer. Nature reviews Cancer. 2011; 11:558-72.
Miller T W, Balko J M, Fox E M, Ghazoui Z, Dunbier A, Anderson H, et al. ERalpha dependent E2F transcription can mediate resistance to estrogen deprivation in human breast cancer. Cancer discovery 2011; 1:338-41.
Marangoni E, Vincent-Salomon A, Auger N, Degeorges A, Assayag F, de Cremoux P, de Plater L, Guyader C, De Pinieux G, Judde J G, Rebucci M, Tran-Perennou C, Sastre-Garau X, Sigal-Zafrani B, Delattre O, Diéras V, Poupon M F. A new model of patient tumor-derived breast cancer xenografts for preclinical assays. Clin Cancer Res. 2007 Jul. 1; 13(13):3989-98.
Cottu P, Bièche I, Assayag F, El Botty R, Chateau-Joubert S, Thuleau A, Bagarre T, Albaud B, Rapinat A, Gentien D, de la Grange P, Sibut V, Vacher S, Hatem R, Servely J L, Fontaine J J, Decaudin D, Pierga J Y, Roman-Roman S, Marangoni E. Acquired resistance to endocrine treatments is associated with tumor-specific molecular changes in patient-derived luminal breast cancer xenografts. Clin Cancer Res. 2014 Aug. 15; 20(16):4314-25. doi: 10.1158/1078-0432.CCR-13-3230. Epub 2014 Jun. 19.
Hatem R, El Botty R, Chateau-Joubert S, Servely J L, Labiod D, de Plater L, Assayag F, Coussy F, Callens C, Vacher S, Reyal F, Cosulich S, Diéras V, Bieche I, Marangoni ETargeting mTOR pathway inhibits tumor growth in different molecular subtypes of triple-negative breast cancers. Oncotarget. 2016 Jul. 26; 7(30):48206-48219. doi: 10.18632/oncotarget.10195.

1. A method for the treatment of an oestrogen receptor positive breast cancer in a subject in need thereof comprising administering to said subject a therapeutically effective amount of an MPS1 inhibitor, wherein:
(i) said subject has previously been treated with an endocrine therapy; and/or
(ii) said breast cancer is resistant to endocrine therapy.

2. A method according to claim 1 wherein the subject has failed previous treatment with the endocrine therapy.

3. A method according to any one of claims 1 and 2 wherein the subject has relapsed during or following treatment with the endocrine therapy.

4. A method according to any one of the preceding claims wherein the subject has experienced disease progression during or following treatment with the endocrine therapy.

5. A method according to any one of the preceding claims wherein the subject has developed an endocrine therapy-resistant breast cancer during or following treatment with the endocrine therapy.

6. A method according to any one of claims 1 to 5 wherein said breast cancer is de novo resistant to endocrine therapy.

7. A method according to any one of the preceding claims wherein the endocrine therapy comprises/essentially consists of/consists of the administration of an endocrine agent.

8. A method according to claim 7 wherein the endocrine agent is selected from one or more of an aromatase inhibitor, a selective oestrogen receptor modulator (SERM) and a selective oestrogen receptor degrader/downregulator (SERD).

9. A method according to any one of the preceding claims wherein the endocrine therapy comprises/essentially consists of/consists of separate, sequential or combined treatment with an aromatase inhibitor and a SERD.

10. A method according to any one of claims 7 and 8 wherein the endocrine therapy comprises/essentially consists of/consists of separate, sequential or combined treatment with a SERM and a SERD.

11. A method according to any one of claims 8 and 9 wherein the aromatase inhibitor is selected from the group consisting of anastrozole, exemestane and letrozole, or pharmaceutically acceptable salts or solvates thereof.

12. A method according to any one of claims 8 and 10 wherein the SERM is tamoxifen, or pharmaceutically acceptable salts or solvates thereof.

13. A method according to any one of claims 8 to 10 wherein the SERD is fulvestrant, or pharmaceutically acceptable salts or solvates thereof.

14. A method according to any one of the preceding claims wherein the subject is pre-menopausal.

15. A method according to any one of claims 1 to 13 wherein the subject is post-menopausal.

16. A method according to any one of the preceding claims wherein (i) the subject has additionally been previously treated with a CDK4/6 inhibitor; and/or (ii) the breast cancer is additionally resistant to treatment with a CDK4/6 inhibitor.

17. A method according to claim 16 wherein the subject has failed treatment with the CDK4/6 inhibitor.

18. A method according to any one of claims 16 and 17 wherein the subject has relapsed during or following treatment with the CDK4/6 inhibitor.

19. A method according to any one of claims 16 to 18 wherein the subject has experienced disease progression during or following treatment with the CDK4/6 inhibitor.

20. A method according any one of the preceding claims wherein the subject has developed a CDK4/6 inhibitor-resistant breast cancer during or following treatment with the with the CDK4/6 inhibitor.

21. A method according any one of claims 1 to 20 wherein the breast cancer is de novo resistant to treatment with a CDK4/6 inhibitor.

22. A method according to any one of claims 16 to 21 wherein the CDK4/6 inhibitor is selected from the group consisting of palbociclib, abemaciclib and ribociclib, or pharmaceutically acceptable salts or solvates thereof.

23. A method according to any one of the preceding claims wherein the method further comprises administering to said subject an endocrine agent either separately, sequentially and/or combination with the MPS1 inhibitor.

24. A method according to claim 19 wherein the endocrine agent is selected from the group consisting of tamoxifen and fulvestrant, or pharmaceutically acceptable salts or solvates thereof.

25. A method according to any preceding claim wherein the compound capable of inhibiting MPS1 is an MPS1 inhibitor, suitably selected from the group consisting of NMS-P715, S 81694 (NMS-P153), AZ3146, BAY 1217389, BAY 1161909, MPS1-IN-3, MPS1-IN-2, CFI-402257, CCT289346, a compound of formula I, a compound of formula II, a compound of formula III and a compound of formula IV, or a pharmaceutically acceptable salt or solvate thereof;
wherein formula I is: 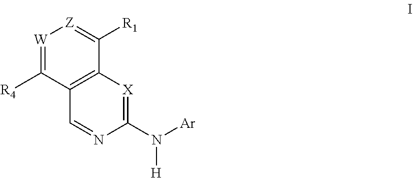
wherein:
W is N or C—R₃;
X is CH or N;
Z is N or C—H;
R₁ is selected from chloro, (1-6C)alkyl, (1-8C)heteroalkyl, aryl, aryl(1-2C)alkyl, heteroaryl, heteroaryl(1-2C)alkyl, heterocyclyl, heterocyclyl(1-2C)alkyl, (3-8C)cycloalkyl, (3-8C)cycloalkyl(1-2C)alkyl, NR₇R₈, OR₉, C(O)R₉, C(O)OR₉, OC(O)R₉, N(R₁₀)OR₉, N(R₁₀)C(O)OR₉, C(O)N(R₁₀)R₉, N(R₁₀)C(O)R₉, S(O)ₚR₉ (where p is 0, 1 or 2), SO₂N(R₁₀)R₉, N(R₁₀)SO₂R₉, N(R₁₀)SOR₉ or SON(R₁₀)R₉;
and wherein R₁ is optionally substituted by one or more substituent groups selected from fluoro, chloro, trifluoromethyl, trifluoromethoxy, cyano, nitro, hydroxy, amino, carboxy, carbamoyl, sulphamoyl, (1-4C)alkyl, (1-4C)alkoxy, S(O)_{q},CH₃ (where q is 0, 1 or 2), methylamino or dimethylamino, aryl, aryl(1-2C)alkyl, heteroaryl, heteroaryl(1-2C)alkyl, heterocyclyl, heterocyclyl(1-2C)alkyl, (3-8C)cycloalkyl, or (3-8C)cycloalkyl(1-2C)alkyl,
and wherein any (1-4C)alkyl, (1-4C)alkoxy, aryl, heteroaryl, heterocyclyl, or (3-8C)cycloalkyl moiety present within a substituent group on R₁ is optionally further substituted by fluoro, chloro, trifluoromethyl, trifluoromethoxy, cyano, nitro, hydroxy, amino, carboxy, carbamoyl, sulphamoyl, (1-4C)alkyl, NRₐR_{b}, ORₐ, C(O)Rₐ, C(O)ORₐ, OC(O)Rₐ, N(R_{b})ORₐ, C(O)N(R_{b})Rₐ, N(R_{b})C(O)Rₐ, S(O)ₚRₐ (where p is 0, 1 or 2), SO₂N(R_{b})Rₐ, or N(R_{b})SO₂Rₐ, wherein Rₐ and R_{b} are each independently selected from H or (1-4C)alkyl;
R₃ is hydrogen, (1-4C)alkyl, (3-6C)cycloalkyl, halo, CF₃, CN and (1-4C)alkoxy;
R₄ is hydrogen, (1-3C)alkyl, (1-3C)alkoxy, fluoro, chloro or CF₃;
Ar has the formula: 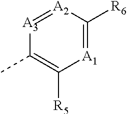
wherein:
(iv) all of A₁, A₂ and A₃ are CH;
(v) one of A₁, A₂ and A₃ is N and the others are CH; or
(vi) two of A₁, A₂ and A₃ are N and the other is CH;
R₅ is selected from hydrogen, cyano, (1-3C)alkyl, (1-3C)fluoroalkyl, (1-3C)alkoxy, (1-3C)fluoroalkoxy, halo, (1-3C)alkanoyl, C(O)NR₁₅R₁₆ or S(O)₂NR₁₅R₁₆, and wherein R₁₅ and R₁₆ are each independently selected from H or (1-3C)alkyl,
and wherein any alkyl or alkoxy moities present within a R₅ substituent group are optionally further substituted by hydroxy or methoxy;
R₆ is selected from halo, trifluoromethyl, trifluoromethoxy, cyano, nitro, hydroxy, amino, carboxy, carbamoyl, sulphamoyl, ureido, (1-6C)alkyl, (2-6C)alkenyl, (2-6C)alkynyl,
or R₆ is a group of the formula:
-L¹-L²-R₁₇
wherein
L¹ is absent or a linker group of the formula —[CR₁₈R₁₉]ₙ· in which n is an integer selected from 1, 2, 3 or 4, and R₁₈ and R₁₉ are each independently selected from hydrogen or (1-2C)alkyl;
L² is absent or is selected from O, S, SO, SO₂, N(R₂₀), C(O), C(O)O, OC(O), CH(OR₂₀), C(O)N(R₂₀), N(R₂₀)C(O), N(R₂₀)C(O)N(R₂₁), S(O)₂N(R₂₀), or N(R₂₁)SO₂, wherein R₂₀ and R₂₁ are each independently selected from hydrogen or (1-2C)alkyl; and
R₁₇ is (1-6C)alkyl, aryl, aryl-(1-6C)alkyl, (3-6C)cycloalkyl, (3-6C)cycloalkyl-(1-4C)alkyl, heteroaryl, heteroaryl-(1-4C)alkyl, heterocyclyl, heterocyclyl-(1-4C)alkyl,
and wherein R₁₇ is optionally further substituted by one or more substituent groups independently selected from oxo, halo, cyano, nitro, hydroxy, NR₂₂R₂₃, (1-4C)alkoxy, (1-4C)alkyl, (3-8C)cycloalkyl, (3-8C)cycloalkyl-(1-3C)alkyl, (1-5C)alkanoyl, (1-5C)alkylsulphonyl, heterocyclyl, heterocyclyl-(1-2C)alkyl, heteroaryl, heteroaryl-(1-2C)alkyl, CONR₂₂R₂₃, and SO₂NR₂₂R₂₃; wherein R₂₂ and R₂₃ are each independently selected from hydrogen, (1-4C)alkyl or (3-6C)cycloalkyl or (3-6C)cycloalkyl(1-2C)alkyl;
and wherein when said substituent group comprises an alkyl, cycloalkyl, heterocyclyl or heteroaryl moiety then said moiety is optionally further substituted by hydroxy, fluoro, chloro, cyano, CF₃, OCF₃, (1-2C)alkyl, (1-2C)alkoxy, SO₂(1-2C)alkyl or NRₑR_{f} (where Rₑ and R_{f} are each independently selected from hydrogen, (1-3C)alkyl, (3-6C)cycloalkyl, or (3-6C)cycloalkyl(1-2C)alkyl);
or R₁₇ is a group having the formula:
-L³-L⁴-R₂₄
L³ is absent or a linker group of the formula —[CR₂₅R₂₆]ₙ— in which n is an integer selected from 1, 2, 3 or 4, and R₂₅ and R₂₆ are each independently selected from hydrogen or (1-2C)alkyl;
L⁴ is absent or is selected from O, S, SO, SO₂, N(R₂₇), C(O), C(O)O, OC(O), CH(OR₂₇), C(O)N(R₂₇), N(R₂₇)C(O), N(R₂₇)C(O)N(R₂₈), S(O)₂N(R₂₇), or N(R₂₈)SO₂, wherein R₂₇ and R₂₈ are each independently selected from hydrogen or (1-2C)alkyl; and
R₂₄ is (1-6C)alkyl, aryl, aryl-(1-6C)alkyl, (3-6C)cycloalkyl, (3-6C)cycloalkyl-(1-4C)alkyl, heteroaryl, heteroaryl-(1-4C)alkyl, heterocyclyl, heterocyclyl-(1-4C)alkyl;
R₈ and R₉ are each independently selected from hydrogen, (1-6C)alkyl, (1-6C)alkoxy, (3-9C)cycloalkyl, (3-9C)cycloalkyl-(1-2C)alkyl, aryl, aryl-(1-2C)alkyl, heterocyclyl, heterocyclyl-(1-2C)alkyl, heteroaryl, heteroaryl-(1-2C)alkyl, and wherein R₈ and R₉ are optionally further substituted by one or more substituents selected from hydroxy, fluoro, chloro, cyano, CF₃, OCF₃ (1-2C)alkyl or (1-2C)alkoxy;
R₇ and R₁₀ are independently selected from hydrogen, (1-6C)alkyl, (3-6C)cycloalkyl, (3-6C)cycloalkyl-(1-2C)alkyl, and wherein R₇ and R₁₀ are optionally further substituted by one or more substituents selected from hydroxy, fluoro, chloro, cyano, CF₃, OCF₃, (1-2C)alkyl or (1-2C)alkoxy;
subject to the proviso that:
X is only N when Z is N;
W is only N when X and Z are both N; and
R₆ is not methoxy when R₁ is S(O)₂R₉ and R₉ is heterocyclyl;
wherein formula II is: 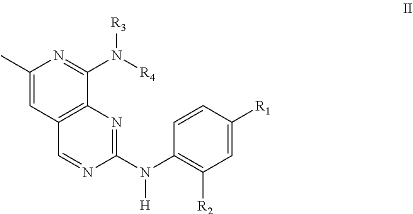
wherein:
R₁ is selected from:
(iii) a 5- or 6-membered heteroaryl optionally substituted with one or more substituents independently selected from halo, trifluoromethyl, difluoromethyl, trifluoromethoxy, difluoromethoxy, cyano, nitro, (1-4C)alkyl, NRₐR_{b}, ORₐ, C(O)Rₐ, C(O)ORₐ, OC(O)Rₐ, N(R_{b})ORₐ, C(O)N(R_{b})Rₐ, N(R_{b})C(O)Rₐ, S(O)ₚRₐ (where p is 0, 1 or 2), SO₂N(R_{b})Rₐ, or N(R_{b})SO₂Rₐ,
wherein Rₐ and R_{b} are each independently selected from H or (1-4C)alkyl, and wherein any alkyl moiety present in the substituent group is optionally further substituted with one or more substituents selected from halo, trifluoromethyl, difluoromethyl, trifluoromethoxy, difluoromethoxy, cyano, nitro, (1-4C)alkyl, 4-7-membered heterocyclyl, NR_{c}R_{d}, OR_{c}, C(O)R_{c}, C(O)OR_{c}, OC(O)R_{c}, N(R_{d})OR_{c}, C(O)N(R_{d})R_{c}, N(R_{d})C(O)R_{c}, S(O)_{q}R_{c} (where q is 0, 1 or 2), SO₂N(R_{d})R_{c}, or N(R_{d})SO₂R_{c}, wherein R_{c} and R_{d} are each independently selected from H or (1-4C)alkyl; or
wherein the 5- or 6-membered heteroaryl is optionally fused to a 4-, 5-, 6- or 7-membered heterocyclic ring, wherein the fused ring system is optionally substituted by one or more substituents independently selected from halo, trifluoromethyl, difluoromethyl, trifluoromethoxy, difluoromethoxy, cyano, nitro, (1-4C)alkyl, NRₖRₗ, ORₖ, C(O)Rₖ, C(O)ORₖ, OC(O)Rₖ, N(Rₗ)ORₖ, C(O)N(Rₗ)Rₖ, N(Rₗ)C(O)Rₖ, S(O)ₚRₖ (where p is 0, 1 or 2), SO₂N(Rₖ)Rₗ, or N(Rₖ)SO₂Rₗ, wherein Rₖ and Rₗ are each independently selected from H or (1-4C)alkyl,
and wherein any alkyl moiety present in the substituent group is optionally further substituted with one or more substituents selected from halo, trifluoromethyl, difluoromethyl, trifluoromethoxy, difluoromethoxy, cyano, nitro, (1-4C)alkyl, 4-7-membered heterocyclyl, NRₘRₙ, ORₘ, C(O)Rₘ, C(O)ORₘ, OC(O)Rₘ, N(Rₙ)ORₘ, C(O)N(Rₙ)Rₘ, N(Rₙ)C(O)Rₘ, S(O)_{q}Rₘ (where q is 0, 1 or 2), SO₂N(Rₙ)Rₘ, or N(Rₙ)SO₂Rₘ, wherein Rₘ and Rₙ are each independently selected from H or (1-4C)alkyl; or
(iv) a group —C(O)N(R_{f})Rₑ— or —S(O)₂N(R_{f})Rₑ—;
wherein Rₑ and R_{f} are each independently selected from H or (1-4C)alkyl which is optionally substituted by halo or (1-2C)alkoxy;
or Rₑ and R_{f} are linked such that, together with the nitrogen atom to which they are attached, they form a 4-, 5- or 6-membered heterocyclic ring, wherein said ring is optionally substituted with one or more substituents independently selected from halo, trifluoromethyl, difluoromethyl, trifluoromethoxy, difluoromethoxy, cyano, nitro, (1-4C)alkyl, NR_{g}Rₕ, OR_{g}, C(O)R_{g}, C(O)OR_{g}, OC(O)R_{g}, N(Rₕ)OR_{g}, C(O)N(Rₕ)R_{g}, N(Rₕ)C(O)R_{g}, S(O)ₚRₕ (where p is 0, 1 or 2), SO₂N(Rₕ)R_{g}, or N(Rₕ)SO₂R_{g}, wherein R_{g} and Rₕ are each independently selected from H or (1-4C)alkyl;
R₂ is selected from hydrogen, fluoro, chloro, (1-3C)alkoxy or (1-3C)fluoroalkoxy;
and either:
(iii) R₃ is selected from hydrogen or (1-3C)alkyl and R₄ is selected from (1-6C)alkyl, (3-9C)cycloalkyl, (3-9C)cycloalkyl-(1-4C)alkyl, aryl, aryl-(1-4C)alkyl, heterocyclyl, heterocyclyl-(1-4C)alkyl, heteroaryl, heteroaryl-(1-4C)alkyl, and wherein R₄ is optionally further substituted by one or more substituents selected from hydroxy, fluoro, chloro, cyano, CF₃, CHF₂, OCF₃, OCHF₂, (1-4C)alkyl, NRₒRₚ, ORₒ, C(O)Rₒ, C(O)ORₚ, OC(O)Rₒ, N(Rₚ)Oₒ, C(O)N(Rₚ)Rₒ, N(Rₚ)C(O)Rₒ, S(O)ₚRₒ (where p is 0, 1 or 2), SO₂N(Rₚ)Rₒ, or N(Rₚ)SO₂Rₒ or (3-6C)cycloalkyl, (3-6C)cycloalkyl-(1-2C)alkyl, a 4, 5 or 6-membered heterocyclyl, a 4, 5 or 6-membered heterocyclyl-(1-2C)alkyl, wherein Rₒ and Rₚ are each independently selected from H or (1-4C)alkyl, (3-6C)cycloalkyl or (3-6C)cycloalkyl-(1-4C)alkyl; or
(iv) R₃ and R₄ are linked such that, together with the nitrogen atom to which they are attached, they form a nitrogen-linked 4-, 5- 6- or 7-membered heterocyclic ring,
wherein said ring is optionally fused to a further 3-, 4-, 5- or 6-membered ring carbocyclic or heterocyclic ring, a 5- or 6-membered heteroaryl ring or a phenyl ring to form a bi-cyclic heterocyclic system, or
linked through a spiro carbon atom to a further 4-, 5- or 6-membered ring carbocyclic or heterocyclic ring to form a spiro bicyclic ring system;
and wherein the heterocyclic ring, bicyclic ring system or spiro bicyclic ring system is optionally substituted by one or more substitAuents independently selected from halo, trifluoromethyl, difluoromethyl, trifluoromethoxy, difluoromethoxy, cyano, nitro, (1-4C)alkyl, NRᵢRⱼ, ORᵢ, C(O)Rᵢ, C(O)ORᵢ, OC(O)Rᵢ, N(Rⱼ)ORᵢ, C(O)N(Rⱼ)Rᵢ , N(Rⱼ)C(O)Rᵢ, S(O)_{q}Rᵢ (where q is 0, 1 or 2), SO₂N(Rⱼ)Rᵢ, or N(Rⱼ)SO₂Rᵢ, wherein Rᵢ and Rⱼ are each independently selected from H or (1-4C)alkyl;
with the proviso that said compound is not one of the following:
N2-(2-methoxy-4-(1-methyl-1H-pyrazol-4-yl)phenyl)-6-methyl-N8-neopentylpyrido[3,4-d]pyrimidine-2,8-diamine;
N2-(4-(1,2-dimethyl-1H-imidazol-5-yl)-2-methoxyphenyl)-N8-(2-methoxy-2-methylpropyl)-6-methylpyrido[3,4-d]pyrimidine-2,8-diamine;
N2-(4-(1,2-dimethyl-1H-imidazol-5-yl)-2-methoxyphenyl)-6-methyl-N8-neopentylpyrido[3,4-d]pyrimidine-2,8-diamine;
N2-(2-methoxy-4-(4-methyl-4H-1,2,4-triazol-3-yl)phenyl)-6-methyl-N8-neopentylpyrido[3,4-d]pyrimidine-2,8-diamine;
N8-(2-methoxy-2-methylpropyl)-N2-(2-methoxy-4-(4-methyl-4H-1,2,4-triazol-3-yl)phenyl)-6-methylpyrido[3,4-d]pyrimidine-2,8-diamine;
N2-(2-methoxy-4-(1-(2-methoxyethyl)-2-methyl-1H-imidazol-5-yl)phenyl)-6-methyl-N8-neopentylpyrido[3,4-d]pyrimidine-2,8-diamine;
N2-(2-ethoxy-4-(4-methyl-4H-1,2,4-triazol-3-yl)phenyl)-6-methyl-N8-neopentylpyrido[3,4-d]pyrimidine-2,8-diamine;
N2-(2-methoxy-4-(4-(methylsulfonyl)piperazin-1-yl)phenyl)-6-methyl-N8-neopentylpyrido[3,4-d]pyrimidine-2,8-diamine;
N2-(4-(1,3-dimethyl-1H-pyrazol-4-yl)-2-methoxyphenyl)-N8-(2-methoxy-2-methylpropyl)-6-methylpyrido[3,4-d]pyrimidine-2,8-diamine;
N-(2-ethoxy-4-(4-methyl-4H-1,2,4-triazol-3-yl)phenyl)-6-methyl-8-(6-oxa-2-azaspiro[3.4]octan-2-yl)pyrido[3,4-d]pyrimidin-2-amine;
N8-(2-methoxy-2-methylpropyl)-N2-(2-methoxy-4-(1-methyl-1H-1,2,4-triazol-5-yl)phenyl)-6-methylpyrido[3,4-d]pyrimidine-2,8-diamine;
N2-(2-(difluoromethoxy)-4-(1-methyl-1H-pyrazol-4-yl)phenyl)-N8-(2-methoxy-2-methylpropyl)-6-methylpyrido[3,4-d]pyrimidine-2,8-diamine;
(4-(3-methoxy-4-((8-((2-methoxy-2-methylpropyl)amino)-6-methylpyrido[3,4-d]pyrimidin-2-yl)amino)phenyl)-1-methyl-1H-pyrazol-5-yl)methanol;
wherein formula III is 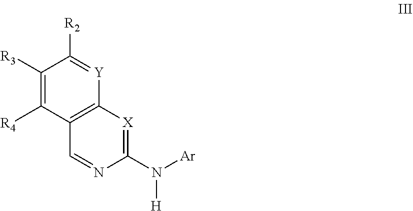
wherein:
X is CH or N;
Y is N or C—H;
R₂ is selected from (1-6C)alkyl, (1-8C)heteroalkyl, aryl, aryl(1-2C)alkyl, a 5 or 6 membered heteroaryl, a 5 or 6 membered heteroaryl(1-2C)alkyl, a 3 to 6 membered heterocyclyl, a 3 to 6 membered heterocyclyl(1-2C)alkyl, (3-8C)cycloalkyl, (3-8C)cycloalkyl(1-2C)alkyl, NR₁₁R₁₂, OR₁₃, C(O)R₁₃, C(O)OR₁₃, OC(O)R₁₃, N(R₁₄)OR₁₃, N(R₁₄)C(O)OR₁₃, C(O)N(R₁₄)R₁₃, N(R₁₄)C(O)R₁₃, S(O)ₓR₁₃ (where x is 0, 1 or 2), SO₂N(R₁₄)R₁₃, or N(R₁₄)SO₂R₁₃;
and wherein R₂ is optionally substituted by one or more substituent groups selected from fluoro, chloro, trifluoromethyl, trifluoromethoxy, cyano, nitro, hydroxy, amino, carboxy, carbamoyl, sulphamoyl, (1-4C)alkyl, (1-4C)alkoxy, S(O)ₓCH₃ (where x is 0, 1 or 2), methylamino or dimethylamino, aryl, aryl(1-2C)alkyl, heteroaryl, heteroaryl(1-2C)alkyl, heterocyclyl, heterocyclyl(1-2C)alkyl, (3-8C)cycloalkyl, or (3-8C)cycloalkyl(1-2C)alkyl,
and wherein any (1-4C)alkyl, (1-4C)alkoxy, aryl, heteroaryl, heterocyclyl, or (3-8C)cycloalkyl moiety present within a substituent group on R₂ is optionally further substituted by fluoro, chloro, trifluoromethyl, trifluoromethoxy, cyano, nitro, hydroxy, amino, carboxy, carbamoyl, sulphamoyl, (1-4C)alkyl, NR_{c}R_{d}, OR_{c}, C(O)R_{c}, C(O)OR_{c}, OC(O)R_{c}, N(R_{d})OR_{c}, C(O)N(R_{d})R_{c}, N(R_{d})C(O)R_{c}, S(O)_{y}R_{c} (where y is 0, 1 or 2), SO₂N(R_{d})R_{c}, or N(R_{d})SO₂R_{c}, wherein R_{c} and R_{d} are each independently selected from H or (1-4C)alkyl;
R₃ is hydrogen, (1-4C)alkyl, (3-6C)cycloalkyl, halo, CF₃, CN and (1-4C)alkoxy;
R₄ is hydrogen, (1-3C)alkyl, fluoro, chloro or CF₃;
Ar has the formula: 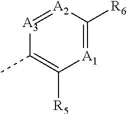
wherein:
(iii) all of A₁, A₂ and A₃ are CH; or
(iv) A₃ is CH and A₁ or A₂ are selected from N or CH;
R₅ is hydrogen, cyano, (1-3C)alkyl, (1-3C)fluoroalkyl, (1-3C)alkoxy, (1-3C)fluoroalkoxy, halo, (1-3C)alkanoyl, C(O)NR₁₅R₁₆ or S(O)₂NR₁₅R₁₆, and wherein R₁₅ and R₁₆ are each independently selected from H or (1-3C)alkyl, and wherein any alkyl or alkoxy moities present within a R₅ substituent group are optionally further substituted by hydroxy or methoxy;
R₆ is halogeno, trifluoromethyl, trifluoromethoxy, cyano, nitro, hydroxy, amino, carboxy, carbamoyl, sulphamoyl, ureido, (1-6C)alkyl, (2-6C)alkenyl, (2-6C)alkynyl,
or R₆ is a group of the formula:
-L¹-L²-R₁₇
wherein
L¹ is absent or a linker group of the formula —[CR₁₈R₁₉]ₙ— in which n is an integer selected from 1, 2, 3 or 4, and R₁₈ and R₁₉ are each independently selected from hydrogen or (1-2C)alkyl;
L² is absent or is selected from O, S, SO, SO₂, N(R₂₀), C(O), C(O)O, OC(O), CH(OR₂₀), C(O)N(R₂₀), N(R₂₀)C(O), N(R₂₀)C(O)N(R₂₁), S(O)₂N(R₂₀), or N(R₂₁)SO₂, wherein R₂₀ and R₂₁ are each independently selected from hydrogen or (1-2C)alkyl; and
R₁₇ is (1-6C)alkyl, aryl, aryl-(1-6C)alkyl, (3-6C)cycloalkyl, (3-6C)cycloalkyl-(1-4C)alkyl, heteroaryl, heteroaryl-(1-4C)alkyl, heterocyclyl, heterocyclyl-(1-4C)alkyl,
and wherein R₁₇ is optionally further substituted by one or more substituent groups independently selected from oxo, halo, cyano, nitro, hydroxy, NR₂₂R₂₃, (1-4C)alkoxy, (1-4C)alkyl, (3-8C)cycloalkyl, (3-8C)cycloalkyl-(1-3C)alkyl, (1-5C)alkanoyl, (1-5C)alkylsulphonyl, heterocyclyl, heterocyclyl-(1-2C)alkyl, heteroaryl, heteroaryl-(1-2C)alkyl, CONR₂₂R₂₃, and SO₂NR₂₂R₂₃; wherein R₂₂ and R₂₃ are each independently selected from hydrogen, (1-4C)alkyl or (3-6C)cycloalkyl or (3-6C)cycloalkyl(1-2C)alkyl; or R₂₂ and R₂₃ can be linked such that, together with the nitrogen atom to which they are attached, they form a 4-6 membered heterocyclic ring ring;
and wherein when said substituent group comprises an alkyl, cycloalkyl, heterocyclyl or heteroaryl moiety then said moiety is optionally further substituted by hydroxy, fluoro, chloro, cyano, CF₃, OCF₃, (1-2C)alkyl, (1-2C)alkoxy, SO₂(1-2C)alkyl or NRₑR_{f} (where Rₑ and R_{f} are each independently selected from hydrogen, (1-3C)alkyl, (3-6C)cycloalkyl, or (3-6C)cycloalkyl(1-2C)alkyl);
or R₁₇ is a group having the formula:
-L³-L⁴-R₂₄
wherein
L³ is absent or a linker group of the formula —[CR₂₅R₂₆]ₙ— in which n is an integer selected from 1, 2, 3 or 4, and R₂₅ and R₂₆ are each independently selected from hydrogen or (1-2C)alkyl;
L⁴ is absent or is selected from O, S, SO, SO₂, N(R₂₇), C(O), C(O)O, OC(O), CH(OR₂₇), C(O)N(R₂₇), N(R₂₇)C(O), N(R₂₇)C(O)N(R₂₈), S(O)₂N(R₂₇), or N(R₂₈)SO₂, wherein R₂₇ and R₂₈ are each independently selected from hydrogen or (1-2C)alkyl; and
R₂₄ is (1-6C)alkyl, aryl, aryl-(1-6C)alkyl, (3-6C)cycloalkyl, (3-6C)cycloalkyl-(1-4C)alkyl, heteroaryl, heteroaryl-(1-4C)alkyl, heterocyclyl, heterocyclyl-(1-4C)alkyl;
R₁₂ is selected from hydrogen, (1-6C)alkyl, (1-6C)alkoxy, (3-6C)cycloalkyl, (3-6C)cycloalkyl-(1-2C)alkyl, aryl, aryl-(1-2C)alkyl, heterocyclyl, heterocyclyl-(1-2C)alkyl, heteroaryl, heteroaryl-(1-2C)alkyl, and wherein R₁₂ is optionally further substituted by one or more substituents selected from hydroxy, fluoro, chloro, cyano, CF₃, OCF₃ (1-2C)alkyl or (1-2C)alkoxy;
R₁₃ is selected from hydrogen, (1-6C)alkyl, (1-6C)alkoxy, (3-6C)cycloalkyl, (3-6C)cycloalkyl-(1-2C)alkyl, aryl, aryl-(1-2C)alkyl, heteroaryl, heteroaryl-(1-2C)alkyl, and wherein R₁₃ is optionally further substituted by one or more substituents selected from hydroxy, fluoro, chloro, cyano, CF₃, OCF₃ (1-2C)alkyl or (1-2C)alkoxy;
R₁₁ and R₁₄ are independently selected from hydrogen, (1-6C)alkyl, (3-6C)cycloalkyl, (3-6C)cycloalkyl-(1-2C)alkyl, and wherein R₁₁ and R₁₄ are optionally further substituted by one or more substituents selected from hydroxy, fluoro, chloro, cyano, CF₃, OCF₃, (1-2C)alkyl or (1-2C)alkoxy;
subject to the proviso that:
X can only be N when Y is N; and
when X and Y are both N, R₃ is selected from H or fluoro and R₂ is not a NR₁₁R₁₂ group;
wherein formula IV is: 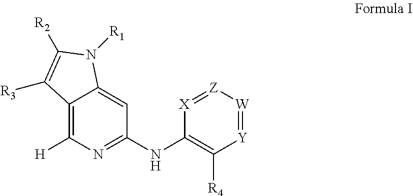
wherein:
R₁ is hydrogen, (1-5C)alkyl, (1-5C)fluoroalkyl, (3-8C)cycloalkyl, (3-8C)cycloalkyl-(1-4C)alkyl, aryl, aryl-(1-4C)alkyl, heteroaryl, heteroaryl-(1-4C)alkyl, —S(O)₂—R^{a},
—C(O)—R^{a}, or —C(O)—O—R^{a}, wherein R^{a} is (1-5C)alkyl, (3-8C)cycloalkyl, (3-8C)cycloalkyl-(1-4C)alkyl, aryl, aryl-(1-4C)alkyl, heteroaryl or heteroaryl-(1-4C)alkyl, and wherein any (1-5C)alkyl, (3-8C)cycloalkyl, (3-8C)cycloalkyl-(1-4C)alkyl, aryl, aryl-(1-4C)alkyl, heteroaryl, heteroaryl-(1-4C)alkyl group present in a R₁ substituent group is optionally substituted by methyl, trifluoromethyl, methoxy, trifluoromethoxy, halo, cyano, nitro, hydroxy, mercapto, amino, carboxy, carbamoyl, or sulphamoyl;
R₂ is an aryl, aryl(1-2C)alkyl, 5- or 6-membered heteroaryl or a 5- or 6-membered heteroaryl(1-2C)alkyl,
wherein R₂ is optionally substituted by one or more substituents selected from halogeno, trifluoromethyl, trifluoromethoxy, cyano, nitro, hydroxy, mercapto, amino, carboxy, carbamoyl, sulphamoyl,
or a group of the formula:
L-L⁰-R^{b}
wherein
L is absent or a linker group of the formula —[CR_{g}Rₕ]ₙ— in which n is an integer selected from 1, 2, 3 or 4, and R_{g} and Rₕ are each independently selected from hydrogen or (1-2C)alkyl;
L⁰ is absent or is selected from O, S, SO, SO₂, N(R^{c}), C(O), C(O)O, OC(O), CH(OR^{c}), C(O)N(R^{c}), N(R^{c})C(O), N(R^{c})C(O)N(R^{d}), SO₂N(R^{c}), or N(R^{c})SO₂, wherein R^{c} and R^{d} are each independently selected from hydrogen or (1-2C)alkyl; and
R^{b} is (1-4C)alkyl, aryl, aryl-(1-4C)alkyl, (3-6C)cycloalkyl, (3-6C)cycloalkyl-(1-4C)alkyl, heteroaryl, heteroaryl-(1-4C)alkyl, heterocyclyl, or heterocyclyl-(1-4C)alkyl;
and wherein R^{b} is optionally further substituted by one or more substituents independently selected from oxo, halogeno, cyano, nitro, hydroxy, NR^{e}R^{f}, (1-5C)alkyl, (1-5C)alkoxy, (1-5C)alkanoyl, (1-5C)sulphonyl or aryl; and wherein R^{e} and R^{f} are each independently selected from hydrogen or (1-4C)alkyl or (3-6C)cycloalkyl-(1-4C)alkyl; or R^{e} and R^{f} can be linked such that, together with the nitrogen atom to which they are attached, they form a 4-7 membered heterocyclic, heteroaryl or carbocyclic ring;
R₃ is H, (1-3C)alkyl, halogeno or CF₃;
R₄ is cyano, (1-3C)alkyl, (1-3C)fluoroalkyl, (1-3C)alkoxy, (1-3C)perfluoroalkoxy, halo, (1-3C)alkanoyl, C(O)NRⁱR^{j}, or S(O)₂NRⁱR^{j}; wherein Rⁱ and R^{j} are each independently selected from H or (1-3C)alkyl;
X is CH or CR₅;
W, Y and Z are each independently selected from N, CH, or CR₅;
R₅ is halogeno, trifluoromethyl, trifluoromethoxy, cyano, nitro, hydroxy, mercapto, amino, carboxy, carbamoyl, sulphamoyl, ureido, (1-6C)alkyl, (2-6C)alkenyl, (2-6C)alkynyl,
or R₅ is a group of the formula:
-L¹-L²-R₇
wherein
L¹ is absent or a linker group of the formula —[CR₈R₉]ₙ— in which n is an integer selected from 1, 2, 3 or 4, and R₈ and R₉ are each independently selected from hydrogen or (1-2C)alkyl;
L² is absent or is selected from O, S, SO, SO₂, N(R₁₀), C(O), C(O)O, OC(O), CH(OR₁₀), C(O)N(R₁₀), N(R₁₀)C(O), N(R₁₀)C(O)N(R₁₁), S(O)₂N(R₁₀), or N(R₁₃)SO₂, wherein R₁₀ and R₁₁ are each independently selected from hydrogen or (1-2C)alkyl; and
R₇ is (1-6C)alkyl, aryl, aryl-(1-6C)alkyl, (3-6C)cycloalkyl, (3-6C)cycloalkyl-(1-6C)alkyl, heteroaryl, heteroaryl-(1-6C)alkyl, heterocyclyl, heterocyclyl-(1-6C)alkyl,
and wherein R₇ is optionally further substituted by one or more substituents independently selected from hydrogen, oxo, halogeno, cyano, nitro, hydroxy, NR₁₂R₁₃, (1-4C)alkoxy, (1-5C)alkyl, (3-8C)cycloalkyl, (3-8C)cycloalkyl-(1-5C)alkyl, aryl, aryl-(1-5C)alkyl, (1-5C)alkanoyl, (1-5C)alkylsulphonyl, heterocyclyl, heterocyclyl-(1-5C)alkyl, heteroaryl, heteroaryl-(1-5C)alkyl, CON R₁₂R₁₃ and SO₂NR₁₂R₁₃;
R₁₂ and R₁₃ are each independently selected from hydrogen or (1-2C)alkyl; or R₁₂ and R₁₃ can be linked such that, together with the nitrogen atom to which they are attached, they form a 4-7 membered heterocyclic or heteroaryl ring;
or either W and Z, W and Y or Z and X are both CR₅ and the R₅ groups on the adjacent carbon atoms are linked such that, together with the carbon atoms to which they are attached, they form a fused 4-7 membered heterocyclic, heteroaryl or carbocyclic ring.

26. A method according to claim 25 wherein the MPS1 inhibitor is selected from the group consisting of NMS-P715, BAY 1217389, BAY 1161909, CCT289346, a compound of formula I, a compound of formula II, a compound of formula III and a compound of formula IV, or pharmaceutically acceptable salts or solvates thereof.

27. A method according to claim 25 wherein the MPS1 inhibitor is selected from the group consisting of a compound of formula I, a compound of formula II, a compound of formula III and a compound of formula IV, or pharmaceutically acceptable salts or solvates thereof.

28. A method according to claim 25 wherein the MPS1 inhibitor is selected from the group consisting of a compound of formula I or a compound of formula II, or pharmaceutically acceptable salts or solvates thereof.

29. A method according to any one of the preceding claims wherein the MPS1 inhibitor is selected from the following:
5-(furan-2-yl)-N-(4-methoxyphenyl)isoquinolin-3-amine;
N-(4-methoxyphenyl)-5-(1-methyl-1H-pyrazol-4-yl)isoquinolin-3-amine;
N-(2-methoxy-4-((1-methylpiperidin-4-yl)oxy)phenyl)-5-(1-methyl-1H-pyrazol-4-yl)isoquinolin-3-amine;
N-(2,4-dimethoxyphenyl)-5-(1-methyl-1H-pyrazol-4-yl)isoquinolin-3-amine;
3-chloro-N, N-dimethyl-4-((5-(1-methyl-1H-pyrazol-4-yl)isoquinolin-3-yl)amino)benzamide;
3-methoxy-N,N-dimethyl-4-((5-(1-methyl-1H-pyrazol-4-yl)isoquinolin-3-yl)amino)benzamide;
(3-methoxy-4-((5-(1-methyl-1H-pyrazol-4-yl)isoquinolin-3-yl)amino)phenyl)(3-methoxyazetidin-1-yl)methanone;
N-(2-chloro-4-(1-methyl-1H-pyrazol-4-yl)phenyl)-5-(1-methyl-1H-pyrazol-4-yl)isoquinolin-3-amine;
(3-chloro-4-((5-(1-methyl-1H-pyrazol-4-yl)isoquinolin-3-yl)amino)phenyl)(3-methoxyazetidin-1-yl)methanone;
(3-methoxy-4-((5-(pyridin-3-yl)isoquinolin-3-yl)amino)phenyl)(3-methoxyazetidin-1-yl)methanone;
N-(4-(3,5-dimethylisoxazol-4-yl)-2-methoxyphenyl)-5-(1-methyl-1H-pyrazol-4-yl)isoquinolin-3-amine;
(3-methoxy-4-((8-(1-methyl-1H-pyrazol-4-yl)pyrido[3,4-d]pyrimidin-2-yl)amino)phenyl)(3-methoxyazetidin-1-yl)methanone;
N-(2-methoxy-4-(1-methyl-1H-pyrazol-4-yl)phenyl)-8-(1-methyl-1H-pyrazol-4-yl)pyrido[3,4-d]pyrimidin-2-amine;
N-(2-chloro-4-(1-methyl-1H-pyrazol-4-yl)phenyl)-8-(1-methyl-1H-pyrazol-4-yl)pyrido[3,4-d]pyrimidin-2-amine;
N-(2-chloro-4-(1-methyl-1H-imidazol-5-yl)phenyl)-5-(1-methyl-1H-pyrazol-4-yl)isoquinolin-3-amine;
N-(2-methoxy-4-(1-methyl-1H-pyrazol-4-yl)phenyl)-5-(1-methyl-1H-pyrazol-4-yl)isoquinolin-3-amine;
(3-methoxy-4-((5-(pyrimidin-5-yl)isoquinolin-3-yl)amino)phenyl)(3-methoxyazetidin-1-yl)methanone;
N-(2-methoxy-4-(1-methyl-1H-imidazol-5-yl)phenyl)-5-(1-methyl-1H-pyrazol-4-yl)isoquinolin-3-amine;
(4-((5-(1,5-dimethyl-1H-pyrazol-4-yl)isoquinolin-3-yl)amino)-3-methoxyphenyl)(3-methoxyazetidin-1-yl)methanone;
(3-methoxy-4-((5-(1-methyl-1H-pyrazol-3-yl)isoquinolin-3-yl)amino)phenyl)(3-methoxyazetidin-1-yl)methanone;
N-(2-chloro-4-(1,2-dimethyl-1H-imidazol-5-yl)phenyl)-5-(1-methyl-1H-pyrazol-4-yl)isoquinolin-3-amine;
N-(4-(1,2-dimethyl-1H-imidazol-5-yl)-2-methoxyphenyl)-5-(1-methyl-1H-pyrazol-4-yl)isoquinolin-3-amine;
N-(2-methoxy-4-(1-methyl-1H-pyrazol-4-yl)phenyl)-8-phenylpyrido[3,4-d]pyrimidin-2-amine;
8-cyclopropyl-N-(2-methoxy-4-(1-methyl-1H-pyrazol-4-yl)phenyl)pyrido[3,4-d]pyrimidin-2-amine;
N-(2-methoxy-5-methyl-4-(1-methyl-1H-pyrazol-4-yl)phenyl)-8-(1-methyl-1H-pyrazol-4-yl)pyrido[3,4-d]pyrimidin-2-amine;
(3-methoxy-4-((5-(1-methyl-1H-pyrazol-5-yl)isoquinolin-3-yl)amino)phenyl)(3-methoxyazetidin-1-yl)methanone;
(4-((5-(1,3-dimethyl-1H-pyrazol-4-yl)isoquinolin-3-yl)amino)-3-methoxyphenyl)(3-methoxyazetidin-1-yl)methanone;
(4-((5-(1-isopropyl-1H-pyrazol-4-yl)isoquinolin-3-yl)amino)-3-methoxyphenyl)(3-methoxyazetidin-1-yl)methanone;
4-((5-(1-methyl-1H-pyrazol-4-yl)isoquinolin-3-yl)amino)-N-(1-methylpiperidin-4-yl)-3-(trifluoromethoxy)benzamide;
(4-((5-(3,5-dimethylisoxazol-4-yl)isoquinolin-3-yl)amino)-3-methoxyphenyl)(3-methoxyazetidin-1-yl)methanone;
(3-methoxy-4-((5-(1-methyl-1H-imidazol-5-yl)isoquinolin-3-yl)amino)phenyl)(3-methoxyazetidin-1-yl)methanone;
N-(2-methoxy-4-(1-methyl-1H-pyrazol-4-yl)phenyl)-8-(pyrrolidin-1-yl)pyrido[3,4-d]pyrimidin-2-amine;
N-(4-(1,2-dimethyl-1H-imidazol-5-yl)-2-methoxyphenyl)-8-(1-methyl-1H-pyrazol-4-yl)pyrido[3,4-d]pyrimidin-2-amine;
tert-butyl 4-(4-(3-((2-methoxy-4-(3-methoxyazetidine-1-carbonyl)phenyl)amino)isoquinolin-5-yl)-1H-pyrazol-1-yl)piperidine-1-carboxylate;
(3-methoxy-4-((5-(1-(piperidin-4-yl)-1H-pyrazol-4-yl)isoquinolin-3-yl)amino)phenyl)(3-methoxyazetidin-1-yl)methanone;
(3-methoxy-4-((5-(1-(1-methylpiperidin-4-yl)-1H-pyrazol-4-yl)isoquinolin-3-yl)amino)phenyl)(3-methoxyazetidin-1-yl)methanone;
(3-methoxy-4-((5-(1-(2-methoxyethyl)-1H-pyrazol-4-yl)isoquinolin-3-yl)amino)phenyl)(3-methoxyazetidin-1-yl)methanone;
N8,N8-diethyl-N2-(2-methoxy-4-(1-methyl-1H-pyrazol-4-yl)phenyl)pyrido[3,4-d]pyrimidine-2,8-diamine;
N8-cyclopentyl-N2-(2-methoxy-4-(1-methyl-1H-pyrazol-4-yl)phenyl)pyrido[3,4-d]pyrimidine-2,8-diamine;
(4-((5-(1-(2-(dimethylamino)ethyl)-1H-pyrazol-4-yl)isoquinolin-3-yl)amino)-3-methoxyphenyl)(3-methoxyazetidin-1-yl)methanone;
N-(2-methoxy-4-(1-methyl-1H-pyrazol-4-yl)phenyl)-5-(1-methyl-1H-pyrazol-4-yl)-2,6-naphthyridin-3-amine;
N-(2-methoxy-4-(1-methyl-1H-pyrazol-4-yl)phenyl)-8-(piperidin-1-yl)pyrido[3,4-d]pyrimidin-2-amine;
N8-cyclohexyl-N2-(2-methoxy-4-(1-methyl-1H-pyrazol-4-yl)phenyl)pyrido[3,4-d]pyrimidine-2,8-diamine;
N-(2-methoxy-4-(1-methyl-1H-pyrazol-4-yl)phenyl)-8-(3-methylpyrrolidin-1-yl)pyrido[3,4-d]pyrimidin-2-amine;
8-(3,3-difluoropyrrolidin-1-yl)-N-(2-methoxy-4-(1-methyl-1H-pyrazol-4-yl)phenyl)pyrido[3,4-d]pyrimidin-2-amine;
N-(4-(1,2-dimethyl-1H-imidazol-5-yl)-2-methoxyphenyl)-5-(1-methyl-1H-pyrazol-4-yl)-2,6-naphthyridin-3-amine;
N8-(cyclopropylmethyl)-N2-(2-methoxy-4-(1-methyl-1H-pyrazol-4-yl)phenyl)pyrido[3,4-d]pyrimidine-2,8-diamine;
8-(1-methyl-1H-pyrazol-4-yl)-N-(2-methyl-4-(1-methyl-1H-pyrazol-4-yl)phenyl)pyrido[3,4-d]pyrimidin-2-amine;
N8-cyclopentyl-N2-(2-methoxy-4-(1-methyl-1H-pyrazol-4-yl)phenyl)-N8-methylpyrido[3,4-d]pyrimidine-2,8-diamine;
N-(2-ethoxy-4-(1-methyl-1H-pyrazol-4-yl)phenyl)-8-(1-methyl-1H-pyrazol-4-yl)pyrido[3,4-d]pyrimidin-2-amine;
N-(2-isopropoxy-4-(1-methyl-1H-pyrazol-4-yl)phenyl)-8-(1-methyl-1H-pyrazol-4-yl)pyrido[3,4-d]pyrimidin-2-amine;
N-(2-(2-methoxyethoxy)-4-(1-methyl-1H-pyrazol-4-yl)phenyl)-8-0-methyl-1H-pyrazol-4-yl)pyrido[3,4-d]pyrimidin-2-amine;
N8-isopentyl-N2-(2-methoxy-4-(1-methyl-1H-pyrazol-4-yl)phenyl)pyrido[3,4-d]pyrimidine-2,8-diamine;
N-(2-methoxy-4-(1-methyl-1H-pyrazol-4-yl)phenyl)-8-morpholinopyrido[3,4-d]pyrimidin-2-amine;
N-(2-methoxy-4-(1-methyl-1H-pyrazol-4-yl)phenyl)-8-(4-methylpiperazin-1-yl)pyrido[3,4-d]pyrimidin-2-amine;
8-(3,3-difluoroazetidin-1-yl)-N-(2-methoxy-4-(1-methyl-1H-pyrazol-4-yl)phenyl)pyrido[3,4-d]pyrimidin-2-amine;
N-(2-methoxy-4-(1-methyl-1H-pyrazol-4-yl)phenyl)-8-(2-methylpyrrolidin-1-yl)pyrido[3,4-d]pyrimidin-2-amine;
N8-isobutyl-N2-(2-methoxy-4-(1-methyl-1H-pyrazol-4-yl)phenyl)pyrido[3,4-d]pyrimidine-2,8-diamine;
8-(cyclohexylthio)-N-(2-methoxy-4-(1-methyl-1H-pyrazol-4-yl)phenyl)pyrido[3,4-d]pyrimidin-2-amine;
N-(2-methoxy-4-(1-methyl-1H-pyrazol-4-yl)phenyl)-8-(6-azaspiro[3.4]octan-6-yl)pyrido[3,4-d]pyrimidin-2-amine;
N8-cyclohexyl-N2-(2-methoxy-4-(1-(2-(4-methylpiperazin-1-yl)ethyl)-1H-pyrazol-4-yl)phenyl)pyrido[3,4-d]pyrimidine-2,8-diamine;
8-(1-ethyl-1H-pyrazol-4-yl)-N-(2-methoxy-4-(1-methyl-1H-pyrazol-4-yl)phenyl)pyrido[3,4-d]pyrimidin-2-amine;
8-(1-isopropyl-1H-pyrazol-4-yl)-N-(2-methoxy-4-(1-methyl-1H-pyrazol-4-yl)phenyl)pyrido[3,4-d]pyrimidin-2-amine;
N-(2-methoxy-4-(1-methyl-1H-pyrazol-4-yl)phenyl)-8-(3-methoxyazetidin-1-yl)pyrido[3,4-d]pyrimidin-2-amine;
N1-(cyclopropylmethyl)-N7-(2-methoxy-4-(1-methyl-1H-pyrazol-4-yl)phenyl)-2,6-naphthyridine-1,7-diamine;
N1-cyclohexyl-N7-(2-methoxy-4-(1-methyl-1H-pyrazol-4-yl)phenyl)-2,6-naphthyridine-1,7-diamine;
N8-cyclohexyl-N2-(4-(1-(2-(dimethylamino)ethyl)-1H-pyrazol-4-yl)-2-methoxyphenyl)pyrido[3,4-d]pyrimidine-2,8-diamine;
N2-(2-methoxy-4-(1-methyl-1H-pyrazol-4-yl)phenyl)-N8-neopentylpyrido[3,4-d]pyrimidine-2,8-diamine;
N2-(2-methoxy-4-(1-methyl-1H-pyrazol-4-yl)phenyl)-N8-(tetrahydro-2H-pyran-4-yl)pyrido[3,4-d]pyrimidine-2,8-diamine;
N8-(cyclopropylmethyl)-N2-(2-methyl-4-(1-methyl-1H-pyrazol-4-yl)phenyl)pyrido[3,4-d]pyrimidine-2,8-diamine;
N8-cyclohexyl-N2-(2-methyl-4-(1-methyl-1H-pyrazol-4-yl)phenyl)pyrido[3,4-d]pyrimidine-2,8-diamine;
N8-(cyclopropylmethyl)-N2-(2-ethoxy-4-(1-methyl-1H-pyrazol-4-yl)phenyl)pyrido[3,4-d]pyrimidine-2,8-diamine;
N8-(cyclohexylmethyl)-N2-(2-methoxy-4-(1-methyl-1H-pyrazol-4-yl)phenyl)pyrido[3,4-d]pyrimidine-2,8-diamine;
2-(4-(4-((8-(cyclohexylamino)pyrido[3,4-d]pyrimidin-2-yl)amino)-3-methoxyphenyl)-1H-pyrazol-1-yl)ethanol;
8-(cyclopropylmethoxy)-N-(2-methoxy-4-(1-methyl-1H-pyrazol-4-yl)phenyl)pyrido[3,4-d]pyrimidin-2-amine;
1-((2-((2-methoxy-4-(1-methyl-1H-pyrazol-4-yl)phenyl)amino)pyrido[3,4-d]pyrimidin-8-yl)amino)-2-methylpropan-2-ol;
N2-(2-methoxy-4-(1-methyl-1H-pyrazol-4-yl)phenyl)-N8-(oxetan-3-ylmethyl)pyrido[3,4-d]pyrimidine-2,8-diamine;
N8-(3,3-dimethylbutan-2-yl)-N2-(2-methoxy-4-(1-methyl-1H-pyrazol-4-yl)phenyl)pyrido[3,4-d]pyrimidine-2,8-diamine;
3-((2-((2-methoxy-4-(1-methyl-1H-pyrazol-4-yl)phenyl)amino)pyrido[3,4-d]pyrimidin-8-yl)amino)-2,2-dimethylpropan-1-ol;
N2-(2-ethoxy-4-(1-methyl-1H-pyrazol-4-yl)phenyl)-N8-(tetrahydro-2H-pyran-4-yl)pyrido[3,4-d]pyrimidine-2,8-diamine;
N2-(2-methoxy-6-morpholinopyridin-3-yl)-N8-neopentylpyrido[3,4-d]pyrimidine-2,8-diamine;
N2-(2-methoxy-6-(methylsulfonyl)pyridin-3-yl)-N8-neopentylpyrido[3,4-d]pyrimidine-2,8-diamine;
N2-(2-methoxy-4-(1-methyl-1H-imidazol-5-yl)phenyl)-N8-neopentylpyrido[3,4-d]pyrimidine-2,8-diamine;
N2-(4-(1,3-dimethyl-1H-pyrazol-4-yl)-2-methoxyphenyl)-N8-neopentylpyrido[3,4-d]pyrimidine-2,8-diamine;
N8-(1-cyclopropylethyl)-N2-(2-methoxy-4-(1-methyl-1H-pyrazol-4-yl)phenyl)pyrido[3,4-d]pyrimidine-2,8-diamine;
2-(4-(3-methoxy-4-((8-((tetrahydro-2H-pyran-4-yl)amino)pyrido[3,4-d]pyrimidin-2-yl)amino)phenyl)-1H-pyrazol-1-yl)ethanol;
N2-(4-(1,5-dimethyl-1H-pyrazol-4-yl)-2-methoxyphenyl)-N8-neopentylpyrido[3,4-d]pyrimidine-2,8-diamine;
(R)-N8-(3,3-dimethylbutan-2-yl)-N2-(2-methoxy-4-(1-methyl-1H-pyrazol-4-yl)phenyl)pyrido[3,4-d]pyrimidine-2,8-diamine;
(S)-N8-(3,3-dimethylbutan-2-yl)-N2-(2-methoxy-4-(1-methyl-1H-pyrazol-4-yl)phenyl)pyrido[3,4-d]pyrimidine-2,8-diamine;
N2-(2-methoxy-4-(1-methyl-1H-pyrazol-4-yl)phenyl)-N8-(tetrahydrofuran-3-yl)pyrido[3,4-d]pyrimidine-2,8-diamine;
N2-(2-methoxy-4-(1-methyl-1H-pyrazol-4-yl)phenyl)-N8-((tetrahydrofuran-3-yl)methyl)pyrido[3,4-d]pyrimidine-2,8-diamine;
1-(2-((2-methoxy-4-(1-methyl-1H-pyrazol-4-yl)phenyl)amino)pyrido[3,4-d]pyrimidin-8-yl)pyrrolidin-3-ol;
N2-(2-methoxy-4-(1-methyl-1H-pyrazol-4-yl)phenyl)-N8-methyl-N8-(tetrahydro-2H-pyran-4-yl)pyrido[3,4-d]pyrimidine-2,8-diamine;
N8-(tert-butyl)-N2-(2-methoxy-4-(1-methyl-1H-pyrazol-4-yl)phenyl)pyrido[3,4-d]pyrimidine-2,8-diamine;
N2-(2-methoxy-4-(1-methyl-1H-pyrazol-4-yl)phenyl)-N8-(1-methylcyclohexyl)pyrido[3,4-d]pyrimidine-2,8-diamine;
8-(1-(2,2-difluoroethyl)-1H-pyrazol-4-yl)-N-(2-methoxy-4-(1-methyl-1H-pyrazol-4-yl)phenyl)pyrido[3,4-d]pyrimidin-2-amine;
N2-(4-(1,2-dimethyl-1H-imidazol-5-yl)-2-methoxyphenyl)-N8-neopentylpyrido[3,4-d]pyrimidine-2,8-diamine;
N2-(2-methoxy-4-(1-methyl-1H-pyrazol-4-yl)phenyl)-6-methyl-N8-neopentylpyrido[3,4-d]pyrimidine-2,8-diamine;
N2-(2-methoxy-4-morpholinophenyl)-N8-neopentylpyrido[3,4-d]pyrimidine-2,8-diamine;
N8-(2,2-difluoropropyl)-N2-(2-methoxy-4-(1-methyl-1H-pyrazol-4-yl)phenyl)pyrido[3,4-d]pyrimidine-2,8-diamine;
N8-(3-methoxy-2,2-dimethylpropyl)-N2-(2-methoxy-4-(1-methyl-1H-pyrazol-4-yl)phenyl)pyrido[3,4-d]pyrimidine-2,8-diamine;
N8-(2-methoxy-2-methylpropyl)-N2-(2-methoxy-4-(1-methyl-1H-pyrazol-4-yl)phenyl)pyrido[3,4-d]pyrimidine-2,8-diamine;
N2-(2-methoxy-4-(1-methyl-1H-pyrazol-4-yl)phenyl)-N8-(2,2,2-trifluoroethyl)pyrido[3,4-d]pyrimidine-2,8-diamine;
N-(2-methoxy-4-(1-methyl-1H-pyrazol-4-yl)phenyl)-8-(2-oxa-6-azaspiro[3.4]octan-6-yl)pyrido[3,4-d]pyrimidin-2-amine;
1-(((2-((2-methoxy-4-(1-methyl-1H-pyrazol-4-yl)phenyl)amino)pyrido[3,4-d]pyrimidin-8-yl)amino)methyl)cyclobutanol;
8-chloro-N-(2-methoxy-4-(1-methyl-1H-pyrazol-4-yl)phenyl)pyrido[3,4-d]pyrimidin-2-amine;
N2-(2-ethyl-4-(1-methyl-1H-pyrazol-4-yl)phenyl)-N8-neopentylpyrido[3,4-d]pyrimidine-2,8-diamine;
N2-(4-(1-methyl-1H-pyrazol-4-yl)-2-(trifluoromethoxy)phenyl)-N8-neopentylpyrido[3,4-d]pyrimidine-2,8-diamine;
N2-(2-methoxy-4-(1-methyl-1H-pyrazol-4-yl)phenyl)-N8-methylpyrido[3,4-d]pyrimidine-2,8-diamine;
N2-(2-methoxy-4-(1-methyl-1H-pyrazol-4-yl)phenyl)-N8,N8-dimethylpyrido[3,4-d]pyrimidine-2,8-diamine;
N-(2-((2-methoxy-4-(1-methyl-1H-pyrazol-4-yl)phenyl)amino)pyrido[3,4-d]pyrimidin-8-yl)-2-methylpropane-2-sulfinamide;
N2-(2-methoxy-4-(4-morpholinopiperidin-1-yl)phenyl)-N8-neopentylpyrido[3,4-d]pyrimidine-2,8-diamine;
N2-(2-methoxy-4-(1-methyl-1H-pyrazol-4-yl)phenyl)-N8-((3-methyloxetan-3-yl)methyl)pyrido[3,4-d]pyrimidine-2,8-diamine;
N2-(2-methoxy-4-(piperidin-1-yl)phenyl)-N8-neopentylpyrido[3,4-d]pyrimidine-2,8-diamine;
N-(2-((2-methoxy-4-(1-methyl-1H-pyrazol-4-yl)phenyl)amino)pyrido[3,4-d]pyrimidin-8-yl)-2-methylpropane-2-sulfonamide;
N2-(2-methoxy-4-(1-methyl-1H-pyrazol-4-yl)phenyl)-N8-(oxetan-3-yl)pyrido[3,4-d]pyrimidine-2,8-diamine;
(1-(3-methoxy-4-((8-(neopentylamino)pyrido[3,4-d]pyrimidin-2-yl)amino)phenyl)piperidin-4-yl)(morpholino)methanone;
N2-(2-methoxy-4-(4-methylpiperazin-1-yl)phenyl)-N8-neopentylpyrido[3,4-d]pyrimidine-2,8-diamine;
1-(((2-((2-methoxy-4-(1-methyl-1H-pyrazol-4-yl)phenyl)amino)pyrido[3,4-d]pyrimidin-8-yl)amino)methyl)cyclopropanol;
N2-(2-methoxy-4-(1-methyl-1H-pyrazol-4-yl)phenyl)-N8-(1-methylpiperidin-4-yl)pyrido[3,4-d]pyrimidine-2,8-diamine;
2-((2-((2-methoxy-4-(1-methyl-1H-pyrazol-4-yl)phenyl)amino)pyrido[3,4-d]pyrimidin-8-yl)amino)-2-methylpropan-1-ol;
N-(2-methoxy-4-(1-methyl-1H-pyrazol-4-yl)phenyl)-8-(2-oxa-6-azaspiro[3.3]heptan-6-yl)pyrido[3,4-d]pyrimidin-2-amine;
N2-(2-methoxy-4-(1-methyl-1H-pyrazol-4-yl)phenyl)-N8-(oxetan-2-ylmethyl)pyrido[3,4-d]pyrimidine-2,8-diamine;
N2-(2-chloro-4-morpholinophenyl)-N8-neopentylpyrido[3,4-d]pyrimidine-2,8-diamine
N2-(2-methoxy-4-(4-methyl-4H-1,2,4-triazol-3-yl)phenyl)-N8-neopentylpyrido[3,4-d]pyrimidine-2,8-diamine;
N2-(2-methoxy-4-(4-(methylsulfonyl)piperazin-1-yl)phenyl)-N8-neopentylpyrido[3,4-d]pyrimidine-2,8-diamine;
N2-(2-methoxy-4-(1-methyl-1H-pyrazol-4-yl)phenyl)-N8-((3-methyltetrahydrofuran-3-yl)methyl)pyrido[3,4-d]pyrimidine-2,8-diamine;
N-(4-(1,5-dimethyl-1H-pyrazol-4-yl)-2-methoxyphenyl)-8-(2-oxa-6-azaspiro[3.4]octan-6-yl)pyrido[3,4-d]pyrimidin-2-amine;
N2-(4-(1,5-dimethyl-1H-pyrazol-4-yl)-2-methoxyphenyl)-N8-((3-methyloxetan-3-yl)methyl)pyrido[3,4-d]pyrimidine-2,8-diamine;
N2-(4-(1,5-dimethyl-1H-pyrazol-4-yl)-2-methoxyphenyl)-N8-(2-methoxy-2-methylpropyl)pyrido[3,4-d]pyrimidine-2,8-diamine;
N2-(4-(1,2-dimethyl-1H-imidazol-5-yl)-2-methoxyphenyl)-N8-(2-methoxy-2-methylpropyl)-6-methylpyrido[3,4-d]pyrimidine-2,8-diamine;
N2-(4-(1,2-dimethyl-1H-imidazol-5-yl)-2-methoxyphenyl)-6-methyl-N8-neopentylpyrido[3,4-d]pyrimidine-2,8-diamine;
N2-(2-methoxy-4-(4-methyl-4H-1,2,4-triazol-3-yl)phenyl)-6-methyl-N8-neopentylpyrido[3,4-d]pyrimidine-2,8-diamine;
N-(2-ethoxy-4-(1-methyl-1H-pyrazol-4-yl)phenyl)-8-(2-oxa-6-azaspiro[3.4]octan-6-yl)pyrido[3,4-d]pyrimidin-2-amine;
2-((2-((2-methoxy-4-(1-methyl-1H-pyrazol-4-yl)phenyl)amino)pyrido[3,4-d]pyrimidin-8-yl)amino)ethanol;
N2-(2-methoxy-4-(1-methyl-1H-pyrazol-4-yl)phenyl)-N8-(2-methoxyethyl)pyrido[3,4-d]pyrimidine-2,8-diamine;
1-((2-((2-methoxy-4-(1-methyl-1H-pyrazol-4-yl)phenyl)amino)pyrido[3,4-d]pyrimidin-8-yl)amino)propan-2-ol;
2-((2-((2-methoxy-4-(1-methyl-1H-pyrazol-4-yl)phenyl)amino)pyrido[3,4-d]pyrimidin-8-yl)amino)propan-1-ol;
N2-(4-(1,5-dimethyl-1H-pyrazol-4-yl)-2-methoxyphenyl)-N8-((3-methyltetrahydrofuran-3-yl)methyl)pyrido[3,4-d]pyrimidine-2,8-diamine;
4-(2-((2-methoxy-4-(1-methyl-1H-pyrazol-4-yl)phenyl)amino)pyrido[3,4-d]pyrimidin-8-yl)thiomorpholine 1,1-dioxide;
N-(2-methoxy-4-(1-methyl-1H-pyrazol-4-yl)phenyl)-8-(7-oxa-2-azaspiro[3.5]nonan-2-yl)pyrido[3,4-d]pyrimidin-2-amine;
N2-(2-methoxy-4-(1-methyl-1H-pyrazol-4-yl)phenyl)-5-methyl-N8-neopentylpyrido[3,4-d]pyrimidine-2,8-diamine;
N-(2-methoxy-4-(1-methyl-1H-pyrazol-4-yl)phenyl)-8-(6-oxa-2-azaspiro[3.4]octan-2-yl)pyrido[3,4-d]pyrimidin-2-amine;
1-(2-((2-methoxy-4-(1-methyl-1H-pyrazol-4-yl)phenyl)amino)pyrido[3,4-d]pyrimidin-8-yl)azetidine-3-carbonitrile;
N-(2-methoxy-4-(1-methyl-1H-pyrazol-4-yl)phenyl)-8-(2-oxa-7-azaspiro[4.4]nonan-7-yl)pyrido[3,4-d]pyrimidin-2-amine;
N-(2-methoxy-4-(1-methyl-1H-pyrazol-4-yl)phenyl)-8-(2-oxa-6-azaspiro[3.5]nonan-6-yl)pyrido[3,4-d]pyrimidin-2-amine;
N8-((3-fluorooxetan-3-yl)methyl)-N2-(2-methoxy-4-(1-methyl-1H-pyrazol-4-yl)phenyl)pyrido[3,4-d]pyrimidine-2,8-diamine;
N-(4-chloro-2-methoxyphenyl)-8-(2-oxa-6-azaspiro[3.4]octan-6-yl)pyrido[3,4-d]pyrimidin-2-amine;
N-(2,4-dichlorophenyl)-8-(2-oxa-6-azaspiro[3.4]octan-6-yl)pyrido[3,4-d]pyrimidin-2-amine;
4-((8-(2-oxa-6-azaspiro[3.4]octan-6-yl)pyrido[3,4-d]pyrimidin-2-yl)amino)-3-methoxybenzonitrile;
N-(2-chloro-4-(methylsulfonyl)phenyl)-8-(2-oxa-6-azaspiro[3.4]octan-6-yl)pyrido[3,4-d]pyrimidin-2-amine;
N-(2-chloro-4-(pyrimidin-5-yl)phenyl)-8-(2-oxa-6-azaspiro[3.4]octan-6-yl) pyrido[3,4-d]pyrimidin-2-amine;
N-(2-chloro-4-(5-methyl-1,3,4-oxadiazol-2-yl)phenyl)-8-(2-oxa-6-azaspiro[3.4]octan-6-yl)pyrido[3,4-d]pyrimidin-2-amine;
N8-(2-methoxy-2-methylpropyl)-N2-(2-methoxy-4-(4-methyl-4H-1,2,4-triazol-3-yl)phenyl)-6-methylpyrido[3,4-d]pyrimidine-2,8-diamine;
6-cyclopropyl-N-(2-methoxy-4-(1-methyl-1H-pyrazol-4-yl)phenyl)-8-(2-oxa-6-azaspiro[3.4]octan-6-yl)pyrido[3,4-d]pyrimidin-2-amine;
2-((2-((2-methoxy-4-(1-methyl-1H-pyrazol-4-yl)phenyl)amino)pyrido[3,4-d]pyrimidin-8-yl)amino)propane-1,3-diol;
3-methoxy-2-((2-((2-methoxy-4-(1-methyl-1H-pyrazol-4-yl)phenyl)amino)pyrido[3,4-d]pyrimidin-8-yl)amino)propan-1-ol;
(3-(((2-((2-methoxy-4-(1-methyl-1H-pyrazol-4-yl)phenyl)amino)pyrido[3,4-d]pyrimidin-8-yl)amino)methyl)oxetan-3-yl)methanol;
(S)-N2-(2-methoxy-4-(1-methyl-1H-pyrazol-4-yl)phenyl)-N8-((3-methyltetrahydrofuran-3-yl)methyl)pyrido[3,4-d]pyrimidine-2,8-diamine;
(R)-N2-(2-methoxy-4-(1-methyl-1H-pyrazol-4-yl)phenyl)-N8-((3-methyltetrahydrofuran-3-yl)methyl)pyrido[3,4-d]pyrimidine-2,8-diamine;
N-(4-chloro-2-fluorophenyl)-8-(2-oxa-6-azaspiro[3.4]octan-6-yl) pyrido[3,4-d]pyrimidin-2-amine;
4-((8-(2-oxa-6-azaspiro[3.4]octan-6-yl)pyrido[3,4-d]pyrimidin-2-yl)amino)-3-chlorobenzonitrile;
N2-(2-methoxy-4-(1-(2-methoxyethyl)-2-methyl-1H-imidazol-5-yl)phenyl)-6-methyl-N8-neopentylpyrido[3,4-d]pyrimidine-2,8-diamine;
N2-(2-ethoxy-4-(4-methyl-4H-1,2,4-triazol-3-yl)phenyl)-6-methyl-N8-neopentylpyrido[3,4-d]pyrimidine-2,8-diamine;
N2-(2-methoxy-4-(4-(methylsulfonyl)piperazin-1-yl)phenyl)-6-methyl-N8-neopentylpyrido[3,4-d]pyrimidine-2,8-diamine;
N-(2-methoxy-4-(1-methyl-1H-pyrazol-4-yl)phenyl)-8-(pyridin-4-yl)pyrido[3,4-d]pyrimidin-2-amine;
N8-(2-methoxy-2-methylpropyl)-N2-(2-methoxy-4-(4-methyl-4H-1,2,4-triazol-3-yl)phenyl)-5-methylpyrido[3,4-d]pyrimidine-2,8-diamine;
N2-(2-methoxy-4-(4-methyl-4H-1,2,4-triazol-3-yl)phenyl)-5-methyl-N8-neopentylpyrido[3,4-d]pyrimidine-2,8-diamine;
N-(2-methoxy-4-(1-methyl-1H-pyrazol-4-yl)phenyl)-8-(2-methylmorpholino)pyrido[3,4-d]pyrimidin-2-amine;
(4-(3-methoxy-4-((8-((2-methoxy-2-methylpropyl)amino)pyrido[3,4-d]pyrimidin-2-yl)amino)phenyl)-1-methyl-1H-pyrazol-5-yl)methanol;
(4-(3-methoxy-4-((8-(((3-methyltetrahydrofuran-3-yl)methyl)amino)pyrido[3,4-d]pyrimidin-2-yl)amino)phenyl)-1-methyl-1H-pyrazol-5-yl)methanol;
N8-(2-methoxy-2-methylpropyl)-N2-(2-methoxy-4-(1-(2-methoxyethyl)-2-methyl-1H-imidazol-5-yl)phenyl)pyrido[3,4-d]pyrimidine-2,8-diamine;
N2-(2-methoxy-4-(1-(2-methoxyethyl)-2-methyl-1H-imidazol-5-yl)phenyl)-N8-((3-methyltetrahydrofuran-3-yl)methyl)pyrido[3,4-d]pyrimidine-2,8-diamine;
N2-(2-ethoxy-4-(4-methyl-4H-1,2,4-triazol-3-yl)phenyl)-N8-(2-methoxy-2-methylpropyl)pyrido[3,4-d]pyrimidine-2,8-diamine;
N2-(2-ethoxy-4-(4-methyl-4H-1,2,4-triazol-3-yl)phenyl)-N8-((3-methyltetrahydrofuran-3-yl)methyl)pyrido[3,4-d]pyrimidine-2,8-diamine;
8-(3,6-dihydro-2H-pyran-4-yl)-N-(2-methoxy-4-(1-methyl-1H-pyrazol-4-yl)phenyl)pyrido[3,4-d]pyrimidin-2-amine;
N8-(2-methoxy-2-methylpropyl)-N2-(2-methoxy-6-(1-methyl-1H-tetrazol-5-yl)pyridin-3-yl)pyrido[3,4-d]pyrimidine-2,8-diamine;
N2-(6-(1,2-dimethyl-1H-imidazol-5-yl)-2-methoxypyridin-3-yl)-N8-(2-methoxy-2-methylpropyl)pyrido[3,4-d]pyrimidine-2,8-diamine;
N8-(2-methoxy-2-methylpropyl)-N2-(2-methoxy-4-(1-methyl-1H-tetrazol-5-yl)phenyl)pyrido[3,4-d]pyrimidine-2,8-diamine;
N-(2-methoxy-4-(1-methyl-1H-pyrazol-4-yl)phenyl)-8-(pyrimidin-5-yl)pyrido[3,4-d]pyrimidin-2-amine;
N2-(2-methoxy-4-(1-methyl-1H-pyrazol-4-yl)phenyl)-N8-(1-(tetrahydrofuran-3-yl)ethyl)pyrido[3,4-d]pyrimidine-2,8-diamine;
N2-(4-(1,3-dimethyl-1H-pyrazol-4-yl)-2-methoxyphenyl)-N8-(2-methoxy-2-methylpropyl)pyrido[3,4-d]pyrimidine-2,8-diamine;
N2-(4-(1,3-dimethyl-1H-pyrazol-4-yl)-2-methoxyphenyl)-N8-((3-methyltetrahydrofuran-3-yl)methyl)pyrido[3,4-d]pyrimidine-2,8-diamine;
N-(2-methoxy-4-(1-methyl-1H-pyrazol-4-yl)phenyl)-8-(4-methoxypiperidin-1-yl)pyrido[3,4-d]pyrimidin-2-amine;
1-(2-((2-methoxy-4-(1-methyl-1H-pyrazol-4-yl)phenyl)amino)pyrido[3,4-d]pyrimidin-8-yl)piperidine-4-carbonitrile;
N-(2-methoxy-4-(1-methyl-1H-pyrazol-4-yl)phenyl)-8-(4-(methylsulfonyl)piperazin-1-yl)pyrido[3,4-d]pyrimidin-2-amine;
N2-(4-(1,3-dimethyl-1H-pyrazol-4-yl)-2-methoxyphenyl)-N8-(2-methoxy-2-methylpropyl)-6-methylpyrido[3,4-d]pyrimidine-2,8-diamine;
N-(2-ethoxy-4-(4-methyl-4H-1,2,4-triazol-3-yl)phenyl)-6-methyl-8-(6-oxa-2-azaspiro[3.4]octan-2-yl)pyrido[3,4-d]pyrimidin-2-amine;
N2-(6-(1,3-dimethyl-1H-pyrazol-4-yl)-2-methoxypyridin-3-yl)-N8-(2-methoxy-2-methylpropyl)pyrido[3,4-d]pyrimidine-2,8-diamine;
N2-(6-(1,5-dimethyl-1H-pyrazol-4-yl)-2-methoxypyridin-3-yl)-N8-(2-methoxy-2-methylpropyl)pyrido[3,4-d]pyrimidine-2,8-diamine;
N8-(2-methoxy-2-methylpropyl)-N2-(2-methoxy-6-(1-methyl-1H-1,2,3-triazol-5-yl)pyridin-3-yl)pyrido[3,4-d]pyrimidine-2,8-diamine;
N8-(2-methoxy-2-methylpropyl)-N2-(2-methoxy-6-(2-methyl-2H-1,2,3-triazol-4-yl)pyridin-3-yl)pyrido[3,4-d]pyrimidine-2,8-diamine;
(3-methoxy-4-((8-((2-methoxy-2-methylpropyl)amino)pyrido[3,4-d]pyrimidin-2-yl)amino)phenyl)(3-methoxyazetidin-1-yl)methanone;
3-methoxy-4-((8-((2-methoxy-2-methylpropyl)amino)pyrido[3,4-d]pyrimidin-2-yl)amino)-N,N-dimethylbenzamide;
(3-methoxy-4-((8-((2-methoxy-2-methylpropyl)amino)pyrido[3,4-d]pyrimidin-2-yl)amino)phenyl)(4-methylpiperazin-1-yl)methanone;
(1-(2-((2-methoxy-4-(1-methyl-1H-pyrazol-4-yl)phenyl)amino)pyrido[3,4-d]pyrimidin-8-yl)pyrrolidin-3-yl)methanol;
(1-(2-((2-methoxy-4-(1-methyl-1H-pyrazol-4-yl)phenyl)amino)pyrido[3,4-d]pyrimidin-8-yl)piperidin-3-yl)methanol;
(4-(2-((2-methoxy-4-(1-methyl-1H-pyrazol-4-yl)phenyl)amino)pyrido[3,4-d]pyrimidin-8-yl)morpholin-2-yl)methanol;
N2-(2-(difluoromethoxy)-4-(1-methyl-1H-pyrazol-4-yl)phenyl)-N8-(2-methoxy-2-methylpropyl)pyrido[3,4-d]pyrimidine-2,8-diamine;
N2-(2-(difluoromethoxy)-4-fluorophenyl)-N8-(2-methoxy-2-methylpropyl)pyrido[3,4-d]pyrimidine-2,8-diamine;
N2-(4-(1-ethyl-1H-pyrazol-4-yl)-2-methoxyphenyl)-N8-(2-methoxy-2-methylpropyl)pyrido[3,4-d]pyrimidine-2,8-diamine;
(3-methoxy-4-((8-(neopentylamino)pyrido[3,4-d]pyrimidin-2-yl)amino)phenyl)(3-methoxyazetidin-1-yl)methanone;
N2-(2-methoxy-4-(tetrahydro-2H-pyran-4-yl)phenyl)-N8-((3-methyltetrahydrofuran-3-yl)methyl)pyrido[3,4-d]pyrimidine-2,8-diamine;
N2-(4-chloro-2-(difluoromethoxy)phenyl)-N8-(2-methoxy-2-methylpropyl)pyrido[3,4-d]pyrimidine-2,8-diamine;
N2-(2-methoxy-4-(4-methyl-4H-1,2,4-triazol-3-yl)phenyl)-5-methyl-N8-((3-methyloxetan-3-yl)methyl)pyrido[3,4-d]pyrimidine-2,8-diamine;
N2-(2-methoxy-4-(4-methyl-4H-1,2,4-triazol-3-yl)phenyl)-5-methyl-N8-((3-methyltetrahydrofuran-3-yl)methyl)pyrido[3,4-d]pyrimidine-2 ,8-diamine;
N-(2-methoxy-4-(4-methyl-4H-1,2,4-triazol-3-yl)phenyl)-5-methyl-8-(6-oxa-2-azaspiro[3.4]octan-2-yl)pyrido[3,4-d]pyrimidin-2-amine;
N8-(2-methoxy-2-methylpropyl)-N2-(2-methoxy-4-(1-methyl-1H-1,2,4-triazol-3-yl)phenyl)-6-methylpyrido[3,4-d]pyrimidine-2,8-diamine;
N2-(2-(difluoromethoxy)-4-(1-methyl-1H-pyrazol-4-yl)phenyl)-N8-(2-methoxy-2-methylpropyl)-6-methylpyrido[3,4-d]pyrimidine-2,8-diamine;
(4-(3-methoxy-4-((84(2-methoxy-2-methylpropyl)amino)-6-methylpyrido[3,4-d]pyrimidin-2-yl)amino)phenyl)-1-methyl-1H-pyrazol-5-yl)methanol;
N2-(2-methoxy-4-(1-methyl-1H-1,2,3-triazol-5-yl)phenyl)-6-methyl-N8-neopentylpyrido[3,4-d]pyrimidine-2,8-diamine;
N2-(2-ethoxy-4-(4-methyl-4H-1,2,4-triazol-3-yl)phenyl)-6-methyl-N8-((3-methyltetrahydrofuran-3-yl)methyl)pyrido[3,4-d]pyrimidine-2,8-diamine;
1-(((2-((2-ethoxy-4-(4-methyl-4H-1,2,4-triazol-3-yl)phenyl)amino)-6-methylpyrido[3,4-d]pyrimidin-8-yl)amino)methyl)cyclobutanol;
1-(2-((2-ethoxy-4-(4-methyl-4H-1,2 ,4-triazol-3-yl)phenyl)amino)-6-methyl pyrido[3,4-d]pyrimidin-8-yl)-3-methylazetidine-3-carbonitrile;
N-(2-ethoxy-4-(4-methyl-4H-1,2,4-triazol-3-yl)phenyl)-6-methyl-8-(7-oxa-2-azaspiro[3.5]nonan-2-yl)pyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)piperidine-4-carbonitrile;
1-(2-((2-ethoxy-4-(4-methyl-4H-1,2,4-triazol-3-yl)phenyl)amino)-6-methylpyrido[3,4-d]pyrimidin-8-yl)-3-methylazetidin-3-ol;
N2-(2-ethoxy-4-(4-methyl-4H-1,2,4-triazol-3-yl)phenyl)-6-methyl-N8-((3-methyloxetan-3-yl)methyl)pyrido[3,4-d]pyrimidine-2,8-diamine;
N2-(2-ethoxy-4-(4-methyl-4H-1,2,4-triazol-3-yl)phenyl)-6-methyl-N8-(tetrahydro-2H-pyran-4-yl)pyrido[3,4-d]pyrimidine-2,8-diamine;
N-(2-ethoxy-4-(4-methyl-4H-1,2,4-triazol-3-yl)phenyl)-6-methyl-8-(2-oxa-7-azaspiro[4.4]nonan-7-yl)pyrido[3,4-d]pyrimidin-2-amine;
1-(((2-((2-ethoxy-4-(4-methyl-4H-1,2,4-triazol-3-yl)phenyl)amino)-6-methylpyrido[3,4-d]pyrimidin-8-yl)amino)methyl)cyclopropanol;
N2-(4-(1,2-dimethyl-1H-imidazol-5-yl)-2-methoxyphenyl)-6-methyl-N8-((3-methyltetrahydrofuran-3-yl)methyl)pyrido[3,4-d]pyrimidine-2,8-diamine;
N-(4-(1,2-dimethyl-1H-imidazol-5-yl)-2-methoxyphenyl)-6-methyl-8-(2-oxa-7-azaspiro[4.4]nonan-7-yl)pyrido[3,4-d]pyrimidin-2-amine;
1-(2-((4-(1,2-dimethyl-1H-imidazol-5-yl)-2-methoxyphenyl)amino)-6-methylpyrido[3,4-d]pyrimidin-8-yl)piperidine-4-carbonitrile;
1-(2-((4-(1,2-dimethyl-1H-imidazol-5-yl)-2-methoxyphenyl)amino)-6-methylpyrido[3,4-d]pyrimidin-8-yl)-3-methylazetidine-3-carbonitrile;
N-(2-ethoxy-4-(4-methyl-4H-1,2,4-triazol-3-yl)phenyl)-6-methyl-8-(1-oxa-6-azaspiro[3.3]heptan-6-yl)pyrido[3,4-d]pyrimidin-2-amine;
N2-(2-methoxy-4-(1-methyl-1H-pyrazol-3-yl)phenyl)-6-methyl-N8-neopentylpyrido[3,4-d]pyrimidine-2,8-diamine;
8-(3,3-difluoroazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(4-methoxypiperidin-1-yl)-6-methylpyrido[3,4-d]pyrimidin-2-amine;
N-(2-ethoxy-4-(4-methyl-4H-1,2,4-triazol-3-yl)phenyl)-8-(3-methoxypiperidin-1-yl)-6-methylpyrido[3,4-d]pyrimidin-2-amine;
N-(2-ethoxy-4-(4-methyl-4H-1,2,4-triazol-3-yl)phenyl)-6-methyl-8-(2-methylmorpholino)pyrido[3,4-d]pyrimidin-2-amine;
N-(2-ethoxy-4-(4-methyl-4H-1,2,4-triazol-3-yl)phenyl)-8-(4-methoxy-4-methylpiperidin-1-yl)-6-methylpyrido[3,4-d]pyrimidin-2-amine;
8-(3-azabicyclo[3.1.0]hexan-3-yl)-N-(2-ethoxy-4-(4-methyl-4H-1,2,4-triazol-3-yl)phenyl)-6-methylpyrido[3,4-d]pyrimidin-2-amine;
8-(3-(dimethylamino)azetidin-1-yl)-N-(2-ethoxy-4-(4-methyl-4H-1,2,4-triazol-3-yl)phenyl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)piperidin-4-ol;
N-(2-ethoxy-4-(4-methyl-4H-1,2,4-triazol-3-yl)phenyl)-8-(3-methoxy-3-methylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylpyrrolidin-3-ol;
1-(2-((2-ethoxy-4-(4-methyl-4H-1,2,4-triazol-3-yl)phenyl)amino)-6-methylpyrido[3,4-d]pyrimidin-8-yl)-4-methylpiperidine-4-carbonitrile;
1-(2-((2-ethoxy-4-(4-methyl-4H-1,2,4-triazol-3-yl)phenyl)amino)-6-methylpyrido[3,4-d]pyrimidin-8-yl)pyrrolidine-3-carbonitrile;
N2-(2-methoxy-4-(1-methyl-1H-imidazol-2-yl)phenyl)-6-methyl-N8-neopentylpyrido[3,4-d]pyrimidine-2,8-diamine;
N2-(2-methoxy-4-(1-methyl-1H-pyrazol-5-yl)phenyl)-6-methyl-N8-neopentylpyrido[3,4-d]pyrimidine-2,8-diamine;
N2-(2-methoxy-4-(oxazol-2-yl) phenyl)-6-methyl-N8-neopentylpyrido[3,4-d]pyrimidine-2,8-diamine;
N-(2-ethoxy-4-(4-methyl-4H-1,2,4-triazol-3-yl)phenyl)-8-(3-methoxypyrrolidin-1-yl)-6-methylpyrido[3,4-d]pyrimidin-2-amine;
8-(3,3-dimethylazetidin-1-yl)-N-(2-ethoxy-4-(4-methyl-4H-1,2,4-triazol-3-yl)phenyl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylpyrrolidine-3-carbonitrile;
8-(2,2-dimethylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-6-methyl-8-(3-(trifluoromethypazetidin-1-yl)pyrido[3,4-d]pyrimidin-2-amine;
N-(2-ethoxy-4-(4-methyl-4H-1,2,4-triazol-3-yl)phenyl)-6-methyl-8-(2-azaspiro[3.3]heptan-2-yl)pyrido[3,4-d]pyrimidin-2-amine;
(R)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
(S)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
N2-(2-ethoxy-4-(4-methyl-4H-1,2,4-triazol-3-yl)phenyl)-N8-((1-methoxycyclobutyl)methyl)-6-methylpyrido[3,4-d]pyrimidine-2,8-diamine;
N2-(2-ethoxy-4-(4-methyl-4H-1,2,4-triazol-3-yl)phenyl)-6-methyl-N8-(1-methylazetidin-3-yl)pyrido[3,4-d]pyrimidine-2,8-diamine;
N2-(2-ethoxy-4-(4-methyl-4H-1,2,4-triazol-3-yl)phenyl)-6-methyl-N8-(oxetan-3-ylmethyl)pyrido[3,4-d]pyrimidine-2,8-diamine;
N-(2-ethoxy-4-(4-methyl-4H-1,2,4-triazol-3-yl)phenyl)-6-methyl-8-(pyrrolidin-1-yl)pyrido[3,4-d]pyrimidin-2-amine;
N-(2-ethoxy-4-(4-methyl-4H-1,2,4-triazol-3-yl)phenyl)-6-methyl-8-(2-azaspiro[3.4]octan-2-yl)pyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-ethylazetidin-3-ol;
N2-(2-ethoxy-4-(4-methyl-4H-1,2,4-triazol-3-yl)phenyl)-6-methyl-N8-(1-methylpiperidin-4-yl)pyrido[3,4-d]pyrimidine-2,8-diamine;
8-(4-(dimethylamino)piperidin-1-yl)-N-(2-ethoxy-4-(4-methyl-4H-1,2,4-triazol-3-yl)phenyl)-6-methylpyrido[3,4-d]pyrimidin-2-amine;
N2-(2-ethoxy-4-(4-methyl-4H-1,2,4-triazol-3-yl)phenyl)-6-methyl-N8-((tetrahydro-2H-pyran-4-yl)methyl)pyrido[3,4-d]pyrimidine-2,8-diamine;
N2-(2-ethoxy-4-(4-methyl-4H-1,2,4-triazol-3-yl)phenyl)-6-methyl-N8-((4-methyltetrahydro-2H-pyran-4-yl)methyl)pyrido[3,4-d]pyrimidine-2,8-diamine;
1-(2-((2-ethoxy-4-(4-methyl-4H-1,2,4-triazol-3-yl)phenyl)amino)-6-methylpyrido[3,4-d]pyrimidin-8-yl)-4-ethylpiperidine-4-carbonitrile;
N2-(2-ethoxy-4-(4-methyl-4H-1,2,4-triazol-3-yl)phenyl)-6-methyl-N8-(2-(3-methyltetrahydrofuran-3-yl)ethyl)pyrido[3,4-d]pyrimidine-2,8-diamine;
N2-(2-ethoxy-4-(4-methyl-4H-1,2,4-triazol-3-yl)phenyl)-6-methyl-N8-(1-(tetrahydro-2H-pyran-4-yl)ethyl)pyrido[3,4-d]pyrimidine-2,8-diamine;
N2-(2-ethoxy-4-(4-methyl-4H-1,2,4-triazol-3-yl)phenyl)-6-methyl-N8-(pentan-3-yl)pyrido[3,4-d]pyrimidine-2,8-diamine;
N2-(2-ethoxy-4-(4-methyl-4H-1,2,4-triazol-3-yl)phenyl)-6-methyl-N8-(tetrahydrofuran-3-yl)pyrido[3,4-d]pyrimidine-2,8-diamine;
8-(3-ethoxy-3-methylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(3-ethyl-3-methoxyazetidin-1-yl)-6-methylpyrido[3,4-c]pyrimidin-2-amine;
8-(3-ethoxy-3-ethylazetidin-1-yl)-N-(2-ethoxy-4-(4-methyl-4H-1,2,4-triazol-3-yl)phenyl)-6-methylpyrido[3,4-c]pyrimidin-2-amine;
N-(2-ethoxy-4-(4-methyl-4H-1,2,4-triazol-3-yl)phenyl)-8-(3-isopropyl-3-methoxyazetidin-1-yl)-6-methylpyrido[3,4-c]pyrimidin-2-amine;
8-(3-ethoxy-3-isopropylazetidin-1-yl)-N-(2-ethoxy-4-(4-methyl-4H-1,2,4-triazol-3-yl)phenyl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-c]pyrimidin-8-yl)-3-ethylazetidine-3-carbonitrile;
1-(2-((2-ethoxy-4-(4-methyl-4H-1,2,4-triazol-3-yl)phenyl)amino)-6-methylpyrido[3,4-c]pyrimidin-8-yl)-3-isopropylazetidine-3-carbonitrile;
1-(2-((2-ethoxy-4-(4-methyl-4H-1,2,4-triazol-3-yl)phenyl)amino)-6-methylpyrido[3,4-c]pyrimidin-8-yl)-2,2,3-trimethylazetidine-3-carbonitrile;
N-(2-ethoxy-4-(4-methyl-4H-1,2,4-triazol-3-yl)phenyl)-8-(3-methoxy-2,2-dimethylazetidin-1-yl)-6-methylpyrido[3,4-c]pyrimidin-2-amine;
N-(2-ethoxy-4-(4-methyl-4H-1,2,4-triazol-3-yl)phenyl)-8-(3-methoxy-2,2,3-trimethylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-2,2-dimethylazetidine-3-carbonitrile;
N2-(2-methoxy-4-(4-methyl-4H-1,2,4-triazol-3-yl)phenyl)-6-methyl-N8-(1-methylpiperidin-4-yl)pyrido[3,4-d]pyrimidine-2,8-diamine;
8-(4-(dimethylamino)piperidin-1-yl)-N-(2-methoxy-4-(4-methyl-4H-1,2,4-triazol-3-yl)phenyl)-6-methylpyrido[3,4-d]pyrimidin-2-amine;
N2-(2-methoxy-4-(4-methyl-4H-1,2,4-triazol-3-yl)phenyl)-6-methyl-N8-((tetrahydro-2H-pyran-4-yl)methyl)pyrido[3,4-d]pyrimidine-2,8-diamine;
N2-(2-methoxy-4-(4-methyl-4H-1,2,4-triazol-3-yl)phenyl)-6-methyl-N8-((4-methyltetrahydro-2H-pyran-4-yl)methyl)pyrido[3,4-d]pyrimidine-2,8-diamine;
4-ethyl-1-(2-((2-methoxy-4-(4-methyl-4H-1,2,4-triazol-3-yl)phenyl)amino)-6-methylpyrido[3,4-d]pyrimidin-8-yl)piperidine-4-carbonitrile;
N2-(2-methoxy-4-(4-methyl-4H-1,2,4-triazol-3-yl)phenyl)-6-methyl-N8-(2-(3-methyltetrahydrofuran-3-yl)ethyl)pyrido[3,4-d]pyrimidine-2,8-diamine;
N2-(2-methoxy-4-(4-methyl-4H-1,2,4-triazol-3-yl)phenyl)-6-methyl-N8-(1-(tetrahydro-2H-pyran-4-yl)ethyl)pyrido[3,4-d]pyrimidine-2,8-diamine;
N2-(2-methoxy-4-(4-methyl-4H-1,2,4-triazol-3-yl)phenyl)-6-methyl-N8-(pentan-3-yl)pyrido[3,4-d]pyrimidine-2,8-diamine;
N2-(2-methoxy-4-(4-methyl-4H-1,2,4-triazol-3-yl)phenyl)-6-methyl-N8-(tetrahydrofuran-3-yl)pyrido[3,4-d]pyrimidine-2,8-diamine;
8-(3-ethoxy-3-methylazetidin-1-yl)-N-(2-methoxy-4-(4-methyl-4H-1,2,4-triazol-3-yl)phenyl)-6-methylpyrido[3,4-d]pyrimidin-2-amine;
8-(3-ethyl-3-methoxyazetidin-1-yl)-N-(2-methoxy-4-(4-methyl-4H-1,2,4-triazol-3-yl)phenyl)-6-methylpyrido[3,4-d]pyrimidin-2-amine;
8-(3-ethoxy-3-ethylazetidin-1-yl)-N-(2-methoxy-4-(4-methyl-4H-1,2,4-triazol-3-yl)phenyl)-6-methylpyrido[3,4-d]pyrimidin-2-amine;
8-(3-isopropyl-3-methoxyazetidin-1-yl)-N-(2-methoxy-4-(4-methyl-4H-1,2,4-triazol-3-yl)phenyl)-6-methylpyrido[3,4-d]pyrimidin-2-amine;
8-(3-ethoxy-3-isopropylazetidin-1-yl)-N-(2-methoxy-4-(4-methyl-4H-1,2,4-triazol-3-yl)phenyl)-6-methylpyrido[3,4-d]pyrimidin-2-amine;
3-ethyl-1-(2-((2-methoxy-4-(4-methyl-4H-1,2,4-triazol-3-yl)phenyl)amino)-6-methylpyrido[3,4-d]pyrimidin-8-yl)azetidine-3-carbonitrile;
3-isopropyl-1-(2-((2-methoxy-4-(4-methyl-4H-1,2,4-triazol-3-yl)phenyl)amino)-6-methylpyrido[3,4-d]pyrimidin-8-yl)azetidine-3-carbonitrile;
1-(2-((2-methoxy-4-(4-methyl-4H-1,2,4-triazol-3-yl)phenyl)amino)-6-methylpyrido[3,4-d]pyrimidin-8-yl)-2,2,3-trimethylazetidine-3-carbonitrile;
8-(3-methoxy-2,2-dimethylazetidin-1-yl)-N-(2-methoxy-4-(4-methyl-4H-1,2,4-triazol-3-yl)phenyl)-6-methylpyrido[3,4-d]pyrimidin-2-amine;
8-(3-methoxy-2,2,3-trimethylazetidin-1-yl)-N-(2-methoxy-4-(4-methyl-4H-1,2,4-triazol-3-yl)phenyl)-6-methylpyrido[3,4-d]pyrimidin-2-amine;
1-(2-((2-methoxy-4-(4-methyl-4H-1,2,4-triazol-3-yl)phenyl)amino)-6-methylpyrido[3,4-d]pyrimidin-8-yl)-2,2-dimethylazetidine-3-carbonitrile;
8-(3,3-dimethylazetidin-1-yl)-N-(2-methoxy-4-(1-methyl-1H-tetrazol-5-yl)phenyl)-6-methylpyrido[3,4-d]pyrimidin-2-amine;
1-(2-((2-methoxy-4-(1-methyl-1H-tetrazol-5-yl)phenyl)amino)-6-methylpyrido[3,4-d]pyrimidin-8-yl)-4-methylpiperidine-4-carbonitrile;
8-(3-methoxy-3-methylazetidin-1-yl)-N-(2-methoxy-4-(1-methyl-1H-tetrazol-5-yl)phenyl)-6-methylpyrido[3,4-d]pyrimidin-2-amine;
N-(2-methoxy-4-(1-methyl-1H-tetrazol-5-yl)phenyl)-8-(4-methoxypiperidin-1-yl)-6-methylpyrido[3,4-d]pyrimidin-2-amine;
N-(2-methoxy-4-(1-methyl-1H-tetrazol-5-yl)phenyl)-8-(4-methoxy-4-methylpiperidin-1-yl)-6-methylpyrido[3,4-d]pyrimidin-2-amine;
N-(2-methoxy-4-(1-methyl-1H-tetrazol-5-yl)phenyl)-6-methyl-8-(1-oxa-6-azaspiro[3.3]heptan-6-yl)pyrido[3,4-d]pyrimidin-2-amine;
1-(2-((2-methoxy-4-(1-methyl-1H-tetrazol-5-yl)phenyl)amino)-6-methylpyrido[3,4-d]pyrimidin-8-yl)-3-methylazetidine-3-carbonitrile;
N2-(2-methoxy-4-(1-methyl-1H-tetrazol-5-yl)phenyl)-6-methyl-N8-((3-methyltetrahydrofuran-3-yl)methyl)pyrido[3,4-d]pyrimidine-2,8-diamine;
N-(2-methoxy-4-(1-methyl-1H-tetrazol-5-yl)phenyl)-6-methyl-8-(2-oxa-7-azaspiro[4.4]nonan-7-yl)pyrido[3,4-d]pyrimidin-2-amine;
N2-(2-methoxy-4-(1-methyl-1H-tetrazol-5-yl)phenyl)-6-methyl-N8-(tetrahydro-2H-pyran-4-yl)pyrido[3,4-d]pyrimidine-2,8-diamine;
N-(2-methoxy-4-(1-methyl-1H-tetrazol-5-yl)phenyl)-6-methyl-8-(7-oxa-2-azaspiro[3.5]nonan-2-yl)pyrido[3,4-d]pyrimidin-2-amine;
N2-(2-methoxy-4-(1-methyl-1H-tetrazol-5-yl)phenyl)-6-methyl-N8-neopentylpyrido[3,4-d]pyrimidine-2,8-diamine;
N-(2-(difluoromethoxy)-4-(4-methyl-4H-1,2,4-triazol-3-yl)phenyl)-8-(3,3-dimethylazetidin-1-yl)-6-methylpyrido[3,4-d]pyrimidin-2-amine;
1-(2-((2-(difluoromethoxy)-4-(4-methyl-4H-1,2,4-triazol-3-yl)phenyl)amino)-6-methylpyrido[3,4-d]pyrimidin-8-yl)-4-methylpiperidine-4-carbonitrile;
N-(2-(difluoromethoxy)-4-(4-methyl-4H-1,2,4-triazol-3-yl)phenyl)-8-(3-methoxy-3-methylazetidin-1-yl)-6-methylpyrido[3,4-d]pyrimidin-2-amine;
N-(2-(difluoromethoxy)-4-(4-methyl-4H-1,2,4-triazol-3-yl)phenyl)-8-(4-methoxypiperidin-1-yl)-6-methylpyrido[3,4-d]pyrimidin-2-amine;
N-(2-(difluoromethoxy)-4-(4-methyl-4H-1,2,4-triazol-3-yl)phenyl)-8-(4-methoxy-4-methylpiperidin-1-yl)-6-methylpyrido[3,4-d]pyrimidin-2-amine;
N-(2-(difluoromethoxy)-4-(4-methyl-4H-1,2,4-triazol-3-yl)phenyl)-6-methyl-8-(1-oxa-6-azaspiro[3.3]heptan-6-yl)pyrido[3,4-d]pyrimidin-2-amine;
1-(2-((2-(difluoromethoxy)-4-(4-methyl-4H-1,2,4-triazol-3-yl)phenyl)amino)-6-methylpyrido[3,4-d]pyrimidin-8-yl)-3-methylazetidine-3-carbonitrile;
N2-(2-(difluoromethoxy)-4-(4-methyl-4H-1,2,4-triazol-3-yl)phenyl)-6-methyl-N8-((3-methyltetrahydrofuran-3-yl)methyl)pyrido[3,4-d]pyrimidine-2,8-diamine;
N-(2-(difluoromethoxy)-4-(4-methyl-4H-1,2,4-triazol-3-yl)phenyl)-6-methyl-8-(2-oxa-7-azaspiro[4.4]nonan-7-yl)pyrido[3,4-d]pyrimidin-2-amine;
N2-(2-(difluoromethoxy)-4-(4-methyl-4H-1,2,4-triazol-3-yl)phenyl)-6-methyl-N8-(tetrahydro-2H-pyran-4-yl)pyrido[3,4-d]pyrimidine-2,8-diamine;
N-(2-(difluoromethoxy)-4-(4-methyl-4H-1,2,4-triazol-3-yl)phenyl)-6-methyl-8-(7-oxa-2-azaspiro[3.5]nonan-2-yl)pyrido[3,4-d]pyrimidin-2-amine;
N2-(2-(difluoromethoxy)-4-(4-methyl-4H-1,2,4-triazol-3-yl)phenyl)-6-methyl-N8-neopentylpyrido[3,4-d]pyrimidine-2,8-diamine;
8-(3,3-dimethylazetidin-1-yl)-N-(4-(4-ethyl-4H-1,2,4-triazol-3-yl)-2-methoxyphenyl)-6-methylpyrido[3,4-d]pyrimidin-2-amine;
1-(2-((4-(4-ethyl-4H-1,2,4-triazol-3-yl)-2-methoxyphenyl)amino)-6-methylpyrido[3,4-d]pyrimidin-8-yl)-4-methylpiperidine-4-carbonitrile;
N-(4-(4-ethyl-4H-1,2,4-triazol-3-yl)-2-methoxyphenyl)-8-(3-methoxy-3-methylazetidin-1-yl)-6-methylpyrido[3,4-d]pyrimidin-2-amine;
N-(4-(4-ethyl-4H-1,2,4-triazol-3-yl)-2-methoxyphenyl)-8-(4-methoxypiperidin-1-yl)-6-methylpyrido[3,4-d]pyrimidin-2-amine;
N-(4-(4-ethyl-4H-1,2,4-triazol-3-yl)-2-methoxyphenyl)-8-(4-methoxy-4-methylpiperidin-1-yl)-6-methylpyrido[3,4-d]pyrimidin-2-amine;
N-(4-(4-ethyl-4H-1,2,4-triazol-3-yl)-2-methoxyphenyl)-6-methyl-8-(1-oxa-6-azaspiro[3.3]heptan-6-yl)pyrido[3,4-d]pyrimidin-2-amine;
1-(2-((4-(4-ethyl-4H-1,2,4-triazol-3-yl)-2-methoxyphenyl)amino)-6-methylpyrido[3,4-d]pyrimidin-8-yl)-3-methylazetidine-3-carbonitrile;
N2-(4-(4-ethyl-4H-1,2,4-triazol-3-yl)-2-methoxyphenyl)-6-methyl-N8-((3-methyltetrahydrofuran-3-yl)methyl)pyrido[3,4-d]pyrimidine-2,8-diamine;
N-(4-(4-ethyl-4H-1,2,4-triazol-3-yl)-2-methoxyphenyl)-6-methyl-8-(2-oxa-7-azaspiro[4.4]nonan-7-yl)pyrido[3,4-d]pyrimidin-2-amine;
N2-(4-(4-ethyl-4H-1,2,4-triazol-3-yl)-2-methoxyphenyl)-6-methyl-N8-(tetrahydro-2H-pyran-4-yl)pyrido[3,4-d]pyrimidine-2,8-diamine;
N-(4-(4-ethyl-4H-1,2,4-triazol-3-yl)-2-methoxyphenyl)-6-methyl-8-(7-oxa-2-azaspiro[3.5]nonan-2-yl)pyrido[3,4-d]pyrimidin-2-amine;
N2-(4-(4-ethyl-4H-1,2,4-triazol-3-yl)-2-methoxyphenyl)-6-methyl-N8-neopentylpyrido[3,4-d]pyrimidine-2,8-diamine;
8-(3,3-dimethylazetidin-1-yl)-N-(2-ethoxy-4-(4-ethyl-4H-1,2,4-triazol-3-yl)phenyl)-6-methylpyrido[3,4-d]pyrimidin-2-amine;
1-(2-((2-ethoxy-4-(4-ethyl-4H-1,2,4-triazol-3-yl)phenyl)amino)-6-methylpyrido[3,4-d]pyrimidin-8-yl)-4-methylpiperidine-4-carbonitrile;
N-(2-ethoxy-4-(4-ethyl-4H-1,2,4-triazol-3-yl)phenyl)-8-(3-methoxy-3-methylazetidin-1-yl)-6-methylpyrido[3,4-d]pyrimidin-2-amine;
N-(2-ethoxy-4-(4-ethyl-4H-1,2,4-triazol-3-yl)phenyl)-8-(4-methoxypiperidin-1-yl)-6-methylpyrido[3,4-d]pyrimidin-2-amine;
N-(2-ethoxy-4-(4-ethyl-4H-1,2,4-triazol-3-yl)phenyl)-8-(4-methoxy-4-methylpiperidin-1-yl)-6-methylpyrido[3,4-d]pyrimidin-2-amine;
N-(2-ethoxy-4-(4-ethyl-4H-1,2,4-triazol-3-yl)phenyl)-6-methyl-8-(1-oxa-6-azaspiro[3.3]heptan-6-yl)pyrido[3,4-d]pyrimidin-2-amine;
1-(2-((2-ethoxy-4-(4-ethyl-4H-1,2,4-triazol-3-yl)phenyl)amino)-6-methylpyrido[3,4-d]pyrimidin-8-yl)-3-methylazetidine-3-carbonitrile;
N2-(2-ethoxy-4-(4-ethyl-4H-1,2,4-triazol-3-yl)phenyl)-6-methyl-N8-((3-methyltetrahydrofuran-3-yl)methyl)pyrido[3,4-d]pyrimidine-2,8-diamine;
N-(2-ethoxy-4-(4-ethyl-4H-1,2,4-triazol-3-yl)phenyl)-6-methyl-8-(2-oxa-7-azaspiro[4.4]nonan-7-yl)pyrido[3,4-d]pyrimidin-2-amine;
N2-(2-ethoxy-4-(4-ethyl-4H-1,2,4-triazol-3-yl)phenyl)-6-methyl-N8-(tetrahydro-2H-pyran-4-yl)pyrido[3,4-d]pyrimidine-2,8-diamine;
N-(2-ethoxy-4-(4-ethyl-4H-1,2,4-triazol-3-yl)phenyl)-6-methyl-8-(7-oxa-2-azaspiro[3.5]nonan-2-yl)pyrido[3,4-d]pyrimidin-2-amine;
N2-(2-ethoxy-4-(4-ethyl-4H-1,2,4-triazol-3-yl)phenyl)-6-methyl-N8-neopentylpyrido[3,4-d]pyrimidine-2,8-diamine;
N-(4-(4,5-dimethyl-4H-1,2,4-triazol-3-yl)-2-methoxyphenyl)-8-(3,3-dimethylazetidin-1-yl)-6-methylpyrido[3,4-d]pyrimidin-2-amine;
1-(2-((4-(4,5-dimethyl-4H-1,2,4-triazol-3-yl)-2-methoxyphenyl)amino)-6-methylpyrido[3,4-d]pyrimidin-8-yl)-4-methylpiperidine-4-carbonitrile;
N-(4-(4,5-dimethyl-4H-1,2,4-triazol-3-yl)-2-methoxyphenyl)-8-(3-methoxy-3-methylazetidin-1-yl)-6-methylpyrido[3,4-d]pyrimidin-2-amine;
N-(4-(4,5-dimethyl-4H-1,2,4-triazol-3-yl)-2-methoxyphenyl)-8-(4-methoxypiperidin-1-yl)-6-methylpyrido[3,4-d]pyrimidin-2-amine;
N-(4-(4,5-dimethyl-4H-1,2,4-triazol-3-yl)-2-methoxyphenyl)-8-(4-methoxy-4-methylpiperidin-1-yl)-6-methylpyrido[3,4-d]pyrimidin-2-amine;
N-(4-(4,5-dimethyl-4H-1,2,4-triazol-3-yl)-2-methoxyphenyl)-6-methyl-8-(1-oxa-6-azaspiro[3.3]heptan-6-yl)pyrido[3,4-d]pyrimidin-2-amine;
1-(2-((4-(4,5-dimethyl-4H-1,2,4-triazol-3-yl)-2-methoxyphenyl)amino)-6-methylpyrido[3,4-d]pyrimidin-8-yl)-3-methylazetidine-3-carbonitrile;
N2-(4-(4,5-dimethyl-4H-1,2,4-triazol-3-yl)-2-methoxyphenyl)-6-methyl-N8-((3-methyltetrahydrofuran-3-yl)methyl)pyrido[3,4-d]pyrimidine-2,8-diamine;
N-(4-(4,5-dimethyl-4H-1,2,4-triazol-3-yl)-2-methoxyphenyl)-6-methyl-8-(2-oxa-7-azaspiro[4.4]nonan-7-yl)pyrido[3,4-d]pyrimidin-2-amine;
N2-(4-(4,5-dimethyl-4H-1,2,4-triazol-3-yl)-2-methoxyphenyl)-6-methyl-N8-(tetrahydro-2H-pyran-4-yl)pyrido[3,4-d]pyrimidine-2,8-diamine;
N-(4-(4,5-dimethyl-4H-1,2,4-triazol-3-yl)-2-methoxyphenyl)-6-methyl-8-(7-oxa-2-azaspiro[3.5]nonan-2-yl)pyrido[3,4-d]pyrimidin-2-amine;
N2-(4-(4,5-dimethyl-4H-1,2,4-triazol-3-yl)-2-methoxyphenyl)-6-methyl-N8-neopentylpyrido[3,4-d]pyrimidine-2,8-diamine;
N-(4-(4,5-dimethyl-4H-1,2,4-triazol-3-yl)-2-ethoxyphenyl)-8-(3,3-dimethylazetidin-1-yl)-6-methylpyrido[3,4-d]pyrimidin-2-amine;
1-(2-((4-(4,5-dimethyl-4H-1,2,4-triazol-3-yl)-2-ethoxyphenyl)amino)-6-methylpyrido[3,4-d]pyrimidin-8-yl)-4-methylpiperidine-4-carbonitrile;
N-(4-(4,5-dimethyl-4H-1,2,4-triazol-3-yl)-2-ethoxyphenyl)-8-(3-methoxy-3-methylazetidin-1-yl)-6-methylpyrido[3,4-d]pyrimidin-2-amine;
N-(4-(4,5-dimethyl-4H-1,2,4-triazol-3-yl)-2-ethoxyphenyl)-8-(4-methoxypiperidin-1-yl)-6-methylpyrido[3,4-d]pyrimidin-2-amine;
N-(4-(4,5-dimethyl-4H-1,2,4-triazol-3-yl)-2-ethoxyphenyl)-8-(4-methoxy-4-methylpiperidin-1-yl)-6-methylpyrido[3,4-d]pyrimidin-2-amine;
N-(4-(4,5-dimethyl-4H-1,2,4-triazol-3-yl)-2-ethoxyphenyl)-6-methyl-8-(1-oxa-6-azaspiro[3.3]heptan-6-yl)pyrido[3,4-d]pyrimidin-2-amine;
1-(2-((4-(4,5-dimethyl-4H-1,2,4-triazol-3-yl)-2-ethoxyphenyl)amino)-6-methylpyrido[3,4-d]pyrimidin-8-yl)-3-methylazetidine-3-carbonitrile;
N2-(4-(4,5-dimethyl-4H-1,2,4-triazol-3-yl)-2-ethoxyphenyl)-6-methyl-N8-((3-methyltetrahydrofuran-3-yl)methyl)pyrido[3,4-d]pyrimidine-2,8-diamine;
N-(4-(4,5-dimethyl-4H-1,2,4-triazol-3-yl)-2-ethoxyphenyl)-6-methyl-8-(2-oxa-7-azaspiro[4.4]nonan-7-yl)pyrido[3,4-d]pyrimidin-2-amine;
N2-(4-(4,5-dimethyl-4H-1,2,4-triazol-3-yl)-2-ethoxyphenyl)-6-methyl-N8-(tetrahydro-2H-pyran-4-yl)pyrido[3,4-d]pyrimidine-2,8-diamine;
N-(4-(4,5-dimethyl-4H-1,2,4-triazol-3-yl)-2-ethoxyphenyl)-6-methyl-8-(7-oxa-2-azaspiro[3.5]nonan-2-yl)pyrido[3,4-d]pyrimidin-2-amine;
N2-(4-(4,5-dimethyl-4H-1,2,4-triazol-3-yl)-2-ethoxyphenyl)-6-methyl-N8-neopentylpyrido[3,4-d]pyrimidine-2,8-diamine;
8-(3,3-dimethylazetidin-1-yl)-N-(2-methoxy-4-(1-methyl-1H-1,2,3-triazol-5-yl)phenyl)-6-methylpyrido[3,4-d]pyrimidin-2-amine;
1-(2-((2-methoxy-4-(1-methyl-1H-1,2,3-triazol-5-yl)phenyl)amino)-6-methylpyrido[3,4-d]pyrimidin-8-yl)-4-methylpiperidine-4-carbonitrile;
8-(3-methoxy-3-methylazetidin-1-yl)-N-(2-methoxy-4-(1-methyl-1H-1,2,3-triazol-5-yl)phenyl)-6-methylpyrido[3,4-d]pyrimidin-2-amine;
N-(2-methoxy-4-(1-methyl-1H-1,2,3-triazol-5-yl)phenyl)-8-(4-methoxypiperidin-1-yl)-6-methylpyrido[3,4-d]pyrimidin-2-amine;
N-(2-methoxy-4-(1-methyl-1H-1,2,3-triazol-5-yl)phenyl)-8-(4-methoxy-4-methylpiperidin-1-yl)-6-methylpyrido[3,4-d]pyrimidin-2-amine;
N-(2-methoxy-4-(1-methyl-1H-1,2,3-triazol-5-yl)phenyl)-6-methyl-8-(1-oxa-6-azaspiro[3.3]heptan-6-yl)pyrido[3,4-d]pyrimidin-2-amine;
1-(2-((2-methoxy-4-(1-methyl-1H-1,2,3-triazol-5-yl)phenyl)amino)-6-methylpyrido[3,4-d]pyrimidin-8-yl)-3-methylazetidine-3-carbonitrile;
N2-(2-methoxy-4-(1-methyl-1H-1,2,3-triazol-5-yl)phenyl)-6-methyl-N8-((3-methyltetrahydrofuran-3-yl)methyl)pyrido[3,4-d]pyrimidine-2,8-diamine;
N-(2-methoxy-4-(1-methyl-1H-1,2,3-triazol-5-yl)phenyl)-6-methyl-8-(2-oxa-7-azaspiro[4.4]nonan-7-yl)pyrido[3,4-d]pyrimidin-2-amine;
N2-(2-methoxy-4-(1-methyl-1H-1 ,2, 3-triazol-5-yl)phenyl)-6-methyl-N8-(tetrahydro-2H-pyran-4-yl)pyrido[3,4-d]pyrimidine-2,8-diamine;
N-(2-methoxy-4-(1-methyl-1H-1,2,3-triazol-5-yl)phenyl)-6-methyl-8-(7-oxa-2-azaspiro[3.5]nonan-2-yl)pyrido[3,4-d]pyrimidin-2-amine;
N-(4-(1,2-dimethyl-1H-imidazol-5-yl)-2-methoxyphenyl)-8-(3,3-dimethylazetidin-1-yl)-6-methylpyrido[3,4-d]pyrimidin-2-amine;
1-(2-((4-(1,2-dimethyl-1H-imidazol-5-yl)-2-methoxyphenyl)amino)-6-methylpyrido[3,4-d]pyrimidin-8-yl)-4-methylpiperidine-4-carbonitrile;
N-(4-(1,2-dimethyl-1H-imidazol-5-yl)-2-methoxyphenyl)-8-(3-methoxy-3-methylazetidin-1-yl)-6-methylpyrido[3,4-d]pyrimidin-2-amine;
N-(4-(1,2-dimethyl-1H-imidazol-5-yl)-2-methoxyphenyl)-8-(4-methoxypiperidin-1-yl)-6-methylpyrido[3,4-d]pyrimidin-2-amine;
N-(4-(1,2-dimethyl-1H-imidazol-5-yl)-2-methoxyphenyl)-8-(4-methoxy-4-methylpiperidin-1-yl)-6-methylpyrido[3,4-d]pyrimidin-2-amine;
N-(4-(1,2-dimethyl-1H-imidazol-5-yl)-2-methoxyphenyl)-6-methyl-8-(1-oxa-6-azaspiro[3.3]heptan-6-yl)pyrido[3,4-d]pyrimidin-2-amine;
1-(2-((4-(1,2-dimethyl-1H-imidazol-5-yl)-2-methoxyphenyl)amino)-6-methylpyrido[3,4-d]pyrimidin-8-yl)-3-methylazetidine-3-carbonitrile;
N-(4-(1,2-dimethyl-1H-imidazol-5-yl)-2-methoxyphenyl)-6-methyl-8-(2-oxa-7-azaspiro[4.4]nonan-7-yl)pyrido[3,4-d]pyrimidin-2-amine;
N2-(4-(1,2-dimethyl-1H-imidazol-5-yl)-2-methoxyphenyl)-6-methyl-N8-(tetrahydro-2H-pyran-4-yl)pyrido[3,4-d]pyrimidine-2,8-diamine;
N-(4-(1,2-dimethyl-1H-imidazol-5-yl)-2-methoxyphenyl)-6-methyl-8-(7-oxa-2-azaspiro[3.5]nonan-2-yl)pyrido[3,4-d]pyrimidin-2-amine;
N-(4-(1,5-dimethyl-1H-imidazol-2-yl)-2-methoxyphenyl)-8-(3,3-dimethylazetidin-1-yl)-6-methylpyrido[3,4-d]pyrimidin-2-amine;
1-(2-((4-(1,5-dimethyl-1H-imidazol-2-yl)-2-methoxyphenyl)amino)-6-methylpyrido[3,4-d]pyrimidin-8-yl)-4-methylpiperidine-4-carbonitrile;
N-(4-(1,5-dimethyl-1H-imidazol-2-yl)-2-methoxyphenyl)-8-(3-methoxy-3-methylazetidin-1-yl)-6-methylpyrido[3,4-d]pyrimidin-2-amine;
N-(4-(1,5-dimethyl-1H-imidazol-2-yl)-2-methoxyphenyl)-8-(4-methoxypiperidin-1-yl)-6-methylpyrido[3,4-d]pyrimidin-2-amine;
N-(4-(1,5-dimethyl-1H-imidazol-2-yl)-2-methoxyphenyl)-8-(4-methoxy-4-methylpiperidin-1-yl)-6-methylpyrido[3,4-d]pyrimidin-2-amine;
N-(4-(1,5-dimethyl-1H-imidazol-2-yl)-2-methoxyphenyl)-6-methyl-8-(1-oxa-6-azaspiro[3.3]heptan-6-yl)pyrido[3,4-d]pyrimidin-2-amine;
1-(2-((4-(1,5-dimethyl-1H-imidazol-2-yl)-2-methoxyphenyl)amino)-6-methylpyrido[3,4-d]pyrimidin-8-yl)-3-methylazetidine-3-carbonitrile;
N2-(4-(1,5-dimethyl-1H-imidazol-2-yl)-2-methoxyphenyl)-6-methyl-N8-((3-methyltetrahydrofuran-3-yl)methyl)pyrido[3,4-d]pyrimidine-2,8-diamine;
N-(4-(1,5-dimethyl-1H-imidazol-2-yl)-2-methoxyphenyl)-6-methyl-8-(2-oxa-7-azaspiro[4.4]nonan-7-yl)pyrido[3,4-d]pyrimidin-2-amine;
N2-(4-(1,5-dimethyl-1H-imidazol-2-yl)-2-methoxyphenyl)-6-methyl-N8-(tetrahydro-2H-pyran-4-yl)pyrido[3,4-d]pyrimidine-2,8-diamine;
N-(4-(1,5-dimethyl-1H-imidazol-2-yl)-2-methoxyphenyl)-6-methyl-8-(7-oxa-2-azaspiro[3.5]nonan-2-yl)pyrido[3,4-d]pyrimidin-2-amine;
N2-(4-(1,5-dimethyl-1H-imidazol-2-yl)-2-methoxyphenyl)-6-methyl-N8-neopentylpyrido[3,4-d]pyrimidine-2,8-diamine;
8-(3,3-dimethylazetidin-1-yl)-N-(2-methoxy-4-(1-methyl-1H-imidazol-2-yl)phenyl)-6-methylpyrido[3,4-d]pyrimidin-2-amine;
1-(2-((2-methoxy-4-(1-methyl-1H-imidazol-2-yl)phenyl)amino)-6-methylpyrido[3,4-d]pyrimidin-8-yl)-4-methylpiperidine-4-carbonitri le;
8-(3-methoxy-3-methylazetidin-1-yl)-N-(2-methoxy-4-(1-methyl-1H-imidazol-2-yl)phenyl)-6-methylpyrido[3,4-d]pyrimidin-2-amine;
N-(2-methoxy-4-(1-methyl-1H-imidazol-2-yl)phenyl)-8-(4-methoxypiperidin-1-yl)-6-methylpyrido[3,4-d]pyrimidin-2-amine;
N-(2-methoxy-4-(1-methyl-1H-imidazol-2-yl)phenyl)-8-(4-methoxy-4-methylpiperidin-1-yl)-6-methylpyrido[3,4-d]pyrimidin-2-amine;
N-(2-methoxy-4-(1-methyl-1H-imidazol-2-yl)phenyl)-6-methyl-8-(1-oxa-6-azaspiro[3.3]heptan-6-yl)pyrido[3,4-d]pyrimidin-2-amine;
1-(2-((2-methoxy-4-(1-methyl-1H-imidazol-2-yl)phenyl)amino)-6-methylpyrido[3,4-d]pyrimidin-8-yl)-3-methylazetidine-3-carbonitrile;
N2-(2-methoxy-4-(1-methyl-1H-imidazol-2-yl)phenyl)-6-methyl-N8-((3-methyltetrahydrofuran-3-yl)methyl)pyrido[3,4-d]pyrimidine-2,8-diamine;
N-(2-methoxy-4-(1-methyl-1H-imidazol-2-yl)phenyl)-6-methyl-8-(2-oxa-7-azaspiro[4.4]nonan-7-yl)pyrido[3,4-d]pyrimidin-2-amine;
N2-(2-methoxy-4-(1-methyl-1H-imidazol-2-yl)phenyl)-6-methyl-N8-(tetrahydro-2H-pyran-4-yl)pyrido[3,4-d]pyrimidine-2,8-diamine;
N-(2-methoxy-4-(1-methyl-1H-imidazol-2-yl)phenyl)-6-methyl-8-(7-oxa-2-azaspiro[3.5]nonan-2-yl)pyrido[3,4-d]pyrimidin-2-amine;
8-(3,3-dimethylazetidin-1-yl)-N-(4-(2,4-dimethyloxazol-5-yl)-2-methoxyphenyl)-6-methylpyrido[3,4-d]pyrimidin-2-amine;
1-(2-((4-(2,4-dimethyloxazol-5-yl)-2-methoxyphenyl)amino)-6-methylpyrido[3,4-d]pyrimidi n-8-yl)-4-methyl piperidine-4-carbonitri le;
N-(4-(2,4-dimethyloxazol-5-yl)-2-methoxyphenyl)-8-(3-methoxy-3-methylazetidin-1-yl)-6-methylpyrido[3,4-d]pyrimidin-2-amine;
N-(4-(2,4-dimethyloxazol-5-yl)-2-methoxyphenyl)-8-(4-methoxypiperidin-1-yl)-6-methylpyrido[3,4-d]pyrimidin-2-amine;
N-(4-(2,4-dimethyloxazol-5-yl)-2-methoxyphenyl)-8-(4-methoxy-4-methylpiperidin-1-yl)-6-methylpyrido[3,4-d]pyrimidin-2-amine;
N-(4-(2,4-dimethyloxazol-5-yl)-2-methoxyphenyl)-6-methyl-8-(1-oxa-6-azaspiro[3.3]heptan-6-yl)pyrido[3,4-d]pyrimidin-2-amine;
1-(2-((4-(2,4-dimethyloxazol-5-yl)-2-methoxyphenyl)amino)-6-methylpyrido[3,4-d]pyrimidin-8-yl)-3-methylazetidine-3-carbonitrile;
N2-(4-(2,4-dimethyloxazol-5-yl)-2-methoxyphenyl)-6-methyl-N8-((3-methyltetrahydrofuran-3-yl)methyl)pyrido[3,4-d]pyrimidine-2,8-diamine;
N-(4-(2,4-dimethyloxazol-5-yl)-2-methoxyphenyl)-6-methyl-8-(2-oxa-7-azaspiro[4.4]nonan-7-yl)pyrido[3,4-d]pyrimidin-2-amine;
N2-(4-(2,4-dimethyloxazol-5-yl)-2-methoxyphenyl)-6-methyl-N8-(tetrahydro-2H-pyran-4-yl)pyrido[3,4-d]pyrimidine-2,8-diamine;
N-(4-(2,4-dimethyloxazol-5-yl)-2-methoxyphenyl)-6-methyl-8-(7-oxa-2-azaspiro[3.5]nonan-2-yl)pyrido[3,4-d]pyrimidin-2-amine;
N2-(4-(2,4-dimethyloxazol-5-yl)-2-methoxyphenyl)-6-methyl-N8-neopentylpyrido[3,4-d]pyrimidine-2,8-diamine;
8-(3,3-dimethylazetidin-1-yl)-N-(4-(2,4-dimethyloxazol-5-yl)-2-ethoxyphenyl)-6-methylpyrido[3,4-d]pyrimidin-2-amine;
1-(2-((4-(2,4-dimethyloxazol-5-yl)-2-ethoxyphenyl)amino)-6-methylpyrido[3,4-d]pyrimidin-8-yl)-4-methylpiperidine-4-carbonitrile;
N-(4-(2,4-dimethyloxazol-5-yl)-2-ethoxyphenyl)-8-(3-methoxy-3-methylazetidin-1-yl)-6-methylpyrido[3,4-d]pyrimidin-2-amine;
N-(4-(2,4-dimethyloxazol-5-yl)-2-ethoxyphenyl)-8-(4-methoxypiperidin-1-yl)-6-methylpyrido[3,4-d]pyrimidin-2-amine;
N-(4-(2,4-dimethyloxazol-5-yl)-2-ethoxyphenyl)-8-(4-methoxy-4-methylpiperidin-1-yl)-6-methylpyrido[3,4-d]pyrimidin-2-amine;
N-(4-(2,4-dimethyloxazol-5-yl)-2-ethoxyphenyl)-6-methyl-8-(1-oxa-6-azaspiro[3.3]heptan-6-yl)pyrido[3,4-d]pyrimidin-2-amine;
1-(2-((4-(2,4-dimethyloxazol-5-yl)-2-ethoxyphenyl)amino)-6-methylpyrido[3,4-d]pyrimidin-8-yl)-3-methylazetidine-3-carbonitrile;
N2-(4-(2,4-dimethyloxazol-5-yl)-2-ethoxyphenyl)-6-methyl-N8-((3-methyltetrahydrofuran-3-yl)methyl)pyrido[3,4-d]pyrimidine-2,8-diamine;
N-(4-(2,4-dimethyloxazol-5-yl)-2-ethoxyphenyl)-6-methyl-8-(2-oxa-7-azaspiro[4.4]nonan-7-yl)pyrido[3,4-d]pyrimidin-2-amine;
N2-(4-(2,4-dimethyloxazol-5-yl)-2-ethoxyphenyl)-6-methyl-N8-(tetrahydro-2H-pyran-4-yl)pyrido[3,4-d]pyrimidine-2,8-diamine;
N-(4-(2,4-dimethyloxazol-5-yl)-2-ethoxyphenyl)-6-methyl-8-(7-oxa-2-azaspiro[3.5]nonan-2-yl)pyrido[3,4-d]pyrimidin-2-amine;
N2-(4-(2,4-dimethyloxazol-5-yl)-2-ethoxyphenyl)-6-methyl-N8-neopentylpyrido[3,4-d]pyrimidine-2,8-diamine;
8-(3,3-dimethylazetidin-1-yl)-N-(4-(2,5-dimethyloxazol-4-yl)-2-methoxyphenyl)-6-methylpyrido[3,4-d]pyrimidin-2-amine;
1-(2-((4-(2,5-dimethyloxazol-4-yl)-2-methoxyphenyl)amino)-6-methylpyrido[3,4-d]pyrimidin-8-yl)-4-methylpiperidine-4-carbonitrile;
N-(4-(2,5-dimethyloxazol-4-yl)-2-methoxyphenyl)-8-(3-methoxy-3-methylazetidin-1-yl)-6-methylpyrido[3,4-d]pyrimidin-2-amine;
N-(4-(2,5-dimethyloxazol-4-yl)-2-methoxyphenyl)-8-(4-methoxypiperidin-1-yl)-6-methylpyrido[3,4-d]pyrimidin-2-amine;
N-(4-(2,5-dimethyloxazol-4-yl)-2-methoxyphenyl)-8-(4-methoxy-4-methylpiperidin-1-yl)-6-methylpyrido[3,4-d]pyrimidin-2-amine;
N-(4-(2,5-dimethyloxazol-4-yl)-2-methoxyphenyl)-6-methyl-8-(1-oxa-6-azaspiro[3.3]heptan-6-yl)pyrido[3,4-d]pyrimidin-2-amine;
1-(2-((4-(2,5-dimethyloxazol-4-yl)-2-methoxyphenyl)amino)-6-methylpyrido[3,4-d]pyrimidin-8-yl)-3-methylazetidine-3-carbonitrile;
N2-(4-(2,5-dimethyloxazol-4-yl)-2-methoxyphenyl)-6-methyl-N8-((3-methyltetrahydrofuran-3-yl)methyl)pyrido[3,4-d]pyrimidine-2,8-diamine;
N-(4-(2,5-dimethyloxazol-4-yl)-2-methoxyphenyl)-6-methyl-8-(2-oxa-7-azaspiro[4.4]nonan-7-yl)pyrido[3,4-d]pyrimidin-2-amine;
N2-(4-(2,5-dimethyloxazol-4-yl)-2-methoxyphenyl)-6-methyl-N8-(tetrahydro-2H-pyran-4-yl)pyrido[3,4-d]pyrimidine-2,8-diamine;
N-(4-(2,5-dimethyloxazol-4-yl)-2-methoxyphenyl)-6-methyl-8-(7-oxa-2-azaspiro[3.5]nonan-2-yl)pyrido[3,4-d]pyrimidin-2-amine;
N2-(4-(2,5-dimethyloxazol-4-yl)-2-methoxyphenyl)-6-methyl-N8-neopentylpyrido[3,4-d]pyrimidine-2,8-diamine;
8-(3,3-dimethylazetidin-1-yl)-N-(4-(2,5-dimethyloxazol-4-yl)-2-ethoxyphenyl)-6-methylpyrido[3,4-d]pyrimidin-2-amine;
1-(2-((4-(2,5-dimethyloxazol-4-yl)-2-ethoxyphenyl)amino)-6-methylpyrido[3,4-d]pyrimidin-8-yl)-4-methylpiperidine-4-carbonitrile;
N-(4-(2,5-dimethyloxazol-4-yl)-2-ethoxyphenyl)-8-(3-methoxy-3-methylazetidin-1-yl)-6-methylpyrido[3,4-d]pyrimidin-2-amine;
N-(4-(2,5-dimethyloxazol-4-yl)-2-ethoxyphenyl)-8-(4-methoxypiperidin-1-yl)-6-methylpyrido[3,4-d]pyrimidin-2-amine;
N-(4-(2,5-dimethyloxazol-4-yl)-2-ethoxyphenyl)-8-(4-methoxy-4-methylpiperidin-1-yl)-6-methylpyrido[3,4-d]pyrimidin-2-amine;
N-(4-(2,5-dimethyloxazol-4-yl)-2-ethoxyphenyl)-6-methyl-8-(1-oxa-6-azaspiro[3.3]heptan-6-yl)pyrido[3,4-d]pyrimidin-2-amine;
1-(2-((4-(2,5-dimethyloxazol-4-yl)-2-ethoxyphenyl)amino)-6-methylpyrido[3,4-d]pyrimidin-8-yl)-3-methylazetidine-3-carbonitrile;
N2-(4-(2,5-dimethyloxazol-4-yl)-2-ethoxyphenyl)-6-methyl-N8-((3-methyltetrahydrofuran-3-yl)methyl)pyrido[3,4-d]pyrimidine-2,8-diamine;
N-(4-(2,5-dimethyloxazol-4-yl)-2-ethoxyphenyl)-6-methyl-8-(2-oxa-7-azaspiro[4.4]nonan-7-yl)pyrido[3,4-d]pyrimidin-2-amine;
N2-(4-(2,5-dimethyloxazol-4-yl)-2-ethoxyphenyl)-6-methyl-N8-(tetrahydro-2H-pyran-4-yl)pyrido[3,4-d]pyrimidine-2,8-diamine;
N-(4-(2,5-dimethyloxazol-4-yl)-2-ethoxyphenyl)-6-methyl-8-(7-oxa-2-azaspiro[3.5]nonan-2-yl)pyrido[3,4-d]pyrimidin-2-amine;
N2-(4-(2,5-dimethyloxazol-4-yl)-2-ethoxyphenyl)-6-methyl-N8-neopentylpyrido[3,4-d]pyrimidine-2,8-diamine.

30. A method according to any preceding claim wherein the MPS1 inhibitor is selected from the following:
N-cyclopropyl-4-(6-(2,3-difluoro-4-methoxyphenoxy)-8-((3,3,3-trifluoropropyl)amino)imidazo[1,2-b]pyridazin-3-yl)-2-methylbenzamide;
(R)-2-(4-fluorophenyl)-N-(4-(2-((2-methoxy-4-(methylsulfonyl)phenyl)amino)-[1,2,4]triazolo[1,5-a]pyridin-6-yl)phenyl)propanamide;
N2-(4-(4,5-dimethyl-4H-1,2,4-triazol-3-yl)-2-ethoxyphenyl)-6-methyl-N8-neopentylpyrido[3,4-d]pyrimidine-2,8-diamine;
(S)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
8-(3,3-dimethylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(3-methoxy-3-methylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylazetidine-3-carbonitrile;
N2-(2-ethoxy-4-(4-methyl-4H-1,2,4-triazol-3-yl)phenyl)-6-methyl-N8-neopentylpyrido[3,4-d]pyrimidine-2,8-diamine;
or a pharmaceutically acceptable salt or solvate thereof.

31. A method according to any one of the preceding claims wherein the MPS1 inhibitor is selected from the following:
N2-(4-(4,5-dimethyl-4H-1,2,4-triazol-3-yl)-2-ethoxyphenyl)-6-methyl-N8-neopentylpyrido[3,4-d]pyrimidine-2,8-diamine;
(S)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
8-(3,3-dimethylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(3-methoxy-3-methylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylazetidine-3-carbonitrile;
N2-(2-ethoxy-4-(4-methyl-4H-1,2,4-triazol-3-yl)phenyl)-6-methyl-N8-neopentylpyrido[3,4-d]pyrimidine-2,8-diamine;
or a pharmaceutically acceptable salt or solvate thereof.

32. An MPS1 inhibitor for use in the treatment of an oestrogen receptor-positive breast cancer in a subject in need thereof, wherein:
(i) the subject has previously been treated with an endocrine therapy; and/or
(ii) said breast cancer is resistant to endocrine therapy.

33. A compound for use according to claim 32 wherein the subject has failed treatment with the endocrine therapy.

34. A compound for use according to any one of claims 32 and 33 wherein the subject has relapsed during or following treatment with the endocrine therapy.

35. A compound for use according to any one of claims 32 to 34 wherein the subject has experienced disease progression during or following treatment with the endocrine therapy.

36. A compound for use according to any one of claims 32 to 35 wherein the subject has developed an endocrine therapy-resistant breast cancer during or following treatment with the endocrine therapy.

37. A compound for use according to any one of claims 32 to 36 wherein the breast cancer is de novo resistant to endocrine therapy.

38. A compound for use according to any one of claims 32 to 37 wherein the endocrine therapy comprises/essentially consists of/consists of the administration of an endocrine agent.

39. A compound for use according to claim 38 wherein the endocrine agent is selected from one or more of an aromatase inhibitor, a selective oestrogen receptor modulator (SERM) and a selective oestrogen receptor degrader/downregulator (SERD).

40. A compound for use according to any one of claims 38 and 39 wherein the endocrine therapy comprises/essentially consists of/consists of separate, sequential or combined treatment with an aromatase inhibitor and a SERD.

41. A compound for use according to any one of claims 38 and 39 wherein the endocrine therapy comprises/essentially consists of/consists of separate, sequential or combined treatment with a SERM and a SERD.

42. A compound for use according to any one of claims 39 and 40 wherein the aromatase inhibitor is selected from the group consisting of anastrozole, exemestane and letrozole, or pharmaceutically acceptable salts or solvates thereof.

43. A compound for use according to any one of claims 39 and 41 wherein the SERM is tamoxifen, or pharmaceutically acceptable salts or solvates thereof.

44. A compound for use according to any one of claims 39 to 41 wherein the SERD is fulvestrant, or pharmaceutically acceptable salts or solvates thereof.

45. A compound for use according to any one of claims 32 to 44 wherein the subject is pre-menopausal.

46. A compound for use according to any one of claims 32 to 44 wherein the subject is post-menopausal.

47. A compound for use according to any one of claims 32 to 46 wherein (i) the subject has additionally been previously treated with a CDK4/6 inhibitor; and/or (ii) the breast cancer is additionally resistant to treatment with a CDK4/6 inhibitor.

48. A compound for use according to claim 47 wherein the subject has failed treatment with the CDK4/6 inhibitor.

49. A compound for use according to any one of claims 47 and 48 wherein the subject has relapsed during or following treatment with the CDK4/6 inhibitor.

50. A compound for use according to any one of claims 47 to 49 wherein the subject has experienced disease progression during or following treatment with the CDK4/6 inhibitor.

51. A compound for use according to any one of claims 47 to 50 wherein the subject has developed a CDK4/6 inhibitor-resistant breast cancer during or following treatment with the with the CDK4/6 inhibitor.

52. A compound for use according to any one of claims 47 to 51 wherein the breast cancer is de novo resistant to treatment with a CDK4/6 inhibitor.

53. A compound for use according to any one of claims 47 to 52 wherein the CDK4/6 inhibitor is selected from the group consisting of palbociclib, abemaciclib and ribociclib, or pharmaceutically acceptable salts or solvates thereof.

54. A compound for use according to any one of claims 32 to 53 wherein the compound capable of inhibiting MPS1 is for separate, sequential and/or combined use with an endocrine agent.

55. A compound for use according to claim 54 wherein the endocrine agent is selected from the group consisting of tamoxifen and fulvestrant, or pharmaceutically acceptable salts or solvates thereof.

56. A compound for use according to any one of claims 32 to 55 wherein the compound capable of inhibiting MPS1 is an MPS1 inhibitor, suitably selected from the group consisting of NMS-P715, S 81694 (NMS-P153), AZ3146, BAY 1217389, BAY 1161909, MPS1-IN-3, MPS1-IN-2, CFI-402257, CCT289346, a compound of formula I, a compound of formula II, a compound of formula III and a compound of formula IV, or a pharmaceutically acceptable salt or solvate thereof, wherein formula I, formula II, formula III and formula IV are as defined in claim 21.

57. A compound for use according to claim 56 wherein the MPS1 inhibitor is selected from the group consisting of NMS-P715, BAY 1217389, BAY 1161909, CCT289346, a compound of formula I, a compound of formula II, a compound of formula III and a compound of formula IV, or pharmaceutically acceptable salts or solvates thereof.

58. A compound for use according to claim 56 wherein the MPS1 inhibitor is selected from the group consisting of a compound of formula I, a compound of formula II, a compound of formula III and a compound of formula IV, or pharmaceutically acceptable salts or solvates thereof.

59. A compound for use according to claim 56 wherein the MPS1 inhibitor is selected from the group consisting of a compound of formula I or a compound of formula II, or pharmaceutically acceptable salts or solvates thereof.

60. A compound for use according to any one of claims 32 to 59 wherein the MPS1 inhibitor is selected from the compounds defined in claim 29.

61. A compound for use according to claim 56 wherein the MPS1 inhibitor is selected from the following:
N-cyclopropyl-4-(6-(2,3-difluoro-4-methoxyphenoxy)-8-((3,3,3-trifluoropropyl)amino)imidazo[1,2-b]pyridazin-3-yl)-2-methylbenzamide;
(R)-2-(4-fluorophenyl)-N-(4-(2-((2-methoxy-4-(methylsulfonyl)phenyl)amino)-[1,2,4]triazolo[1,5-a]pyridin-6-yl)phenyl)propanamide;
N2-(4-(4,5-dimethyl-4H-1,2,4-triazol-3-yl)-2-ethoxyphenyl)-6-methyl-N8-neopentylpyrido[3,4-d]pyrimidine-2,8-diamine;
(S)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
8-(3,3-dimethylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(3-methoxy-3-methylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylazetidine-3-carbonitrile;
N2-(2-ethoxy-4-(4-methyl-4H-1,2,4-triazol-3-yl)phenyl)-6-methyl-N8-neopentylpyrido[3,4-d]pyrimidine-2,8-diamine;
or a pharmaceutically acceptable salt or solvate thereof.

62. A compound for use according to claim 61 wherein the MPS1 inhibitor is selected from the following:
N2-(4-(4,5-dimethyl-4H-1,2,4-triazol-3-yl)-2-ethoxyphenyl)-6-methyl-N8-neopentylpyrido[3,4-d]pyrimidine-2,8-diamine;
(S)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
8-(3,3-dimethylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(3-methoxy-3-methylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylazetidine-3-carbonitrile;
N2-(2-ethoxy-4-(4-methyl-4H-1,2,4-triazol-3-yl)phenyl)-6-methyl-N8-neopentylpyrido[3,4-d]pyrimidine-2,8-diamine;
or a pharmaceutically acceptable salt or solvate thereof.

63. Use of an M PS1 inhibitor in the manufacture of a medicament for the treatment of an oestrogen receptor positive breast cancer in a subject in need thereof, wherein:
(i) the subject has been previously treated with an endocrine therapy; and/or
(ii) said breast cancer is resistant to endocrine therapy.

64. Use according to claim 63 wherein the subject has failed treatment with the endocrine therapy.

65. Use according to any one of claims 63 and 64 wherein the subject has relapsed during or following treatment with the endocrine therapy.

66. Use according to any one of claims 63 to 65 wherein the subject has experienced disease progression during or following treatment with the endocrine therapy.

67. Use according to any one of claims 63 to 66 wherein the subject has developed an endocrine therapy-resistant breast cancer during or following treatment with the endocrine therapy.

68. Use according to any one of claims 63 to 67 wherein the breast cancer is de novo resistant to endocrine therapy.

69. Use according to any one of claims 63 to 68 wherein the endocrine therapy comprises/essentially consists of/consists of the administration of an endocrine agent.

70. Use according to claim 69 wherein the endocrine agent is selected from one or more of an aromatase inhibitor, a selective oestrogen receptor modulator (SERM) and a selective oestrogen receptor degrader/downregulator (SERD).

71. Use according to any one of claims 63 to 70 wherein the endocrine therapy comprises/essentially consists of/consists of separate, sequential or combined treatment with an aromatase inhibitor and a SERD.

72. Use according to any one of claims 63 to 70 wherein the endocrine therapy comprises/essentially consists of/consists of separate, sequential or combined treatment with a SERM and a SERD.

73. Use according to any one of claims 70 and 71 wherein the aromatase inhibitor is selected from the group consisting of anastrozole, exemestane and letrozole, or pharmaceutically acceptable salts or solvates thereof.

74. Use according to any one of claims 70 and 72 wherein the SERM is tamoxifen, or pharmaceutically acceptable salts or solvates thereof.

75. Use according to any one of claims 70 to 72 wherein the SERD is fulvestrant, or pharmaceutically acceptable salts or solvates thereof.

76. Use according to any one of claims 63 to 75 wherein the subject is pre-menopausal.

77. Use according to any one of claims 63 to 75 wherein the subject is post-menopausal.

78. Use according to any one of claims 63 to 77 wherein (i) the subject has additionally been previously treated with a CDK4/6 inhibitor; and/or (ii) the breast cancer is additionally resistant to treatment with a CDK4/6 inhibitor.

79. Use according to claim 78 wherein the subject has failed treatment with the CDK4/6 inhibitor.

80. Use according to any one of claims 78 and 79 wherein the subject has relapsed during or following treatment with the CDK4/6 inhibitor.

81. Use according to any one of claims 78 to 80 wherein the subject has experienced disease progression during or following treatment with the CDK4/6 inhibitor.

82. Use according to any one of claims 78 to 81 wherein the subject has developed a CDK4/6 inhibitor-resistant breast cancer during or following treatment with the CDK4/6 inhibitor.

83. Use according to any one of claims 78 to 82 wherein the breast cancer is de novo resistant to treatment with a CDK4/6 inhibitor.

84. Use according to any one of claims 78 to 83 wherein the CDK4/6 inhibitor is selected from the group consisting of palbociclib, abemaciclib and ribociclib, or pharmaceutically acceptable salts or solvates thereof.

85. Use according to any one of claims 63 to 84 wherein the compound capable of inhibiting MPS1 is for separate, sequential and/or combined use with an endocrine agent.

86. Use according to claim 85 wherein the endocrine agent is selected from the group consisting of tamoxifen and fulvestrant, or pharmaceutically acceptable salts or solvates thereof.

87. Use according to any one of claims 63 to 86 wherein the compound capable of inhibiting MPS1 is an MPS1 inhibitor, suitably selected from the group consisting of NMS-P715, S 81694 (NMS-P153), AZ3146, BAY 1217389, BAY 1161909, MPS1-IN-3, MPS1-IN-2, CFI-402257, CCT289346, a compound of formula I, a compound of formula II, a compound of formula III and a compound of formula IV, or a pharmaceutically acceptable salt or solvate thereof; wherein formula I, formula II, formula III and formula IV are as defined in claim 21.

88. Use according to claim 87 wherein the MPS1 inhibitor is selected from the group consisting of NMS-P715, BAY 1217389, BAY 1161909, CCT289346, a compound of formula I, a compound of formula II, a compound of formula III and a compound of formula IV, or pharmaceutically acceptable salts or solvates thereof.

89. Use according to claim 87 wherein the MPS1 inhibitor is selected from the group consisting of a compound of formula I, a compound of formula II, a compound of formula III and a compound of formula IV, or pharmaceutically acceptable salts or solvates thereof.

90. Use according to claim 89 wherein the MPS1 inhibitor is selected from the group consisting of a compound of formula I or a compound of formula II, or pharmaceutically acceptable salts or solvates thereof.

91. Use according to any one of claims 63 to 90 wherein the MPS1 inhibitor is selected from the compounds defined in claim 29.

92. Use according to claim 87 wherein the MPS1 inhibitor is selected from the following:
N-cyclopropyl-4-(6-(2,3-difluoro-4-methoxyphenoxy)-8-((3,3,3-trifluoropropyl)amino)imidazo[1,2-b]pyridazin-3-yl)-2-methylbenzamide;
(R)-2-(4-fluorophenyl)-N-(4-(2-((2-methoxy-4-(methylsulfonyl)phenyl)amino)-[1,2,4]triazolo[1,5-a]pyridin-6-yl)phenyl)propanamide;
N2-(4-(4,5-dimethyl-4H-1,2,4-triazol-3-yl)-2-ethoxyphenyl)-6-methyl-N8-neopentylpyrido[3,4-d]pyrimidine-2,8-diamine;
(S)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
8-(3,3-dimethylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(3-methoxy-3-methylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylazetidine-3-carbonitrile;
N2-(2-ethoxy-4-(4-methyl-4H-1,2,4-triazol-3-yl)phenyl)-6-methyl-N8-neopentylpyrido[3,4-d]pyrimidine-2,8-diamine;
or a pharmaceutically acceptable salt or solvate thereof.

93. Use according to claim 92 wherein the MPS1 inhibitor is selected from the following:
N2-(4-(4,5-dimethyl-4H-1,2,4-triazol-3-yl)-2-ethoxyphenyl)-6-methyl-N8-neopentylpyrido[3,4-d]pyrimidine-2,8-diamine;
(S)-N8-(3,3-dimethylbutan-2-yl)-N2-(2-ethoxy-4-(4-methyl-4H-1,2,4-triazol-3-yl)phenyl)-6-methylpyrido[3,4-d]pyrimidine-2,8-diamine;
8-(3,3-dimethylazetidin-1-yl)-N-(2-ethoxy-4-(4-methyl-4H-1,2,4-triazol-3-yl)phenyl)-6-methylpyrido[3,4-d]pyrimidin-2-amine;
N-(2-ethoxy-4-(4-methyl-4H-1,2,4-triazol-3-yl)phenyl)-8-(3-methoxy-3-methylazetidin-1-yl)-6-methylpyrido[3,4-d]pyrimidin-2-amine;
1-(2-((2-ethoxy-4-(4-methyl-4H-1,2,4-triazol-3-yl)phenyl)amino)-6-methylpyrido[3,4-d]pyrimidin-8-yl)-3-methylazetidine-3-carbonitrile;
N2-(2-ethoxy-4-(4-methyl-4H-1,2,4-triazol-3-yl)phenyl)-6-methyl-N8-neopentylpyrido[3,4-d]pyrimidine-2,8-diamine;
or a pharmaceutically acceptable salt or solvate thereof.
